(19) United States
(12) Patent Application Publication
(10) Pub. No.: US 2019/0000860 A1
(43) Pub. Date: Jan. 3, 2019

(54) USE OF COMPOSITIONS MODULATING CHROMATIN STRUCTURE FOR GRAFT VERSUS HOST DISEASE (GVHD)

(71) Applicants: Dana-Farber Cancer Institute, Inc., Boston, MA (US); Regents of the University of Minnesota, Minneapolis, MN (US)

(72) Inventors: James E. Bradner, Weston, MA (US); Bruce Blazar, Golden Valley, MN (US); Ryan Flynn, Minneapolis, MN (US); June Qi, Sharon, MA (US)

(73) Assignees: Dana-Farber Cancer Institute, Inc., Boston, MA (US); Regents of the University of Minnesota, Minneapolis, MN (US)

(21) Appl. No.: 15/524,707

(22) PCT Filed: Nov. 6, 2015
(86) PCT No.: PCT/US2015/059551

§ 371 Date: May 5, 2017

Related U.S. Application Data
(60) Provisional application No. 62/076,358, filed on Nov. 6, 2014.

(51) Int. Cl. A61K 31/551; A61K 31/58; A61K 31/496; A61K 31/4439; A61K 31/444; A61K 45/06; A61K 31/427; A61P 37/00

(52) U.S. Cl. CPC A61K 31/551; A61K 31/58; A61K 31/496; A61P 37/00; A61K 31/444; A61K 45/06; A61K 31/427; A61K 31/4439

(57) ABSTRACT

In some aspects, the instant disclosure relates to methods of treating chronic graft versus host disease (cGVHD). In some embodiments, the method comprises administering to a subject in need thereof a EZH2 inhibitor, a Bcl6 inhibitor and/or BRD4 inhibitor. The present disclosure is based, at least in part, on the discovery that enhancer of zeste homolog 2 (EZH2) inhibitors, B-cell lymphoma 6 protein (Bcl6) inhibitors and/or bromodomain-containing protein 4 (BRD4) inhibitors can be used to treat chronic graft versus host disease (cGVHD).

### RELATED APPLICATIONS

This application claims priority under 35 U.S.C. § 119(e) to U.S. provisional application, U.S. Ser. No. 62/076,358, filed Nov. 6, 2014, which is incorporated by reference herein.

### GOVERNMENT SUPPORT

This invention was made with government support under grant numbers CA128972 (K08), CA142106 (P01), AI056299 (P01), AU112613(R01) and AI007313 (T32) awarded by the National Institutes of Health. The government has certain rights in the invention.

### BACKGROUND OF INVENTION

Graft versus host disease (GVHD) is a potentially serious complication of allogeneic tissue transplant and blood transfusion. The underlying cause of GVHD is the presence of functional immune cells, such as white blood cells, in the transplanted tissue (graft). For example, T-cells present in the graft may recognize recipient (host) tissue as a “foreign” antigen and attack host cells. The host cells cannot defend against the attack by the graft because of the immuno-compromised status of the transplant recipient. GVHD is classified into two forms, acute and chronic. Acute GVHD (aGVHD) normally occurs within 100 days post-transplant or transfusion. Chronic GVHD (cGVHD) normally occurs more than 100 days post-transplant or transfusion. Treatment options for cGVHD are limited. Glucocorticoids are the current first line therapeutic option. However, high doses of immune-suppressive steroids can raise a patient's risk of infection or cancer-relapse; patients may also be refractory to steroid treatment regimens. Thus, new ways of treating cGVHD are needed.

### BRIEF SUMMARY OF INVENTION

The present disclosure is based, at least in part, on the discovery that enhancer of zeste homolog 2 (EZH2) inhibitors, B-cell lymphoma 6 protein (Bcl6) inhibitors and/or bromodomain-containing protein 4 (BRD4) inhibitors can be used to treat chronic graft versus host disease (cGVHD).

Accordingly, some aspects of the disclosure provide a method for treating chronic graft-versus-host disease (cGVHD), the method comprising administering to a subject in need thereof an enhancer of zeste homolog 2 (EZH2) inhibitor, a B-cell lymphoma 6 protein (Bcl6) inhibitor and/or a bromodomain-containing protein 4 (BRD4) inhibitor in an amount effective to treat cGVHD.

Some aspects of the present disclosure provide a method for improving pulmonary function in a subject receiving an allogeneic transplant, the method comprising administering to a subject in need thereof an enhancer of zeste homolog 2 (EZH2) inhibitor, a B-cell lymphoma 6 protein (Bcl6) inhibitor and/or a bromodomain-containing protein 4 (BRD4) inhibitor in an amount effective to improve pulmonary function.

In the embodiments, the EZH2 inhibitor is a small molecule, peptide, peptide mimetic, protein or a portion thereof, antibody, or nucleic acid. In the embodiments, the Bcl6 inhibitor is a small molecule, peptide, peptide mimetic, protein or a portion thereof, antibody, or nucleic acid. In the embodiments, the BRD4 inhibitor is a small molecule, peptide, peptide mimetic, protein or a portion thereof, antibody, or nucleic acid.

In the embodiments, the EZH2 inhibitor is a compound of Formula (I):

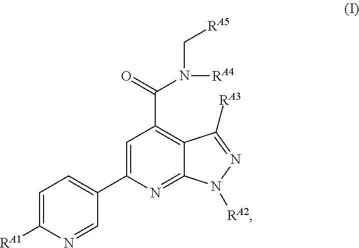

or a pharmaceutically acceptable salt, solvate, hydrate, polymorph, co-crystal, tautomer, stereoisomer, isotopically labeled derivative, or prodrug thereof, wherein:

R^{A1} is halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{a}, —N(R^{a})₂, —SR^{a}, —CN, —SCN, —C(═NR^{a})R^{a}, —C(═NR^{a})OR^{a}, —C(═NR^{a})N(R^{a})₂, —C(═O)R^{a}, —C(═O)OR^{a}, —C(═O)N(R^{a})₂, —NO₂, —NR^{a}C(═O)R^{a}, —NR^{a}C(═O)OR^{a}, —NR^{a}C(═O)N(R^{a})₂, —OC(═O)R^{a}, —OC(═O)OR^{a}, —OC(═O)N(R^{a})₂, or

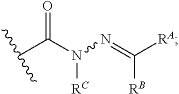

each instance of R^{a} is independently hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, an oxygen protecting group when attached to an oxygen atom, or a sulfur protecting group when attached to a sulfur atom, or two instances of R^{a} are joined to form a substituted or unsubstituted, heterocyclic ring, or substituted or unsubstituted, heteroaryl ring;

R^{A2} is hydrogen, substituted or unsubstituted acyl, substituted or unsubstituted alkyl, substituted or unsubstituted carbocyclyl, substituted or unsubstituted heterocyclyl, a nitrogen protecting group, or a warhead;

R^{A3} is hydrogen, halogen, substituted or unsubstituted C₁₋₆ alkyl, —OR^{a}, —N(R^{a})₂, or a warhead;

R^{A4} is hydrogen, substituted or unsubstituted C₁₋₆ alkyl, or a nitrogen protecting group;

R^{A5} is of the formula:

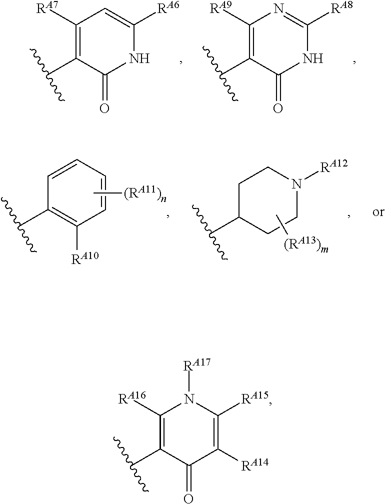

wherein:
R^{A6} is hydrogen, halogen, substituted or unsubstituted C₁₋₆ alkyl, —OR^{a}, or —N(R^{a})₂;
R^{A7} is hydrogen, halogen, substituted or unsubstituted C₂₋₆ alkyl, substituted or unsubstituted, 3- to 7-membed, monocyclic carbocyclyl comprising 0, 1, or 2 double bonds in the carbocyclic ring system, —OR^{a}, or —N(R^{a})₂;
R^{A8} is hydrogen, halogen, substituted or unsubstituted C₁₋₆ alkyl, —OR^{a}, or —N(R^{a})₂;
R^{A9} is hydrogen, halogen, substituted or unsubstituted C₁₋₆ alkyl, substituted or unsubstituted, 3- to 7-membed, monocyclic carbocyclyl comprising 0, 1, or 2 double bonds in the carbocyclic ring system, —OR^{a}, or —N(R^{a})₂;
R^{A10} is —OR^{a}, —N(R^{a})₂, or a warhead;
each instance of R^{A11} is independently halogen, substituted or unsubstituted C₁₋₆ alkyl, substituted or unsubstituted, 3- to 7-membed, monocyclic carbocyclyl comprising 0, 1, or 2 double bonds in the carbocyclic ring system, —OR^{a}, or —N(R^{a})₂;
n is 0, 1, 2, 3, or 4;
R^{A12} is hydrogen, substituted or unsubstituted C₁₋₆ alkyl, a nitrogen protecting group, or a warhead;
each instance of R^{A13} is independently halogen, substituted or unsubstituted C₁₋₆ alkyl, substituted or unsubstituted, 3- to 7-membed, monocyclic carbocyclyl comprising 0, 1, or 2 double bonds in the carbocyclic ring system, —OR^{a}, or —N(R^{a})₂;
m is 0, 1, 2, 3, 4, 5, 6, 7, 8, or 9;
R^{A14} is hydrogen, halogen, substituted or unsubstituted C₁₋₆ alkyl, —OR^{a}, or —N(R^{a})₂;
R^{A15} is hydrogen, halogen, substituted or unsubstituted C₁₋₆ alkyl, —OR^{a}, or —N(R^{a})₂;
R^{A16} is hydrogen, halogen, substituted or unsubstituted C₂₋₆ alkyl, substituted or unsubstituted, 3- to 7-membed, monocyclic carbocyclyl comprising 0, 1, or 2 double bonds in the carbocyclic ring system, —OR^{a}, or —N(R^{a})₂; and
R^{A17} is hydrogen, substituted or unsubstituted acyl, substituted or unsubstituted C₁₋₆ alkyl, a nitrogen protecting group, or a warhead.

In the embodiments, the EZH2 inhibitor is a compound of the formula:

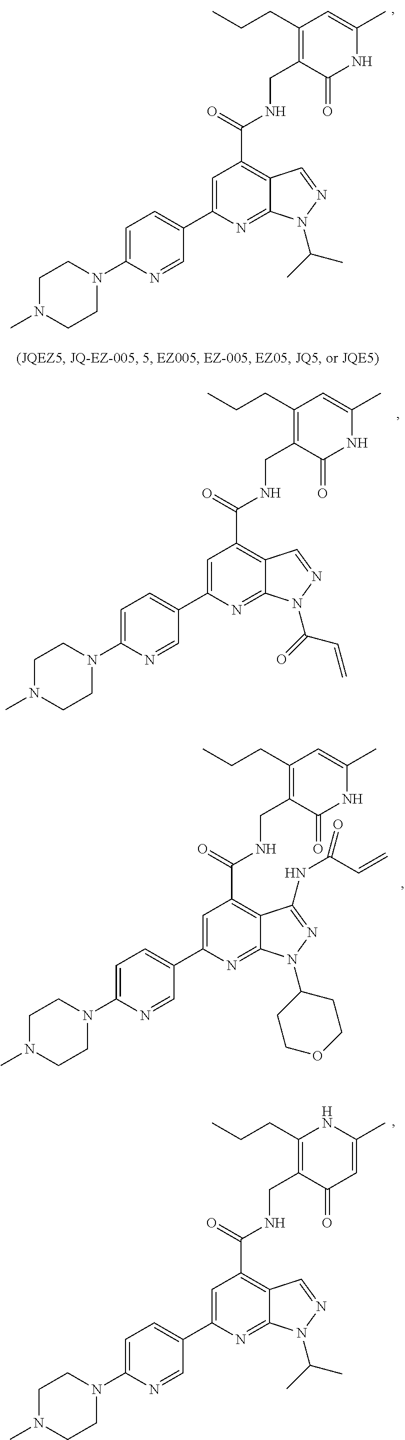

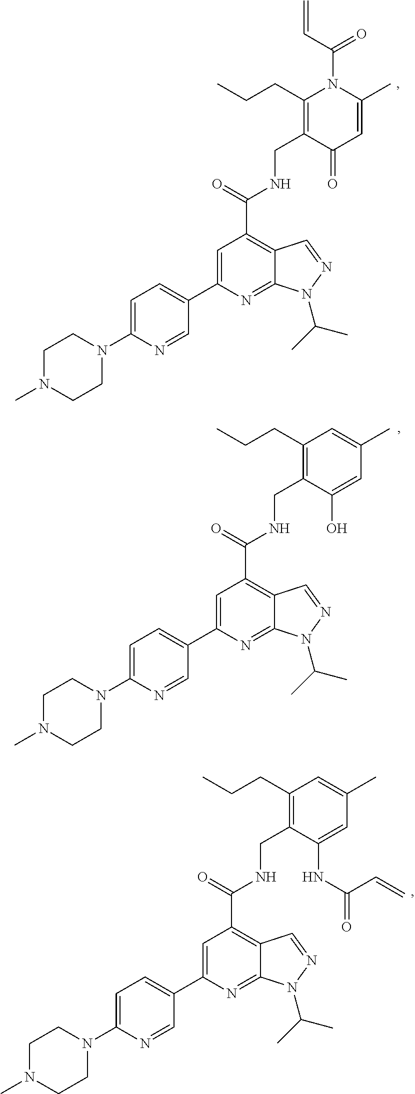

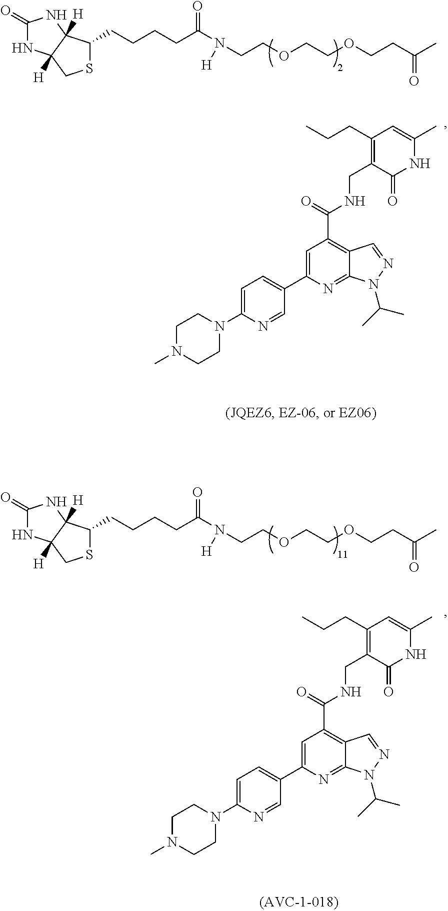

or pharmaceutically acceptable salt, solvate, hydrate, polymorph, co-crystal, tautomer, stereoisomer, isotopically labeled derivative, or prodrug thereof.

In certain embodiments, the EZH2 inhibitor is a compound of the formula:

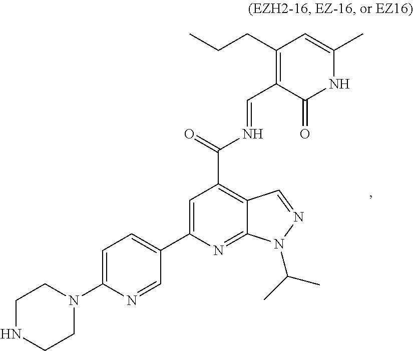 [SMILES: CCCc1cc(C)[nH]c(=O)c1/C=N/C(=O)c1cc(-c2ccc(N3CCNCC3)nc2)nc2c1cnn2C(C)C]

or a pharmaceutically acceptable salt, solvate, hydrate, polymorph, co-crystal, tautomer, stereoisomer, isotopically labeled derivative, or prodrug thereof.

In some embodiments, the EZH2 inhibitor is a compound of Formula (II):

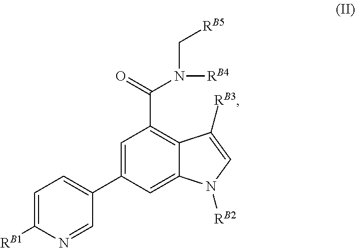

or a pharmaceutically acceptable salt, solvate, hydrate, polymorph, co-crystal, tautomer, stereoisomer, isotopically labeled derivative, and prodrug thereof, wherein:

R^{B1} is halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{b}, —N(R^{b})₂, —SR^{b}, —CN, —SCN, —C(═NR^{b})R^{b}, —C(═NR^{b})OR^{b}, —C(═NR^{b})N(R^{b})₂, —C(═O)R^{b}, —C(═O)OR^{b}, —C(═O)N(R^{b})₂, —NO₂, —NR^{b}C(═O)R^{b}, —NR^{b}C(═O)OR^{b}, —NR^{b}C(═O)N(R^{b})₂, —OC(═O)R^{b}, —OC(═O)OR^{b}, —OC(═O)N(R^{b})₂, or

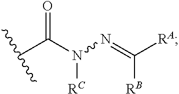

each instance of R^{b} is independently hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, an oxygen protecting group when attached to an oxygen atom, or a sulfur protecting group when attached to a sulfur atom, or two instances of R^{b} are joined to form a substituted or unsubstituted, heterocyclic ring, or substituted or unsubstituted, heteroaryl ring;

R^{B2} is hydrogen, substituted or unsubstituted acyl, substituted or unsubstituted alkyl, substituted or unsubstituted carbocyclyl, substituted or unsubstituted heterocyclyl, a nitrogen protecting group, or a warhead; and

R^{B3} is hydrogen, halogen, substituted or unsubstituted C₁₋₆ alkyl, —OR^{b}, —N(R^{b})₂, or a warhead;

R^{B4} is hydrogen, substituted or unsubstituted C₁₋₆ alkyl, or a nitrogen protecting group; and

R^{B5} is of the formula:

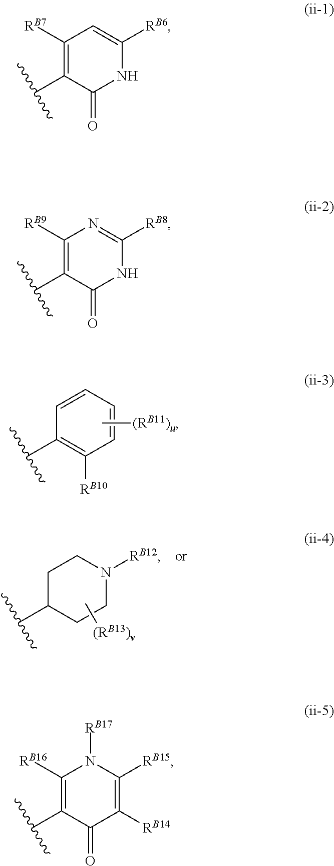

wherein:
R^{B6} is hydrogen, halogen, substituted or unsubstituted C₁₋₆ alkyl, or —N(R^{b})₂;
R^{B7} is hydrogen, halogen, substituted or unsubstituted C₂₋₆ alkyl, or substituted or unsubstituted, 3- to 7-membed, monocyclic carbocyclyl comprising 0, 1, or 2 double bonds in the carbocyclic ring system;
R^{B8} is hydrogen, halogen, substituted or unsubstituted C₁₋₆ alkyl, or —N(R^{b})₂;
R^{B9} is hydrogen, halogen, substituted or unsubstituted C₁₋₆ alkyl, or substituted or unsubstituted, 3- to 7-membed, monocyclic carbocyclyl comprising 0, 1, or 2 double bonds in the carbocyclic ring system;
R^{B10} is —OR^{b}, —N(R^{b})₂, or a warhead;
each instance of R^{B11} is independently halogen, substituted or unsubstituted C₁₋₆ alkyl, substituted or unsubstituted, 3- to 7-membed, monocyclic carbocyclyl comprising 0, 1, or 2 double bonds in the carbocyclic ring system, or —N(R^{b})₂;
u is 0, 1, 2, 3, or 4;
R^{B12} is hydrogen, substituted or unsubstituted C₁₋₆ alkyl, a nitrogen protecting group, or a warhead;
each instance of R^{B13} is independently halogen, substituted or unsubstituted C₁₋₆ alkyl, substituted or unsubstituted, 3- to 7-membed, monocyclic carbocyclyl comprising 0, 1, or 2 double bonds in the carbocyclic ring system, or —N(R^{b})₂;
v is 0, 1, 2, 3, 4, 5, 6, 7, 8, or 9;
R^{B14} is hydrogen, halogen, substituted or unsubstituted C₁₋₆ alkyl, —OR^{b}, or —N(R^{b})₂;
R^{B15} is hydrogen, halogen, substituted or unsubstituted C₁₋₆ alkyl, —OR^{b}, or —N(R^{b})₂;
R^{B16} is hydrogen, halogen, substituted or unsubstituted C₂₋₆ alkyl, substituted or unsubstituted, 3- to 7-membed, monocyclic carbocyclyl comprising 0, 1, or 2 double bonds in the carbocyclic ring system, —OR^{b}, or —N(R^{b})₂; and
R^{B17} is hydrogen, substituted or unsubstituted acyl, substituted or unsubstituted C₁₋₆ alkyl, a nitrogen protecting group, or a warhead.

In some embodiments, the EZH2 inhibitor is a compound is of formula:

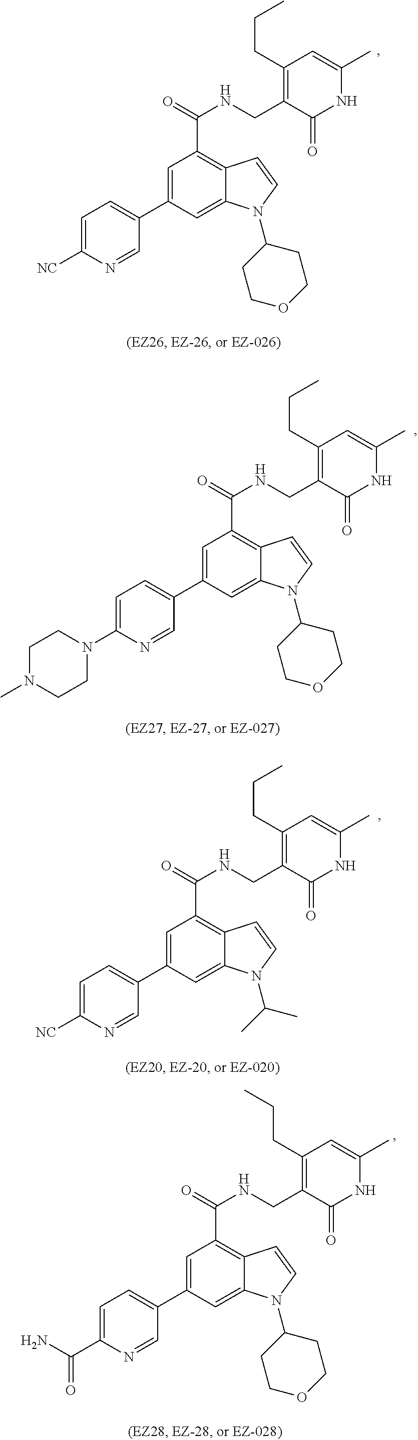

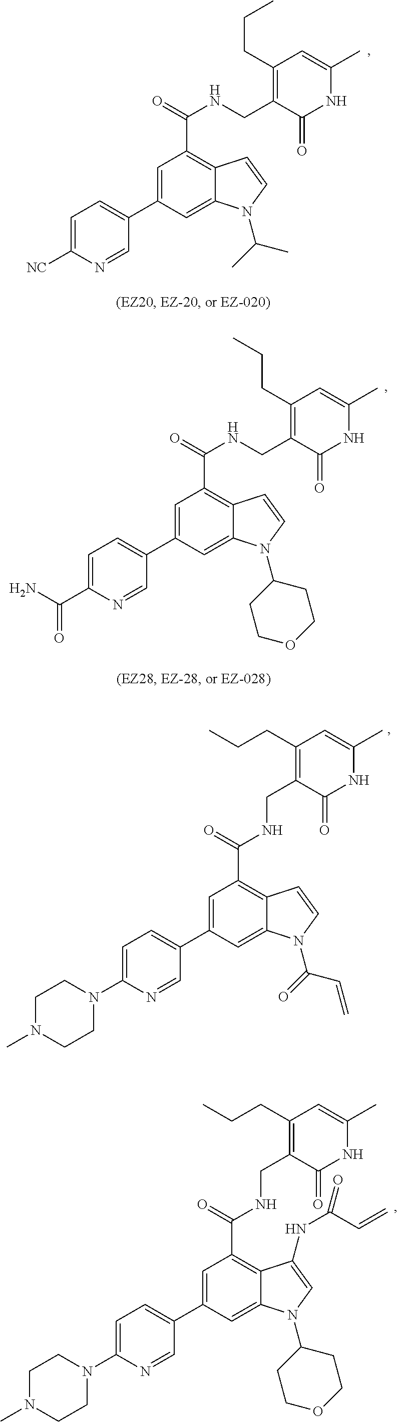

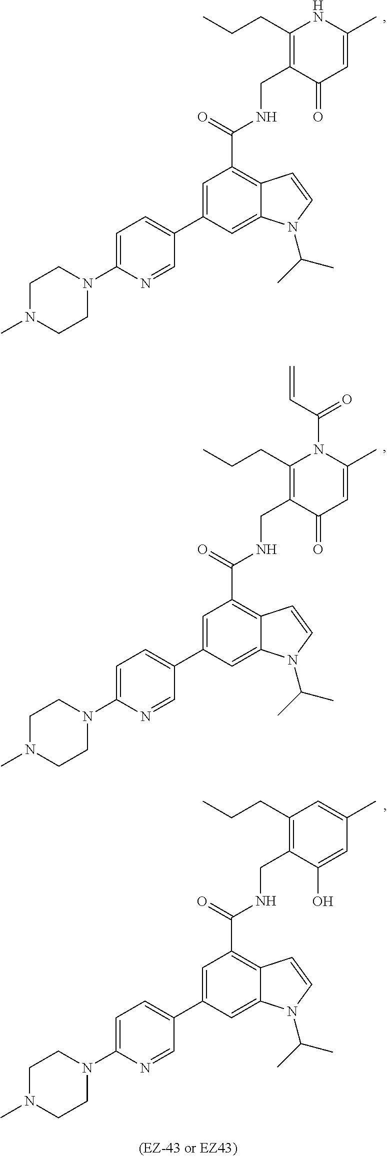

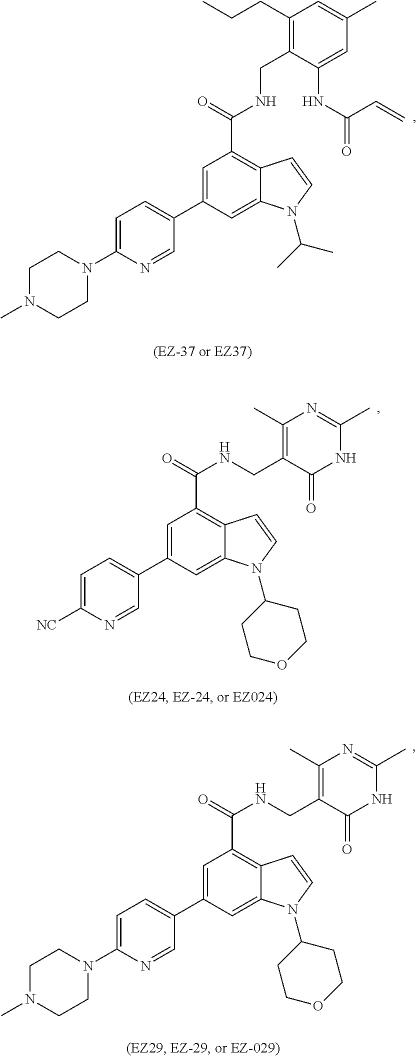

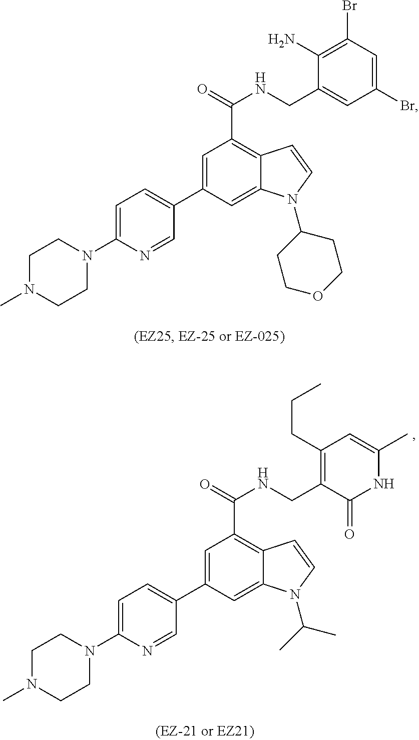

or a pharmaceutically acceptable salt, solvate, hydrate, polymorph, co-crystal, tautomer, stereoisomer, isotopically labeled derivative, or prodrug thereof.

In certain embodiments, the EZH2 inhibitor is a compound of the formula:

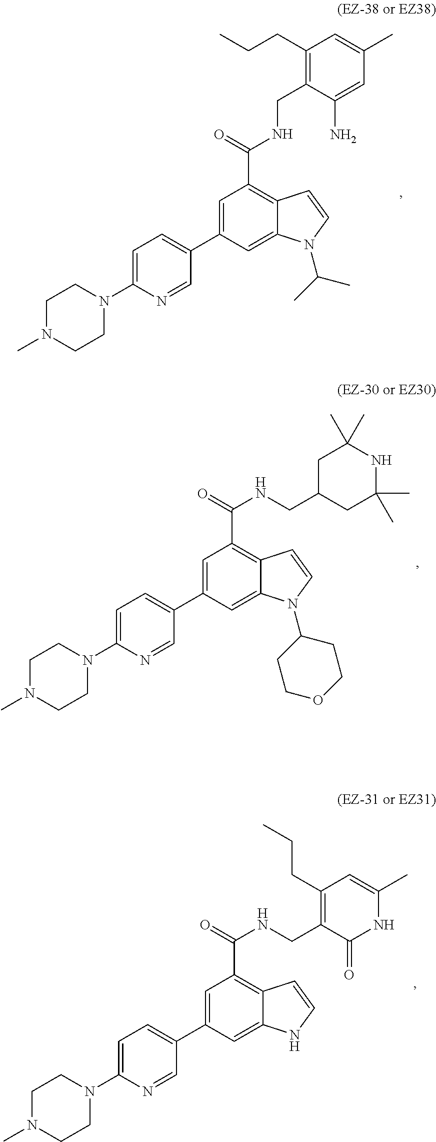

or a pharmaceutically acceptable salt, solvate, hydrate, polymorph, co-crystal, tautomer, stereoisomer, isotopically labeled derivative, or prodrug thereof.

In certain embodiments, the EZH2 inhibitor is a compound of the formula:

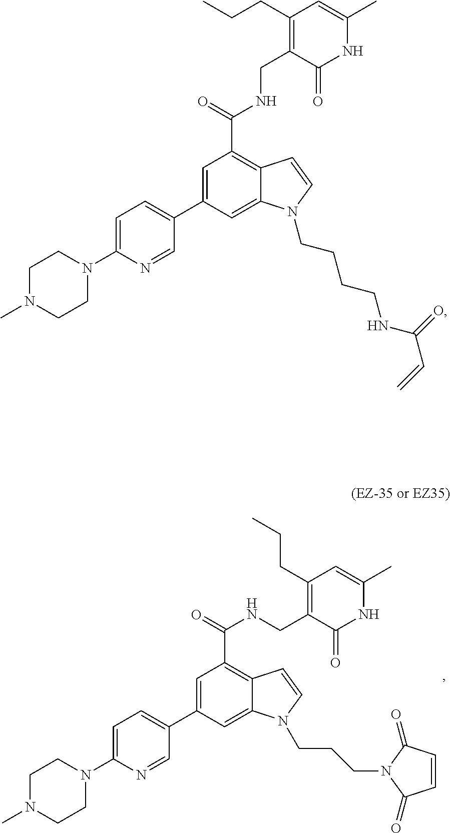 [SMILES: C=CC(=O)NCCCCn1ccc2c(C(=O)NCc3c(CCC)cc(C)[nH]c3=O)cc(-c3ccc(N4CCN(C)CC4)nc3)cc21 ; CCCc1cc(C)[nH]c(=O)c1CNC(=O)c1cc(-c2ccc(N3CCN(C)CC3)nc2)cc2c1ccn2CCCN1C(=O)C=CC1=O]

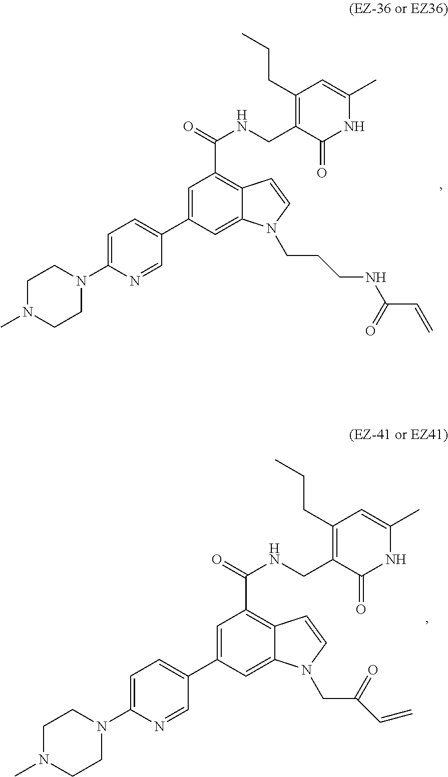

or a pharmaceutically acceptable salt, solvate, hydrate, polymorph, co-crystal, tautomer, stereoisomer, isotopically labeled derivative, or prodrug thereof.

In some embodiments, the Bcl6 inhibitor is a compound of the formula:

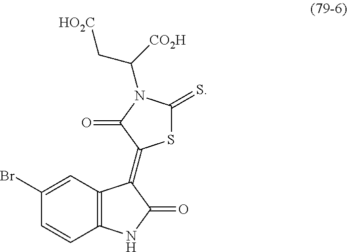 [SMILES: O=C(O)CC(C(=O)O)N1C(=O)/C(=C2/C(=O)Nc3ccc(Br)cc32)SC1=S]

In some embodiments, the BRD4 inhibitor is JQ1 and/or its analog.

In the embodiments, the allogeneic transplant is selected from the group consisting of cells, tissue, blood and organ. In the embodiments, the cells are stem cells, optionally human stem cells. In the embodiments, the allogeneic transplant comprises non-T-cell-depleted tissue.

In the embodiments, the enhancer of zeste homolog 2 (EZH2) inhibitor, the B-cell lymphoma 6 protein (Bcl6) inhibitor and/or the bromodomain-containing protein 4 (BRD4) inhibitor are administered to the subject prior the allogeneic transplant.

In the embodiments, the enhancer of zeste homolog 2 (EZH2) inhibitor, the B-cell lymphoma 6 protein (Bcl6) inhibitor and/or the bromodomain-containing protein 4 (BRD4) inhibitor are administered to the subject after the allogenic transplant.

In the embodiments, the EZH2 inhibitor, the Bcl6 inhibitor and/or the BRD4 inhibitor are administered to the subject at least one week, one month, two months, three months, four months, five months, six months, seven months, either months, nine months, ten months, eleven months, 1 year, 2 years or 3 years after the allogenic transplant. In the embodiments, the EZH2 inhibitor, the Bcl6 inhibitor and/or the BRD4 inhibitor are administered to the subject at least 100 days after the allogenic transplant.

These and other aspects of the inventions, as well as various advantages and utilities will be apparent with reference to the Detailed Description. Each aspect of the invention can encompass various embodiments as will be understood.

### BRIEF DESCRIPTION OF DRAWINGS

FIG. 1 demonstrates improved pulmonary function in mice receiving wild-type bone marrow (BM) and either wild-type spleen-derived T-cells, “S (WT)” or Bcl6 knockout spleen-derived-T-cells, “S (Bcl6 KO)”.

FIG. 2 demonstrates a decrease in T Follicular Helper Cells and Germinal Center B cells after administration of Bcl6 KO T-cells compared to WT spleen-derived T-cells, “S (WT)”.

FIG. 3 demonstrates a decrease in collagen deposition in the lungs of mice after administration of Bcl6 KO T-cells compared to WT spleen-derived T-cells, “S (WT)”.

FIG. 4 demonstrates decreased Ig deposition in the lungs of mice transplanted with Bcl6 KO T-cells compared to mice transplanted with WT spleen-derived T-cells, “S (WT)”.

FIG. 5 demonstrates improved pulmonary function in mice receiving enhancer of zeste homolog 2 knock out bone marrow (EZH2 KO BM) compared to mice receiving wild-type spleen-derived T-cells, “S (WT)”.

FIG. 6 demonstrates improved pulmonary function in mice receiving wild-type bone marrow (WT BM) with EZH2 KO spleen-derived T-cells, “S (EZH2 KO)” compared to mice receiving wild-type spleen-derived T-cells, “S (WT)”.

FIG. 7 demonstrates decreased frequency of germinal centers in the spleens of mice transplanted with EZH2 KO spleen-derived T-cells, “S (EZH2 KO)” compared to mice receiving wild-type spleen-derived T-cells, “S (WT)”.

FIG. 8 demonstrates that administration of UNC1999 after transplant of BM and T-cells is toxic. Mice administered BM alone had 100% survival after 60 days (circles). Mice administered BM, T-cells and vehicle (squares) or JQ5 (down arrows) had >60% survival after 60 days. Mice administered UNC1999 had <40% survival after 60 days (up arrows). Survival of mice administered Bcl6 inhibitor 79-6 was 100% (not shown). The arrow on the X-axis at Time 28 indicates treatment of mice began 28 days post-transplant.

FIG. 9 demonstrates that after transplant of BM and T-cells, treatment with an EZH2 inhibitor improves pulmonary function in mice.

FIG. 10 demonstrates that after transplant of BM and T-cells, EZH2 inhibitor-treated mice show a decrease in collagen deposition in the lungs.

FIG. 11 demonstrates that after transplant of BM and T-cells, mice treated with Bcl6 79-6 peptide show improved pulmonary function.

FIG. 12 demonstrates that after transplant of BM and T-cells, the spleens of mice treated with Bcl6 79-6 peptide show decreased germinal center B cells.

FIG. 13 demonstrates that after transplant of BM and T-cells, the lungs of mice treated with Bcl6 79-6 peptide show decreased collagen deposition.

FIG. 14 demonstrates that BRD4 inhibitor JQ1 improves pulmonary function when mice with cGVHD are treated with JQ1.

FIG. 15 demonstrates that after transplant of BM and T-cells, the lungs of mice treated with JQ1 show decreased collagen deposition.

FIG. 16 demonstrates that mice treated with JQ5 had a decrease in resistance and elastance with an increase in compliance. EZH2 KO BM showed similar results. When Tregs specifically had EZH2 KO, there was increased disease similar to the cGVHD control mice (column 2), but this was overcome by Ezh2 inhibition with JQ5.

FIG. 17 demonstrates that T follicular helper cells were decreased in mice therapeutically treated with JQ5 or in animals that had EZH2 KO BM. T follicular helpers were not decreased in Treg specific KO of EZH2.

FIG. 18 demonstrates that the frequency of Germinal Center B cells was similar to the cGVHD control animals, but the number of Germinal Center B cells in JQ5 treated mice was significantly decreased.

FIG. 19 demonstrates a decrease in resistance and elastance with an increase in compliance, decreased Germinal Center B cells, and decreased collagen in Trichrome staining when mice were treated with Bcl6 79-6.

FIG. 20 demonstrates that mice that were transplanted with bone marrow that does not express Bcl6 in B-cells did not develop pathogenic pulmonary function, as demonstrated by a decrease in resistance and elastance compared to the chronic GVHD controls along with an increase in compliance.

FIG. 21 shows exemplary aldehydes and ketones

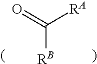

useful in preparing the hydrazides described herein.

### DETAILED DESCRIPTION OF INVENTION

### Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987. The disclosure is not intended to be limited in any manner by the exemplary listing of substituents described herein.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E. L. Eliel, Ed., Univ. of Notre Dame Press, Notre Dame, Ind. 1972). The disclosure additionally encompasses compounds described herein as individual isomers substantially free of other isomers, and alternatively, as mixtures of various isomers.

When a range of values is listed, it is intended to encompass each value and sub range within the range. For example “C₁₋₆” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆.

The term “aliphatic” includes both saturated and unsaturated, straight chain (i.e., unbranched), branched, acyclic, cyclic, or polycyclic aliphatic hydrocarbons, which are optionally substituted with one or more functional groups. As will be appreciated by one of ordinary skill in the art, “aliphatic” is intended herein to include, but is not limited to, alkyl, alkenyl, alkynyl, cycloalkyl, cycloalkenyl, and cycloalkynyl moieties. Thus, the term “alkyl” includes straight, branched and cyclic alkyl groups. An analogous convention applies to other generic terms such as “alkenyl”, “alkynyl”, and the like. Furthermore, the terms “alkyl”, “alkenyl”, “alkynyl”, and the like encompass both substituted and unsubstituted groups. In certain embodiments, “lower alkyl” is used to indicate those alkyl groups (cyclic, acyclic, substituted, unsubstituted, branched or unbranched) having 1-6 carbon atoms.

In certain embodiments, the alkyl, alkenyl, and alkynyl groups employed in the disclosure contain 1-20 aliphatic carbon atoms. In certain other embodiments, the alkyl, alkenyl, and alkynyl groups employed in the disclosure contain 1-10 aliphatic carbon atoms. In yet other embodiments, the alkyl, alkenyl, and alkynyl groups employed in the disclosure contain 1-8 aliphatic carbon atoms. In still other embodiments, the alkyl, alkenyl, and alkynyl groups employed in the disclosure contain 1-6 aliphatic carbon atoms. In yet other embodiments, the alkyl, alkenyl, and alkynyl groups employed in the disclosure contain 1-4 carbon atoms. Illustrative aliphatic groups thus include, but are not limited to, for example, methyl, ethyl, n-propyl, isopropyl, cyclopropyl, —CH₂-cyclopropyl, vinyl, allyl, n-butyl, sec-butyl, isobutyl, tert-butyl, cyclobutyl, —CH₂-cyclobutyl, n-pentyl, sec-pentyl, isopentyl, tert-pentyl, cyclopentyl, —CH₂-cyclopentyl, n-hexyl, sec-hexyl, cyclohexyl, —CH₂-cyclohexyl moieties and the like, which again, may bear one or more substituents. Alkenyl groups include, but are not limited to, for example, ethenyl, propenyl, butenyl, 1-methyl-2-buten-1-yl, and the like. Representative alkynyl groups include, but are not limited to, ethynyl, 2-propynyl (propargyl), 1-propynyl, and the like.

The term “alkyl” refers to a radical of a straight-chain or branched saturated hydrocarbon group having from 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, iso-butyl), pentyl (C₅) (e.g., n-pentyl, 3-pentanyl, amyl, neopentyl, 3-methyl-2-butanyl, tertiary amyl), and hexyl (C₆) (e.g., n-hexyl). Additional examples of alkyl groups include n-heptyl (C₇), n-octyl (C₈), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₀ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu), unsubstituted isobutyl (i-Bu)). In certain embodiments, the alkyl group is a substituted C₁₋₁₀ alkyl (such as substituted C₁₋₆ alkyl, e.g., —CF₃, Bn).

“Alkenyl” refers to a radical of a straight-chain or branched hydrocarbon group having from 2 to 20 carbon atoms, one or more carbon-carbon double bonds, and no triple bonds (“C₂₋₂₀ alkenyl”). In some embodiments, an alkenyl group has 2 to 10 carbon atoms (“C₂₋₁₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently optionally substituted, i.e., unsubstituted (an “unsubstituted alkenyl”) or substituted (a “substituted alkenyl”) with one or more substituents. In certain embodiments, the alkenyl group is unsubstituted C₂₋₁₀ alkenyl. In certain embodiments, the alkenyl group is substituted C₂₋₁₀ alkenyl. In an alkenyl group, a C═C double bond for which the stereochemistry is not specified (e.g., —CH═CHCH₃ or

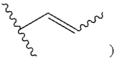

may be an (E)- or (Z)-double bond.

“Alkynyl” refers to a radical of a straight-chain or branched hydrocarbon group having from 2 to 20 carbon atoms, one or more carbon-carbon triple bonds, and optionally one or more double bonds (“C₂₋₂₀ alkynyl”). In some embodiments, an alkynyl group has 2 to 10 carbon atoms (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently optionally substituted, i.e., unsubstituted (an “unsubstituted alkynyl”) or substituted (a “substituted alkynyl”) with one or more substituents. In certain embodiments, the alkynyl group is unsubstituted C₂₋₁₀ alkynyl. In certain embodiments, the alkynyl group is substituted C₂₋₁₀ alkynyl.

“Carbocyclyl” or “carbocyclic” refers to a radical of a nonaromatic cyclic hydrocarbon group having from 3 to 10 ring carbon atoms (“C₃₋₁₀ carbocyclyl”) and zero heteroatoms in the nonaromatic ring system. In some embodiments, a carbocyclyl group has 3 to 8 ring carbon atoms (“C₃₋₈ carbocyclyl”). In some embodiments, a carbocyclyl group has 3 to 6 ring carbon atoms (“C₃₋₆ carbocyclyl”). In some embodiments, a carbocyclyl group has 3 to 6 ring carbon atoms (“C₃₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or contain a fused, bridged or spiro ring system such as a bicyclic system (“bicyclic carbocyclyl”) and can be saturated or can be partially unsaturated. “Carbocyclyl” also includes ring systems wherein the carbocyclic ring, as defined above, is fused with one or more aryl or heteroaryl groups wherein the point of attachment is on the carbocyclic ring, and in such instances, the number of carbons continue to designate the number of carbons in the carbocyclic ring system. Unless otherwise specified, each instance of a carbocyclyl group is independently optionally substituted, i.e., unsubstituted (an “unsubstituted carbocyclyl”) or substituted (a “substituted carbocyclyl”) with one or more substituents. In certain embodiments, the carbocyclyl group is unsubstituted C₃₋₁₀ carbocyclyl. In certain embodiments, the carbocyclyl group is substituted C₃₋₁₀ carbocyclyl.

In some embodiments, “carbocyclyl” is a monocyclic, saturated carbocyclyl group having from 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is unsubstituted C₃₋₁₀ cycloalkyl. In certain embodiments, the cycloalkyl group is substituted C₃₋₁₀ cycloalkyl.

“Heterocyclyl” or “heterocyclic” refers to a radical of a 3-to 10-membered nonaromatic ring system having ring carbon atoms and 1 to 4 ring heteroatoms, wherein each heteroatom is independently selected from nitrogen, oxygen, sulfur, boron, phosphorus, and silicon (“3-10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged, or spiro ring system, such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclic ring, as defined above, is fused with one or more carbocyclyl groups wherein the point of attachment is either on the carbocyclyl or heterocyclic ring, or ring systems wherein the heterocyclic ring, as defined above, is fused with one or more aryl or heteroaryl groups, wherein the point of attachment is on the heterocyclic ring, and in such instances, the number of ring members continue to designate the number of ring members in the heterocyclic ring system. Unless otherwise specified, each instance of heterocyclyl is independently optionally substituted, i.e., unsubstituted (an “unsubstituted heterocyclyl”) or substituted (a “substituted heterocyclyl”) with one or more substituents. In certain embodiments, the heterocyclyl group is unsubstituted 3-10 membered heterocyclyl. In certain embodiments, the heterocyclyl group is substituted 3-10 membered heterocyclyl.

In some embodiments, a heterocyclyl group is a 5-10 membered nonaromatic ring system having ring carbon atoms and 1-4 ring heteroatoms, wherein each heteroatom is independently selected from nitrogen, oxygen, sulfur, boron, phosphorus, and silicon (“5-10 membered heterocyclyl”). In some embodiments, a heterocyclyl group is a 5-8 membered non aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered nonaromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has one ring heteroatom selected from nitrogen, oxygen, and sulfur.

Exemplary 3-membered heterocyclyl groups containing one heteroatom include, without limitation, azirdinyl, oxiranyl, thiiranyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, and dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

“Aryl” refers to a radical of a monocyclic or polycyclic (e.g., bicyclic or tricyclic) 4n+2 aromatic ring system (e.g., having 6, 10, or 14 pi electrons shared in a cyclic array) having 6-14 ring carbon atoms and zero heteroatoms provided in the aromatic ring system (“C₆₋₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently optionally substituted, i.e., unsubstituted (an “unsubstituted aryl”) or substituted (a “substituted aryl”) with one or more substituents. In certain embodiments, the aryl group is unsubstituted C₆₋₁₄ aryl. In certain embodiments, the aryl group is substituted C₆₋₁₄ aryl.

“Aralkyl” is a subset of alkyl and aryl and refers to an optionally substituted alkyl group substituted by an optionally substituted aryl group. In certain embodiments, the aralkyl is optionally substituted benzyl. In certain embodiments, the aralkyl is benzyl. In certain embodiments, the aralkyl is optionally substituted phenethyl. In certain embodiments, the aralkyl is phenethyl.

“Heteroaryl” refers to a radical of a 5-10 membered monocyclic or bicyclic 4n+2 aromatic ring system (e.g., having 6 or 10 pi electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently optionally substituted, i.e., unsubstituted (an “unsubstituted heteroaryl”) or substituted (a “substituted heteroaryl”) with one or more substituents. In certain embodiments, the heteroaryl group is unsubstituted 5-14 membered heteroaryl. In certain embodiments, the heteroaryl group is substituted 5-14 membered heteroaryl.

Exemplary 5-membered heteroaryl groups containing one heteroatom include, without limitation, pyrrolyl, furanyl, and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl.

“Heteroaralkyl” is a subset of alkyl and heteroaryl and refers to an optionally substituted alkyl group substituted by an optionally substituted heteroaryl group.

“Unsaturated” or “partially unsaturated” refers to a group that includes at least one double or triple bond. A “partially unsaturated” ring system is further intended to encompass rings having multiple sites of unsaturation, but is not intended to include aromatic groups (e.g., aryl or heteroaryl groups). Likewise, “saturated” refers to a group that does not contain a double or triple bond, i.e., contains all single bonds.

Alkyl, alkenyl, alkynyl, carbocyclyl, heterocyclyl, aryl, and heteroaryl groups, which are divalent bridging groups, are further referred to using the suffix ene, e.g., alkylene, alkenylene, alkynylene, carbocyclylene, heterocyclylene, arylene, and heteroarylene.

An atom, moiety, or group described herein may be unsubstituted or substituted, as valency permits, unless otherwise provided expressly. The term “optionally substituted” refers to substituted or unsubstituted.

A group is optionally substituted unless expressly provided otherwise. The term “optionally substituted” refers to being substituted or unsubstituted. In certain embodiments, alkyl, alkenyl, alkynyl, carbocyclyl, heterocyclyl, aryl, and heteroaryl groups are optionally substituted (e.g., “substituted” or “unsubstituted” alkyl, “substituted” or “unsubstituted” alkenyl, “substituted” or “unsubstituted” alkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, any of the substituents described herein that results in the formation of a stable compound. The present disclosure contemplates any and all such combinations in order to arrive at a stable compound. For purposes of this disclosure, heteroatoms such as nitrogen may have hydrogen substituents and/or any suitable substituent as described herein which satisfy the valencies of the heteroatoms and results in the formation of a stable moiety. In certain embodiments, the substituent is a carbon atom substituent. In certain embodiments, the substituent is a nitrogen atom substituent. In certain embodiments, the substituent is an oxygen atom substituent. In certain embodiments, the substituent is a sulfur atom substituent.

Exemplary carbon atom substituents include, but are not limited to, halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)₂R^{aa}, —OP(═O)₂R^{aa}, —P(═O)(R^{aa})₂, —OP(═O)(R^{aa})₂, —OP(═O)(OR^{cc})₂, —P(═O)₂N(R^{bb})₂, —OP(═O)₂N(R^{bb})₂, —P(═O)(NR^{bb})₂, —OP(═O)(NR^{bb})₂, —NR^{bb}P(═O)(OR^{cc})₂, —NR^{bb}P(═O)(NR^{bb})₂, —P(R^{cc})₂, —P(R^{cc})₃, —OP(R^{cc})₂, —OP(R^{cc})₃, —B(R^{aa})₂, —B(OR^{cc})₂, —BR^{aa}(OR^{cc}), C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, carbocyclyl, heterocyclyl, aryl, and heteroaryl is independently substituted with 0, 1, 2, 3, 4, or 5 R^{dd} groups; or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc};

each instance of R^{aa} is, independently, selected from C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;

each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;

each instance of R^{CC} is, independently, selected from hydrogen, C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{CC} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;

each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)₂R^{ee}, —P(═O)(R^{ee})₂, —OP(═O)(R^{ee})₂, —OP (═O) (OR^{ee})₂, C₁ alkyl, C₁₋₆ perhaloalkyl, C₂₋₆ alkenyl, C₂₋₆ alkynyl, C₃₋₁₀ carbocyclyl, 3-10 membered heterocyclyl, C₆₋₁₀ aryl, 5-10 membered heteroaryl, wherein each alkyl, alkenyl, alkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S;

each instance of R^{ee} is, independently, selected from C₁₋₆ alkyl, C₁₋₆ perhaloalkyl, C₂₋₆ alkenyl, C₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, carbocyclyl, heterocyclyl, aryl, and heteroaryl is independently substituted with 0, 1, 2, 3, 4, or 5 R^{gg} groups;

each instance of R^{ff} is, independently, selected from hydrogen, C₁₋₆ alkyl, C₁₋₆ perhaloalkyl, C₂₋₆ alkenyl, C₂₋₆ alkynyl, C₃₋₁₀ carbocyclyl, 3-10 membered heterocyclyl, C₆₋₁₀ aryl and 5-10 membered heteroaryl, or two R^{ff} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{gg} groups; and

each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl) (C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃-C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)₂(C₁₋₆ alkyl), —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₆ perhaloalkyl, C₂₋₆ alkenyl, C₂₋₆ alkynyl, C₃₋₁₀ carbocyclyl, C₆₋₁₀ aryl, 3-10 membered heterocyclyl, 5-10 membered heteroaryl; or two geminal R^{gg} substituents can be joined to form ═O or ═S; wherein X⁻ is a counterion.

A “counterion” or “anionic counterion” is a negatively charged group associated with a positively charged group in order to maintain electronic neutrality. An anionic counterion may be monovalent (i.e., including one formal negative charge). An anionic counterion may also be multivalent (i.e., including more than one formal negative charge), such as divalent or trivalent. Exemplary counterions include halide ions (e.g., F⁻, Cr⁻, Br⁻, I⁻), NO₃⁻, ClO₄⁻, OH⁻, H₂PO₄⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), carboxylate ions (e.g., acetate, ethanoate, propanoate, benzoate, glycerate, lactate, tartrate, glycolate, and the like), BF₄⁻, PF₄⁻, PF₆⁻, AsF₆⁻, SbF₆⁻, B[3,5-(CF₃)₂C₆H₃]₄]⁻, BPh₄⁻, Al(OC(CF₃)₃)₄⁻, and a carborane anion (e.g., CB₁₁H₁₂⁻ or (HCB₁₁Me₅Br₆)⁻).

“Halo” or “halogen” refers to fluorine (fluoro, —F), chlorine (chloro, —Cl), bromine (bromo, —Br), or iodine (iodo, —I).

“Acyl” refers to a moiety selected from the group consisting of —C(═O)R^{aa}, —CHO, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —C(═O)NR^{bb}SO₂R^{aa}, —C(═S)N(R^{bb})₂, —C(═O)SR^{aa}, or —C(═S)SR^{aa}, wherein R^{aa} and R^{bb} are as defined herein.

Nitrogen atoms can be substituted or unsubstituted as valency permits, and include primary, secondary, tertiary, and quaternary nitrogen atoms. Exemplary nitrogen atom substituents include, but are not limited to, hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{CC} groups attached to a nitrogen atom are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc}, and R^{dd} are as defined above.

In certain embodiments, the substituent present on a nitrogen atom is a nitrogen protecting group (also referred to as an amino protecting group). Nitrogen protecting groups include, but are not limited to, —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc} and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

For example, nitrogen protecting groups such as amide groups (e.g., —C(═O)R^{aa}) include, but are not limited to, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide, and o-(benzoyloxymethyl)benzamide.

Nitrogen protecting groups such as carbamate groups (e.g., —C(═O)OR^{aa}) include, but are not limited to, methyl carbamate, ethyl carbamante, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC or Boc), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

Nitrogen protecting groups such as sulfonamide groups (e.g., —S(═O)₂R^{aa}) include, but are not limited to, p-toluenesulfonamide (Ts), benzenesulfonamide, 2,3,6,-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Other nitrogen protecting groups include, but are not limited to, phenothiazinyl-(10)-acyl derivative, N′-p-toluenesulfonylaminoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys).

Exemplary oxygen atom substituents include, but are not limited to, —R^{aa}, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃, —P(═O)₂R^{aa}, —P(═O)(R^{aa})₂, —P(═O)(OR^{cc})₂, —P(═O)₂N(R^{bb})₂, and —P(═O)(NR^{bb})₂, wherein R^{aa}, R^{bb}, and R^{cc} are as defined herein. In certain embodiments, the oxygen atom substituent present on an oxygen atom is an oxygen protecting group (also referred to as a hydroxyl protecting group). Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference. Exemplary oxygen protecting groups include, but are not limited to, methyl, t-butyloxycarbonyl (BOC or Boc), methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodisulfuran-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

Exemplary sulfur atom substituents include, but are not limited to, —R^{aa}, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃, —P(═O)₂R^{aa}, —P(═O)(R^{aa})₂, —P(═O)(OR^{cc})₂, —P(═O)₂N(R^{bb})₂, and —P(═O)(NR^{bb})₂, wherein R^{aa}, R^{bb}, and R^{cc} are as defined herein. In certain embodiments, the sulfur atom substituent present on a sulfur atom is a sulfur protecting group (also referred to as a thiol protecting group). Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

The term “leaving group” is given its ordinary meaning in the art of synthetic organic chemistry and refers to an atom or a group capable of being displaced by a nucleophile. Examples of suitable leaving groups include, but are not limited to, halogen (such as F, Cl, Br, or I (iodine)), alkoxycarbonyloxy, aryloxycarbonyloxy, alkanesulfonyloxy, arenesulfonyloxy, alkyl-carbonyloxy (e.g., acetoxy), arylcarbonyloxy, aryloxy, methoxy, N,O-dimethylhydroxylamino, pixyl, and haloformates. In some cases, the leaving group is a sulfonic acid ester, such as toluenesulfonate (tosylate, —OTs), methanesulfonate (mesylate, —OMs), p-bromobenzenesulfonyloxy (brosylate, —OBs), —OS(═O)₂(CF₂)₃CF₃ (nonaflate, —ONf), or trifluoromethanesulfonate (triflate, —OTf). In some cases, the leaving group is a brosylate, such as p-bromobenzenesulfonyloxy. In some cases, the leaving group is a nosylate, such as 2-nitrobenzenesulfonyloxy. In some embodiments, the leaving group is a sulfonate-containing group. In some embodiments, the leaving group is a tosylate group. The leaving group may also be a phosphineoxide (e.g., formed during a Mitsunobu reaction) or an internal leaving group such as an epoxide or cyclic sulfate. Other non-limiting examples of leaving groups are water, ammonia, alcohols, ether moieties, thioether moieties, zinc halides, magnesium moieties, diazonium salts, and copper moieties.

A “hydrocarbon chain” refers to a substituted or unsubstituted divalent alkyl, alkenyl, or alkynyl group. A hydrocarbon chain includes (1) one or more chains of carbon atoms immediately between the two radicals of the hydrocarbon chain; (2) optionally one or more hydrogen atoms on the chain(s) of carbon atoms; and (3) optionally one or more substituents (“non-chain substituents,” which are not hydrogen) on the chain(s) of carbon atoms. A chain of carbon atoms consists of consecutively connected carbon atoms (“chain atoms”) and does not include hydrogen atoms or heteroatoms. However, a non-chain substituent of a hydrocarbon chain may include any atoms, including hydrogen atoms, carbon atoms, and heteroatoms. For example, hydrocarbon chain —C^{A}H(C^{B}H₂C^{C}H₃)— includes one chain atom C^{A}, one hydrogen atom on C^{A}, and non-chain substituent —(C^{B}H₂C^{C}H₃). The term “Cₓ hydrocarbon chain,” wherein x is a positive integer, refers to a hydrocarbon chain that includes x number of chain atom(s) between the two radicals of the hydrocarbon chain. If there is more than one possible value of x, the smallest possible value of x is used for the definition of the hydrocarbon chain.

For example, —CH(C₂H₅)— is a C₁ hydrocarbon chain, and

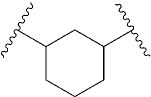 [SMILES: *C1CCCC(*)C1]

is a C₃ hydrocarbon chain. When a range of values is used, the meaning of the range is as described herein. For example, a C₃₋₁₀ hydrocarbon chain refers to a hydrocarbon chain where the number of chain atoms of the shortest chain of carbon atoms immediately between the two radicals of the hydrocarbon chain is 3, 4, 5, 6, 7, 8, 9, or 10. A hydrocarbon chain may be saturated (e.g., —(CH₂)₄—). A hydrocarbon chain may also be unsaturated and include one or more C═C and/or C≡C bonds anywhere in the hydrocarbon chain. For instance, —CH═CH(CH₂)₂—, —CH₂—C≡C—CH₂—, and —C≡C—CH═CH— are all examples of a unsubstituted and unsaturated hydrocarbon chain. In certain embodiments, the hydrocarbon chain is unsubstituted (e.g., —C≡C— or —(CH₂)₄—). In certain embodiments, the hydrocarbon chain is substituted (e.g., —CH(C₂H₅)— and —CF₂—). Any two substituents on the hydrocarbon chain may be joined to form an optionally substituted carbocyclyl, optionally substituted heterocyclyl, optionally substituted aryl, or optionally substituted heteroaryl ring. For instance,

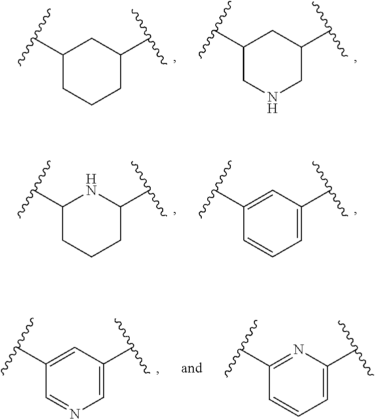 [SMILES: *C1CCCC(*)C1 ; *C1CNCC(*)C1 ; *C1CCCC(*)N1 ; *c1cccc(*)c1 ; *c1cncc(*)c1 ; *c1cccc(*)n1]

are all examples of a hydrocarbon chain. In contrast, in certain embodiments,

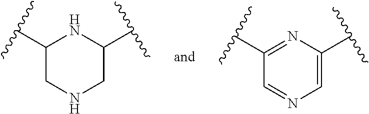 [SMILES: *C1CNCC(*)N1 ; *c1cncc(*)n1]

are not within the scope of the hydrocarbon chains described herein. When a chain atom of a Cₓ hydrocarbon chain is replaced with a heteroatom, the resulting group is referred to as a Cₓ hydrocarbon chain wherein a chain atom is replaced with a heteroatom, as opposed to a Cₓ₋₁ hydrocarbon chain. For example,

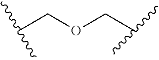 [SMILES: *COC*]

is a C₃ hydrocarbon chain wherein one chain atom is replaced with an oxygen atom.

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, Berge et al., describe pharmaceutically acceptable salts in detail in J. Pharmaceutical Sciences, 1977, 66, 1-19, incorporated herein by reference. Pharmaceutically acceptable salts of the compounds described herein include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid, and perchloric acid or with organic acids such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄ alkyl)₄⁻ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

The term “solvate” refers to forms of the compound, or a salt thereof, that are associated with a solvent, usually by a solvolysis reaction. This physical association may include hydrogen bonding. Conventional solvents include water, methanol, ethanol, acetic acid, DMSO, THF, diethyl ether, and the like. The compounds described herein may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolatable solvates. Representative solvates include hydrates, ethanolates, and methanolates.

The term “hydrate” refers to a compound that is associated with water. Typically, the number of the water molecules contained in a hydrate of a compound is in a definite ratio to the number of the compound molecules in the hydrate. Therefore, a hydrate of a compound may be represented, for example, by the general formula R.x H₂O, wherein R is the compound, and x is a number greater than 0. A given compound may form more than one type of hydrate, including, e.g., monohydrates (x is 1), lower hydrates (x is a number greater than 0 and smaller than 1, e.g., hemihydrates (R.0.5H₂O)), and polyhydrates (x is a number greater than 1, e.g., dihydrates (R.2H₂O) and hexahydrates (R.6H₂O)).

The term “tautomers” or “tautomeric” refers to two or more interconvertable compounds resulting from at least one formal migration of a hydrogen atom and at least one change in valency (e.g., a single bond to a double bond, a triple bond to a single bond, or vice versa). The exact ratio of the tautomers depends on several factors, including temperature, solvent, and pH. Tautomerizations (i.e., the reaction providing a tautomeric pair) may catalyzed by acid or base. Exemplary tautomerizations include keto-to-enol, amide-to-imide, lactam-to-lactim, enamine-to-imine, and enamine-to-(a different enamine) tautomerizations.

It is also to be understood that compounds that have the same molecular formula but differ in the nature or sequence of bonding of their atoms or the arrangement of their atoms in space are termed “isomers”. Isomers that differ in the arrangement of their atoms in space are termed “stereoisomers”.

Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The term “polymorphs” refers to a crystalline form of a compound (or a salt, hydrate, or solvate thereof). All polymorphs have the same elemental composition. Different crystalline forms usually have different X-ray diffraction patterns, infrared spectra, melting points, density, hardness, crystal shape, optical and electrical properties, stability, and solubility. Recrystallization solvent, rate of crystallization, storage temperature, and other factors may cause one crystal form to dominate. Various polymorphs of a compound can be prepared by crystallization under different conditions.

The term “prodrugs” refers to compounds that have cleavable groups and become by solvolysis or under physiological conditions the compounds described herein, which are pharmaceutically active in vivo. Such examples include, but are not limited to, choline ester derivatives and the like, N-alkylmorpholine esters and the like. Other derivatives of the compounds described herein have activity in both their acid and acid derivative forms, but in the acid sensitive form often offer advantages of solubility, tissue compatibility, or delayed release in the mammalian organism (see, Bundgard, H., Design of Prodrugs, pp. 7-9, 21-24, Elsevier, Amsterdam 1985). Prodrugs include acid derivatives well known to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides, and anhydrides derived from acidic groups pendant on the compounds described herein are particular prodrugs. In some cases it is desirable to prepare double ester type prodrugs such as (acyloxy)alkyl esters or ((alkoxycarbonyl)oxy)alkylesters. C₁-C₈ alkyl, C₂-C₈ alkenyl, C₂-C₈ alkynyl, aryl, C₇-C₁₂ substituted aryl, and C₇-C₁₂ arylalkyl esters of the compounds described herein may be preferred.

The term “small molecule” refers to molecules, whether naturally-occurring or artificially created (e.g., via chemical synthesis) that have a relatively low molecular weight. Typically, a small molecule is an organic compound (i.e., it contains carbon). The small molecule may contain multiple carbon-carbon bonds, stereocenters, and other functional groups (e.g., amines, hydroxyl, carbonyls, and heterocyclic rings, etc.). In certain embodiments, the molecular weight of a small molecule is not more than about 1,000 g/mol, not more than about 900 g/mol, not more than about 800 g/mol, not more than about 700 g/mol, not more than about 600 g/mol, not more than about 500 g/mol, not more than about 400 g/mol, not more than about 300 g/mol, not more than about 200 g/mol, or not more than about 100 g/mol. In certain embodiments, the molecular weight of a small molecule is at least about 100 g/mol, at least about 200 g/mol, at least about 300 g/mol, at least about 400 g/mol, at least about 500 g/mol, at least about 600 g/mol, at least about 700 g/mol, at least about 800 g/mol, or at least about 900 g/mol, or at least about 1,000 g/mol. Combinations of the above ranges (e.g., at least about 200 g/mol and not more than about 500 g/mol) are also possible. In certain embodiments, the small molecule is a therapeutically active agent such as a drug (e.g., a molecule approved by the U.S. Food and Drug Administration as provided in the Code of Federal Regulations (C.F.R.)). The small molecule may also be complexed with one or more metal atoms and/or metal ions. In this instance, the small molecule is also referred to as a “small organometallic molecule.” Preferred small molecules are biologically active in that they produce a biological effect in animals, preferably mammals, more preferably humans. Small molecules include, but are not limited to, radionuclides and imaging agents. In certain embodiments, the small molecule is a drug. Preferably, though not necessarily, the drug is one that has already been deemed safe and effective for use in humans or animals by the appropriate governmental agency or regulatory body. For example, drugs approved for human use are listed by the FDA under 21 C.F.R. §§ 330.5, 331 through 361, and 440 through 460, incorporated herein by reference; drugs for veterinary use are listed by the FDA under 21 C.F.R. §§ 500 through 589, incorporated herein by reference. All listed drugs are considered acceptable for use in accordance with the present invention.

The present disclosure is based, at least in part, on the discovery that enhancer of zeste homolog 2 (EZH2) inhibitors, B-cell lymphoma 6 protein (Bcl6) inhibitors and/or bromodomain-containing protein 4 (BRD4) inhibitors can be used to treat chronic graft versus host disease (cGVHD). Accordingly, aspects of the disclosure relate to methods for treating cGVHD, and methods for improving pulmonary function in subjects receiving allogeneic transplant.

Some aspects of the present disclosure relate to method for treating chronic graft-versus-host disease (cGVHD), the method comprising administering to a subject in need thereof an enhancer of zeste homolog 2 (EZH2) inhibitor, a B-cell lymphoma 6 protein (Bcl6) inhibitor and/or a bromodomain-containing protein 4 (BRD4) inhibitor in an amount effective to treat cGVHD.

As used herein, “cGVHD” refers to a medical complication of allogeneic transplant resulting in the attack of the transplant recipient (host cells) by immune cells present in the transplanted cells or tissue (graft). For example, white blood cells (including T-cells) present in a graft may recognize host tissue as antigenic and induce an immune response against the host tissue, which is incapable of defending the attack because of its immuno-compromised status.

Chronic graft versus host disease (cGVHD) usually occurs more than 100 days after a subject receives an allogeneic tissue transplant or an un-irradiated blood transfusion. As used herein, “allogeneic” refers to tissue transplants that are genetically dissimilar or immunologically incompatible to the transplant host. In some embodiments, the allogeneic transplant tissue is a cell or cells. In some embodiments, the cell or cells are blood cells. In some embodiments, the cell or cells are stem cells, optionally human stem cells. In some embodiments, the stem cells are hematopoietic stem cells or bone-marrow derived stem cells. In some embodiments, the allogeneic transplant tissue is an organ. Non-limiting examples of organs that are capable of being transplanted are lung, heart, kidney, liver, pancreas, intestine, stomach, cornea, skin, and bone.

cGVHD target organs include skin, eyes, liver, mouth, gastrointestinal tract, neuromuscular system, lungs, or genitourinary tract. In some embodiments, cGVHD develops in a subject at the location of the allogeneic tissue transplant. In some embodiments, cGVHD develops in a subject at a location remote to the location of the allogeneic tissue transplant. Common signs and symptoms associated with cGVHD are known in the art and include, but are not limited to, rash, raised or discolored areas of the skin, dry eyes or vision changes, dry mouth, white patches on the inside of the mouth, pain or sensitivity to spicy foods, shortness of breath, compromised pulmonary function (e.g. increase in lung resistance, increase in lung elastance, decrease in lung compliance) compared to a subject without cGVHD, bronchiolitis obliterans, deposition of collagen in the lung, deposition of Ig in the lung, difficulty swallowing, pain with swallowing, weight loss, fatigue, muscle weakness, muscle pain. CGVHD may also present in the immune system, resulting in lymphocyte depletion and/or formation of germinal centers that secrete alloantibodies. As used herein, “alloantibody” refers to an antibody that reacts with an antigen from a genetically different individual of the same species. For example, in cGVHD the introduction of a graft containing immune cells causes the production of alloantibodies against the host tissue.

In some aspects, the instant disclosure relates to methods of treating cGVHD in a subject in need thereof by administering an effective amount of at least one of the compounds described herein. A subject in need of treatment is a subject identified as having cGVHD, i.e., the subject has been diagnosed by a physician (e.g., using methods well known in the art) as having cGVHD. In some embodiments, the subject in need of treatment is a subject suspected of having or developing cGVHD, such as a subject presenting one or more symptoms indicative of cGVHD. The term “subject in need of treatment” further includes subjects who are at risk of developing cGVHD, for example, subjects who are going to receive an allogenenic transplant or subjects who have had acute GVHD.

In some embodiments, the subject develops cGVHD as a result of allogeneic tissue transfer. In some embodiments, the subject develops cGVHD as a result of allogeneic blood transfusion. In some embodiments, the subject develops cGVHD as a result of allogeneic peripheral blood transfusion. In some embodiments, the subject has undergone an allogeneic tissue transplant but has not yet developed cGVHD. In some embodiments, the EZH2 inhibitor, Bcl6 inhibitor and/or BRD4 inhibitor are administered to the subject prior to receiving the allogeneic transplant. In some embodiments, the EZH2 inhibitor, Bcl6 inhibitor and/or BRD4 inhibitor are administered to the subject at least 1 month, 1 week, 3 days, 2, days, or 1 day prior to receiving the allogeneic transplant. In some embodiments, the EZH2 inhibitor, Bcl6 inhibitor and/or BRD4 inhibitor are administered to the subject after receiving the allogeneic transplant. In some embodiments, the EZH2 inhibitor, Bcl6 inhibitor and/or BRD4 inhibitor are administered to the subject at least one week, one month, two months, three months, four months, five months, six months, seven months, either months, nine months, ten months, eleven months, 1 year, 2 years or 3 years after the allogeneic transplant. In some embodiments, the EZH2 inhibitor, Bcl6 inhibitor and/or BRD4 inhibitor are administered to the subject at least 100 days after the allogeneic transplant.

A “subject” to which administration is contemplated includes, but is not limited to, humans; commercially relevant mammals such as cattle, pigs, horses, sheep, goats, cats, and/or dogs) and birds (e.g., commercially relevant birds such as chickens, ducks, geese, and/or turkeys). In some embodiments, the subject is a mammal. In some embodiments, the subject is a human. In some embodiments, the subject is a non-human primate.

As used herein, the terms “treatment,” “treat,” and “treating” refer to reversing, alleviating, delaying the onset of, or inhibiting the progression of cGVHD. Treatment includes ameliorating existing signs and symptoms of cGVHD, preventing additional symptoms, ameliorating or preventing the underlying causes of symptoms, preventing the severity of the condition or reversing the condition, at least partially. Thus, in some embodiments, treatment of cGVHD results in improved pulmonary function in a subject. In some embodiments, treatment of cGVHD results in decreased deposition of collagen into the lungs of a subject. In some embodiments, treatment of cGVHD results in decreased formation of germinal centers in a subject. In some embodiments, the compounds disclosed herein prevent the development of the signs and symptoms of cGVHD in a subject receiving an allogeneic transplant.

Some aspects of the present disclosure relate to methods for improving pulmonary function in a subject receiving an allogeneic transplant, the method comprising administering to a subject in need thereof an enhancer of zeste homolog 2 (EZH2) inhibitor, a B-cell lymphoma 6 protein (Bcl6) inhibitor and/or a bromodomain-containing protein 4 (BRD4) inhibitor in an amount effective to improve pulmonary function.

Pulmonary complications significantly contribute to late mortality after allogeneic transplant. The compounds described herein may be used to ameliorate the signs and symptoms of reduced pulmonary function experienced by a subject receiving an allogeneic transplant. The signs and symptoms of reduced pulmonary function include, but are not limited to, increased shortness of breath, fever, cough, sputum, hypoxemia. In some embodiments, an improvement in the pulmonary function may be determined using at least one pulmonary function test (PFT). The terms “pulmonary function test” and “PFT” as used herein refers to a series of diagnostic studies used to examine the severity of lung disease. The term “PFT” includes but is not limited to a spirogram (obstructive disease gauge), a lung volume determination (restrictive disease gauge), and a diffusion capacity test. In some embodiments, the at least one pulmonary function test comprises a test selected from the group consisting of a forced vital capacity test (FVC), a forced expiratory volume in one second test (FEV1.0) and a diffuse capacity of lungs for carbon monoxide test (DLCO).

As used herein, the terms “forced vital capacity test” and “FVC” refer to determinations of the volume of air expired with maximal force. It is usually measured along with expiratory flow rates in simple spirometry.

The terms “forced expiratory volume in one second test” and “FEV1.0” as used herein, refer to determinations of the volume of air forcefully expired during the first second after a full breath (normally accounts for >75% of the FVC). This value is recorded both as an absolute value and as a percentage of the FVC (FEV 1% FVC).

As used herein, the terms “diffuse capacity of lungs for carbon monoxide test” “DLCO” refer to determinations of carbon monoxide absorption upon in respiration. For the “DLCO” test, a patient inspires a known small amount of carbon monoxide, holds his breath for ten seconds, then exhales. A sample of alveolar (end-expired) gas is analyzed for carbon monoxide, and the amount absorbed during that breath is then calculated and expressed as mL/min/mm Hg.

It will be understood by those skilled in the art that any mode of administration, vehicle or carrier conventionally employed and which is inert with respect to the active agent may be utilized for preparing and administering the compositions disclosed herein. Illustrative of such methods, vehicles and carriers are those described, for example, in Remington's Pharmaceutical Sciences, 4th ed. (1970), the disclosure of which is incorporated herein by reference. Those skilled in the art, having been exposed to the principles of the invention, will experience no difficulty in determining suitable and appropriate vehicles, excipients and carriers or in compounding the active ingredients therewith to form the pharmaceutical compositions of the invention.

An “effective amount” refers to an amount sufficient to elicit the desired biological response, i.e., treating cGVHD. As will be appreciated by those of ordinary skill in this art, the effective amount of the compounds described herein may vary depending on such factors as the desired biological endpoint, the pharmacokinetics of the compound, the condition being treated, the mode of administration, and the age and health of the subject. An effective amount includes, but is not limited to, that amount necessary to slow, reduce, inhibit, ameliorate or reverse one or more symptoms associated with cGVHD. For example, in the treatment of cGVHD, such terms may refer to improved pulmonary function (e.g. increase in lung resistance, increase in lung elastance, decrease in lung compliance).

An effective amount of a compound may vary from about 0.001 mg/kg to about 1000 mg/kg in one or more dose administrations, for one or several days (depending on the mode of administration). In certain embodiments, the effective amount varies from about 0.001 mg/kg to about 1000 mg/kg, from about 0.01 mg/kg to about 750 mg/kg, from about 0.1 mg/kg to about 500 mg/kg, from about 1.0 mg/kg to about 250 mg/kg, and from about 10.0 mg/kg to about 150 mg/kg. One of ordinary skill in the art would be able to determine empirically an appropriate therapeutically effective amount.

In some aspects, the instant disclosure relates to a method for treating chronic graft-versus-host disease (cGVHD), the method comprising administering to a subject in need thereof an enhancer of zeste homolog 2 (EZH2) inhibitor, a B-cell lymphoma 6 protein (Bcl6) inhibitor and/or a bromodomain-containing protein 4 (BRD4) inhibitor in an amount effective to treat cGVHD.

A core catalytic component of the Polycomb-group (PcG) protein complex family, enhancer of zeste homolog 2 (EZH2) is a histone methyltransferase that that catalyzes the di- and tri-methylation at histone H3 lysine 27 (H3K27me2/3), thereby silencing gene expression. The catalytic site of EZH2 is present within a SET domain, a highly conserved sequence motif (named after Su(var)3-9, Enhancer of Zeste, Trithorax) that is found in several chromatin-associated proteins. EZH2 plays a critical role in normal development and EZH2 deficient mice die at early stage of embryo due to the failure of implantation and gastrulation. This protein associates with the embryonic ectoderm development protein, the VAV1 oncoprotein, and the X-linked nuclear protein (XNP). This protein may also play a role in the hematopoietic and central nervous systems.

B-cell lymphoma 6 protein (Bcl6) is a zinc finger transcription factor that acts as a transcriptional repressor. Bcl6 is a master regulator for differentiation of follicular T-cell (T_{FH}), which are required for the formation of germinal centers.

The BET (bromodomain and extra-terminal) proteins are four closely related bromodomain-containing proteins (BRD2, BRD3, BRD4, and BRDT) which constitute a subset of the larger family of 47 bromodomain-containing proteins. Bromodomains are acetyl-lysine binding pockets that target bromodomain-containing proteins to histones and thereby affect chromatin structure and function. The binding of BET protein bromodomains to chromatin regulates gene expression and small molecule inhibition of that binding produces selective effects on gene expression. Non-limiting examples BET bromodomain inhibitors, known in the art, include JQ1 and its analogs which have been described in US 2013/0184264, the disclosure of which is incorporated herein by reference. Thus, in some embodiments, the methods, and pharmaceutical compositions of the present disclosure comprise the BET bromodomain inhibitors described in US 2013/0184264, and incorporated herein by reference. The present disclosure further encompasses pharmaceutically acceptable salts of such compounds. In some embodiments, the methods, and pharmaceutical compositions of the present disclosure comprise BRD4 inhibitors, such as JQ1 and/or its analogs which have been described in US 2013/0184264.

Germinal center (GC) formation, immunoglobulin deposition and increased T follicular helper cells are required for cGVHD (Flynn et al. Blood. 2014 Jun. 19; 123(25):3988-98). Without wishing to be bound by any particular theory, it is believed that EZH2 inhibitors, Bcl6 inhibitors and/or BRD4 inhibitors decrease T follicular helper cells and germinal center (GC) formation, thereby treating/preventing cGVHD. As used herein an “inhibitor” refers to the ability of a compound to reduce (e.g., slow, halt) or prevent the biological activity of EZH2, Bcl6 or BRD4 in a cell relative to vehicle. In some embodiments, inhibitors of EZH2, Bcl6 or BRD4 include, but are not limited to, small molecules, peptides, peptidomimetics, antibodies or nucleic acids.

### Compounds

Several examples of EZH2 inhibitors are described herein. In some embodiments, the EZH2 inhibitor is a compound of Formula (I):

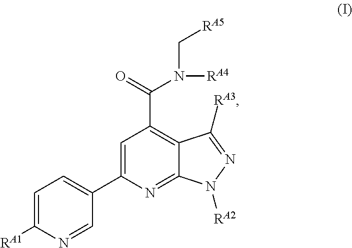

and pharmaceutically acceptable salts, solvates, hydrates, polymorphs, co-crystals, tautomers, stereoisomers, isotopically labeled derivatives, and prodrugs thereof, wherein:

R^{A1} is halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{a}, —N(R^{a})₂, —SR^{a}, —CN, —SCN, —C(═NR^{a})R^{a}, —C(═NR^{a})OR^{a}, —C(═NR^{a})N(R^{a})₂, —C(═O)R^{a}, —C(═O)OR^{a}, —C(═O)N(R^{a})₂, —NO₂, —NR^{a}C(═O)R^{a}, —NR^{a}C(═O)OR^{a}, —NR^{a}C(═O)N(R^{a})₂, —OC(═O)R^{a}, —OC(═O)OR^{a}, —OC(═O)N(R^{a})₂, or

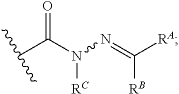

each instance of R^{a} is independently hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, an oxygen protecting group when attached to an oxygen atom, or a sulfur protecting group when attached to a sulfur atom, or two instances of R^{a} are joined to form a substituted or unsubstituted, heterocyclic ring, or substituted or unsubstituted, heteroaryl ring;

R^{A} is hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;

R^{B} is hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;

or R^{A} and R^{B} are joined to form a substituted or unsubstituted, carbocyclic ring, or a substituted or unsubstituted, heterocyclic ring;

R^{C} is hydrogen, substituted or unsubstituted C₁₋₆ alkyl, or a nitrogen protecting group;

R^{A2} is hydrogen, substituted or unsubstituted acyl, substituted or unsubstituted alkyl, substituted or unsubstituted carbocyclyl, substituted or unsubstituted heterocyclyl, a nitrogen protecting group, or a warhead;

R^{A3} is hydrogen, halogen, substituted or unsubstituted C₁₋₆ alkyl, —OR^{a}, —N(R^{a})₂, or a warhead;

R^{A4} is hydrogen, substituted or unsubstituted C₁₋₆ alkyl, or a nitrogen protecting group; and

R^{A5} is of the formula:

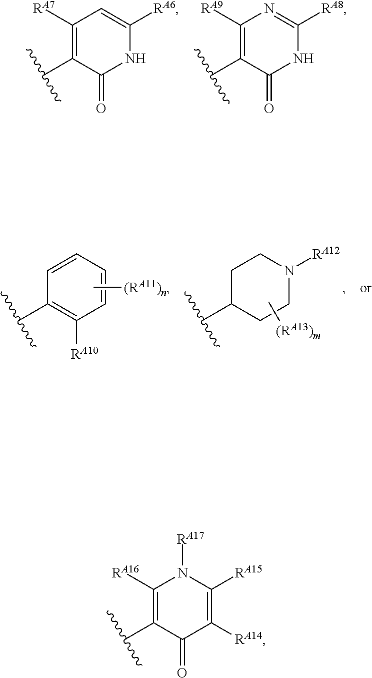

wherein:
R^{A6} is hydrogen, halogen, substituted or unsubstituted C₁₋₆ alkyl, —OR^{a}, or —N(R^{a})₂;
R^{A7} is hydrogen, halogen, substituted or unsubstituted C₂₋₆ alkyl, substituted or unsubstituted, 3- to 7-membed, monocyclic carbocyclyl comprising 0, 1, or 2 double bonds in the carbocyclic ring system, —OR^{a}, or —N(R^{a})₂;
R^{A8} is hydrogen, halogen, substituted or unsubstituted C₁₋₆ alkyl, —OR^{a}, or —N(R^{a})₂;
R^{A9} is hydrogen, halogen, substituted or unsubstituted C₁₋₆ alkyl, substituted or unsubstituted, 3- to 7-membed, monocyclic carbocyclyl comprising 0, 1, or 2 double bonds in the carbocyclic ring system, —OR^{a}, or —N(R^{a})₂;
R^{A10} is —OR^{a}, —N(R^{a})₂, or a warhead;
each instance of R^{A11} is independently halogen, substituted or unsubstituted C₁₋₆ alkyl, substituted or unsubstituted, 3- to 7-membed, monocyclic carbocyclyl comprising 0, 1, or 2 double bonds in the carbocyclic ring system, —OR^{a}, or —N(R^{a})₂;
n is 0, 1, 2, 3, or 4;
R^{A12} is hydrogen, substituted or unsubstituted C₁₋₆ alkyl, a nitrogen protecting group, or a warhead;
each instance of R^{A13} is independently halogen, substituted or unsubstituted C₁₋₆ alkyl, substituted or unsubstituted, 3- to 7-membed, monocyclic carbocyclyl comprising 0, 1, or 2 double bonds in the carbocyclic ring system, —OR^{a}, or —N(R^{a})₂;
m is 0, 1, 2, 3, 4, 5, 6, 7, 8, or 9;
R^{A14} is hydrogen, halogen, substituted or unsubstituted C₁₋₆ alkyl, —OR^{a}, or —N(R^{a})₂;
R^{A15} is hydrogen, halogen, substituted or unsubstituted C₁₋₆ alkyl, —OR^{a}, or —N(R^{a})₂;
R^{A16} is hydrogen, halogen, substituted or unsubstituted C₂₋₆ alkyl, substituted or unsubstituted, 3- to 7-membed, monocyclic carbocyclyl comprising 0, 1, or 2 double bonds in the carbocyclic ring system, —OR^{a}, or —N(R^{a})₂; and
R^{A17} is hydrogen, substituted or unsubstituted acyl, substituted or unsubstituted C₁₋₆ alkyl, a nitrogen protecting group, or a warhead.

In some embodiments, the EZH2 inhibitor is a compound of Formula (I):

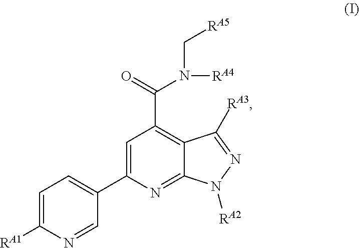

or a pharmaceutically acceptable salt, solvate, hydrate, polymorph, co-crystal, tautomer, stereoisomer, isotopically labeled derivative, or prodrug thereof, wherein:

R^{A1} is halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{a}, —N(R^{a})₂, —SR^{a}, —CN, —SCN, —C(═NR^{a})R^{a}, —C(═NR^{a})OR^{a}, —C(═NR^{a})N(R^{a})₂, —C(═O)R^{a}, —C(═O)OR^{a}, —C(═O)N(R^{a})₂, —NO₂, —NR^{a}C(═O)R^{a}, —NR^{a}C(═O)OR^{a}, —NR^{a}C(═O)N(R^{a})₂, —OC(═O)R^{a}, —OC(═O)OR^{a}, or —OC(═O)N(R^{a})₂;

each instance of R^{a} is independently hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, an oxygen protecting group when attached to an oxygen atom, or a sulfur protecting group when attached to a sulfur atom, or two instances of R^{a} are joined to form a substituted or unsubstituted, heterocyclic ring, or substituted or unsubstituted, heteroaryl ring;

R^{A2} is hydrogen, substituted or unsubstituted acyl, substituted or unsubstituted alkyl, substituted or unsubstituted carbocyclyl, substituted or unsubstituted heterocyclyl, or a nitrogen protecting group;

R^{A3} is hydrogen, halogen, substituted or unsubstituted C₁₋₆ alkyl, —OR^{a}, or —N(R^{a})₂;

R^{A4} is hydrogen, substituted or unsubstituted C₁₋₆ alkyl, or a nitrogen protecting group; and

R^{A5} is of the formula:

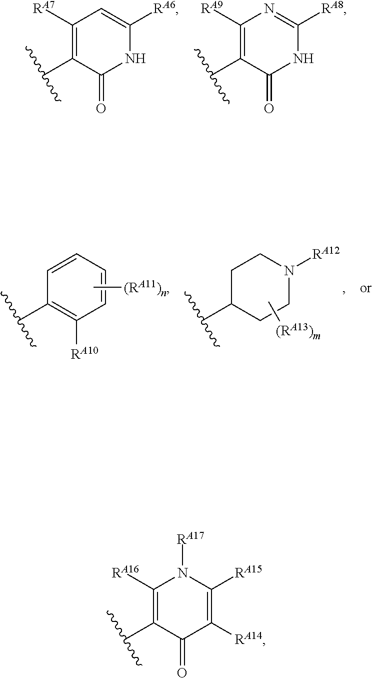

wherein:
R^{A6} is hydrogen, halogen, substituted or unsubstituted C₁₋₆ alkyl, —OR^{a}, or —N(R^{a})₂;
R^{A7} is hydrogen, halogen, substituted or unsubstituted C₂₋₆ alkyl, substituted or unsubstituted, 3- to 7-membed, monocyclic carbocyclyl comprising 0, 1, or 2 double bonds in the carbocyclic ring system, —OR^{a}, or —N(R^{a})₂;
R^{A8} is hydrogen, halogen, substituted or unsubstituted C₁₋₆ alkyl, —OR^{a}, or —N(R^{a})₂;
R^{A9} is hydrogen, halogen, substituted or unsubstituted C₁₋₆ alkyl, substituted or unsubstituted, 3- to 7-membed, monocyclic carbocyclyl comprising 0, 1, or 2 double bonds in the carbocyclic ring system, —OR^{a}, or —N(R^{a})₂;
R^{A10} is —OR^{a} or —N(R^{a})₂;
each instance of R^{A11} is independently halogen, substituted or unsubstituted C₁₋₆ alkyl, substituted or unsubstituted, 3- to 7-membed, monocyclic carbocyclyl comprising 0, 1, or 2 double bonds in the carbocyclic ring system, —OR^{a}, or —N(R^{a})₂;
n is 0, 1, 2, 3, or 4;
R^{A12} is hydrogen, substituted or unsubstituted C₁₋₆ alkyl, or a nitrogen protecting group;
each instance of R^{A13} is independently halogen, substituted or unsubstituted C₁₋₆ alkyl, substituted or unsubstituted, 3- to 7-membed, monocyclic carbocyclyl comprising 0, 1, or 2 double bonds in the carbocyclic ring system, —OR^{a}, or —N(R^{a})₂;
m is 0, 1, 2, 3, 4, 5, 6, 7, 8, or 9;
R^{A14} is hydrogen, halogen, substituted or unsubstituted C₁₋₆ alkyl, —OR^{a}, or —N(R^{a})₂;
R^{A15} is hydrogen, halogen, substituted or unsubstituted C₁₋₆ alkyl, —OR^{a}, or —N(R^{a})₂;
R^{A16} is hydrogen, halogen, substituted or unsubstituted C₂₋₆ alkyl, substituted or unsubstituted, 3- to 7-membed, monocyclic carbocyclyl comprising 0, 1, or 2 double bonds in the carbocyclic ring system, —OR^{a}, or —N(R^{a})₂; and
R^{A17} is hydrogen, substituted or unsubstituted acyl, substituted or unsubstituted C₁₋₆ alkyl, or a nitrogen protecting group.

Formula (I) includes substituent R^{A1} on the pyridinyl ring. In certain embodiments, R^{A1} is halogen (e.g., F, Cl, Br, or I). In certain embodiments, R^{A1} is substituted or unsubstituted alkyl (e.g., substituted or unsubstituted C₁₋₆ alkyl). In certain embodiments, R^{A1} is Me. In certain embodiments, R^{A1} is —CF₃, Bn, Et, perfluoroethyl, Pr, perfluoropropyl, Bu, or perfluorobutyl. In certain embodiments, R^{A1} is substituted or unsubstituted alkenyl (e.g., substituted or unsubstituted C₂₋₆ alkenyl). In certain embodiments, R^{A1} is substituted or unsubstituted alkynyl (e.g., substituted or unsubstituted C₁₋₆ alkynyl). In certain embodiments, R^{A1} is substituted or unsubstituted carbocyclyl (e.g., substituted or unsubstituted, 3- to 7-membered, monocyclic carbocyclyl comprising zero, one, or two double bonds in the carbocyclic ring system). In certain embodiments, R^{A1} is substituted or unsubstituted heterocyclyl (e.g., substituted or unsubstituted, 3- to 7-membed, monocyclic heterocyclyl comprising zero, one, or two double bonds in the heterocyclic ring system, wherein one, two, or three atoms in the heterocyclic ring system are independently nitrogen, oxygen, or sulfur). In certain embodiments, R^{A1} is substituted or unsubstituted piperazinyl. In certain embodiments, R^{A1} is of the formula:

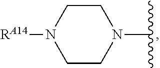

wherein R^{A14} is hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, or a nitrogen protecting group. In certain embodiments, R^{A1} is of the formula:

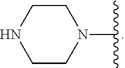 [SMILES: *N1CCNCC1]

In certain embodiments, R^{A1} is of the formula:

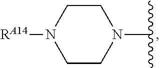

wherein R^{A14} is substituted or unsubstituted C₁₋₆ alkyl. In certain embodiments, R^{A1} is of the formula:

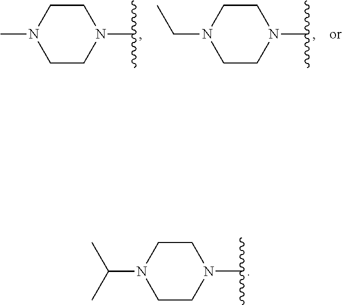

In certain embodiments, R^{A1} is of the formula:

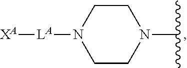

wherein L^{A} is a bond or substituted or unsubstituted C₁₋₁₀₀ hydrocarbon chain, optionally wherein one or more chain atoms of the hydrocarbon chain are independently replaced with —O—, —S—, or —NR^{a}—; and X^{A} is a small molecule, peptide, protein, or polynucleotide. In certain embodiments, R^{A1} is of the formula:

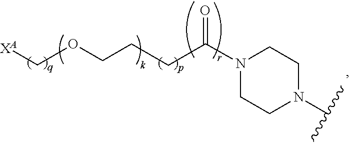

wherein r is 0 or 1, k is an integer between 0 and 11, inclusive, p is an integer between 0 and 10, inclusive, and q is an integer between 0 and 10, inclusive. In certain embodiments, k is an integer between 3 and 11, inclusive, p is 0, and q is 0, 1, 2, 3, 4, 5, or 6. In certain embodiments, X^{A} is a small molecule. In certain embodiments, X^{A} is a small molecule drug (e.g.,

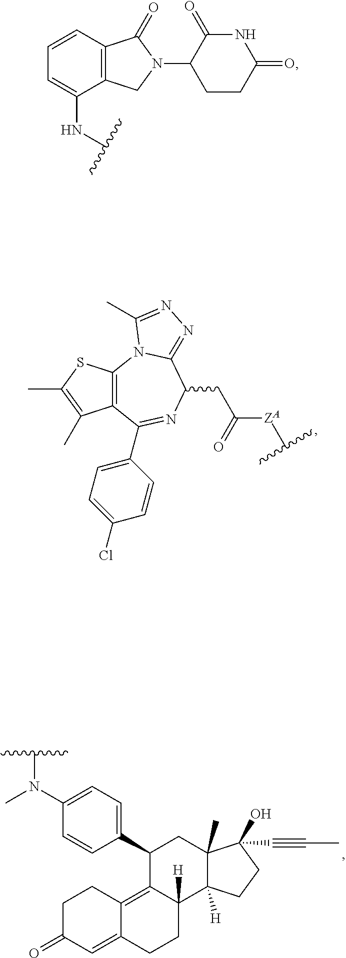

wherein Z^{A} is —O— or —NH—, or an additional pharmaceutical agent described herein that is a small molecule). In certain embodiments, X^{A} is a small molecule label (e.g., a biotin moiety (e.g.,

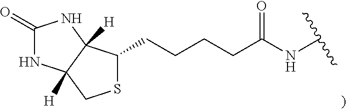 [SMILES: *NC(=O)CCCC[C@@H]1SC[C@@H]2NC(=O)N[C@@H]21]

or a small molecule fluorophore). In certain embodiments, R^{A1} is substituted or unsubstituted oxetanyl, substituted or unsubstituted tetrahydrofuranyl, substituted or unsubstituted pyrrolidinyl, substituted or unsubstituted tetrahydropyranyl, substituted or unsubstituted piperidinyl, substituted or unsubstituted morpholinyl, substituted or unsubstituted azepanyl, or substituted or unsubstituted diazepanyl. In certain embodiments, R^{A1} is of the formula:

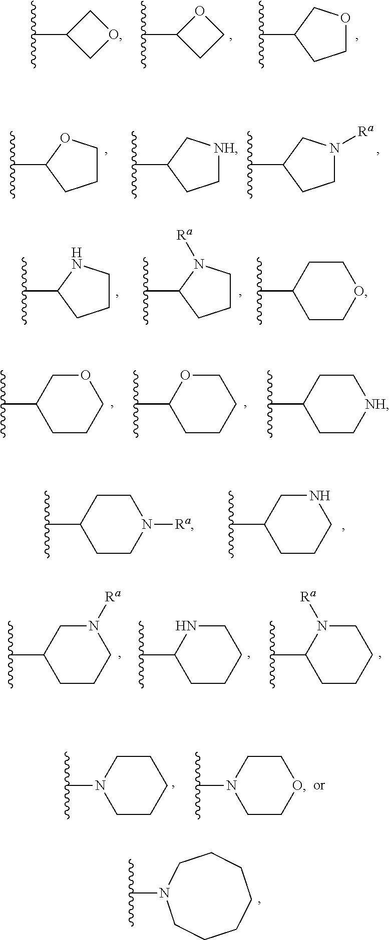

wherein each instance of R^{a} is independently unsubstituted C₁₋₆ alkyl (e.g., Me)). In certain embodiments, R^{A1} is substituted or unsubstituted aryl (e.g., substituted or unsubstituted, 6- to 10-membered aryl). In certain embodiments, R^{A1} is substituted or unsubstituted phenyl. In certain embodiments, R^{A1} is substituted or unsubstituted heteroaryl (e.g., substituted or unsubstituted, 5- to 6-membed, monocyclic heteroaryl, wherein one, two, three, or four atoms in the heteroaryl ring system are independently nitrogen, oxygen, or sulfur). In certain embodiments, R^{A1} is —OR^{a} (e.g., —OH, —O(substituted or unsubstituted C₁₋₆ alkyl) (e.g., —OMe, —OEt, —OPr, —OBu, or —OBn), or —O(substituted or unsubstituted phenyl) (e.g., —OPh)). In certain embodiments, R^{A1} is —SR^{a} (e.g., —SH, —S(substituted or unsubstituted C₁₋₆ alkyl) (e.g., —SMe, —SEt, —SPr, —SBu, or —SBn), or —S(substituted or unsubstituted phenyl) (e.g., —SPh)). In certain embodiments, R^{A1} is —N(R^{a})₂ (e.g., —NH₂, —NH(substituted or unsubstituted C₁₋₆ alkyl) (e.g., —NHMe), or —N(substituted or unsubstituted C₁₋₆ alkyl)-(substituted or unsubstituted C₁₋₆ alkyl) (e.g., —NMe₂)). In certain embodiments, R^{A1} is —CN. In certain embodiments, R^{A1} is —SCN or —NO₂. In certain embodiments, R^{A1} is —C(═NR^{a})R^{a}, —C(═NR^{a})OR^{a}, or —C(═NR^{a})N(R^{a})₂. In certain embodiments, R^{A1} is —C(═O)R^{a} (e.g., —C(═O)(substituted or unsubstituted alkyl) or —C(═O)(substituted or unsubstituted phenyl)). In certain embodiments, R^{A1} is —C(═O)OR^{a} (e.g., —C(═O)OH, —C(═O)O(substituted or unsubstituted alkyl) (e.g., —C(═O)OMe), or —C(═O)O(substituted or unsubstituted phenyl)). In certain embodiments, R^{A1} is —C(═O)N(R^{a})₂ (e.g., —C(═O)NH₂, —C(═O)NH(substituted or unsubstituted alkyl), —C(═O)NH(substituted or unsubstituted phenyl), —C(═O)N(substituted or unsubstituted alkyl)-(substituted or unsubstituted alkyl), or —C(═O)N(substituted or unsubstituted phenyl)-(substituted or unsubstituted alkyl)). In certain embodiments, R^{A1} is —NR^{a}C(═O)R^{a} (e.g., —NHC(═O)Me). In certain embodiments, R^{A1} is —NR^{a}C(═O)OR^{a} or —NR^{a}C(═O)N(R^{a})₂. In certain embodiments, R^{A1} is —OC(═O)R^{a}, —OC(═O)OR^{a}, or —OC(═O)N(R^{a})₂. In certain embodiments, R^{A1} is substituted or unsubstituted alkyl, —OR, —N(R^{a})₂, —C(═O)OR^{a}, or —NR^{a}C(═O)R^{a}. In certain embodiments, R^{A1} is unsubstituted C₁₋₆ alkyl, —OMe, —NH₂, —N(Me)₂, —C(═O)OH, —C(═O)OMe, or —NHC(═O)Me. In certain embodiments, R^{A1} is

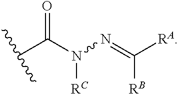

A compound described herein that includes a moiety of the formula:

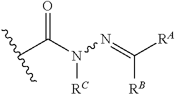

is a hydrazide.

The moiety

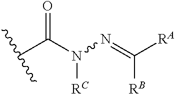

of any one of Formulae (I) and (II) includes substituents R^{A}, R^{B}, and R^{C}. In certain embodiments, R^{A} is H. In certain embodiments, R^{A} is substituted acyl. In certain embodiments, R^{A} is unsubstituted acyl. In certain embodiments, R^{A} is acetyl. In certain embodiments, R^{A} is —C(═O)R^{c} (e.g., —C(═O)(substituted or unsubstituted alkyl)). In certain embodiments, R^{A} is —C(═O)OR^{c} (e.g., —C(═O)O(substituted or unsubstituted alkyl) or —C(═O)OH). In certain embodiments, R^{A} is —C(═O)N(R^{c})₂, (e.g., —C(═O)NH₂, —C(═O)NH(substituted or unsubstituted alkyl), or —C(═O)N(substituted or unsubstituted alkyl)₂). In certain embodiments, R^{A} is unsubstituted alkyl. In certain embodiments, R^{A} is substituted alkyl. In certain embodiments, R^{A} is unsubstituted C₁₋₆ alkyl. In certain embodiments, R^{A} is substituted C₁₋₆ alkyl. In certain embodiments, R^{A} is C₁₋₆ alkyl substituted with at least one halogen. In certain embodiments, R^{A} is —CH₃. In certain embodiments, R^{A} is substituted methyl. In certain embodiments, R^{A} is —CH₂F, —CHF₂, or —CF₃. In certain embodiments, R^{A} is Et, substituted ethyl, Pr, substituted propyl, Bu, or substituted butyl. In certain embodiments, R^{A} is unsubstituted alkenyl. In certain embodiments, R^{A} is substituted alkenyl. In certain embodiments, R^{A} is unsubstituted C₁₋₆ alkenyl. In certain embodiments, R^{A} is substituted C₁₋₆ alkenyl. In certain embodiments, R^{A} is unsubstituted alkynyl. In certain embodiments, R^{A} is substituted alkynyl. In certain embodiments, R^{A} is unsubstituted C₁₋₆ alkynyl. In certain embodiments, R^{A} is substituted C₁₋₆ alkynyl. In certain embodiments, R^{A} is substituted carbocyclyl. In certain embodiments, R^{A} is unsubstituted carbocyclyl. In certain embodiments, R^{A} is saturated carbocyclyl. In certain embodiments, R^{A} is unsaturated carbocyclyl. In certain embodiments, R^{A} is 3- to 8-membered, monocyclic carbocyclyl, optionally including 1, 2, or 3 double bonds in the carbocyclic ring system. In certain embodiments, R^{A} is 5- to 14-membered, bicyclic carbocyclyl, optionally including 1, 2, 3, or 4 double bonds in the carbocyclic ring system. In certain embodiments, R^{A} is 5- to 20-membered, tricyclic carbocyclyl, optionally including 1, 2, 3, 4, or 5 double bonds in the carbocyclic ring system. In certain embodiments, R^{A} is 5- to 26-membered, tetracyclic carbocyclyl, optionally including 1, 2, 3, 4, 5, or 6 double bonds in the carbocyclic ring system. In certain embodiments, R^{A} is substituted heterocyclyl. In certain embodiments, R^{A} is unsubstituted heterocyclyl. In certain embodiments, R^{A} is saturated heterocyclyl. In certain embodiments, R^{A} is unsaturated heterocyclyl. In certain embodiments, R^{A} is 3- to 8-membered, monocyclic heterocyclyl, optionally including 1 or 2 double bonds in the heterocyclic ring system, wherein 1, 2, or 3 atoms in the heterocyclic ring system are independently nitrogen, oxygen, or sulfur. In certain embodiments, R^{A} is 5- to 14-membered, bicyclic heterocyclyl, optionally including 1, 2, or 3 double bonds in the heterocyclic ring system, wherein 1, 2, 3, or 4 atoms in the heterocyclic ring system are independently nitrogen, oxygen, or sulfur. In certain embodiments, R^{A} is 5- to 20-membered, tricyclic heterocyclyl, optionally including 1, 2, 3, or 4 double bonds in the heterocyclic ring system, wherein 1, 2, 3, 4, or 5 atoms in the heterocyclic ring system are independently nitrogen, oxygen, or sulfur. In certain embodiments, R^{A} is substituted aryl. In certain embodiments, R^{A} is unsubstituted aryl. In certain embodiments, R^{A} is 6- to 14-membered aryl. In certain embodiments, R^{A} is 6- to 10-membered aryl. In certain embodiments, R^{A} is substituted phenyl. In certain embodiments, R^{A} is unsubstituted phenyl. In certain embodiments, R^{A} is substituted naphthyl. In certain embodiments, R^{A} is unsubstituted naphthyl. In certain embodiments, R^{A} is substituted heteroaryl. In certain embodiments, R^{A} is unsubstituted heteroaryl. In certain embodiments, R^{A} is 5- to 6-membered, monocyclic heteroaryl, wherein 1, 2, 3, or 4 atoms in the heteroaryl ring system are independently nitrogen, oxygen, or sulfur. In certain embodiments, R^{A} is 8- to 10-membered, bicyclic heteroaryl, wherein 1, 2, 3, or 4 atoms in the heteroaryl ring system are independently nitrogen, oxygen, or sulfur.

In certain embodiments, R^{A} is a moiety shown in Table 1A. In certain embodiments, R^{A} is a moiety shown in Table 1B.

TABLE 1A
Exemplary R^{A} moieties

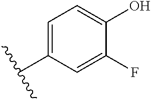 [SMILES: *c1ccc(O)c(F)c1]

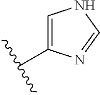 [SMILES: *c1c[nH]cn1]

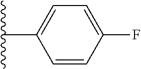 [SMILES: *c1ccc(F)cc1]

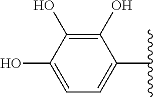 [SMILES: *c1ccc(O)c(O)c1O]

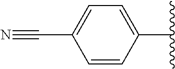 [SMILES: *c1ccc(C#N)cc1]

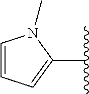 [SMILES: *c1cccn1C]

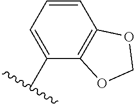 [SMILES: *c1cccc2c1OCO2]

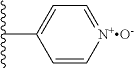

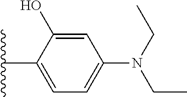 [SMILES: *c1ccc(N(CC)CC)cc1O]

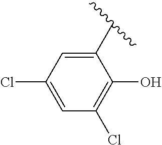 [SMILES: *c1cc(Cl)cc(Cl)c1O]

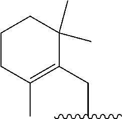 [SMILES: *CC1=C(C)CCCC1(C)C]

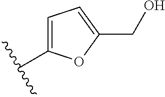 [SMILES: *c1ccc(CO)o1]

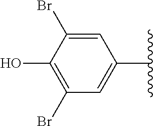 [SMILES: *c1cc(Br)c(O)c(Br)c1]

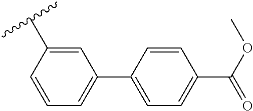 [SMILES: *c1cccc(-c2ccc(C(=O)OC)cc2)c1]

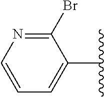 [SMILES: *c1cccnc1Br]

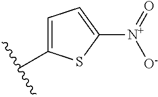 [SMILES: *c1ccc([N+](=O)[O-])s1]

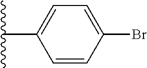 [SMILES: *c1ccc(Br)cc1]

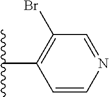 [SMILES: *c1ccncc1Br]

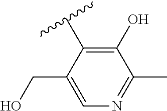 [SMILES: *c1c(CO)cnc(C)c1O]

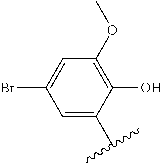 [SMILES: *c1cc(Br)cc(OC)c1O]

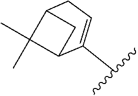 [SMILES: *C1=CCC2CC1C2(C)C]

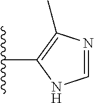 [SMILES: *c1[nH]cnc1C]

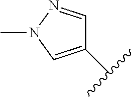 [SMILES: *c1cnn(C)c1]

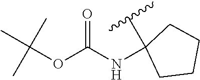 [SMILES: *C1(NC(=O)OC(C)(C)C)CCCC1]

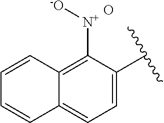

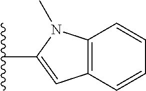 [SMILES: *c1cc2ccccc2n1C]

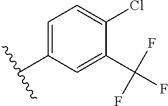 [SMILES: *c1ccc(Cl)c(C(F)(F)F)c1]

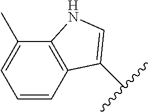 [SMILES: *c1c[nH]c2c(C)cccc12]

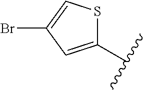 [SMILES: *c1cc(Br)cs1]

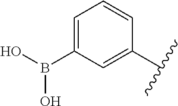 [SMILES: *c1cccc(B(O)O)c1]

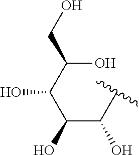 [SMILES: *[C@H](O)[C@@H](O)[C@H](O)[C@H](O)CO]

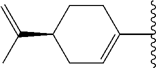 [SMILES: *C1=CC[C@@H](C(=C)C)CC1]

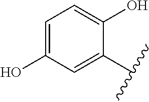 [SMILES: *c1cc(O)ccc1O]

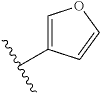 [SMILES: *c1ccoc1]

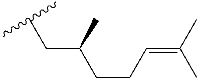 [SMILES: *C[C@@H](C)CCC=C(C)C]

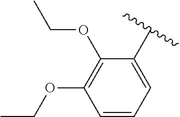 [SMILES: *c1cccc(OCC)c1OCC]

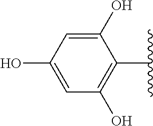 [SMILES: *c1c(O)cc(O)cc1O]

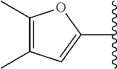 [SMILES: *c1cc(C)c(C)o1]

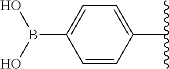 [SMILES: *c1ccc(B(O)O)cc1]

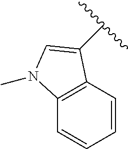 [SMILES: *c1cn(C)c2ccccc12]

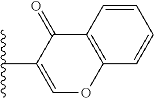 [SMILES: *c1coc2ccccc2c1=O]

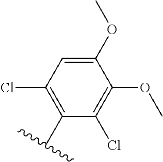 [SMILES: *c1c(Cl)cc(OC)c(OC)c1Cl]

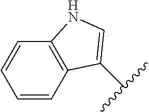 [SMILES: *c1c[nH]c2ccccc12]

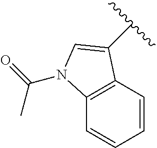 [SMILES: *c1cn(C(C)=O)c2ccccc12]

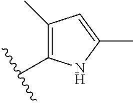 [SMILES: *c1[nH]c(C)cc1C]

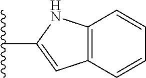 [SMILES: *c1cc2ccccc2[nH]1]

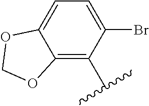 [SMILES: *c1c(Br)ccc2c1OCO2]

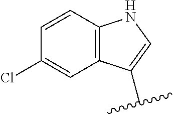 [SMILES: *c1c[nH]c2ccc(Cl)cc12]

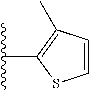 [SMILES: *c1sccc1C]

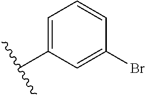 [SMILES: *c1cccc(Br)c1]

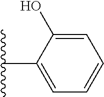 [SMILES: *c1ccccc1O]

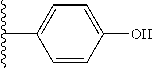 [SMILES: *c1ccc(O)cc1]

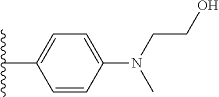 [SMILES: *c1ccc(N(C)CCO)cc1]

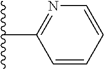 [SMILES: *c1ccccn1]

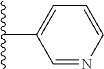 [SMILES: *c1cccnc1]

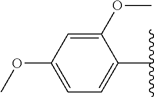

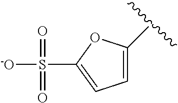

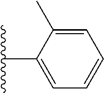 [SMILES: *c1ccccc1C]

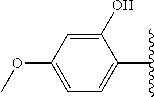 [SMILES: *c1ccc(OC)cc1O]

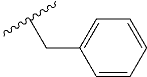 [SMILES: *Cc1ccccc1]

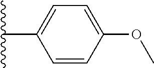 [SMILES: *c1ccc(OC)cc1]

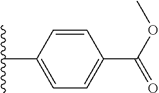 [SMILES: *c1ccc(C(=O)OC)cc1]

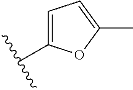 [SMILES: *c1ccc(C)o1]

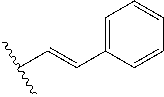 [SMILES: */C=C/c1ccccc1]

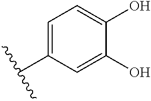 [SMILES: *c1ccc(O)c(O)c1]

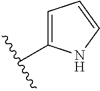 [SMILES: *c1ccc[nH]1]

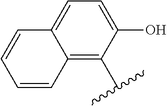 [SMILES: *c1c(O)ccc2ccccc12]

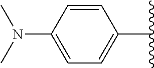 [SMILES: *c1ccc(N(C)C)cc1]

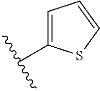 [SMILES: *c1cccs1]

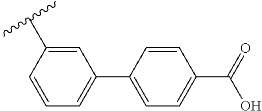 [SMILES: *c1cccc(-c2ccc(C(=O)O)cc2)c1]

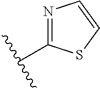 [SMILES: *c1nccs1]

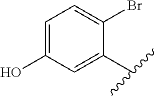 [SMILES: *c1cc(O)ccc1Br]

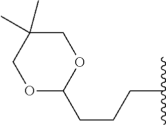 [SMILES: *CCCC1OCC(C)(C)CO1]

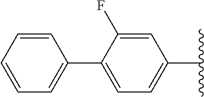 [SMILES: *c1ccc(-c2ccccc2)c(F)c1]

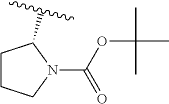 [SMILES: *[C@H]1CCCN1C(=O)OC(C)(C)C]

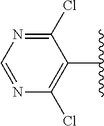 [SMILES: *c1c(Cl)ncnc1Cl]

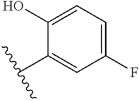 [SMILES: *c1cc(F)ccc1O]

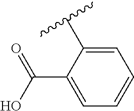 [SMILES: *c1ccccc1C(=O)O]

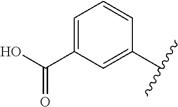 [SMILES: *c1cccc(C(=O)O)c1]

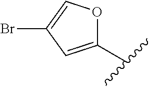 [SMILES: *c1cc(Br)co1]

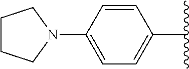 [SMILES: *c1ccc(N2CCCC2)cc1]

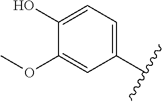 [SMILES: *c1ccc(O)c(OC)c1]

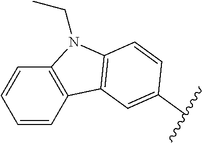 [SMILES: *c1ccc2c(c1)c1ccccc1n2CC]

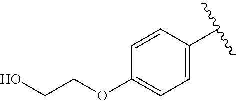 [SMILES: *c1ccc(OCCO)cc1]

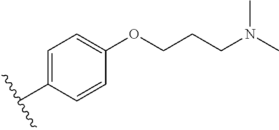 [SMILES: *c1ccc(OCCCN(C)C)cc1]

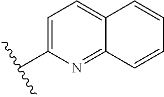 [SMILES: *c1ccc2ccccc2n1]

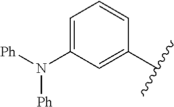 [SMILES: *c1cccc(N(c2ccccc2)c2ccccc2)c1]

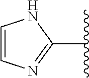 [SMILES: *c1ncc[nH]1]

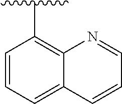 [SMILES: *c1cccc2cccnc12]

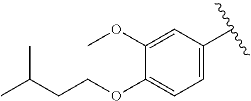 [SMILES: *c1ccc(OCCC(C)C)c(OC)c1]

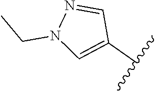 [SMILES: *c1cnn(CC)c1]

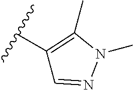 [SMILES: *c1cnn(C)c1C]

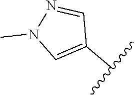 [SMILES: *c1cnn(C)c1]

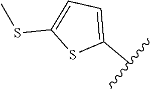 [SMILES: *c1ccc(SC)s1]

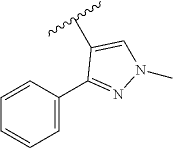 [SMILES: *c1cn(C)nc1-c1ccccc1]

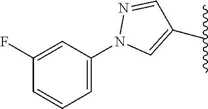 [SMILES: *c1cnn(-c2cccc(F)c2)c1]

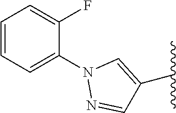 [SMILES: *c1cnn(-c2ccccc2F)c1]

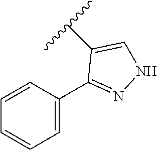 [SMILES: *c1c[nH]nc1-c1ccccc1]

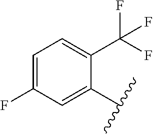 [SMILES: *c1cc(F)ccc1C(F)(F)F]

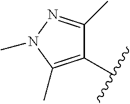 [SMILES: *c1c(C)nn(C)c1C]

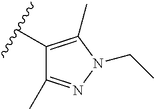 [SMILES: *c1c(C)nn(CC)c1C]

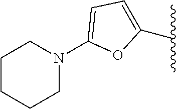 [SMILES: *c1ccc(N2CCCCC2)o1]

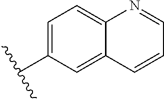 [SMILES: *c1ccc2ncccc2c1]

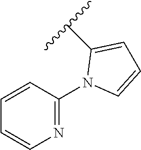 [SMILES: *c1cccn1-c1ccccn1]

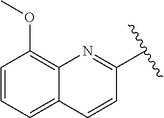 [SMILES: *c1ccc2cccc(OC)c2n1]

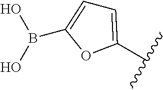 [SMILES: *c1ccc(B(O)O)o1]

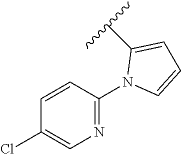 [SMILES: *c1cccn1-c1ccc(Cl)cn1]

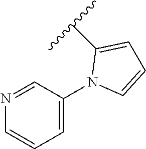 [SMILES: *c1cccn1-c1cccnc1]

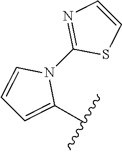 [SMILES: *c1cccn1-c1nccs1]

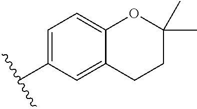 [SMILES: *c1ccc2c(c1)CCC(C)(C)O2]

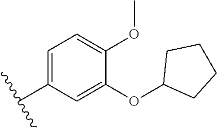 [SMILES: *c1ccc(OC)c(OC2CCCC2)c1]

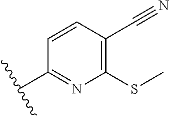 [SMILES: *c1ccc(C#N)c(SC)n1]

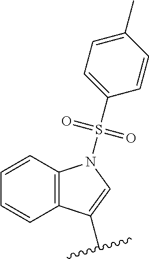 [SMILES: *c1cn(S(=O)(=O)c2ccc(C)cc2)c2ccccc12]

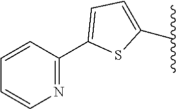 [SMILES: *c1ccc(-c2ccccn2)s1]

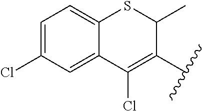 [SMILES: *C1=C(Cl)c2cc(Cl)ccc2SC1C]

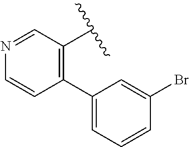 [SMILES: *c1cnccc1-c1cccc(Br)c1]

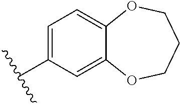 [SMILES: *c1ccc2c(c1)OCCCO2]

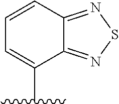 [SMILES: *c1cccc2nsnc12]

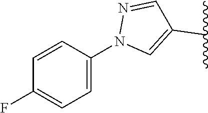 [SMILES: *c1cnn(-c2ccc(F)cc2)c1]

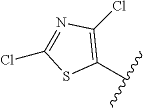 [SMILES: *c1sc(Cl)nc1Cl]

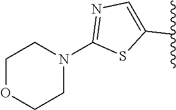 [SMILES: *c1cnc(N2CCOCC2)s1]

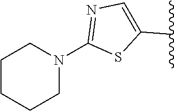 [SMILES: *c1cnc(N2CCCCC2)s1]

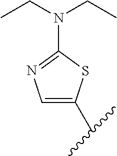 [SMILES: *c1cnc(N(CC)CC)s1]

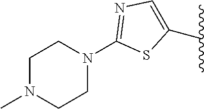 [SMILES: *c1cnc(N2CCN(C)CC2)s1]

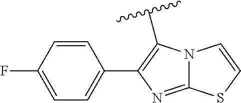 [SMILES: *c1c(-c2ccc(F)cc2)nc2sccn12]

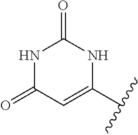 [SMILES: *c1cc(=O)[nH]c(=O)[nH]1]

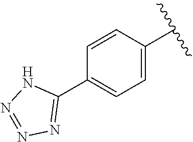 [SMILES: *c1ccc(-c2nnn[nH]2)cc1]

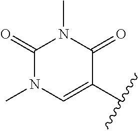 [SMILES: *c1cn(C)c(=O)n(C)c1=O]

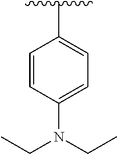 [SMILES: *c1ccc(N(CC)CC)cc1]

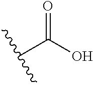 [SMILES: *C(=O)O]

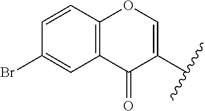 [SMILES: *c1coc2ccc(Br)cc2c1=O]

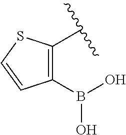 [SMILES: *c1sccc1B(O)O]

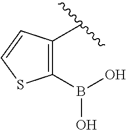 [SMILES: *c1ccsc1B(O)O]

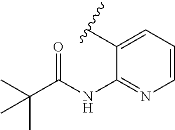 [SMILES: *c1cccnc1NC(=O)C(C)(C)C]

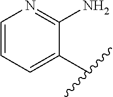 [SMILES: *c1cccnc1N]

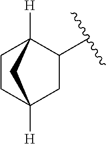 [SMILES: *C1C[C@@H]2CC[C@H]1C2]

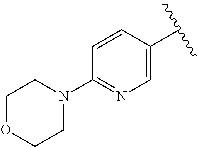 [SMILES: *c1ccc(N2CCOCC2)nc1]

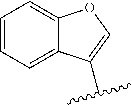 [SMILES: *c1coc2ccccc12]

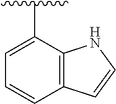 [SMILES: *c1cccc2cc[nH]c12]

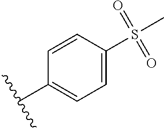 [SMILES: *c1ccc(S(C)(=O)=O)cc1]

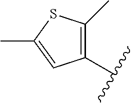 [SMILES: *c1cc(C)sc1C]

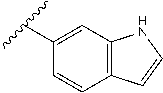 [SMILES: *c1ccc2cc[nH]c2c1]

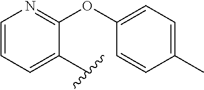 [SMILES: *c1cccnc1Oc1ccc(C)cc1]

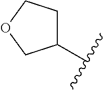 [SMILES: *C1CCOC1]

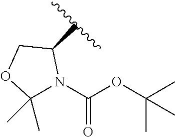 [SMILES: *[C@@H]1COC(C)(C)N1C(=O)OC(C)(C)C]

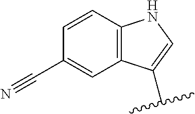 [SMILES: *c1c[nH]c2ccc(C#N)cc12]

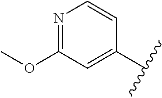 [SMILES: *c1ccnc(OC)c1]

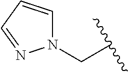 [SMILES: *Cn1cccn1]

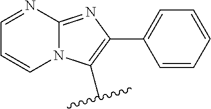 [SMILES: *c1c(-c2ccccc2)nc2ncccn12]

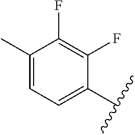 [SMILES: *c1ccc(C)c(F)c1F]

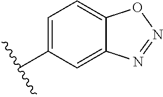 [SMILES: *c1ccc2onnc2c1]

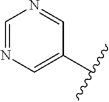 [SMILES: *c1cncnc1]

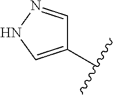 [SMILES: *c1cn[nH]c1]

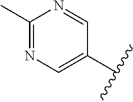 [SMILES: *c1cnc(C)nc1]

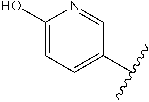 [SMILES: *c1ccc(O)nc1]

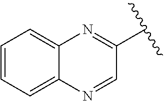 [SMILES: *c1cnc2ccccc2n1]

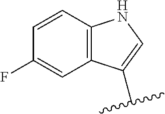 [SMILES: *c1c[nH]c2ccc(F)cc12]

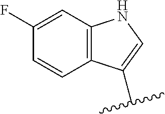 [SMILES: *c1c[nH]c2cc(F)ccc12]

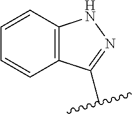 [SMILES: *c1n[nH]c2ccccc12]

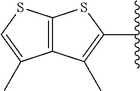 [SMILES: *c1sc2scc(C)c2c1C]

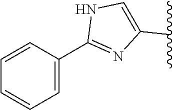 [SMILES: *c1c[nH]c(-c2ccccc2)n1]

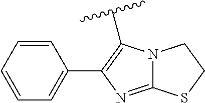 [SMILES: *c1c(-c2ccccc2)nc2n1CCS2]

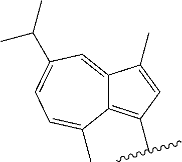 [SMILES: *c1cc(C)c2cc(C(C)C)ccc(C)c1-2]

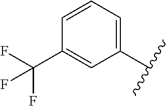 [SMILES: *c1cccc(C(F)(F)F)c1]

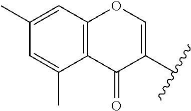 [SMILES: *c1coc2cc(C)cc(C)c2c1=O]

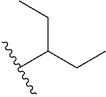 [SMILES: *C(CC)CC]

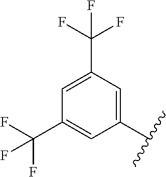 [SMILES: *c1cc(C(F)(F)F)cc(C(F)(F)F)c1]

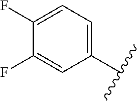 [SMILES: *c1ccc(F)c(F)c1]

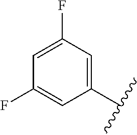 [SMILES: *c1cc(F)cc(F)c1]

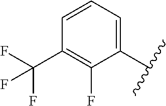 [SMILES: *c1cccc(C(F)(F)F)c1F]

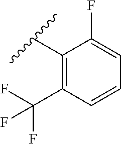 [SMILES: *c1c(F)cccc1C(F)(F)F]

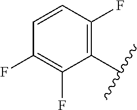 [SMILES: *c1c(F)ccc(F)c1F]

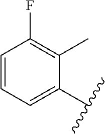 [SMILES: *c1cccc(F)c1C]

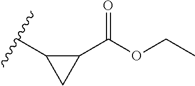 [SMILES: *C1CC1C(=O)OCC]

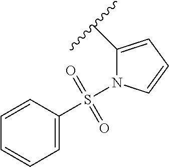 [SMILES: *c1cccn1S(=O)(=O)c1ccccc1]

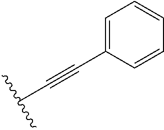 [SMILES: *C#Cc1ccccc1]

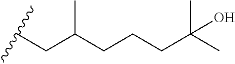 [SMILES: *CC(C)CCCC(C)(C)O]

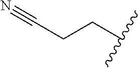 [SMILES: *CCC#N]

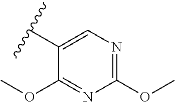

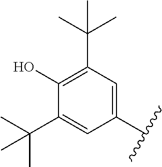 [SMILES: *c1cc(C(C)(C)C)c(O)c(C(C)(C)C)c1]

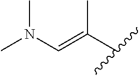 [SMILES: */C(C)=C/N(C)C]

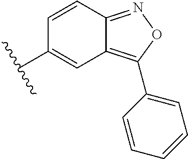 [SMILES: *c1ccc2noc(-c3ccccc3)c2c1]

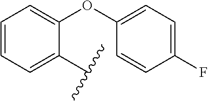 [SMILES: *c1ccccc1Oc1ccc(F)cc1]

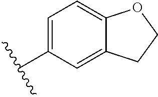 [SMILES: *c1ccc2c(c1)CCO2]

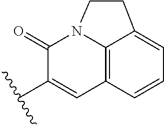 [SMILES: *c1cc2cccc3c2n(c1=O)CC3]

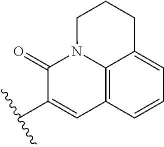 [SMILES: *c1cc2cccc3c2n(c1=O)CCC3]

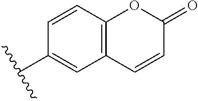 [SMILES: *c1ccc2oc(=O)ccc2c1]

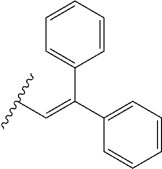 [SMILES: *C=C(c1ccccc1)c1ccccc1]

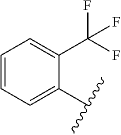 [SMILES: *c1ccccc1C(F)(F)F]

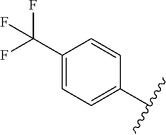 [SMILES: *c1ccc(C(F)(F)F)cc1]

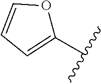 [SMILES: *c1ccco1]

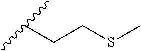 [SMILES: *CCSC]

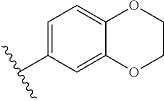 [SMILES: *c1ccc2c(c1)OCCO2]

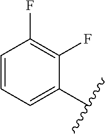 [SMILES: *c1cccc(F)c1F]

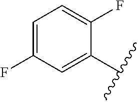 [SMILES: *c1cc(F)ccc1F]

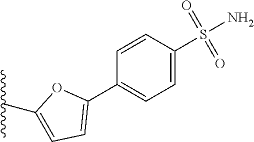 [SMILES: *c1ccc(-c2ccc(S(N)(=O)=O)cc2)o1]

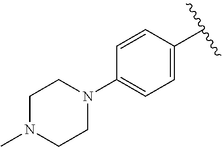 [SMILES: *c1ccc(N2CCN(C)CC2)cc1]

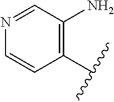 [SMILES: *c1ccncc1N]

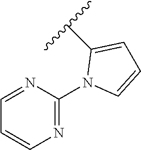 [SMILES: *c1cccn1-c1ncccn1]

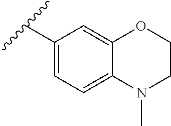 [SMILES: *c1ccc2c(c1)OCCN2C]

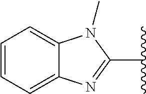 [SMILES: *c1nc2ccccc2n1C]

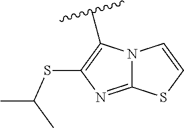 [SMILES: *c1c(SC(C)C)nc2sccn12]

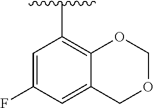 [SMILES: *c1cc(F)cc2c1OCOC2]

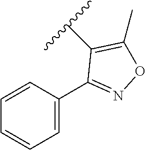 [SMILES: *c1c(-c2ccccc2)noc1C]

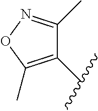 [SMILES: *c1c(C)noc1C]

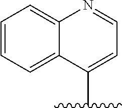 [SMILES: *c1ccnc2ccccc12]

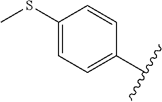 [SMILES: *c1ccc(SC)cc1]

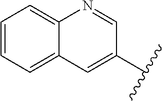 [SMILES: *c1cnc2ccccc2c1]

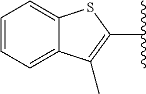 [SMILES: *c1sc2ccccc2c1C]

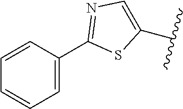 [SMILES: *c1cnc(-c2ccccc2)s1]

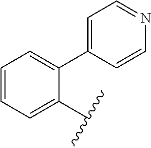 [SMILES: *c1ccccc1-c1ccncc1]

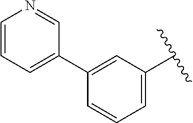 [SMILES: *c1cccc(-c2cccnc2)c1]

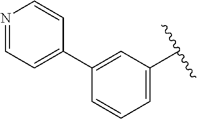 [SMILES: *c1cccc(-c2ccncc2)c1]

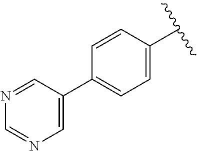 [SMILES: *c1ccc(-c2cncnc2)cc1]

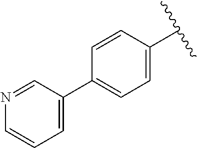 [SMILES: *c1ccc(-c2cccnc2)cc1]

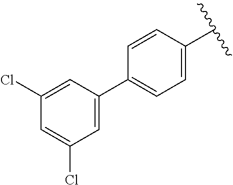 [SMILES: *c1ccc(-c2cc(Cl)cc(Cl)c2)cc1]

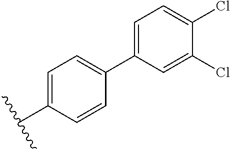 [SMILES: *c1ccc(-c2ccc(Cl)c(Cl)c2)cc1]

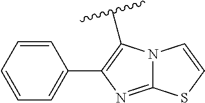 [SMILES: *c1c(-c2ccccc2)nc2sccn12]

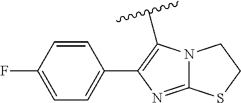 [SMILES: *c1c(-c2ccc(F)cc2)nc2n1CCS2]

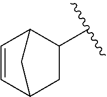 [SMILES: *C1CC2C=CC1C2]

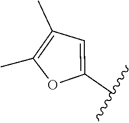 [SMILES: *c1cc(C)c(C)o1]

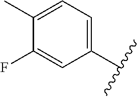 [SMILES: *c1ccc(C)c(F)c1]

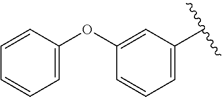 [SMILES: *c1cccc(Oc2ccccc2)c1]

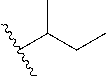 [SMILES: *C(C)CC]

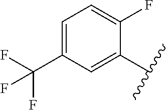 [SMILES: *c1cc(C(F)(F)F)ccc1F]

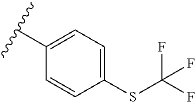 [SMILES: *c1ccc(SC(F)(F)F)cc1]

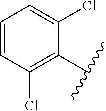 [SMILES: *c1c(Cl)cccc1Cl]

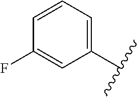 [SMILES: *c1cccc(F)c1]

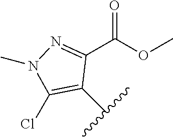

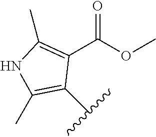

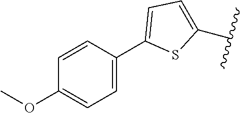 [SMILES: *c1ccc(-c2ccc(OC)cc2)s1]

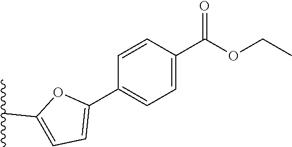 [SMILES: *c1ccc(-c2ccc(C(=O)OCC)cc2)o1]

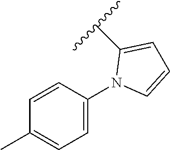 [SMILES: *c1cccn1-c1ccc(C)cc1]

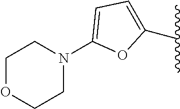 [SMILES: *c1ccc(N2CCOCC2)o1]

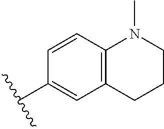 [SMILES: *c1ccc2c(c1)CCCN2C]

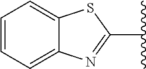 [SMILES: *c1nc2ccccc2s1]

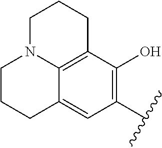 [SMILES: *c1cc2c3c(c1O)CCCN3CCC2]

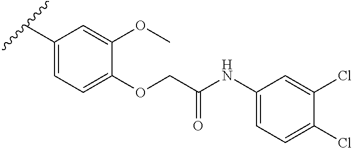 [SMILES: *c1ccc(OCC(=O)Nc2ccc(Cl)c(Cl)c2)c(OC)c1]

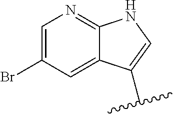 [SMILES: *c1c[nH]c2ncc(Br)cc12]

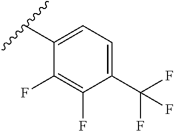 [SMILES: *c1ccc(C(F)(F)F)c(F)c1F]

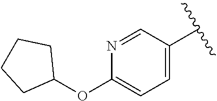 [SMILES: *c1ccc(OC2CCCC2)nc1]

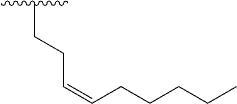 [SMILES: *CC/C=C\CCCCC]

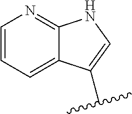 [SMILES: *c1c[nH]c2ncccc12]

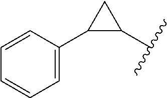 [SMILES: *C1CC1c1ccccc1]

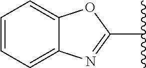 [SMILES: *c1nc2ccccc2o1]

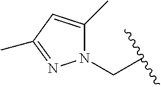 [SMILES: *Cn1nc(C)cc1C]

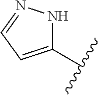 [SMILES: *c1ccn[nH]1]

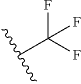 [SMILES: *C(F)(F)F]

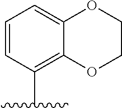 [SMILES: *c1cccc2c1OCCO2]

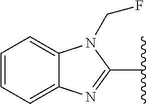 [SMILES: *c1nc2ccccc2n1CF]

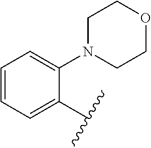 [SMILES: *c1ccccc1N1CCOCC1]

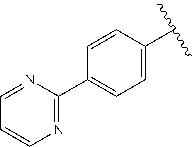 [SMILES: *c1ccc(-c2ncccn2)cc1]

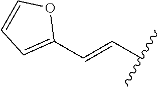 [SMILES: */C=C/c1ccco1]

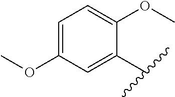

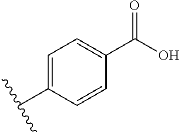 [SMILES: *c1ccc(C(=O)O)cc1]

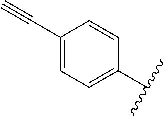 [SMILES: *c1ccc(C#C)cc1]

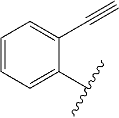 [SMILES: *c1ccccc1C#C]

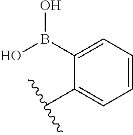 [SMILES: *c1ccccc1B(O)O]

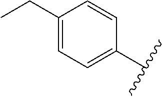 [SMILES: *c1ccc(CC)cc1]

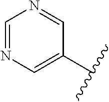 [SMILES: *c1cncnc1]

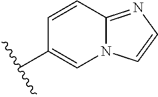 [SMILES: *c1ccc2nccn2c1]

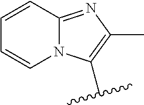 [SMILES: *c1c(C)nc2ccccn12]

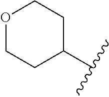 [SMILES: *C1CCOCC1]

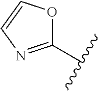 [SMILES: *c1ncco1]

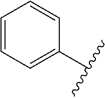 [SMILES: *c1ccccc1]

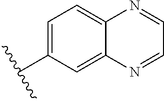 [SMILES: *c1ccc2nccnc2c1]

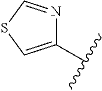 [SMILES: *c1cscn1]

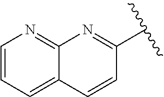 [SMILES: *c1ccc2cccnc2n1]

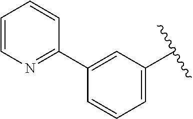 [SMILES: *c1cccc(-c2ccccn2)c1]

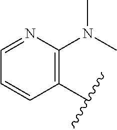 [SMILES: *c1cccnc1N(C)C]

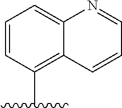 [SMILES: *c1cccc2ncccc12]

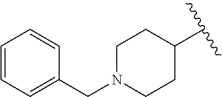 [SMILES: *C1CCN(Cc2ccccc2)CC1]

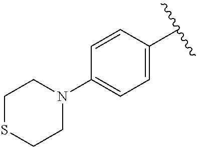 [SMILES: *c1ccc(N2CCSCC2)cc1]

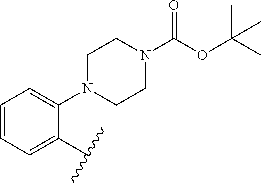 [SMILES: *c1ccccc1N1CCN(C(=O)OC(C)(C)C)CC1]

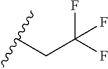 [SMILES: *CC(F)(F)F]

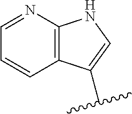 [SMILES: *c1c[nH]c2ncccc12]

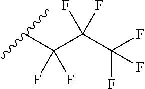 [SMILES: *C(F)(F)C(F)(F)C(F)(F)F]

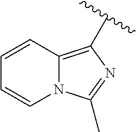 [SMILES: *c1nc(C)n2ccccc12]

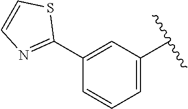 [SMILES: *c1cccc(-c2nccs2)c1]

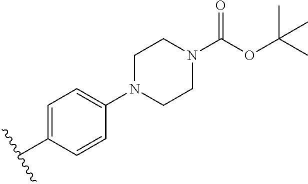 [SMILES: *c1ccc(N2CCN(C(=O)OC(C)(C)C)CC2)cc1]

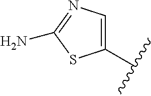 [SMILES: *c1cnc(N)s1]

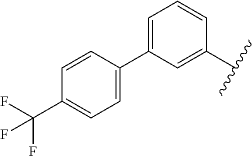 [SMILES: *c1cccc(-c2ccc(C(F)(F)F)cc2)c1]

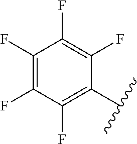 [SMILES: *c1c(F)c(F)c(F)c(F)c1F]

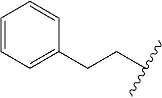 [SMILES: *CCc1ccccc1]

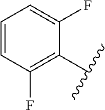 [SMILES: *c1c(F)cccc1F]

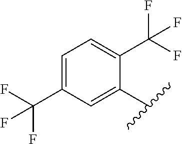 [SMILES: *c1cc(C(F)(F)F)ccc1C(F)(F)F]

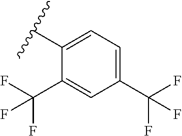 [SMILES: *c1ccc(C(F)(F)F)cc1C(F)(F)F]

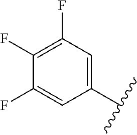 [SMILES: *c1cc(F)c(F)c(F)c1]

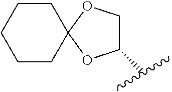 [SMILES: *[C@H]1COC2(CCCCC2)O1]

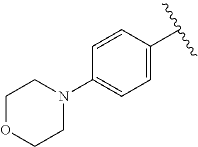 [SMILES: *c1ccc(N2CCOCC2)cc1]

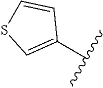 [SMILES: *c1ccsc1]

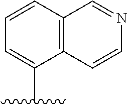 [SMILES: *c1cccc2cnccc12]

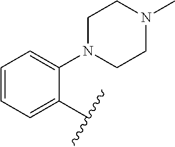 [SMILES: *c1ccccc1N1CCN(C)CC1]

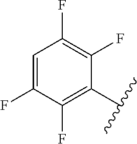 [SMILES: *c1c(F)c(F)cc(F)c1F]

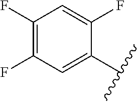 [SMILES: *c1cc(F)c(F)cc1F]

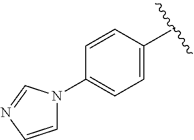

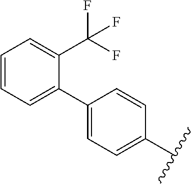 [SMILES: *c1ccc(-c2ccccc2C(F)(F)F)cc1]

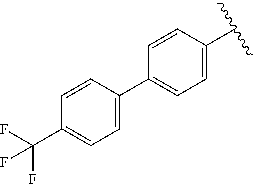 [SMILES: *c1ccc(-c2ccc(C(F)(F)F)cc2)cc1]

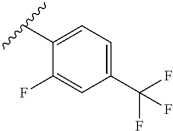 [SMILES: *c1ccc(C(F)(F)F)cc1F]

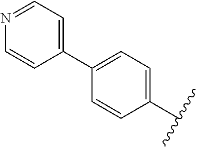 [SMILES: *c1ccc(-c2ccncc2)cc1]

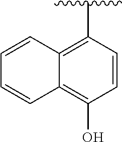 [SMILES: *c1ccc(O)c2ccccc12]

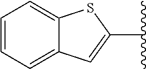 [SMILES: *c1cc2ccccc2s1]

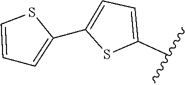 [SMILES: *c1ccc(-c2cccs2)s1]

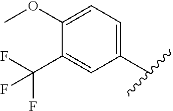 [SMILES: *c1ccc(OC)c(C(F)(F)F)c1]

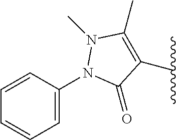 [SMILES: *c1c(C)n(C)n(-c2ccccc2)c1=O]

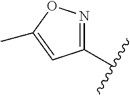 [SMILES: *c1cc(C)on1]

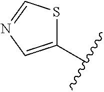 [SMILES: *c1cncs1]

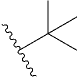 [SMILES: *C(C)(C)C]

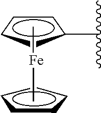

TABLE 1B
Exemplary R^{A} and R^{B} moieties

| R^{A} or R^{B} | R^{B} or R^{A} |
|---|---|
| 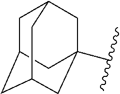 [SMILES: *C12CC3CC(CC(C3)C1)C2] | 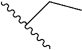 [SMILES: *CC] |
| 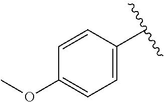 [SMILES: *c1ccc(OC)cc1] | 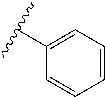 [SMILES: *c1ccccc1] |
| 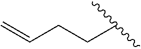 [SMILES: *CCC=C] | 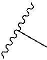 |
| 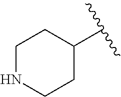 [SMILES: *C1CCNCC1] | 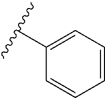 [SMILES: *c1ccccc1] |
| 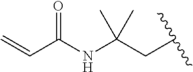 [SMILES: *CC(C)(C)NC(=O)C=C] | 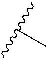 |
| 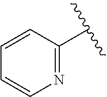 [SMILES: *c1ccccn1] | 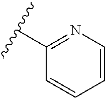 [SMILES: *c1ccccn1] |
| 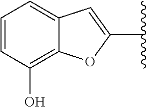 [SMILES: *c1cc2cccc(O)c2o1] | 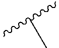 |
| 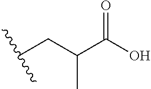 [SMILES: *CC(C)C(=O)O] | 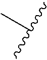 |
| 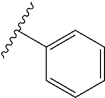 [SMILES: *c1ccccc1] | 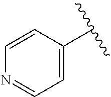 [SMILES: *c1ccncc1] |
| 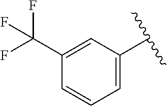 [SMILES: *c1cccc(C(F)(F)F)c1] | 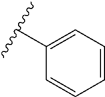 [SMILES: *c1ccccc1] |
| 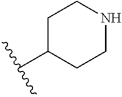 [SMILES: *C1CCNCC1] | 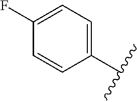 [SMILES: *c1ccc(F)cc1] |
| 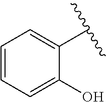 [SMILES: *c1ccccc1O] | 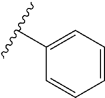 [SMILES: *c1ccccc1] |
| 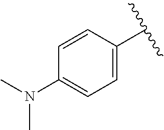 [SMILES: *c1ccc(N(C)C)cc1] | 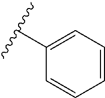 [SMILES: *c1ccccc1] |
| 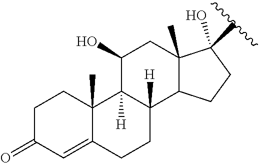 | 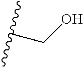 [SMILES: *CO] |
| 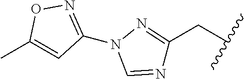 [SMILES: *Cc1ncn(-c2cc(C)on2)n1] | 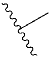 |

An R^{A} group may independently include one or more substituents R^{c}. In certain embodiments, all instances of R^{c} are the same. In certain embodiments, two instances of R^{c} are different from each other. In certain embodiments, at least one instance of R^{c} is H. In certain embodiments, each instance of R^{c} is H. In certain embodiments, at least one instance of R^{c} is substituted or unsubstituted acyl (e.g., acetyl). In certain embodiments, at least one instance of R^{c} is substituted or unsubstituted alkyl (e.g., substituted or unsubstituted C₁₋₆ alkyl). In certain embodiments, at least one instance of R^{c} is —CH₃. In certain embodiments, at least one instance of R^{c} is —CF₃, unsubstituted ethyl, perfluoroethyl, unsubstituted propyl, perfluoropropyl, unsubstituted butyl, or perfluorobutyl. In certain embodiments, at least one instance of R^{c} is substituted or unsubstituted alkenyl (e.g., substituted or unsubstituted C₁₋₆ alkenyl). In certain embodiments, at least one instance of R^{c} is substituted or unsubstituted alkynyl (e.g., substituted or unsubstituted C₁₋₆ alkynyl). In certain embodiments, at least one instance of R^{c} is substituted or unsubstituted carbocyclyl (e.g., substituted or unsubstituted, 3- to 8-membered, monocyclic carbocyclyl, optionally including 1, 2, or 3 double bonds in the carbocyclic ring system; or substituted or unsubstituted, 5- to 14-membered, bicyclic carbocyclyl, optionally including 1, 2, 3, or 4 double bonds in the carbocyclic ring system). In certain embodiments, at least one instance of R^{c} is substituted or unsubstituted heterocyclyl (e.g., substituted or unsubstituted, 3- to 8-membered, monocyclic heterocyclyl, optionally including 1 or 2 double bonds in the heterocyclic ring system, wherein 1, 2, or 3 atoms in the heterocyclic ring system are independently nitrogen, oxygen, or sulfur; or substituted or unsubstituted, 5- to 14-membered, bicyclic heterocyclyl, optionally including 1, 2, or 3 double bonds in the heterocyclic ring system, wherein 1, 2, 3, or 4 atoms in the heterocyclic ring system are independently nitrogen, oxygen, or sulfur). In certain embodiments, at least one instance of R^{c} is substituted or unsubstituted aryl (e.g., substituted or unsubstituted, 6- to 10-membered aryl). In certain embodiments, at least one instance of R^{c} is substituted or unsubstituted phenyl. In certain embodiments, at least one instance of R^{c} is substituted or unsubstituted heteroaryl (e.g., substituted or unsubstituted, 5- to 6-membered, monocyclic heteroaryl, or substituted or unsubstituted, 8- to 10-membered, bicyclic heteroaryl, wherein 1, 2, 3, or 4 atoms in the heteroaryl ring system are independently nitrogen, oxygen, or sulfur). In certain embodiments, at least one instance of R^{c} is a nitrogen protecting group (e.g., Bn, Boc, Cbz, Fmoc, trifluoroacetyl, triphenylmethyl, acetyl, or Ts) when attached to a nitrogen atom. In certain embodiments, R^{c} is an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom. In certain embodiments, R^{c} is a sulfur protecting group (e.g., acetamidomethyl, t-Bu, 3-nitro-2-pyridine sulfenyl, 2-pyridine-sulfenyl, or triphenylmethyl) when attached to a sulfur atom. In certain embodiments, two instances of R^{c} are joined to form a substituted or unsubstituted heterocyclic ring (e.g., substituted or unsubstituted, 3- to 8-membered, monocyclic heterocyclic ring, optionally including 1 or 2 double bonds in the heterocyclic ring system, wherein 1, 2, or 3 atoms in the heterocyclic ring system are independently nitrogen, oxygen, or sulfur). In certain embodiments, two instances of R^{c} are joined to form a substituted or unsubstituted heteroaryl ring (e.g., substituted or unsubstituted, 5- to 6-membered, monocyclic heteroaryl ring, wherein 1, 2, 3, or 4 atoms in the heteroaryl ring system are independently nitrogen, oxygen, or sulfur).

In certain embodiments, R^{B} is H. In certain embodiments, R^{B} is substituted acyl. In certain embodiments, R^{B} is unsubstituted acyl. In certain embodiments, R^{B} is acetyl. In certain embodiments, R^{B} is —C(═O)R^{d} (e.g., —C(═O)(substituted or unsubstituted alkyl)). In certain embodiments, R^{B} is —C(═O)OR^{d} (e.g., —C(═O)O(substituted or unsubstituted alkyl) or —C(═O)OH). In certain embodiments, R^{B} is —C(═O)N(R^{d})₂, (e.g., —C(═O)NH₂, —C(═O)NH(substituted or unsubstituted alkyl), or —C(═O)N(substituted or unsubstituted alkyl)₂). In certain embodiments, R^{B} is unsubstituted alkyl. In certain embodiments, R^{B} is substituted alkyl. In certain embodiments, R^{B} is unsubstituted C₁₋₆ alkyl. In certain embodiments, R^{B} is substituted C₁₋₆ alkyl. In certain embodiments, R^{B} is C₁₋₆ alkyl substituted with at least one halogen. In certain embodiments, R^{B} is CH₃. In certain embodiments, R^{B} is substituted methyl. In certain embodiments, R^{B} is —CH₂F, —CHF₂, or —CF₃. In certain embodiments, R^{B} is ethyl. In certain embodiments, R^{B} is propyl. In certain embodiments, R^{B} is butyl. In certain embodiments, R^{B} is pentyl. In certain embodiments, R^{B} is hexyl. In certain embodiments, R^{B} is unsubstituted alkenyl. In certain embodiments, R^{B} is substituted alkenyl. In certain embodiments, R^{B} is unsubstituted C₁₋₆ alkenyl. In certain embodiments, R^{B} is substituted C₁₋₆ alkenyl. In certain embodiments, R^{B} is unsubstituted alkynyl. In certain embodiments, R^{B} is substituted alkynyl. In certain embodiments, R^{B} is unsubstituted C₁₋₆ alkynyl. In certain embodiments, R^{B} is substituted C₁₋₆ alkynyl. In certain embodiments, R^{B} is substituted carbocyclyl. In certain embodiments, R^{B} is unsubstituted carbocyclyl. In certain embodiments, R^{B} is saturated carbocyclyl. In certain embodiments, R^{B} is unsaturated carbocyclyl. In certain embodiments, R^{B} is 3- to 8-membered, monocyclic carbocyclyl, optionally including 1, 2, or 3 double bonds in the carbocyclic ring system. In certain embodiments, R^{B} is 5- to 14-membered, bicyclic carbocyclyl, optionally including 1, 2, 3, or 4 double bonds in the carbocyclic ring system. In certain embodiments, R^{B} is 5- to 20-membered, tricyclic carbocyclyl, optionally including 1, 2, 3, 4, or 5 double bonds in the carbocyclic ring system. In certain embodiments, R^{B} is 5- to 26-membered, tetracyclic carbocyclyl, optionally including 1, 2, 3, 4, 5, or 6 double bonds in the carbocyclic ring system. In certain embodiments, R^{B} is substituted heterocyclyl. In certain embodiments, R^{B} is unsubstituted heterocyclyl. In certain embodiments, R^{B} is saturated heterocyclyl. In certain embodiments, R^{B} is unsaturated heterocyclyl. In certain embodiments, R^{B} is 3- to 8-membered, monocyclic heterocyclyl, optionally including 1 or 2 double bonds in the heterocyclic ring system, wherein 1, 2, or 3 atoms in the heterocyclic ring system are independently nitrogen, oxygen, or sulfur. In certain embodiments, R^{B} is 5- to 14-membered, bicyclic heterocyclyl, optionally including 1, 2, or 3 double bonds in the heterocyclic ring system, wherein 1, 2, 3, or 4 atoms in the heterocyclic ring system are independently nitrogen, oxygen, or sulfur. In certain embodiments, R^{B} is 5- to 20-membered, tricyclic heterocyclyl, optionally including 1, 2, 3, or 4 double bonds in the heterocyclic ring system, wherein 1, 2, 3, 4, or 5 atoms in the heterocyclic ring system are independently nitrogen, oxygen, or sulfur. In certain embodiments, R^{B} is substituted aryl. In certain embodiments, R^{B} is unsubstituted aryl. In certain embodiments, R^{B} is 6- to 14-membered aryl. In certain embodiments, R^{B} is 6- to 10-membered aryl. In certain embodiments, R^{B} is substituted phenyl. In certain embodiments, R^{B} is unsubstituted phenyl. In certain embodiments, R^{B} is substituted naphthyl. In certain embodiments, R^{B} is unsubstituted naphthyl. In certain embodiments, R^{B} is substituted heteroaryl. In certain embodiments, R^{B} is unsubstituted heteroaryl. In certain embodiments, R^{B} is 5- to 6-membered, monocyclic heteroaryl, wherein 1, 2, 3, or 4 atoms in the heteroaryl ring system are independently nitrogen, oxygen, or sulfur. In certain embodiments, R^{B} is 8- to 10-membered, bicyclic heteroaryl, wherein 1, 2, 3, or 4 atoms in the heteroaryl ring system are independently nitrogen, oxygen, or sulfur.

In certain embodiments, R^{B} is a moiety shown in Table 1A. In certain embodiments, R^{B} is a moiety shown in Table 1B.

An R^{B} group may independently include one or more substituents R^{d}. In certain embodiments, all instances of R^{d} are the same. In certain embodiments, two instances of R^{d} are different from each other. In certain embodiments, at least one instance of R^{d} is H. In certain embodiments, each instance of R^{d} is H. In certain embodiments, at least one instance of R^{d} is substituted or unsubstituted acyl (e.g., acetyl). In certain embodiments, at least one instance of R^{d} is substituted or unsubstituted alkyl (e.g., substituted or unsubstituted C₁₋₆ alkyl). In certain embodiments, at least one instance of R^{d} is —CH₃. In certain embodiments, at least one instance of R^{d} is —CF₃, unsubstituted ethyl, perfluoroethyl, unsubstituted propyl, perfluoropropyl, unsubstituted butyl, or perfluorobutyl. In certain embodiments, at least one instance of R^{d} is substituted or unsubstituted alkenyl (e.g., substituted or unsubstituted C₁₋₆ alkenyl). In certain embodiments, at least one instance of R^{d} is substituted or unsubstituted alkynyl (e.g., substituted or unsubstituted C₁₋₆ alkynyl). In certain embodiments, at least one instance of R^{d} is substituted or unsubstituted carbocyclyl (e.g., substituted or unsubstituted, 3- to 8-membered, monocyclic carbocyclyl, optionally including 1, 2, or 3 double bonds in the carbocyclic ring system; or substituted or unsubstituted, 5- to 14-membered, bicyclic carbocyclyl, optionally including 1, 2, 3, or 4 double bonds in the carbocyclic ring system). In certain embodiments, at least one instance of R^{d} is substituted or unsubstituted heterocyclyl (e.g., substituted or unsubstituted, 3- to 8-membered, monocyclic heterocyclyl, optionally including 1 or 2 double bonds in the heterocyclic ring system, wherein 1, 2, or 3 atoms in the heterocyclic ring system are independently nitrogen, oxygen, or sulfur; or substituted or unsubstituted, 5- to 14-membered, bicyclic heterocyclyl, optionally including 1, 2, or 3 double bonds in the heterocyclic ring system, wherein 1, 2, 3, or 4 atoms in the heterocyclic ring system are independently nitrogen, oxygen, or sulfur). In certain embodiments, at least one instance of R^{d} is substituted or unsubstituted aryl (e.g., substituted or unsubstituted, 6- to 10-membered aryl). In certain embodiments, at least one instance of R^{d} is substituted or unsubstituted phenyl. In certain embodiments, at least one instance of R^{d} is substituted or unsubstituted heteroaryl (e.g., substituted or unsubstituted, 5- to 6-membered, monocyclic heteroaryl, or substituted or unsubstituted, 8- to 10-membered, bicyclic heteroaryl, wherein 1, 2, 3, or 4 atoms in the heteroaryl ring system are independently nitrogen, oxygen, or sulfur). In certain embodiments, at least one instance of R^{d} is a nitrogen protecting group (e.g., Bn, Boc, Cbz, Fmoc, trifluoroacetyl, triphenylmethyl, acetyl, or Ts) when attached to a nitrogen atom. In certain embodiments, R^{d} is an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom. In certain embodiments, R^{d} is a sulfur protecting group (e.g., acetamidomethyl, t-Bu, 3-nitro-2-pyridine sulfenyl, 2-pyridine-sulfenyl, or triphenylmethyl) when attached to a sulfur atom. In certain embodiments, two instances of R^{d} are joined to form a substituted or unsubstituted heterocyclic ring (e.g., substituted or unsubstituted, 3- to 8-membered, monocyclic heterocyclic ring, optionally including 1 or 2 double bonds in the heterocyclic ring system, wherein 1, 2, or 3 atoms in the heterocyclic ring system are independently nitrogen, oxygen, or sulfur). In certain embodiments, two instances of R^{d} are joined to form a substituted or unsubstituted heteroaryl ring (e.g., substituted or unsubstituted, 5- to 6-membered, monocyclic heteroaryl ring, wherein 1, 2, 3, or 4 atoms in the heteroaryl ring system are independently nitrogen, oxygen, or sulfur).

In certain embodiments, R^{A} and R^{B} are the same. In certain embodiments, R^{A} and R^{B} are different from each other. In certain embodiments, R^{A} is a moiety shown in an entry of Table 1B, and R^{B} is the other moiety shown in the entry.

In certain embodiments, R^{A} and R^{B} are joined to form a substituted or unsubstituted, saturated or unsaturated, carbocyclic ring. In certain embodiments, R^{A} and R^{B} are joined to form a 3- to 8-membered, monocyclic carbocyclic ring, optionally including 1, 2, or 3 double bonds in the carbocyclic ring system. In certain embodiments, R^{A} and R^{B} are joined to form a 5- to 14-membered, bicyclic carbocyclic ring, optionally including 1, 2, 3, or 4 double bonds in the carbocyclic ring system. In certain embodiments, R^{A} and R^{B} are joined to form a 5- to 20-membered, tricyclic carbocyclic ring, optionally including 1, 2, 3, 4, or 5 double bonds in the carbocyclic ring system.

In certain embodiments, R^{A} and R^{B} are joined to form a substituted or unsubstituted, saturated or unsaturated, heterocyclic ring. In certain embodiments, R^{A} and R^{B} are joined to form a 3- to 8-membered, monocyclic heterocyclic ring, optionally including 1 or 2 double bonds in the heterocyclic ring system, wherein 1, 2, or 3 atoms in the heterocyclic ring system are independently nitrogen, oxygen, or sulfur. In certain embodiments, R^{A} and R^{B} are joined to form a 5- to 14-membered, bicyclic heterocyclic ring, optionally including 1, 2, or 3 double bonds in the heterocyclic ring system, wherein 1, 2, 3, or 4 atoms in the heterocyclic ring system are independently nitrogen, oxygen, or sulfur. In certain embodiments, R^{A} and R^{B} are joined to form a 5- to 20-membered, tricyclic heterocyclic ring, optionally including 1, 2, 3, 4, or 5 double bonds in the heterocyclic ring system, wherein 1, 2, 3, 4, or 5 atoms in the heterocyclic ring system are independently nitrogen, oxygen, or sulfur.

In certain embodiments, R^{A} and R^{B} are joined to form a moiety shown in Table 1C.

TABLE 1C

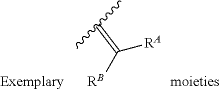

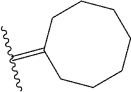 [SMILES: *=C1CCCCCCC1]

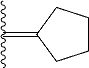 [SMILES: *=C1CCCC1]

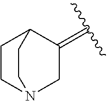 [SMILES: *=C1CN2CCC1CC2]

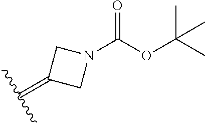 [SMILES: *=C1CN(C(=O)OC(C)(C)C)C1]

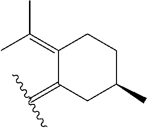 [SMILES: *=C1C[C@H](C)CCC1=C(C)C]

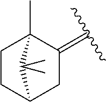 [SMILES: *=C1C[C@H]2CC[C@]1(C)C2(C)C]

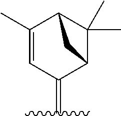

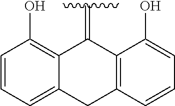 [SMILES: *=C1c2c(O)cccc2Cc2cccc(O)c21]

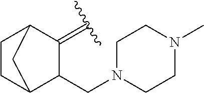 [SMILES: *=C1C2CCC(C2)C1CN1CCN(C)CC1]

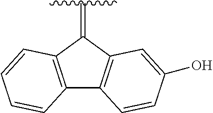 [SMILES: *=C1c2ccccc2-c2ccc(O)cc21]

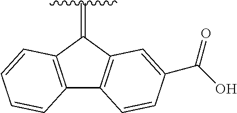 [SMILES: *=C1c2ccccc2-c2ccc(C(=O)O)cc21]

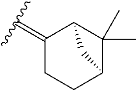 [SMILES: *=C1CC[C@H]2C[C@@H]1C2(C)C]

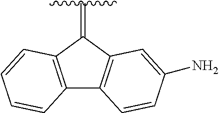 [SMILES: *=C1c2ccccc2-c2ccc(N)cc21]

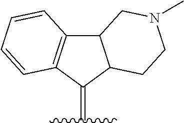 [SMILES: *=C1c2ccccc2C2CN(C)CCC12]

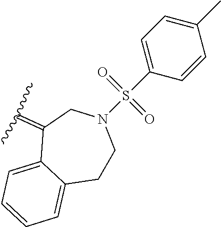 [SMILES: *=C1CN(S(=O)(=O)c2ccc(C)cc2)CCc2ccccc21]

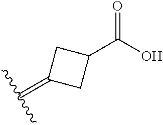 [SMILES: *=C1CC(C(=O)O)C1]

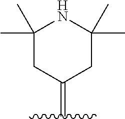

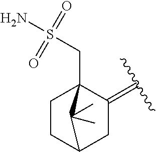 [SMILES: *=C1CC2CC[C@@]1(CS(N)(=O)=O)C2(C)C]

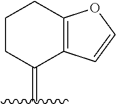 [SMILES: *=C1CCCc2occc21]

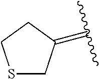 [SMILES: *=C1CCSC1]

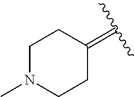 [SMILES: *=C1CCN(C)CC1]

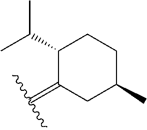 [SMILES: *=C1C[C@H](C)CC[C@H]1C(C)C]

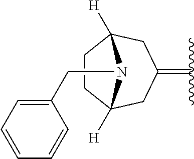 [SMILES: *=C1C[C@H]2CC[C@@H](C1)N2Cc1ccccc1]

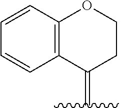 [SMILES: *=C1CCOc2ccccc21]

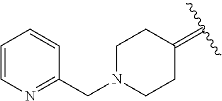 [SMILES: *=C1CCN(Cc2ccccn2)CC1]

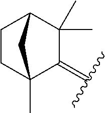 [SMILES: *=C1C(C)(C)[C@H]2CC[C@]1(C)C2]

In certain embodiments, R^{C} is H. In certain embodiments, R^{C} is substituted or unsubstituted C₁₋₆ alkyl (e.g., Me, —CF₃, unsubstituted ethyl, perfluoroethyl, unsubstituted propyl, perfluoropropyl, unsubstituted butyl, or perfluorobutyl). In certain embodiments, R^{C} is Me. In certain embodiments, R^{C} is a nitrogen protecting group (e.g., Bn, Boc, Cbz, Fmoc, trifluoroacetyl, triphenylmethyl, acetyl, or Ts).

Formula (I) may include one or more instances of substituent R^{a}. When Formula (I) includes two or more instances of R^{a}, any two instances of R^{a} may be the same or different from each other. In certain embodiments, at least one instance of R^{a} is H. In certain embodiments, each instance of R^{a} is H. In certain embodiments, at least one instance of R^{a} is substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, a nitrogen protecting group (e.g., Bn, Boc, Cbz, Fmoc, trifluoroacetyl, triphenylmethyl, acetyl, or Ts) when attached to a nitrogen atom, an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom, or two instances of R^{a} are joined to form a substituted or unsubstituted, heterocyclic ring, or substituted or unsubstituted, heteroaryl ring.

Formula (I) includes substituent R^{A2} at the 1-position of the 2,7-diazaindolyl ring. In certain embodiments, R^{A2} is H. In certain embodiments, R^{A2} is substituted or unsubstituted acyl. In certain embodiments, R^{A2} is —C(═O)R^{a}, optionally wherein R^{a} is substituted or unsubstituted C₁₋₆ alkyl (e.g., Me) or substituted or unsubstituted C₂₋₆ alkenyl. In certain embodiments, R^{A2} is —C(═O)R^{a}, wherein R^{a} is substituted or unsubstituted vinyl. In certain embodiments, R^{A2} is —C(═O)CH═CH₂. In certain embodiments, R^{A2} is —C(═O)OR^{a}, optionally wherein R^{a} is H, substituted or unsubstituted C₁₋₆ alkyl (e.g., Me), or an oxygen protecting group (e.g., silyl, TBDPS, TBDMS, TIPS, TES, TMS, MOM, THP, t-Bu, Bn, allyl, acetyl, pivaloyl, or benzoyl). In certain embodiments, R^{A2} is —C(═O)N(R^{a})₂, optionally wherein each instance of R^{a} is independently H, substituted or unsubstituted C₁₋₆ alkyl (e.g., Me), or a nitrogen protecting group (e.g., Bn, Boc, Cbz, Fmoc, trifluoroacetyl, triphenylmethyl, acetyl, or Ts). In certain embodiments, R^{A2} is substituted or unsubstituted alkyl (e.g., substituted or unsubstituted C₁₋₆ alkyl). In certain embodiments, R^{A2} is Me. In certain embodiments, R^{A2} is Et. In certain embodiments, R^{A2} is n-Pr. In certain embodiments, R^{A2} is i-Pr. In certain embodiments, R^{A2} is Bu (e.g., n-Bu, i-Bu, sec-Bu, or t-Bu). In certain embodiments, R^{A2} is unsubstituted pentyl (e.g., unsubstituted n-pentyl, unsubstituted t-pentyl, unsubstituted neopentyl, unsubstituted isopentyl, unsubstituted sec-pentyl, or unsubstituted 3-pentyl). In certain embodiments, R^{A2} is sec-Bu, t-Bu, or unsubstituted 3-pentyl. In certain embodiments, R^{A2} is —CF₃, Bn, perfluoroethyl, perfluoropropyl, perfluorobutyl, or perfluoropentyl. In certain embodiments, R^{A2} is —CH₂C(═O)—NH—N═C(R^{a})₂. In certain embodiments, R^{A2} is substituted or unsubstituted carbocyclyl (e.g., substituted or unsubstituted, 3- to 7-membered, monocyclic carbocyclyl comprising zero, one, or two double bonds in the carbocyclic ring system). In certain embodiments, R^{A2} is substituted or unsubstituted cyclopropyl. In certain embodiments, R^{A2} is unsubstituted cyclopropyl. In certain embodiments, R^{A2} is substituted or unsubstituted cyclobutyl. In certain embodiments, R^{A2} is substituted or unsubstituted cyclopentyl. In certain embodiments, R^{A2} is unsubstituted cyclopropyl, unsubstituted cyclobutyl, or unsubstituted cyclopentyl. In certain embodiments, R^{A2} is substituted or unsubstituted heterocyclyl (e.g., substituted or unsubstituted, 3- to 7-membed, monocyclic heterocyclyl comprising zero, one, or two double bonds in the heterocyclic ring system, wherein one, two, or three atoms in the heterocyclic ring system are independently nitrogen, oxygen, or sulfur). In certain embodiments, R^{A2} is substituted or unsubstituted tetrahydropyranyl. In certain embodiments, R^{A2} is of the formula:

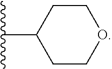 [SMILES: *C1CCOCC1]

In certain embodiments, R^{A2} is of the formula:

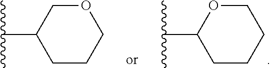 [SMILES: *C1CCCOC1 ; *C1CCCCO1]

In certain embodiments, R^{A2} is substituted or unsubstituted oxetanyl, substituted or unsubstituted tetrahydrofuranyl, substituted or unsubstituted pyrrolidinyl, substituted or unsubstituted piperidinyl, substituted or unsubstituted morpholinyl, or substituted or unsubstituted piperazinyl. In certain embodiments, R^{A2} is of the formula:

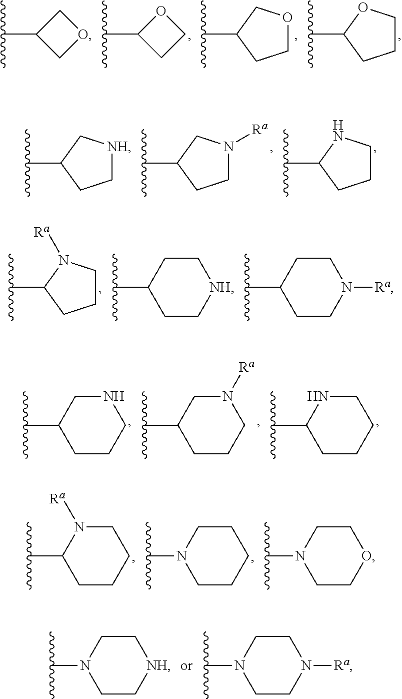

wherein each instance of R^{a} is independently unsubstituted C₁₋₆ alkyl (e.g., Me)). In certain embodiments, R^{A2} is a nitrogen protecting group (e.g., Bn, Boc, Cbz, Fmoc, trifluoroacetyl, triphenylmethyl, acetyl, or Ts). In certain embodiments, R^{A2} is Boc. In certain embodiments, R^{A2} is a warhead.

Any one of Formulae (I) and (II) may include one or more warheads independently selected from the group consisting of:

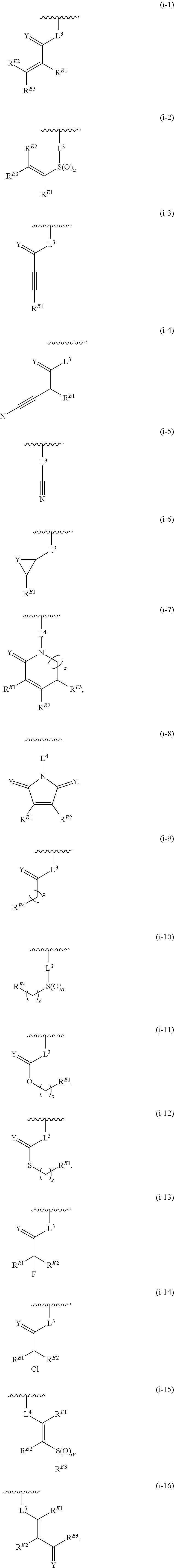

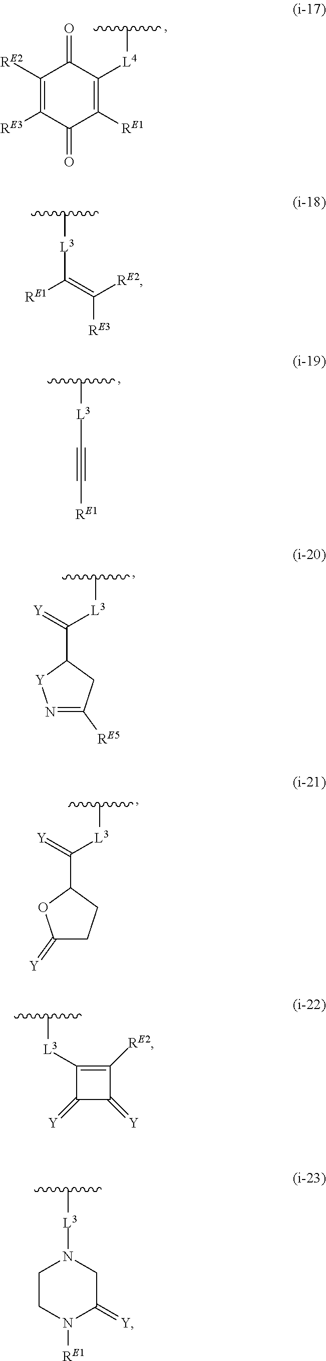

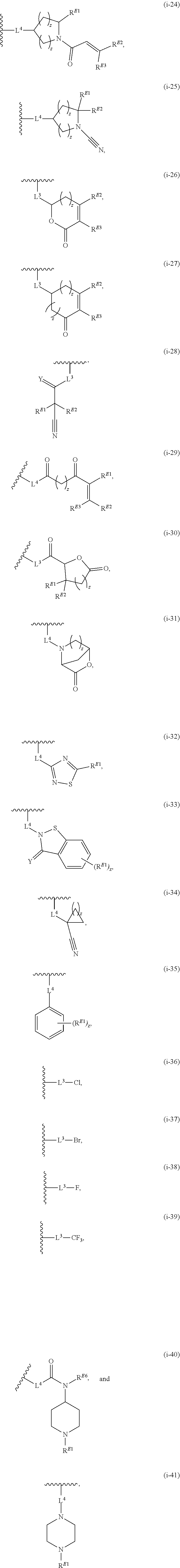

wherein:

L³ is a bond or an optionally substituted C₁₋₄ hydrocarbon chain, optionally wherein one or more carbon units of the hydrocarbon chain are independently replaced with —C═O—, —O—, —S—, —NR^{L3a}—, —NR^{L3a}C(═O)—, —C(═O)NR^{L3a}—, —SC(═O)—, —C(═O)S—, —OC(═O)—, —C(═O)O—, —NR^{L3a}C(═S)—, —C(═S)NR^{L3a}—, trans-CR^{L3b}═CR^{L3b}—, cis-CR^{L3b}═CR^{L3b}—, —C≡C—, —S(═O)—, —S(═O)O—, —OS(═O)—, —S(═O)NR^{L3a}—, —NR^{L3a}S(═O)—, —S(═O)₂—, —S(═O)₂O—, —OS(═O)₂—, —S(═O)₂NR^{L3a}—, or —NR^{L3a}S(═O)₂—, wherein R^{L3a} is hydrogen, substituted or unsubstituted C₁₋₆ alkyl, or a nitrogen protecting group, and wherein each occurrence of R^{L3b} is independently hydrogen, halogen, optionally substituted alkyl, optionally substituted alkenyl, optionally substituted alkynyl, optionally substituted carbocyclyl, optionally substituted heterocyclyl, optionally substituted aryl, or optionally substituted heteroaryl, or two R^{L3b} groups are joined to form an optionally substituted carbocyclic or optionally substituted heterocyclic ring;

L⁴ is a bond or an optionally substituted, branched or unbranched C₁₋₆ hydrocarbon chain;

each of R^{E1}, R^{E2}, and R^{E3} is independently hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —CN, —CH₂OR^{EE}, —CH₂N(R^{EE})₂, —CH₂SR^{EE}, —OR^{EE}, —N(R^{EE})₂, —Si(R^{EE})₃, or —SR^{EE}, wherein each occurrence of R^{EE} is independently hydrogen, optionally substituted alkyl, optionally substituted alkoxy, optionally substituted alkenyl, optionally substituted alkynyl, optionally substituted carbocyclyl, optionally substituted heterocyclyl, optionally substituted aryl, or optionally substituted heteroaryl, or two R^{EE} groups are joined to form an optionally substituted heterocyclic ring; or R^{E1} and R^{E3}, or R^{E2} and R^{E3}, or R^{E1} and R^{E2} are joined to form an optionally substituted carbocyclic or optionally substituted heterocyclic ring;

R^{E4} is a leaving group;

R^{E5} is halogen;

R^{E6} is hydrogen, substituted or unsubstituted C₁₋₆ alkyl, or a nitrogen protecting group;

each instance of Y is independently O, S, or NR^{E7}, wherein R^{E7} is hydrogen, substituted or unsubstituted C₁₋₆ alkyl, or a nitrogen protecting group;

a is 1 or 2; and

each instance of z is independently 0, 1, 2, 3, 4, 5, or 6, as valency permits.

In certain embodiments, any one of Formulae (I) and (II) does not include a warhead. In certain embodiments, any one of Formulae (I) and (II) includes one warhead. In certain embodiments, any one of Formulae (I) and (II) includes two or more warheads. When Formula (I) or (II) includes two or more warheads, any two of the warheads may be the same or different from each other. In certain embodiments, at least one warhead is of Formula (i-1a):

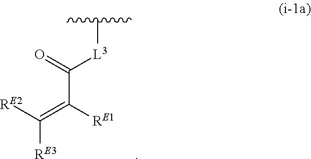

In certain embodiments, at least one warhead is of Formula (i-1b):

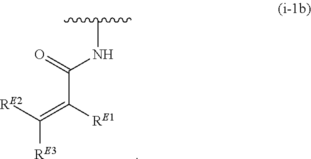

In certain embodiments, at least one warhead is of Formula (i-1c):

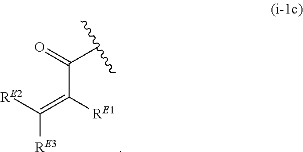

In certain embodiments, at least one warhead is of Formula (i-1d):

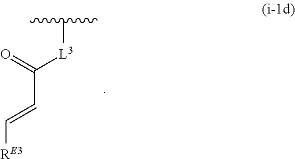

In certain embodiments, at least one warhead is of Formula (i-1e):

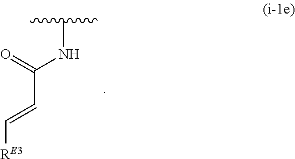

In certain embodiments, at least one warhead is of Formula (i-1f):

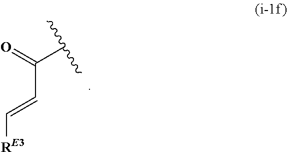

In certain embodiments, at least one warhead is of Formula (i-1g):

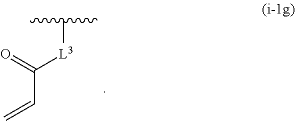

In certain embodiments, at least one warhead is

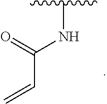 [SMILES: *NC(=O)C=C]

In certain embodiments, at least one warhead is

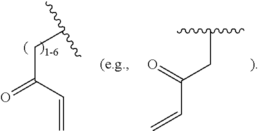

In certain embodiments, at least one warhead is

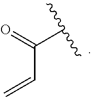 [SMILES: *C(=O)C=C]

In certain embodiments, at least one warhead is

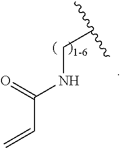

In certain embodiments, at least one warhead is of Formula (i-1h):

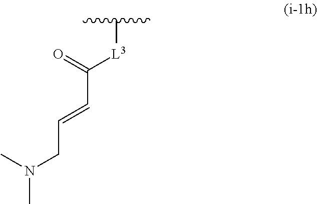

(e.g.,

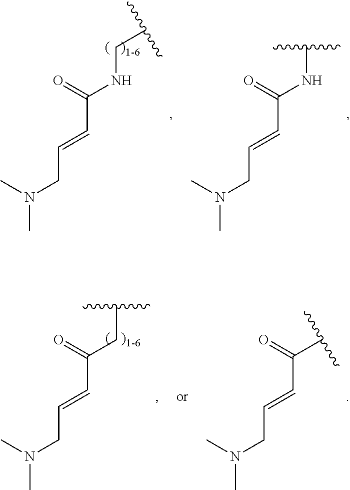

In certain embodiments, at least one warhead is

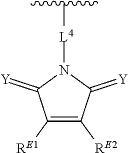

(e.g.,

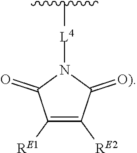

In certain embodiments, at least one warhead is

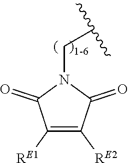

(e.g.,

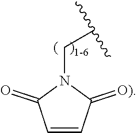

In certain embodiments, at least one warhead is

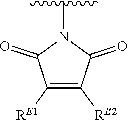

(e.g.,

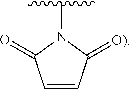

Formula (I) includes substituent R^{A3} at the 3-position of the 2,7-diazaindolyl ring. In certain embodiments, R^{A3} is H. In certain embodiments, R^{A3} is halogen (e.g., F, Cl, Br, or I). In certain embodiments, R^{A3} is substituted or unsubstituted C₁₋₆ alkyl. In certain embodiments, R^{A3} is Me. In certain embodiments, R^{A3} is —CF₃, Bn, Et, perfluoroethyl, Pr, perfluoropropyl, Bu, or perfluorobutyl. In certain embodiments, R^{A3} is —OR^{a}, optionally wherein R^{a} is H, substituted or unsubstituted C₁₋₆ alkyl (e.g., Me), substituted or unsubstituted acyl, or an oxygen protecting group (e.g., silyl, TBDPS, TBDMS, TIPS, TES, TMS, MOM, THP, t-Bu, Bn, allyl, acetyl, pivaloyl, or benzoyl). In certain embodiments, R^{A3} is —OC(═O)R^{a}, optionally wherein R^{a} is H, substituted or unsubstituted C₁₋₆ alkyl (e.g., Me), or substituted or unsubstituted C₂₋₆ alkenyl (e.g., substituted or unsubstituted vinyl). In certain embodiments, R^{A3} is —OC(═O)CH═CH₂. In certain embodiments, R^{A3} is —N(R^{a})₂, optionally wherein each instance of R^{a} is independently H, substituted or unsubstituted C₁₋₆ alkyl (e.g., Me), substituted or unsubstituted acyl, or a nitrogen protecting group (e.g., Bn, Boc, Cbz, Fmoc, trifluoroacetyl, triphenylmethyl, acetyl, or Ts). In certain embodiments, R^{A3} is —N(R^{a})C(═O)R^{a}, optionally wherein each instance of R^{a} is independently H, substituted or unsubstituted C₁₋₆ alkyl (e.g., Me), or substituted or unsubstituted C₂₋₆ alkenyl (e.g., substituted or unsubstituted vinyl). In certain embodiments, R^{A3} is —NHC(═O)CH═CH₂. In certain embodiments, R^{A3} is a warhead.

Formula (I) includes substituent R^{A4} on a nitrogen atom. In certain embodiments, R^{A4} is H. In certain embodiments, R^{A4} is substituted or unsubstituted C₁₋₆ alkyl. In certain embodiments, R^{A4} is Me. In certain embodiments, R^{A4} is —CF₃, Bn, Et, perfluoroethyl, Pr, perfluoropropyl, Bu, or perfluorobutyl. In certain embodiments, R^{A4} is a nitrogen protecting group (e.g., Bn, Boc, Cbz, Fmoc, trifluoroacetyl, triphenylmethyl, acetyl, or Ts).

Formula (I) includes substituent R^{A5}. In certain embodiments, R^{A5} is of the formula:

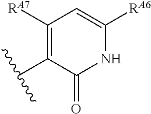

(e.g.,

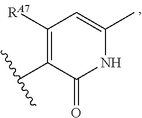

wherein R^{A7} is Et, Pr, or Bu). In certain embodiments, R^{A5} is of the formula:

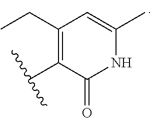 [SMILES: *c1c(CC)cc(C)[nH]c1=O]

In certain embodiments, R^{A5} is of the formula:

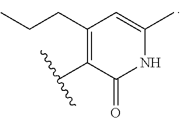 [SMILES: *c1c(CCC)cc(C)[nH]c1=O]

In certain embodiments, R^{A6} is H. In certain embodiments, R^{A6} is halogen (e.g., F, Cl, Br, or I). In certain embodiments, R^{A6} is substituted or unsubstituted C₁₋₆ alkyl. In certain embodiments, R^{A6} is Me. In certain embodiments, R^{A6} is substituted methyl (e.g., —CF₃ or Bn). In certain embodiments, R^{A6} is Et, substituted ethyl (e.g., perfluoroethyl), Pr, substituted propyl (e.g., perfluoropropyl), Bu, or substituted butyl (e.g., perfluorobutyl). In certain embodiments, R^{A6} is —OR^{a} (e.g., —OH or —O(substituted or unsubstituted C₁₋₆ alkyl) (e.g., —OMe)). In certain embodiments, R^{A6} is —N(R^{a})₂, optionally wherein each instance of R^{a} is independently hydrogen, substituted or unsubstituted C₁₋₆ alkyl, or a nitrogen protecting group (e.g., Bn, Boc, Cbz, Fmoc, trifluoroacetyl, triphenylmethyl, acetyl, or Ts). In certain embodiments, R^{A6} is —NH₂, —NHMe, or —N(Me)₂. In certain embodiments, R^{A7} is H. In certain embodiments, R^{A7} is halogen (e.g., F, Cl, Br, or I). In certain embodiments, R^{A7} is substituted or unsubstituted C₂₋₆ alkyl. In certain embodiments, R^{A7} is Et. In certain embodiments, R^{A7} is substituted ethyl (e.g., perfluoroethyl). In certain embodiments, R^{A7} is n-Pr. In certain embodiments, R^{A7} is i-Pr. In certain embodiments, R^{A7} is substituted propyl (e.g., perfluoropropyl). In certain embodiments, R^{A7} is Bu or unsubstituted pentyl. In certain embodiments, R^{A7} is substituted butyl (e.g., perfluorobutyl) or substituted pentyl (e.g., perfluoropentyl). In certain embodiments, R^{A7} is substituted or unsubstituted, 3- to 7-membed, monocyclic carbocyclyl comprising 0, 1, or 2 double bonds in the carbocyclic ring system. In certain embodiments, R^{A7} is substituted or unsubstituted cyclopropyl, substituted or unsubstituted cyclobutyl, or substituted or unsubstituted cyclopentyl. In certain embodiments, R^{A7} is —OR^{a} (e.g., —OH or —O(substituted or unsubstituted C₁₋₆ alkyl) (e.g., —OMe)). In certain embodiments, R^{A7} is —N(R^{a})₂, optionally wherein each instance of R^{a} is independently hydrogen, substituted or unsubstituted C₁₋₆ alkyl, or a nitrogen protecting group (e.g., Bn, Boc, Cbz, Fmoc, trifluoroacetyl, triphenylmethyl, acetyl, or Ts). In certain embodiments, R^{A7} is —NH₂, —NHMe, —NHEt, —N(Me)₂, or —N(Et)₂. In certain embodiments, R^{A7} is substituted or unsubstituted cyclopropyl or —N(R^{a})₂, wherein each instance of R^{a} is independently hydrogen, substituted or unsubstituted C₁₋₆ alkyl, or a nitrogen protecting group (e.g., Bn, Boc, Cbz, Fmoc, trifluoroacetyl, triphenylmethyl, acetyl, or Ts).

In certain embodiments, R^{A5} is of the formula:

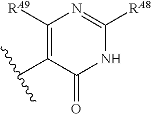

(e.g.,

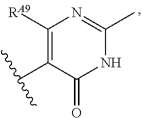

wherein R^{A9} is Me, Et, Pr, or Bu). The moiety of the formula:

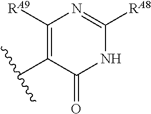

also includes its tautomeric form

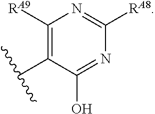

In certain embodiments, R^{A5} is of the formula:

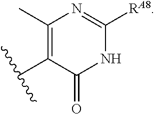

In certain embodiments, R^{A5} is of the formula:

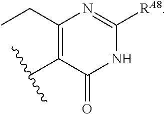

In certain embodiments, R^{A5} is of the formula:

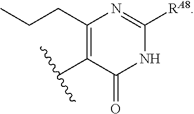

In certain embodiments, R^{A8} is H. In certain embodiments, R^{A8} is halogen (e.g., F, Cl, Br, or I). In certain embodiments, R^{A8} is substituted or unsubstituted C₁₋₆ alkyl. In certain embodiments, R^{A8} is Me. In certain embodiments, R^{A8} is substituted methyl (e.g., —CF₃ or Bn). In certain embodiments, R^{A8} is Et, substituted ethyl (e.g., perfluoroethyl), Pr, substituted propyl (e.g., perfluoropropyl), Bu, or substituted butyl (e.g., perfluorobutyl). In certain embodiments, R^{A8} is —OR^{a} (e.g., —OH or —O(substituted or unsubstituted C₁₋₆ alkyl) (e.g., —OMe)). In certain embodiments, R^{A8} is —N(R^{a})₂, optionally wherein each instance of R^{a} is independently hydrogen, substituted or unsubstituted C₁₋₆ alkyl, or a nitrogen protecting group (e.g., Bn, Boc, Cbz, Fmoc, trifluoroacetyl, triphenylmethyl, acetyl, or Ts). In certain embodiments, R^{A8} is —NH₂, —NHMe, or —N(Me)₂. In certain embodiments, R^{A9} is H. In certain embodiments, R^{A9} is halogen (e.g., F, Cl, Br, or I). In certain embodiments, R^{A9} is substituted or unsubstituted C₁₋₆ alkyl. In certain embodiments, R^{A9} is Me. In certain embodiments, R^{A9} is substituted methyl (e.g., —CF₃ or Bn). In certain embodiments, R^{A9} is Et. In certain embodiments, R^{A9} is substituted ethyl (e.g., perfluoroethyl). In certain embodiments, R^{A9} is n-Pr. In certain embodiments, R^{A9} is i-Pr. In certain embodiments, R^{A9} is substituted propyl (e.g., perfluoropropyl). In certain embodiments, R^{A9} is Bu or unsubstituted pentyl. In certain embodiments, R^{A9} is substituted butyl (e.g., perfluorobutyl) or substituted pentyl (e.g., perfluoropentyl). In certain embodiments, R^{A9} is substituted or unsubstituted, 3- to 7-membed, monocyclic carbocyclyl comprising 0, 1, or 2 double bonds in the carbocyclic ring system. In certain embodiments, R^{A9} is substituted or unsubstituted cyclopropyl, substituted or unsubstituted cyclobutyl, or substituted or unsubstituted cyclopentyl. In certain embodiments, R^{A9} is —OR^{a} (e.g., —OH or —O(substituted or unsubstituted C₁₋₆ alkyl) (e.g., —OMe)). In certain embodiments, R^{A9} is —N(R^{a})₂, optionally wherein each instance of R^{a} is independently hydrogen, substituted or unsubstituted C₁₋₆ alkyl, or a nitrogen protecting group (e.g., Bn, Boc, Cbz, Fmoc, trifluoroacetyl, triphenylmethyl, acetyl, or Ts). In certain embodiments, R^{A9} is —NH₂, —NHMe, —NHEt, —N(Me)₂, or —N(Et)₂. In certain embodiments, R^{A9} is substituted or unsubstituted cyclopropyl, —OR^{a}, or —N(R^{a})₂, wherein each instance of R^{a} is independently hydrogen, substituted or unsubstituted C₁₋₆ alkyl, an oxygen protecting group when attached to an oxygen atom, or a nitrogen protecting group when attached to a nitrogen atom.

In certain embodiments, R^{A5} is of the formula:

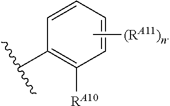

In certain embodiments, R^{A5} is of the formula:

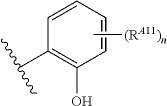

(e.g.,

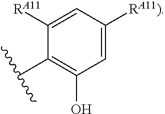

In certain embodiments, R^{A5} is of the formula:

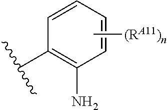

(e.g.,

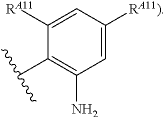

In certain embodiments, R^{A5} is of the formula:

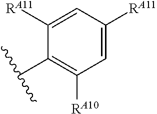

(e.g.,

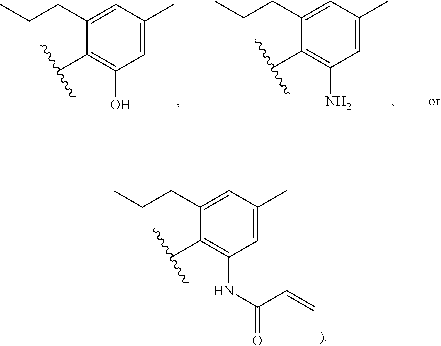

In certain embodiments, R^{A5} is of the formula:

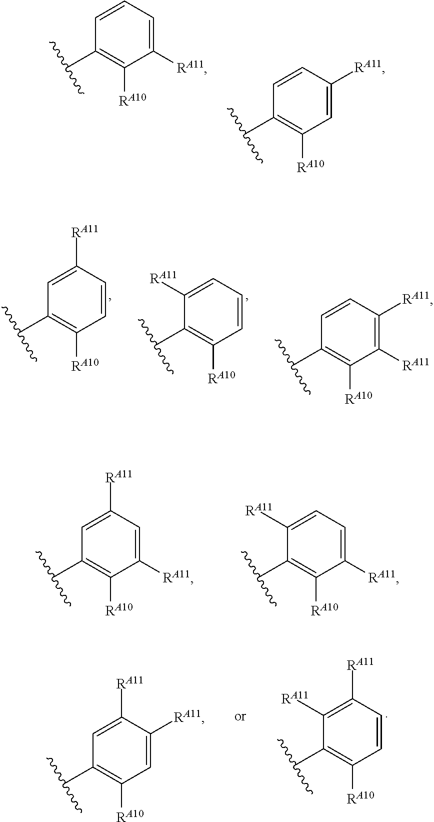

In certain embodiments, R^{A5} is of the formula:

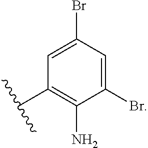 [SMILES: *c1cc(Br)cc(Br)c1N]

In certain embodiments, R^{A10} is —OR^{a} (e.g., —OH). In certain embodiments, R^{A10} is —N(R^{a})₂. In certain embodiments, R^{A10} is —NH₂. In certain embodiments, R^{A10} is —NHR^{a}, wherein R^{a} is substituted or unsubstituted C₁₋₆ alkyl or a nitrogen protecting group (e.g., Bn, Boc, Cbz, Fmoc, trifluoroacetyl, triphenylmethyl, acetyl, or Ts). In certain embodiments, R^{A10} is a warhead. In certain embodiments, R^{A10} is —NHC(═O)R^{a}, optionally wherein R^{a} is substituted or unsubstituted vinyl. In certain embodiments, R^{A10} is —NHC(═O)CH═CH₂. When Formula (I) includes two or more instances of R^{A11}, any two instances of R^{A11} may be the same or different from each other. In certain embodiments, at least one instance of R^{A11} is halogen. In certain embodiments, at least one instance of R^{A11} is Br. In certain embodiments, at least one instance of R^{A11} is F, Cl, or I. In certain embodiments, at least one instance of R^{A11} is substituted or unsubstituted C₁₋₆ alkyl. In certain embodiments, at least one instance of R^{A11} is Me. In certain embodiments, at least one instance of R^{A11} is substituted methyl (e.g., —CF₃ or Bn). In certain embodiments, at least one instance of R^{A11} is Et. In certain embodiments, at least one instance of R^{A11} is substituted ethyl (e.g., perfluoroethyl). In certain embodiments, at least one instance of R^{A11} is n-Pr. In certain embodiments, at least one instance of R^{A11} is i-Pr. In certain embodiments, at least one instance of R^{A11} is substituted propyl (e.g., perfluoropropyl). In certain embodiments, at least one instance of R^{A11} is Me, Et, or n-Pr. In certain embodiments, at least one instance of R^{A11} is Bu or unsubstituted pentyl. In certain embodiments, at least one instance of R^{A11} is substituted butyl (e.g., perfluorobutyl) or substituted pentyl (e.g., perfluoropentyl). In certain embodiments, at least one instance of R^{A11} is substituted or unsubstituted, 3- to 7-membed, monocyclic carbocyclyl comprising 0, 1, or 2 double bonds in the carbocyclic ring system. In certain embodiments, at least one instance of R^{A11} is substituted or unsubstituted cyclopropyl, substituted or unsubstituted cyclobutyl, or substituted or unsubstituted cyclopentyl. In certain embodiments, at least one instance of R^{A11} is —OR^{a} (e.g., —OH or —O(substituted or unsubstituted C₁₋₆ alkyl) (e.g., —OMe)). In certain embodiments, at least one instance of R^{A11} is —N(R^{a})₂, optionally wherein each instance of R^{a} is independently hydrogen, substituted or unsubstituted C₁₋₆ alkyl, or a nitrogen protecting group (e.g., Bn, Boc, Cbz, Fmoc, trifluoroacetyl, triphenylmethyl, acetyl, or Ts). In certain embodiments, at least one instance of R^{A11} is —NH₂, —NHMe, —NHEt, —N(Me)₂, or —N(Et)₂. In certain embodiments, at least one instance of R^{A11} is substituted or unsubstituted C₁₋₆ alkyl, substituted or unsubstituted cyclopropyl, —OR^{a}, or —N(R^{a})₂, wherein each instance of R^{a} is independently hydrogen, substituted or unsubstituted C₁₋₆ alkyl, an oxygen protecting group when attached to an oxygen atom, or a nitrogen protecting group when attached to a nitrogen atom. In certain embodiments, n is 0. In certain embodiments, n is 1. In certain embodiments, n is 2. In certain embodiments, n is 3. In certain embodiments, n is 4.

In certain embodiments, R^{A5} is of the formula:

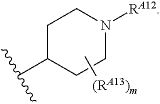

In certain embodiments, R^{A5} is of the formula:

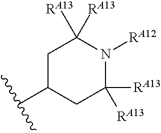

(e.g.,

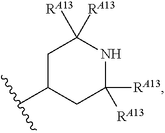

such as

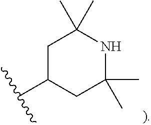 [SMILES: *C1CC(C)(C)NC(C)(C)C1]

In certain embodiments, R^{A12} is H. In certain embodiments, R^{A12} is substituted or unsubstituted C₁₋₆ alkyl. In certain embodiments, R^{A12} is Me. In certain embodiments, R^{A12} is —CF₃, Bn, Et, perfluoroethyl, Pr, perfluoropropyl, Bu, or perfluorobutyl. In certain embodiments, R^{A12} is a nitrogen protecting group (e.g., Bn, Boc, Cbz, Fmoc, trifluoroacetyl, triphenylmethyl, acetyl, or Ts). In certain embodiments, R^{A12} is a warhead. When Formula (I) includes two or more instances of R^{A13}, any two instances of R^{A13} may be the same or different from each other. In certain embodiments, at least one instance of R^{A13} is halogen (e.g., F, Cl, Br, or I). In certain embodiments, at least one instance of R^{A13} is substituted or unsubstituted C₁₋₆ alkyl. In certain embodiments, at least one instance of R^{A13} is Me. In certain embodiments, at least one instance of R^{A13} is substituted methyl (e.g., —CF₃ or Bn). In certain embodiments, at least one instance of R^{A13} is Et. In certain embodiments, at least one instance of R^{A13} is substituted ethyl (e.g., perfluoroethyl). In certain embodiments, at least one instance of R^{A13} is n-Pr. In certain embodiments, at least one instance of R^{A13} is i-Pr. In certain embodiments, at least one instance of R^{A13} is substituted propyl (e.g., perfluoropropyl). In certain embodiments, at least one instance of R^{A13} is Me, Et, or n-Pr. In certain embodiments, at least one instance of R^{A13} is Bu or unsubstituted pentyl. In certain embodiments, at least one instance of R^{A13} is substituted butyl (e.g., perfluorobutyl) or substituted pentyl (e.g., perfluoropentyl). In certain embodiments, at least one instance of R^{A13} is substituted or unsubstituted, 3- to 7-membed, monocyclic carbocyclyl comprising 0, 1, or 2 double bonds in the carbocyclic ring system. In certain embodiments, at least one instance of R^{A13} is substituted or unsubstituted cyclopropyl, substituted or unsubstituted cyclobutyl, or substituted or unsubstituted cyclopentyl. In certain embodiments, at least one instance of R^{A13} is —OR^{a} (e.g., —OH or —O(substituted or unsubstituted C₁₋₆ alkyl) (e.g., —OMe)). In certain embodiments, at least one instance of R^{A13} is —N(R^{a})₂, optionally wherein each instance of R^{a} is independently hydrogen, substituted or unsubstituted C₁₋₆ alkyl, or a nitrogen protecting group (e.g., Bn, Boc, Cbz, Fmoc, trifluoroacetyl, triphenylmethyl, acetyl, or Ts). In certain embodiments, at least one instance of R^{A13} is —NH₂, —NHMe, —NHEt, —N(Me)₂, or —N(Et)₂. In certain embodiments, at least one instance of R^{A13} is halogen, substituted or unsubstituted cyclopropyl, —OR^{a}, or —N(R^{a})₂, wherein each instance of R^{a} is independently hydrogen, substituted or unsubstituted C₁₋₆ alkyl, an oxygen protecting group when attached to an oxygen atom, or a nitrogen protecting group when attached to a nitrogen atom. In certain embodiments, m is 0. In certain embodiments, m is 1. In certain embodiments, m is 2. In certain embodiments, m is 3, 4, 5, 6, 7, or 8. In certain embodiments, m is 9.

Formula (I) includes substituent R^{A5}. In certain embodiments, R^{A5} is of the formula:

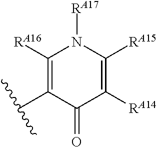

(e.g.,

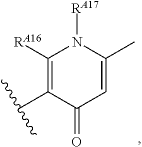

optionally wherein R^{A16} is Et, Pr, or Bu). In certain embodiments, R^{A5} is of the formula:

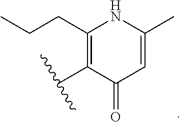 [SMILES: *c1c(CCC)[nH]c(C)cc1=O]

In certain embodiments, R^{A5} is of the formula:

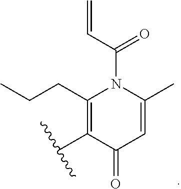 [SMILES: *c1c(CCC)n(C(=O)C=C)c(C)cc1=O]

In certain embodiments, R^{A14} is H. In certain embodiments, R^{A14} is halogen (e.g., F, Cl, Br, or I). In certain embodiments, R^{A14} is substituted or unsubstituted C₁₋₆ alkyl. In certain embodiments, R^{A14} is Me. In certain embodiments, R^{A14} is substituted methyl (e.g., —CF₃ or Bn). In certain embodiments, R^{A14} is Et, substituted ethyl (e.g., perfluoroethyl), Pr, substituted propyl (e.g., perfluoropropyl), Bu, or substituted butyl (e.g., perfluorobutyl). In certain embodiments, R^{A14} is —OR^{a} (e.g., —OH or —O(substituted or unsubstituted C₁₋₆ alkyl) (e.g., —OMe)). In certain embodiments, R^{A14} is —N(R^{a})₂, optionally wherein each instance of R^{a} is independently hydrogen, substituted or unsubstituted C₁₋₆ alkyl, or a nitrogen protecting group (e.g., Bn, Boc, Cbz, Fmoc, trifluoroacetyl, triphenylmethyl, acetyl, or Ts). In certain embodiments, R^{A14} is —NH₂, —NHMe, or —N(Me)₂. In certain embodiments, R^{A15} is H. In certain embodiments, R^{A15} is halogen (e.g., F, Cl, Br, or I). In certain embodiments, R^{A15} is substituted or unsubstituted C₁₋₆ alkyl. In certain embodiments, R^{A15} is Me. In certain embodiments, R^{A15} is substituted methyl (e.g., —CF₃ or Bn). In certain embodiments, R^{A15} is Et, substituted ethyl (e.g., perfluoroethyl), Pr, substituted propyl (e.g., perfluoropropyl), Bu, or substituted butyl (e.g., perfluorobutyl). In certain embodiments, R^{A15} is —OR^{a} (e.g., —OH or —O(substituted or unsubstituted C₁₋₆ alkyl) (e.g., —OMe)). In certain embodiments, R^{A15} is —N(R^{a})₂, optionally wherein each instance of R^{a} is independently hydrogen, substituted or unsubstituted C₁₋₆ alkyl, or a nitrogen protecting group (e.g., Bn, Boc, Cbz, Fmoc, trifluoroacetyl, triphenylmethyl, acetyl, or Ts). In certain embodiments, R^{A15} is —NH₂, —NHMe, or —N(Me)₂. In certain embodiments, R^{A16} is H. In certain embodiments, R^{A16} is halogen (e.g., F, Cl, Br, or I). In certain embodiments, R^{A16} is substituted or unsubstituted C₂₋₆ alkyl. In certain embodiments, R^{A16} is Et. In certain embodiments, R^{A16} is substituted ethyl (e.g., perfluoroethyl). In certain embodiments, R^{A16} is n-Pr. In certain embodiments, R^{A16} is i-Pr. In certain embodiments, R^{A16} is substituted propyl (e.g., perfluoropropyl). In certain embodiments, R^{A16} is Bu or unsubstituted pentyl. In certain embodiments, R^{A16} is substituted butyl (e.g., perfluorobutyl) or substituted pentyl (e.g., perfluoropentyl). In certain embodiments, R^{A16} is substituted or unsubstituted, 3- to 7-membed, monocyclic carbocyclyl comprising 0, 1, or 2 double bonds in the carbocyclic ring system. In certain embodiments, R^{A16} is substituted or unsubstituted cyclopropyl, substituted or unsubstituted cyclobutyl, or substituted or unsubstituted cyclopentyl. In certain embodiments, R^{A16} is —OR^{a} (e.g., —OH or —O(substituted or unsubstituted C₁₋₆ alkyl) (e.g., —OMe)). In certain embodiments, R^{A16} is —N(R^{a})₂, optionally wherein each instance of R^{a} is independently hydrogen, substituted or unsubstituted C₁₋₆ alkyl, or a nitrogen protecting group (e.g., Bn, Boc, Cbz, Fmoc, trifluoroacetyl, triphenylmethyl, acetyl, or Ts). In certain embodiments, R^{A16} is —NH₂, —NHMe, —NHEt, —N(Me)₂, or —N(Et)₂. In certain embodiments, R^{A16} is substituted or unsubstituted cyclopropyl or —N(R^{a})₂, wherein each instance of R^{a} is independently hydrogen, substituted or unsubstituted C₁₋₆ alkyl, or a nitrogen protecting group (e.g., Bn, Boc, Cbz, Fmoc, trifluoroacetyl, triphenylmethyl, acetyl, or Ts). In certain embodiments, R^{A17} is H. In certain embodiments, R^{A17} is substituted or unsubstituted acyl. In certain embodiments, R^{A17} is —C(═O)R^{a}, optionally wherein R^{a} is substituted or unsubstituted C₁₋₆ alkyl (e.g., Me) or substituted or unsubstituted C₂₋₆ alkenyl. In certain embodiments, R^{A17} is a warhead. In certain embodiments, R^{A17} is —C(═O)R^{a}, wherein R^{a} is substituted or unsubstituted vinyl. In certain embodiments, R^{A17} is —C(═O)CH═CH₂. In certain embodiments, R^{A17} is —C(═O)OR^{a}, optionally wherein R^{a} is H, substituted or unsubstituted C₁₋₆ alkyl (e.g., Me), or an oxygen protecting group (e.g., silyl, TBDPS, TBDMS, TIPS, TES, TMS, MOM, THP, t-Bu, Bn, allyl, acetyl, pivaloyl, or benzoyl). In certain embodiments, R^{A17} is —C(═O)N(R^{a})₂, optionally wherein each instance of R^{a} is independently H, substituted or unsubstituted C₁₋₆ alkyl (e.g., Me), or a nitrogen protecting group (e.g., Bn, Boc, Cbz, Fmoc, trifluoroacetyl, triphenylmethyl, acetyl, or Ts). In certain embodiments, R^{A17} is substituted or unsubstituted C₁₋₆ alkyl. In certain embodiments, R^{A17} is Me. In certain embodiments, R^{A17} is —CF₃, Bn, Et, perfluoroethyl, Pr, perfluoropropyl, Bu, or perfluorobutyl. In certain embodiments, R^{A17} is a nitrogen protecting group (e.g., Bn, Boc, Cbz, Fmoc, trifluoroacetyl, triphenylmethyl, acetyl, or Ts).

In certain embodiments, the compound of Formula (I) is of the formula:

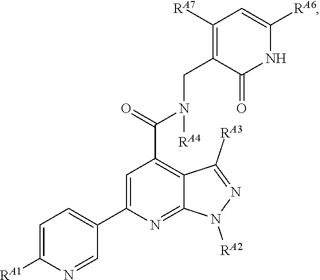

or a pharmaceutically acceptable salt, solvate, hydrate, polymorph, co-crystal, tautomer, stereoisomer, isotopically labeled derivative, or prodrug thereof.

In certain embodiments, the compound of Formula (I) is of the formula:

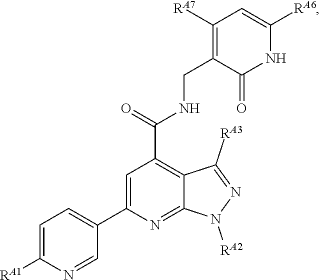

or a pharmaceutically acceptable salt, solvate, hydrate, polymorph, co-crystal, tautomer, stereoisomer, isotopically labeled derivative, or prodrug thereof.

In certain embodiments, the compound of Formula (I) is of the formula:

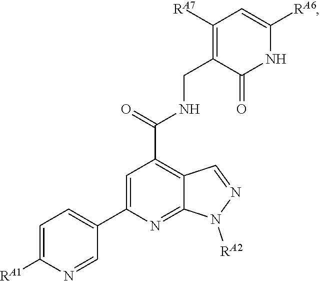

or a pharmaceutically acceptable salt, solvate, hydrate, polymorph, co-crystal, tautomer, stereoisomer, isotopically labeled derivative, or prodrug thereof.

In certain embodiments, the compound of Formula (I) is of the formula:

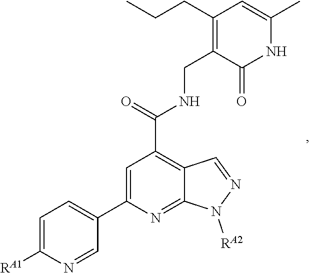

or a pharmaceutically acceptable salt, solvate, hydrate, polymorph, co-crystal, tautomer, stereoisomer, isotopically labeled derivative, or prodrug thereof.

In certain embodiments, the compound of Formula (I) is of the formula:

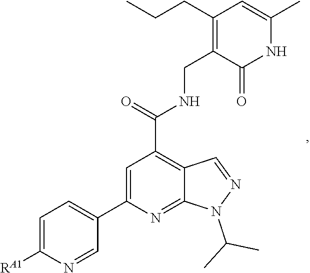

or a pharmaceutically acceptable salt, solvate, hydrate, polymorph, co-crystal, tautomer, stereoisomer, isotopically labeled derivative, or prodrug thereof.

In certain embodiments, the compound of Formula (I) is of the formula:

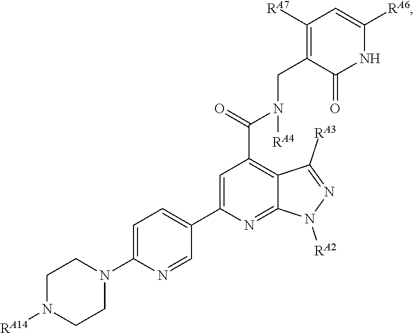

or a pharmaceutically acceptable salt, solvate, hydrate, polymorph, co-crystal, tautomer, stereoisomer, isotopically labeled derivative, or prodrug thereof, wherein R^{A14} is hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, or a nitrogen protecting group.

In certain embodiments, the compound of Formula (I) is of the formula:

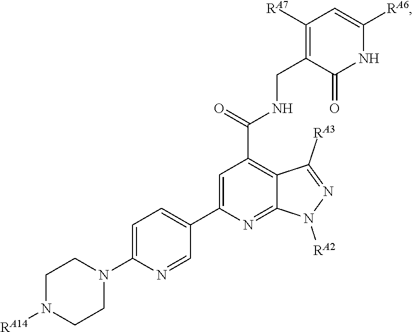

or a pharmaceutically acceptable salt, solvate, hydrate, polymorph, co-crystal, tautomer, stereoisomer, isotopically labeled derivative, or prodrug thereof.

In certain embodiments, the compound of Formula (I) is of the formula:

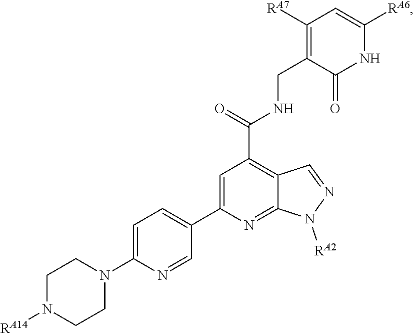

or a pharmaceutically acceptable salt, solvate, hydrate, polymorph, co-crystal, tautomer, stereoisomer, isotopically labeled derivative, or prodrug thereof.

In certain embodiments, the compound of Formula (I) is of the formula:

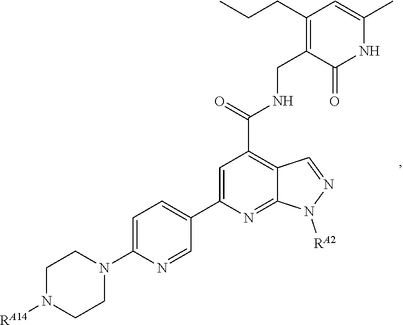

or a pharmaceutically acceptable salt, solvate, hydrate, polymorph, co-crystal, tautomer, stereoisomer, isotopically labeled derivative, or prodrug thereof.

In certain embodiments, the compound of Formula (I) is of the formula:

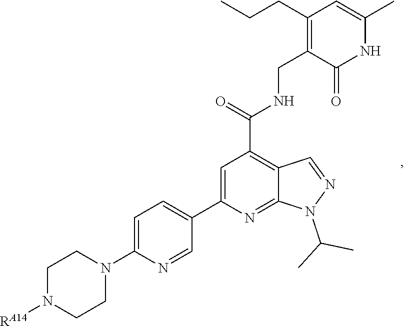

or a pharmaceutically acceptable salt, solvate, hydrate, polymorph, co-crystal, tautomer, stereoisomer, isotopically labeled derivative, or prodrug thereof.

In certain embodiments, the compound of Formula (I) is of the formula:

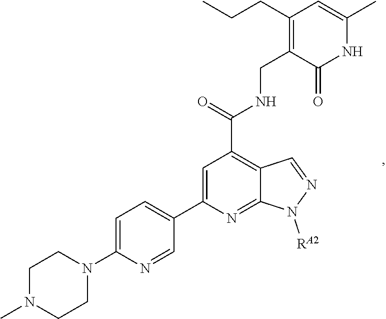

or a pharmaceutically acceptable salt, solvate, hydrate, polymorph, co-crystal, tautomer, stereoisomer, isotopically labeled derivative, or prodrug thereof, wherein R^{A2} is a nitrogen protecting group (e.g., Boc).

In certain embodiments, the compound of Formula (I) is of the formula:

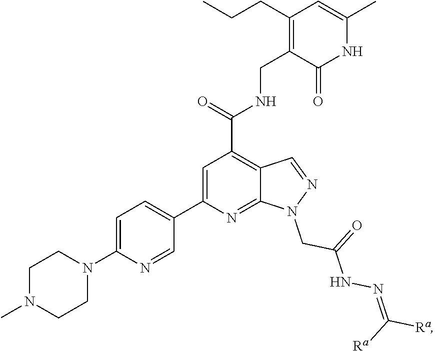

or a pharmaceutically acceptable salt, solvate, hydrate, polymorph, co-crystal, tautomer, stereoisomer, isotopically labeled derivative, or prodrug thereof.

In certain embodiments, the compound of Formula (I) is of the formula:

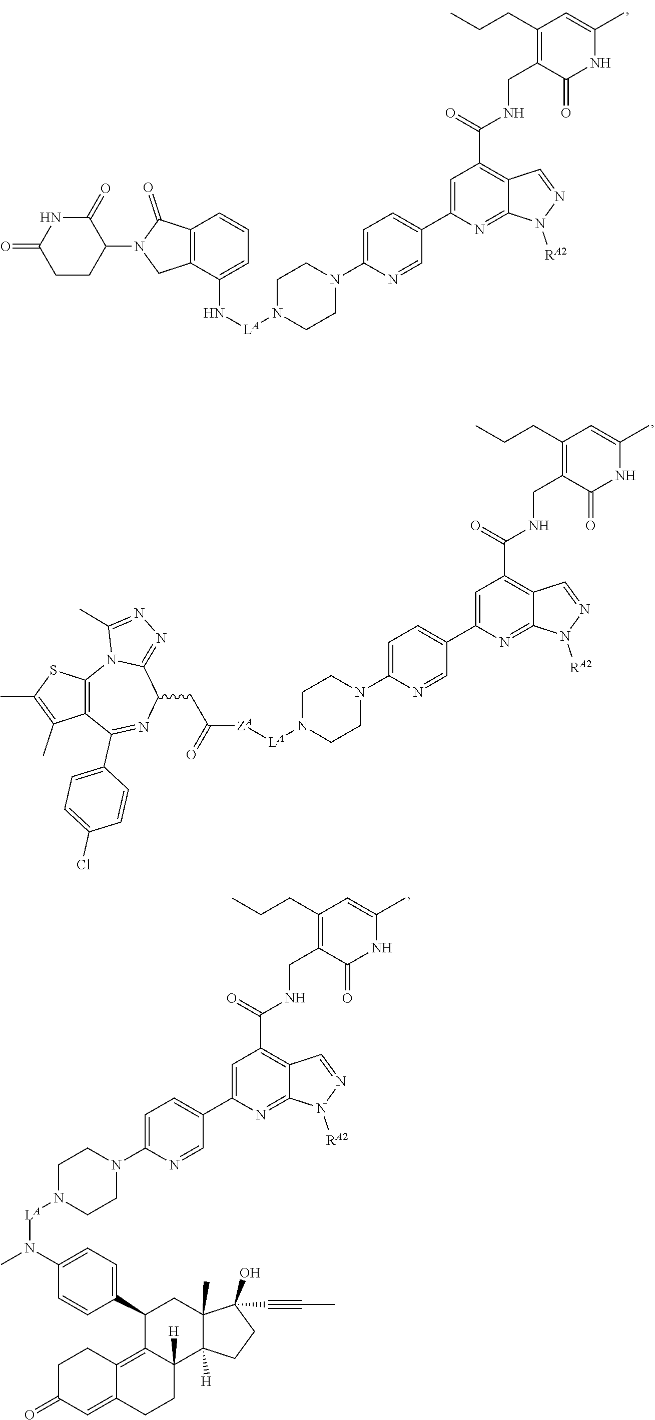

or a pharmaceutically acceptable salt, solvate, hydrate, polymorph, co-crystal, tautomer, stereoisomer, isotopically labeled derivative, or prodrug thereof, optionally wherein each instance of R^{A2} is i-Pr.

In certain embodiments, the compound of Formula (I) is of the formula:

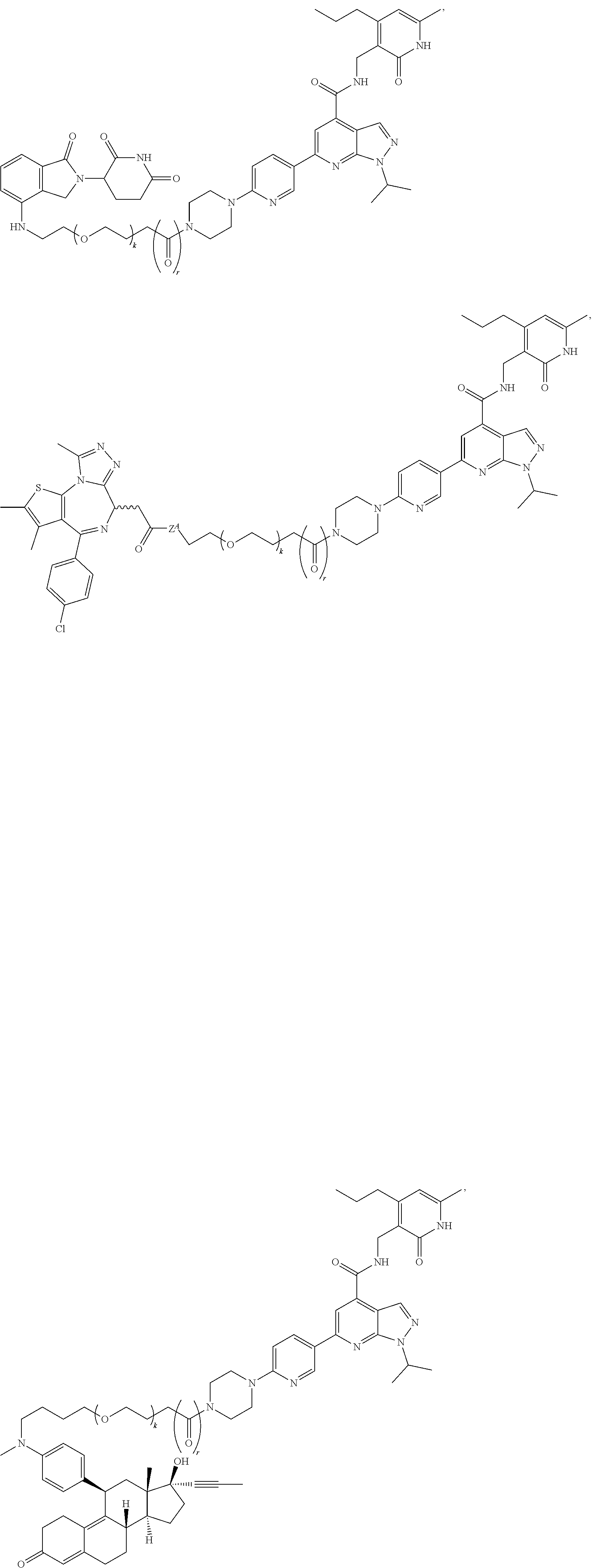

or a pharmaceutically acceptable salt, solvate, hydrate, polymorph, co-crystal, tautomer, stereoisomer, isotopically labeled derivative, or prodrug thereof, wherein each instance of k is independently an integer between 3 and 11, inclusive.

In certain embodiments, the compound of Formula (I) is of the formula:

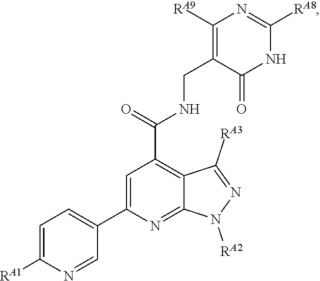

or a pharmaceutically acceptable salt, solvate, hydrate, polymorph, co-crystal, tautomer, stereoisomer, isotopically labeled derivative, or prodrug thereof.

In certain embodiments, the compound of Formula (I) is of the formula:

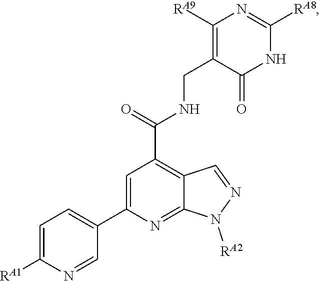

or a pharmaceutically acceptable salt, solvate, hydrate, polymorph, co-crystal, tautomer, stereoisomer, isotopically labeled derivative, or prodrug thereof.

In certain embodiments, the compound of Formula (I) is of the formula:

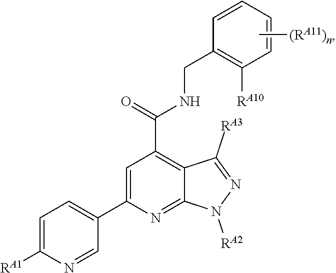

or a pharmaceutically acceptable salt, solvate, hydrate, polymorph, co-crystal, tautomer, stereoisomer, isotopically labeled derivative, or prodrug thereof.

In certain embodiments, the compound of Formula (I) is of the formula:

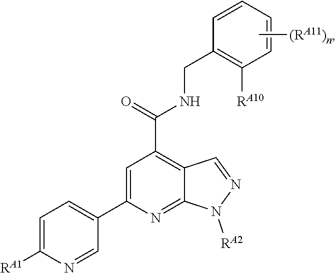

or a pharmaceutically acceptable salt, solvate, hydrate, polymorph, co-crystal, tautomer, stereoisomer, isotopically labeled derivative, or prodrug thereof.

In certain embodiments, the compound of Formula (I) is of the formula:

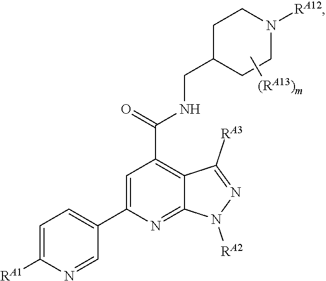

or a pharmaceutically acceptable salt, solvate, hydrate, polymorph, co-crystal, tautomer, stereoisomer, isotopically labeled derivative, or prodrug thereof.

In certain embodiments, the compound of Formula (I) is of the formula:

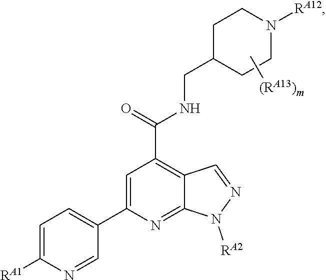

or a pharmaceutically acceptable salt, solvate, hydrate, polymorph, co-crystal, tautomer, stereoisomer, isotopically labeled derivative, or prodrug thereof.

In certain embodiments, the compound of Formula (I) is of the formula:

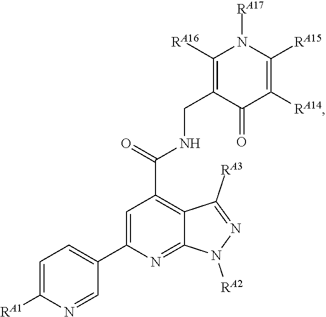

or a pharmaceutically acceptable salt, solvate, hydrate, polymorph, co-crystal, tautomer, stereoisomer, isotopically labeled derivative, or prodrug thereof.

In certain embodiments, the compound of Formula (I) is of the formula:

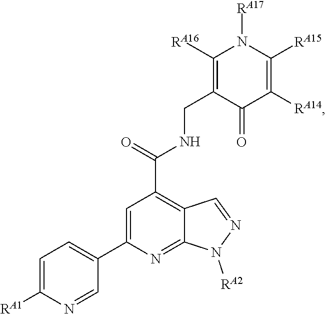

or a pharmaceutically acceptable salt, solvate, hydrate, polymorph, co-crystal, tautomer, stereoisomer, isotopically labeled derivative, or prodrug thereof.

Exemplary compounds of Formula (I) include, but are not limited to:

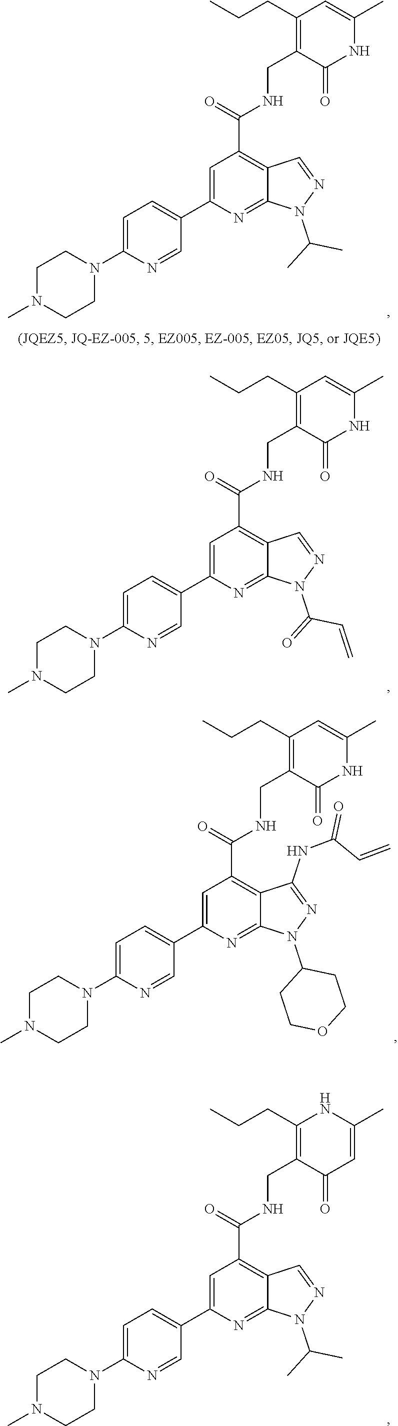

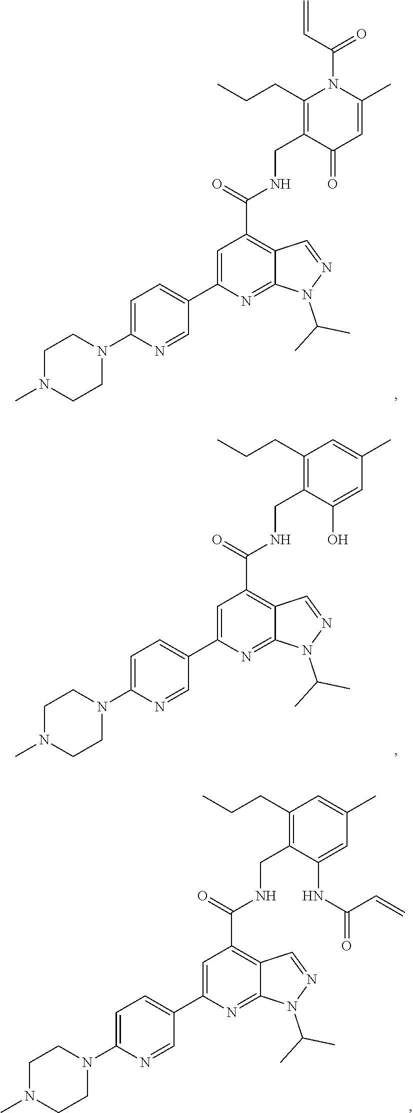

and pharmaceutically acceptable salts, solvates, hydrates, polymorphs, co-crystals, tautomers, stereoisomers, isotopically labeled derivatives, and prodrugs thereof.

Exemplary compounds of Formula (I) further include, but are not limited to:

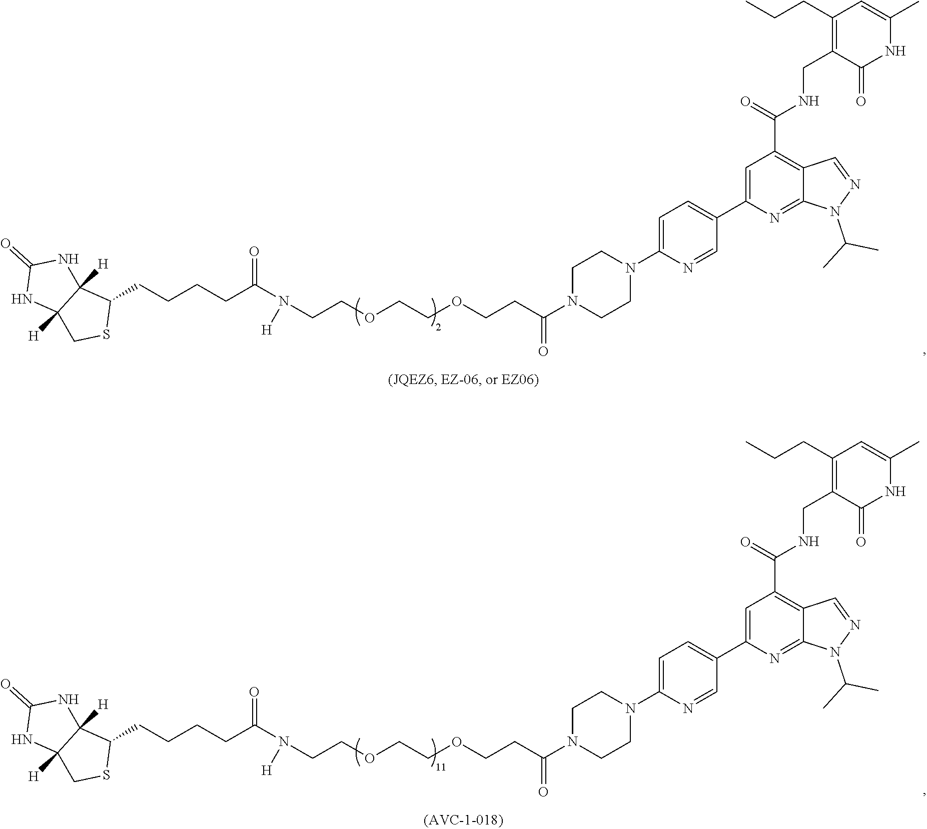

and pharmaceutically acceptable salts, solvates, hydrates, polymorphs, co-crystals, tautomers, stereoisomers, isotopically labeled derivatives, and prodrugs thereof.

In certain embodiments, the EZH2 inhibitor is a compound of the formula:

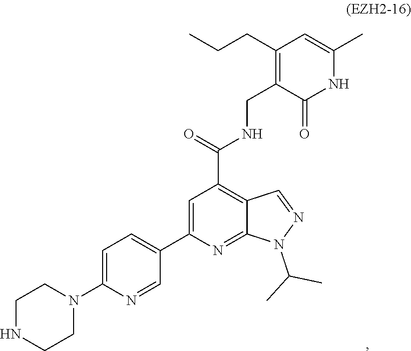 [SMILES: CCCc1cc(C)[nH]c(=O)c1CNC(=O)c1cc(-c2ccc(N3CCNCC3)nc2)nc2c1cnn2C(C)C ; ","]

or a pharmaceutically acceptable salt, solvate, hydrate, polymorph, co-crystal, tautomer, stereoisomer, isotopically labeled derivative, or prodrug thereof.

In certain embodiments, the EZH2 inhibitor is a compound of Formula (II):

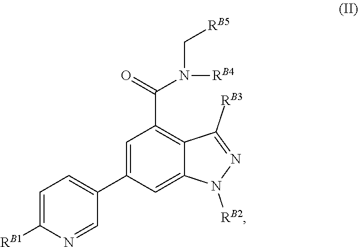

or a pharmaceutically acceptable salt, solvate, hydrate, polymorph, co-crystal, tautomer, stereoisomer, isotopically labeled derivative, or prodrug thereof, wherein:

R^{B1} is halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{b}, —N(R^{b})₂, —SR^{b}, —CN, —SCN, —C(═NR^{b})R^{b}, —C(═NR^{b})OR^{b}, —C(═NR^{b})N(R^{b})₂, —C(═O)R^{b}, —C(═O)OR^{b}, —C(═O)N(R^{b})₂, —NO₂, —NR^{b}C(═O)R^{b}, —NR^{b}C(═O)OR^{b}, —NR^{b}C(═O)N(R^{b})₂, —OC(═O)R^{b}, —OC(═O)OR^{b}, —OC(═O)N(R^{b})₂, or

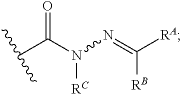

each instance of R^{b} is independently hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, an oxygen protecting group when attached to an oxygen atom, or a sulfur protecting group when attached to a sulfur atom, or two instances of R^{b} are joined to form a substituted or unsubstituted, heterocyclic ring, or substituted or unsubstituted, heteroaryl ring;

R^{A} is hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;

R^{B} is hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;

or R^{A} and R^{B} are joined to form a substituted or unsubstituted, carbocyclic ring, or a substituted or unsubstituted, heterocyclic ring;

R^{C} is hydrogen, substituted or unsubstituted C₁₋₆ alkyl, or a nitrogen protecting group;

R^{B2} is hydrogen, substituted or unsubstituted acyl, substituted or unsubstituted alkyl, substituted or unsubstituted carbocyclyl, substituted or unsubstituted heterocyclyl, a nitrogen protecting group, or a warhead;

R^{B3} is hydrogen, halogen, substituted or unsubstituted C₁₋₆ alkyl, —OR^{b}, —N(R^{b})₂, or a warhead;

R^{B4} is hydrogen, substituted or unsubstituted C₁₋₆ alkyl, or a nitrogen protecting group; and

R^{B5} is of the formula:

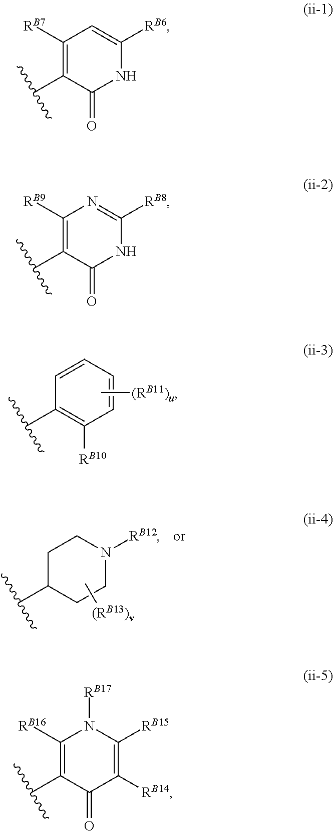

wherein:

R^{B6} is hydrogen, halogen, substituted or unsubstituted C₁₋₆ alkyl, or —N(R^{b})₂;

R^{B7} is hydrogen, halogen, substituted or unsubstituted C₂₋₆ alkyl, or substituted or unsubstituted, 3- to 7-membed, monocyclic carbocyclyl comprising 0, 1, or 2 double bonds in the carbocyclic ring system;

R^{B8} is hydrogen, halogen, substituted or unsubstituted C₁₋₆ alkyl, or —N(R^{b})₂;

R^{B9} is hydrogen, halogen, substituted or unsubstituted C₁₋₆ alkyl, or substituted or unsubstituted, 3- to 7-membed, monocyclic carbocyclyl comprising 0, 1, or 2 double bonds in the carbocyclic ring system;

R^{B10} is —OR^{b}, —N(R^{b})₂, or a warhead;

each instance of R^{B11} is independently halogen, substituted or unsubstituted C₁₋₆ alkyl, substituted or unsubstituted, 3- to 7-membed, monocyclic carbocyclyl comprising 0, 1, or 2 double bonds in the carbocyclic ring system, or —N(R^{b})₂;

u is 0, 1, 2, 3, or 4;

R^{B12} is hydrogen, substituted or unsubstituted C₁₋₆ alkyl, a nitrogen protecting group, or a warhead;

each instance of R^{B13} is independently halogen, substituted or unsubstituted C₁₋₆ alkyl, substituted or unsubstituted, 3- to 7-membed, monocyclic carbocyclyl comprising 0, 1, or 2 double bonds in the carbocyclic ring system, or —N(R^{b})₂;

v is 0, 1, 2, 3, 4, 5, 6, 7, 8, or 9;

R^{B14} is hydrogen, halogen, substituted or unsubstituted C₁₋₆ alkyl, —OR^{b}, or —N(R^{b})₂;

R^{B15} is hydrogen, halogen, substituted or unsubstituted C₁₋₆ alkyl, —OR^{b}, or —N(R^{b})₂;

R^{B16} is hydrogen, halogen, substituted or unsubstituted C₂₋₆ alkyl, substituted or unsubstituted, 3- to 7-membed, monocyclic carbocyclyl comprising 0, 1, or 2 double bonds in the carbocyclic ring system, —OR^{b}, or —N(R^{b})₂; and

R^{B17} is hydrogen, substituted or unsubstituted acyl, substituted or unsubstituted C₁₋₆ alkyl, a nitrogen protecting group, or a warhead.

In certain embodiments, the EZH2 inhibitor is a compound of Formula (II):

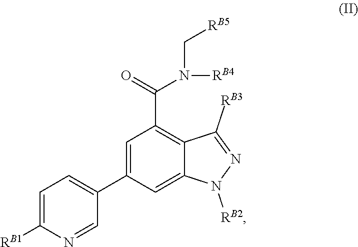

or a pharmaceutically acceptable salt, solvate, hydrate, polymorph, co-crystal, tautomer, stereoisomer, isotopically labeled derivative, or prodrug thereof, wherein:

R^{B1} is halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{b}, —N(R^{b})₂, —SR^{b}, —CN, —SCN, —C(═NR^{b})R^{b}, —C(═NR^{b})OR^{b}, —C(═NR^{b})N(R^{b})₂, —C(═O)R^{b}, —C(═O)OR^{b}, —C(═O)N(R^{b})₂, —NO₂, —NR^{b}C(═O)R^{b}, —NR^{b}C(═O)OR^{b}, —NR^{b}C(═O)N(R^{b})₂, —OC(═O)R^{b}, —OC(═O)OR^{b}, or —OC(═O)N(R^{b})₂;

each instance of R^{b} is independently hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, an oxygen protecting group when attached to an oxygen atom, or a sulfur protecting group when attached to a sulfur atom, or two instances of R^{b} are joined to form a substituted or unsubstituted, heterocyclic ring, or substituted or unsubstituted, heteroaryl ring;

R^{B2} is hydrogen, substituted or unsubstituted acyl, substituted or unsubstituted alkyl, substituted or unsubstituted carbocyclyl, substituted or unsubstituted heterocyclyl, or a nitrogen protecting group;

R^{B3} is hydrogen, halogen, substituted or unsubstituted C₁₋₆ alkyl, —OR^{b}, or —N(R^{b})₂;

R^{B4} is hydrogen, substituted or unsubstituted C₁₋₆ alkyl, or a nitrogen protecting group; and

R^{B5} is of the formula:

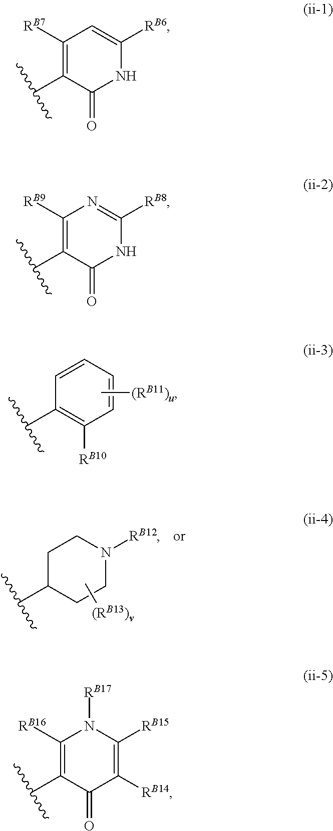

wherein:

R^{B6} is hydrogen, halogen, substituted or unsubstituted C₁₋₆ alkyl, or —N(R^{b})₂;

R^{B7} is hydrogen, halogen, substituted or unsubstituted C₂₋₆ alkyl, or substituted or unsubstituted, 3- to 7-membed, monocyclic carbocyclyl comprising 0, 1, or 2 double bonds in the carbocyclic ring system;

R^{B8} is hydrogen, halogen, substituted or unsubstituted C₁₋₆ alkyl, or —N(R^{b})₂;

R^{B9} is hydrogen, halogen, substituted or unsubstituted C₁₋₆ alkyl, or substituted or unsubstituted, 3- to 7-membed, monocyclic carbocyclyl comprising 0, 1, or 2 double bonds in the carbocyclic ring system;

R^{B10} is —OR^{b} or —N(R^{b})₂;

each instance of R^{B11} is independently halogen, substituted or unsubstituted C₁₋₆ alkyl, substituted or unsubstituted, 3- to 7-membed, monocyclic carbocyclyl comprising 0, 1, or 2 double bonds in the carbocyclic ring system, or —N(R^{b})₂;

u is 0, 1, 2, 3, or 4;

R^{B12} is hydrogen, substituted or unsubstituted C₁₋₆ alkyl, or a nitrogen protecting group;

each instance of R^{B13} is independently halogen, substituted or unsubstituted C₁₋₆ alkyl, substituted or unsubstituted, 3- to 7-membed, monocyclic carbocyclyl comprising 0, 1, or 2 double bonds in the carbocyclic ring system, or —N(R^{b})₂;

v is 0, 1, 2, 3, 4, 5, 6, 7, 8, or 9;

R^{B14} is hydrogen, halogen, substituted or unsubstituted C₁₋₆ alkyl, —OR^{b}, or —N(R^{b})₂;

R^{B15} is hydrogen, halogen, substituted or unsubstituted C₁₋₆ alkyl, —OR^{b}, or —N(R^{b})₂;

R^{B16} is hydrogen, halogen, substituted or unsubstituted C₂₋₆ alkyl, substituted or unsubstituted, 3- to 7-membed, monocyclic carbocyclyl comprising 0, 1, or 2 double bonds in the carbocyclic ring system, —OR^{b}, or —N(R^{b})₂; and

R^{B17} is hydrogen, substituted or unsubstituted acyl, substituted or unsubstituted C₁₋₆ alkyl, or a nitrogen protecting group.

Formula (II) includes substituent R^{B1} on the pyridinyl ring. In certain embodiments, R^{B1} is halogen (e.g., F, Cl, Br, or I). In certain embodiments, R^{B1} is substituted or unsubstituted alkyl (e.g., substituted or unsubstituted C₁₋₆ alkyl). In certain embodiments, R^{B1} is Me. In certain embodiments, R^{B1} is —CF₃, Bn, Et, perfluoroethyl, Pr, perfluoropropyl, Bu, or perfluorobutyl. In certain embodiments, R^{B1} is substituted or unsubstituted alkenyl (e.g., substituted or unsubstituted C₂₋₆ alkenyl). In certain embodiments, R^{B1} is substituted or unsubstituted alkynyl (e.g., substituted or unsubstituted C₁₋₆ alkynyl). In certain embodiments, R^{B1} is substituted or unsubstituted carbocyclyl (e.g., substituted or unsubstituted, 3- to 7-membered, monocyclic carbocyclyl comprising zero, one, or two double bonds in the carbocyclic ring system). In certain embodiments, R^{B1} is substituted or unsubstituted heterocyclyl (e.g., substituted or unsubstituted, 3- to 7-membed, monocyclic heterocyclyl comprising zero, one, or two double bonds in the heterocyclic ring system, wherein one, two, or three atoms in the heterocyclic ring system are independently nitrogen, oxygen, or sulfur). In certain embodiments, R^{B1} is substituted or unsubstituted piperazinyl. In certain embodiments, R^{B1} is of the formula:

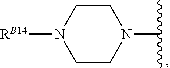

wherein R^{B14} is hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, or a nitrogen protecting group. In certain embodiments, R^{B1} is of the formula:

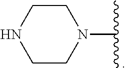 [SMILES: *N1CCNCC1 ; "."]

In certain embodiments, R^{B1} is of the formula:

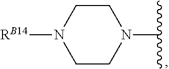

wherein R^{B14} is substituted or unsubstituted C₁₋₆ alkyl. In certain embodiments, R^{B1} is of the formula:

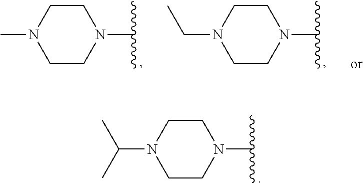

In certain embodiments, R^{B1} is of the formula:

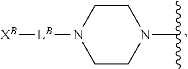

wherein L^{B} is a bond or substituted or unsubstituted C₁₋₁₀₀ hydrocarbon chain, optionally wherein one or more chain atoms of the hydrocarbon chain are independently replaced with —O—, —S—, or —NR^{b}; and X^{B} is a small molecule, peptide, protein, or polynucleotide. In certain embodiments, R^{B1} is of the formula:

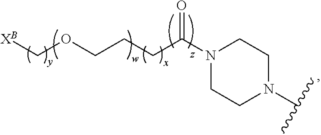

wherein z is 0 or 1, w is an integer between 0 and 11, inclusive, x is an integer between 0 and 10, inclusive, and y is an integer between 0 and 10, inclusive. In certain embodiments, w is an integer between 3 and 11, inclusive, x is 0, and y is 0, 1, 2, 3, 4, 5, or 6. In certain embodiments, X^{B} is a small molecule. In certain embodiments, X^{B} is a small molecule drug (e.g.,

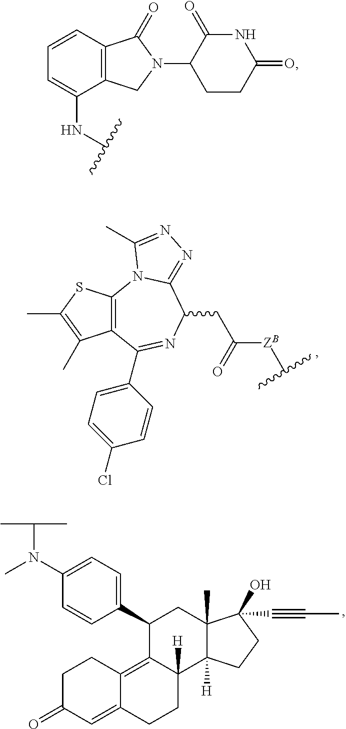

wherein Z^{B} is —O— or —NH—, or an additional pharmaceutical agent described herein that is a small molecule). In certain embodiments, X^{B} is a small molecule label (e.g., a biotin moiety (e.g.,

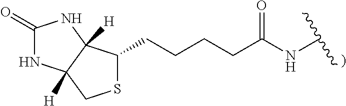 [SMILES: *NC(=O)CCCC[C@@H]1SC[C@@H]2NC(=O)N[C@@H]21]

or a small molecule fluorophore). In certain embodiments, R^{B1} is substituted or unsubstituted oxetanyl, substituted or unsubstituted tetrahydrofuranyl, substituted or unsubstituted pyrrolidinyl, substituted or unsubstituted tetrahydropyranyl, substituted or unsubstituted piperidinyl, substituted or unsubstituted morpholinyl, substituted or unsubstituted azepanyl, or substituted or unsubstituted diazepanyl. In certain embodiments, R^{B1} is of the formula:

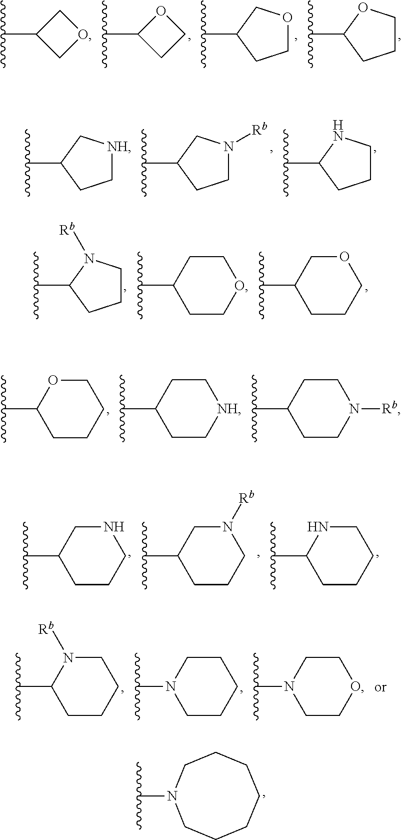

wherein each instance of R^{b} is independently unsubstituted C₁₋₆ alkyl (e.g., Me)). In certain embodiments, R^{B1} is substituted or unsubstituted aryl (e.g., substituted or unsubstituted, 6- to 10-membered aryl). In certain embodiments, R^{B1} is substituted or unsubstituted phenyl. In certain embodiments, R^{B1} is substituted or unsubstituted heteroaryl (e.g., substituted or unsubstituted, 5- to 6-membed, monocyclic heteroaryl, wherein one, two, three, or four atoms in the heteroaryl ring system are independently nitrogen, oxygen, or sulfur). In certain embodiments, R^{B1} is —OR^{b} (e.g., —OH, —O(substituted or unsubstituted C₁₋₆ alkyl) (e.g., —OMe, —OEt, —OPr, —OBu, or —OBn), or —O(substituted or unsubstituted phenyl) (e.g., —OPh)). In certain embodiments, R^{B1} is —SR^{b} (e.g., —SH, —S(substituted or unsubstituted C₁₋₆ alkyl) (e.g., —SMe, —SEt, —SPr, —SBu, or —SBn), or —S(substituted or unsubstituted phenyl) (e.g., —SPh)). In certain embodiments, R^{B1} is —N(R^{b})₂ (e.g., —NH₂, —NH(substituted or unsubstituted C₁₋₆ alkyl) (e.g., —NHMe), or —N(substituted or unsubstituted C₁₋₆ alkyl)-(substituted or unsubstituted C₁₋₆ alkyl) (e.g., —NMe₂)). In certain embodiments, R^{B1} is —CN. In certain embodiments, R^{B1} is —SCN or —NO₂. In certain embodiments, R^{B1} is —C(═NR^{b})R^{b}, —C(═NR^{b})OR^{b}, or —C(═NR^{b})N(R^{b})₂. In certain embodiments, R^{B1} is —C(═O)R^{b} (e.g., —C(═O)(substituted or unsubstituted alkyl) or —C(═O)(substituted or unsubstituted phenyl)). In certain embodiments, R^{B1} is —C(═O)OR^{b} (e.g., —C(═O)OH, —C(═O)O(substituted or unsubstituted alkyl) (e.g., —C(═O)OMe), or —C(═O)O(substituted or unsubstituted phenyl)). In certain embodiments, R^{B1} is —C(═O)N(R^{b})₂ (e.g., —C(═O)NH₂, —C(═O)NH(substituted or unsubstituted alkyl), —C(═O)NH(substituted or unsubstituted phenyl), —C(═O)N(substituted or unsubstituted alkyl)-(substituted or unsubstituted alkyl), or —C(═O)N(substituted or unsubstituted phenyl)-(substituted or unsubstituted alkyl)). In certain embodiments, R^{B1} is —NR^{b}C(═O)R^{b} (e.g., —NHC(═O)Me). In certain embodiments, R^{B1} is —NR^{b}C(═O)OR^{b} or —NR^{b}C(═O)N(R^{b})₂. In certain embodiments, R^{B1} is —OC(═O)R^{b}, —OC(═O)OR^{b}, or —OC(═O)N(R^{b})₂. In certain embodiments, R^{B1} is substituted or unsubstituted alkyl, —OR^{b}, —N(R^{b})₂, —C(═O)OR^{b}, or —NR^{b}C(═O)R^{b}. In certain embodiments, R^{B1} is unsubstituted C₁₋₆ alkyl, —OMe, —NH₂, —N(Me)₂, —C(═O)OH, —C(═O)OMe, or —NHC(═O)Me. In certain embodiments, R^{B1} is

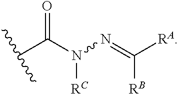

Formula (II) may include one or more instances of substituent R^{b}. When Formula (II) includes two or more instances of R^{b}, any two instances of R^{b} may be the same or different from each other. In certain embodiments, at least one instance of R^{b} is H. In certain embodiments, each instance of R^{b} is H. In certain embodiments, at least one instance of R^{b} is substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, a nitrogen protecting group (e.g., Bn, Boc, Cbz, Fmoc, trifluoroacetyl, triphenylmethyl, acetyl, or Ts) when attached to a nitrogen atom, an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom, or two instances of R^{b} are joined to form a substituted or unsubstituted, heterocyclic ring, or substituted or unsubstituted, heteroaryl ring.

Formula (II) includes substituent R^{B2} at the 1-position of the indolyl ring. In certain embodiments, R^{B2} is H. In certain embodiments, R^{B2} is substituted or unsubstituted acyl. In certain embodiments, R^{B2} is —C(═O)R^{b}, optionally wherein R^{b} is substituted or unsubstituted C₁₋₆ alkyl (e.g., Me) or substituted or unsubstituted C₂₋₆ alkenyl. In certain embodiments, R^{B2} is —C(═O)R^{b}, wherein R^{b} is substituted or unsubstituted vinyl. In certain embodiments, R^{B2} is —C(═O)CH═CH₂. In certain embodiments, R^{B2} is —C(═O)OR^{b}, optionally wherein R^{b} is H, substituted or unsubstituted C₁₋₆ alkyl (e.g., Me), or an oxygen protecting group (e.g., silyl, TBDPS, TBDMS, TIPS, TES, TMS, MOM, THP, t-Bu, Bn, allyl, acetyl, pivaloyl, or benzoyl). In certain embodiments, R^{B2} is —C(═O)N(R^{b})₂, optionally wherein each instance of R^{b} is independently H, substituted or unsubstituted C₁₋₆ alkyl (e.g., Me), or a nitrogen protecting group (e.g., Bn, Boc, Cbz, Fmoc, trifluoroacetyl, triphenylmethyl, acetyl, or Ts). In certain embodiments, R^{B2} is substituted or unsubstituted alkyl (e.g., substituted or unsubstituted C₁₋₆ alkyl). In certain embodiments, R^{B2} is Me. In certain embodiments, R^{B2} is Et. In certain embodiments, R^{B2} is n-Pr. In certain embodiments, R^{B2} is i-Pr. In certain embodiments, R^{B2} is Bu (e.g., n-Bu, i-Bu, sec-Bu, or t-Bu). In certain embodiments, R^{B2} is unsubstituted pentyl (e.g., unsubstituted n-pentyl, unsubstituted t-pentyl, unsubstituted neopentyl, unsubstituted isopentyl, unsubstituted sec-pentyl, or unsubstituted 3-pentyl). In certain embodiments, R^{B2} is sec-Bu, t-Bu, or unsubstituted 3-pentyl. In certain embodiments, R^{B2} is —CF₃, Bn, perfluoroethyl, perfluoropropyl, perfluorobutyl, or perfluoropentyl. In certain embodiments, R^{B2} is —CH₂C(═O)—NH—N═C(R^{b})₂. In certain embodiments, R^{B2} is substituted or unsubstituted carbocyclyl (e.g., substituted or unsubstituted, 3- to 7-membered, monocyclic carbocyclyl comprising zero, one, or two double bonds in the carbocyclic ring system). In certain embodiments, R^{B2} is substituted or unsubstituted cyclopropyl. In certain embodiments, R^{B2} is unsubstituted cyclopropyl. In certain embodiments, R^{B2} is substituted or unsubstituted cyclobutyl. In certain embodiments, R^{B2} is substituted or unsubstituted cyclopentyl. In certain embodiments, R^{B2} is unsubstituted cyclopropyl, unsubstituted cyclobutyl, or unsubstituted cyclopentyl. In certain embodiments, R^{B2} is substituted or unsubstituted heterocyclyl (e.g., substituted or unsubstituted, 3- to 7-membed, monocyclic heterocyclyl comprising zero, one, or two double bonds in the heterocyclic ring system, wherein one, two, or three atoms in the heterocyclic ring system are independently nitrogen, oxygen, or sulfur). In certain embodiments, R^{B2} is substituted or unsubstituted tetrahydropyranyl. In certain embodiments, R^{B2} is of the formula:

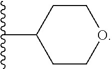 [SMILES: *C1CCOCC1]

In certain embodiments, R^{B2} is of the formula:

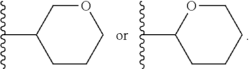

In certain embodiments, R^{B2} is substituted or unsubstituted oxetanyl, substituted or unsubstituted tetrahydrofuranyl, substituted or unsubstituted pyrrolidinyl, substituted or unsubstituted piperidinyl, substituted or unsubstituted morpholinyl, or substituted or unsubstituted piperazinyl. In certain embodiments, R^{B2} is of the formula:

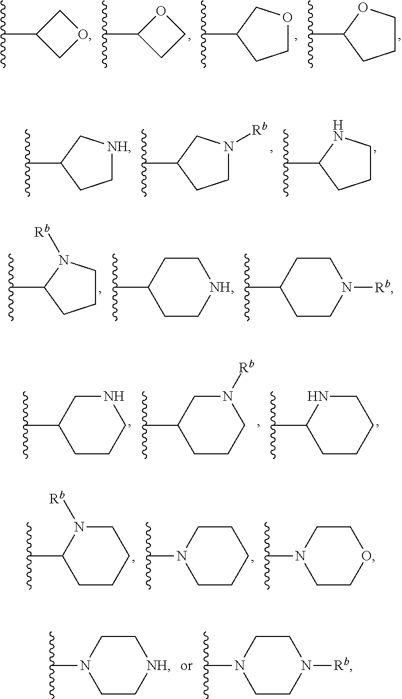

wherein each instance of R^{b} is independently unsubstituted C₁₋₆ alkyl (e.g., Me)). In certain embodiments, R^{B2} is a nitrogen protecting group (e.g., Bn, Boc, Cbz, Fmoc, trifluoroacetyl, triphenylmethyl, acetyl, or Ts). In certain embodiments, R^{B2} is Boc. In certain embodiments, R^{B2} is a warhead.

Formula (II) includes substituent R^{B3} at the 3-position of the indolyl ring. In certain embodiments, R^{B3} is H. In certain embodiments, R^{B3} is halogen (e.g., F, Cl, Br, or I). In certain embodiments, R^{B3} is substituted or unsubstituted C₁₋₆ alkyl. In certain embodiments, R^{B3} is Me. In certain embodiments, R^{B3} is —CF₃, Bn, Et, perfluoroethyl, Pr, perfluoropropyl, Bu, or perfluorobutyl. In certain embodiments, R^{B3} is —OR^{b}, optionally wherein R^{b} is H, substituted or unsubstituted C₁₋₆ alkyl (e.g., Me), substituted or unsubstituted acyl, or an oxygen protecting group (e.g., silyl, TBDPS, TBDMS, TIPS, TES, TMS, MOM, THP, t-Bu, Bn, allyl, acetyl, pivaloyl, or benzoyl). In certain embodiments, R^{B3} is —OC(═O)R^{b}, optionally wherein R^{b} is H, substituted or unsubstituted C₁₋₆ alkyl (e.g., Me), or substituted or unsubstituted C₂₋₆ alkenyl (e.g., substituted or unsubstituted vinyl). In certain embodiments, R^{B3} is —OC(═O)CH═CH₂. In certain embodiments, R^{B3} is —N(R^{b})₂, optionally wherein each instance of R^{b} is independently H, substituted or unsubstituted C₁₋₆ alkyl (e.g., Me), substituted or unsubstituted acyl, or a nitrogen protecting group (e.g., Bn, Boc, Cbz, Fmoc, trifluoroacetyl, triphenylmethyl, acetyl, or Ts). In certain embodiments, R^{B3} is —N(R^{b})C(═O)R^{b}, optionally wherein each instance of R^{b} is independently H, substituted or unsubstituted C₁₋₆ alkyl (e.g., Me), or substituted or unsubstituted C₂₋₆ alkenyl (e.g., substituted or unsubstituted vinyl). In certain embodiments, R^{B3} is —NHC(═O)CH═CH₂. In certain embodiments, R^{B3} is a warhead.

Formula (II) includes substituent R^{B4} on a nitrogen atom. In certain embodiments, R^{B4} is H. In certain embodiments, R^{B4} is substituted or unsubstituted C₁₋₆ alkyl. In certain embodiments, R^{B4} is Me. In certain embodiments, R^{B4} is —CF₃, Bn, Et, perfluoroethyl, Pr, perfluoropropyl, Bu, or perfluorobutyl. In certain embodiments, R^{B4} is a nitrogen protecting group (e.g., Bn, Boc, Cbz, Fmoc, trifluoroacetyl, triphenylmethyl, acetyl, or Ts).

Formula (II) includes substituent R^{B5}. In certain embodiments, R^{B5} is of Formula (ii-1):

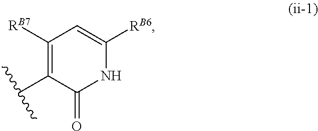

(e.g.,

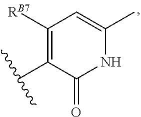

wherein R^{B7} is Et, Pr, or Bu). In certain embodiments, R^{B5} is of the formula:

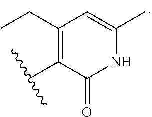 [SMILES: *c1c(CC)cc(C)[nH]c1=O]

In certain embodiments, R^{B5} is of the formula:

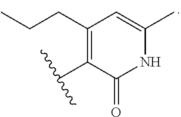 [SMILES: *c1c(CCC)cc(C)[nH]c1=O]

In certain embodiments, R^{B6} is H. In certain embodiments, R^{B6} is halogen (e.g., F, Cl, Br, or I). In certain embodiments, R^{B6} is substituted or unsubstituted C₁₋₆ alkyl. In certain embodiments, R^{B6} is Me. In certain embodiments, R^{B6} is substituted methyl (e.g., —CF₃ or Bn). In certain embodiments, R^{B6} is Et, substituted ethyl (e.g., perfluoroethyl), Pr, substituted propyl (e.g., perfluoropropyl), Bu, or substituted butyl (e.g., perfluorobutyl). In certain embodiments, R^{B6} is —OR^{b} (e.g., —OH or —O(substituted or unsubstituted C₁₋₆ alkyl) (e.g., —OMe)). In certain embodiments, R^{B6} is —N(R^{b})₂, optionally wherein each instance of R^{b} is independently hydrogen, substituted or unsubstituted C₁₋₆ alkyl, or a nitrogen protecting group (e.g., Bn, Boc, Cbz, Fmoc, trifluoroacetyl, triphenylmethyl, acetyl, or Ts). In certain embodiments, R^{B6} is —NH₂, —NHMe, or —N(Me)₂. In certain embodiments, R^{B7} is H. In certain embodiments, R^{B7} is halogen (e.g., F, Cl, Br, or I). In certain embodiments, R^{B7} is substituted or unsubstituted C₂₋₆ alkyl. In certain embodiments, R^{B7} is Et. In certain embodiments, R^{B7} is substituted ethyl (e.g., perfluoroethyl). In certain embodiments, R^{B7} is n-Pr. In certain embodiments, R^{B7} is i-Pr. In certain embodiments, R^{B7} is substituted propyl (e.g., perfluoropropyl). In certain embodiments, R^{B7} is Bu or unsubstituted pentyl. In certain embodiments, R^{B7} is substituted butyl (e.g., perfluorobutyl) or substituted pentyl (e.g., perfluoropentyl). In certain embodiments, R^{B7} is substituted or unsubstituted, 3- to 7-membed, monocyclic carbocyclyl comprising 0, 1, or 2 double bonds in the carbocyclic ring system. In certain embodiments, R^{B7} is substituted or unsubstituted cyclopropyl, substituted or unsubstituted cyclobutyl, or substituted or unsubstituted cyclopentyl. In certain embodiments, R^{B7} is —OR^{b} (e.g., —OH or —O(substituted or unsubstituted C₁₋₆ alkyl) (e.g., —OMe)). In certain embodiments, R^{B7} is —N(R^{b})₂, optionally wherein each instance of R^{b} is independently hydrogen, substituted or unsubstituted C₁₋₆ alkyl, or a nitrogen protecting group (e.g., Bn, Boc, Cbz, Fmoc, trifluoroacetyl, triphenylmethyl, acetyl, or Ts). In certain embodiments, R^{B7} is —NH₂, —NHMe, —NHEt, —N(Me)₂, or —N(Et)₂. In certain embodiments, R^{B7} is substituted or unsubstituted cyclopropyl or —N(R^{b})₂, wherein each instance of R^{b} is independently hydrogen, substituted or unsubstituted C₁₋₆ alkyl, or a nitrogen protecting group (e.g., Bn, Boc, Cbz, Fmoc, trifluoroacetyl, triphenylmethyl, acetyl, or Ts).
In certain embodiments, R^{B5} is of the formula:

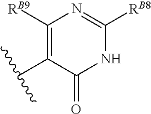

(e.g.,

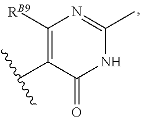

wherein R^{B9} is Me, Et, Pr, or Bu). The moiety of the formula:

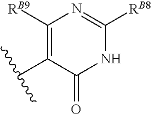

also includes its tautomeric form

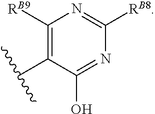

In certain embodiments, R^{B5} is of the formula:

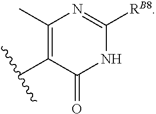

In certain embodiments, R^{B5} is of the formula:

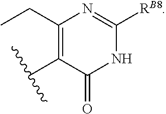

In certain embodiments, R^{B5} is of the formula:

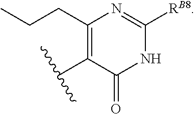

In certain embodiments, R^{B8} is H. In certain embodiments, R^{B8} is halogen (e.g., F, Cl, Br, or I). In certain embodiments, R^{B8} is substituted or unsubstituted C₁₋₆ alkyl. In certain embodiments, R^{B8} is Me. In certain embodiments, R^{B8} is substituted methyl (e.g., —CF₃ or Bn). In certain embodiments, R^{B8} is Et, substituted ethyl (e.g., perfluoroethyl), Pr, substituted propyl (e.g., perfluoropropyl), Bu, or substituted butyl (e.g., perfluorobutyl). In certain embodiments, R^{B8} is —OR^{b} (e.g., —OH or —O(substituted or unsubstituted C₁₋₆ alkyl) (e.g., —OMe)). In certain embodiments, R^{B8} is —N(R^{b})₂, optionally wherein each instance of R^{b} is independently hydrogen, substituted or unsubstituted C₁₋₆ alkyl, or a nitrogen protecting group (e.g., Bn, Boc, Cbz, Fmoc, trifluoroacetyl, triphenylmethyl, acetyl, or Ts). In certain embodiments, R^{B8} is —NH₂, —NHMe, or —N(Me)₂. In certain embodiments, R^{B9} is H. In certain embodiments, R^{B9} is halogen (e.g., F, Cl, Br, or I). In certain embodiments, R^{B9} is substituted or unsubstituted C₁₋₆ alkyl. In certain embodiments, R^{B9} is Me. In certain embodiments, R^{B9} is substituted methyl (e.g., —CF₃ or Bn). In certain embodiments, R^{B9} is Et. In certain embodiments, R^{B9} is substituted ethyl (e.g., perfluoroethyl). In certain embodiments, R^{B9} is n-Pr. In certain embodiments, R^{B9} is i-Pr. In certain embodiments, R^{B9} is substituted propyl (e.g., perfluoropropyl). In certain embodiments, R^{B9} is Bu or unsubstituted pentyl. In certain embodiments, R^{B9} is substituted butyl (e.g., perfluorobutyl) or substituted pentyl (e.g., perfluoropentyl). In certain embodiments, R^{B9} is substituted or unsubstituted, 3- to 7-membed, monocyclic carbocyclyl comprising 0, 1, or 2 double bonds in the carbocyclic ring system. In certain embodiments, R^{B9} is substituted or unsubstituted cyclopropyl, substituted or unsubstituted cyclobutyl, or substituted or unsubstituted cyclopentyl. In certain embodiments, R^{B9} is —OR^{b} (e.g., —OH or —O(substituted or unsubstituted C₁₋₆ alkyl) (e.g., —OMe)). In certain embodiments, R^{B9} is —N(R^{b})₂, optionally wherein each instance of R^{b} is independently hydrogen, substituted or unsubstituted C₁₋₆ alkyl, or a nitrogen protecting group (e.g., Bn, Boc, Cbz, Fmoc, trifluoroacetyl, triphenylmethyl, acetyl, or Ts). In certain embodiments, R^{B9} is —NH₂, —NHMe, —NHEt, —N(Me)₂, or —N(Et)₂. In certain embodiments, R^{B9} is substituted or unsubstituted cyclopropyl, —OR^{b}, or —N(R^{b})₂, wherein each instance of R^{b} is independently hydrogen, substituted or unsubstituted C₁₋₆ alkyl, an oxygen protecting group when attached to an oxygen atom, or a nitrogen protecting group when attached to a nitrogen atom.
In certain embodiments, R^{B5} is of the formula:

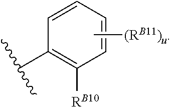

In certain embodiments, R^{B5} is of the formula:

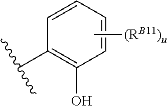

(e.g.,

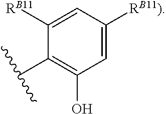

In certain embodiments, R^{B5} is of the formula:

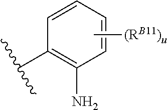

(e.g.,

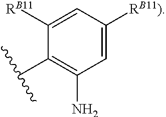

In certain embodiments, R^{B5} is of the formula:

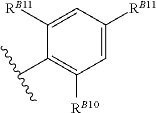

(e.g.,

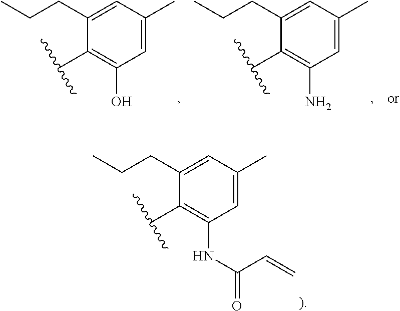

In certain embodiments, R^{B5} is of the formula:

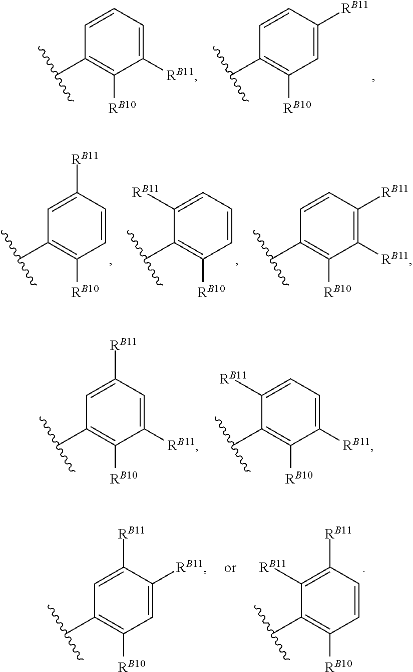

In certain embodiments, R^{B5} is of the formula:

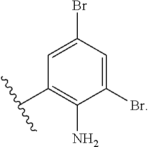 [SMILES: *c1cc(Br)cc(Br)c1N]

In certain embodiments, R^{B10} is —OR^{b} (e.g., —OH). In certain embodiments, R^{B10} is —N(R^{b})₂. In certain embodiments, R^{B10} is —NH₂. In certain embodiments, R^{B10} is —NHR^{b}, wherein R^{b} is substituted or unsubstituted C₁₋₆ alkyl or a nitrogen protecting group (e.g., Bn, Boc, Cbz, Fmoc, trifluoroacetyl, triphenylmethyl, acetyl, or Ts). In certain embodiments, R^{B10} is a warhead. In certain embodiments, R^{B10} is —NHC(═O)R^{b}, optionally wherein R^{b} is substituted or unsubstituted vinyl. In certain embodiments, R^{B10} is —NHC(═O)CH═CH₂. When Formula (II) includes two or more instances of R^{B11}, any two instances of R^{B11} may be the same or different from each other. In certain embodiments, at least one instance of R^{B11} is halogen. In certain embodiments, at least one instance of R^{B11} is Br. In certain embodiments, at least one instance of R^{B11} is F, Cl, or I. In certain embodiments, at least one instance of R^{B11} is substituted or unsubstituted C₁₋₆ alkyl. In certain embodiments, at least one instance of R^{B11} is Me. In certain embodiments, at least one instance of R^{B11} is substituted methyl (e.g., —CF₃ or Bn). In certain embodiments, at least one instance of R^{B11} is Et. In certain embodiments, at least one instance of R^{B11} is substituted ethyl (e.g., perfluoroethyl). In certain embodiments, at least one instance of R^{B11} is n-Pr. In certain embodiments, at least one instance of R^{B11} is i-Pr. In certain embodiments, at least one instance of R^{B11} is substituted propyl (e.g., perfluoropropyl). In certain embodiments, at least one instance of R^{B11} is Me, Et, or n-Pr. In certain embodiments, at least one instance of R^{B11} is Bu or unsubstituted pentyl. In certain embodiments, at least one instance of R^{B11} is substituted butyl (e.g., perfluorobutyl) or substituted pentyl (e.g., perfluoropentyl). In certain embodiments, at least one instance of R^{B11} is substituted or unsubstituted, 3- to 7-membed, monocyclic carbocyclyl comprising 0, 1, or 2 double bonds in the carbocyclic ring system. In certain embodiments, at least one instance of R^{B11} is substituted or unsubstituted cyclopropyl, substituted or unsubstituted cyclobutyl, or substituted or unsubstituted cyclopentyl. In certain embodiments, at least one instance of R^{B11} is —OR^{b} (e.g., —OH or —O(substituted or unsubstituted C₁₋₆ alkyl) (e.g., —OMe)). In certain embodiments, at least one instance of R^{B11} is —N(R^{b})₂, optionally wherein each instance of R^{b} is independently hydrogen, substituted or unsubstituted C₁₋₆ alkyl, or a nitrogen protecting group (e.g., Bn, Boc, Cbz, Fmoc, trifluoroacetyl, triphenylmethyl, acetyl, or Ts). In certain embodiments, at least one instance of R^{B11} is —NH₂, —NHMe, —NHEt, —N(Me)₂, or —N(Et)₂. In certain embodiments, at least one instance of R^{B11} is substituted or unsubstituted C₁₋₆ alkyl, substituted or unsubstituted cyclopropyl, —OR^{b}, or —N(R^{b})₂, wherein each instance of R^{b} is independently hydrogen, substituted or unsubstituted C₁₋₆ alkyl, an oxygen protecting group when attached to an oxygen atom, or a nitrogen protecting group when attached to a nitrogen atom. In certain embodiments, u is 0. In certain embodiments, u is 1. In certain embodiments, u is 2. In certain embodiments, u is 3. In certain embodiments, u is 4.

In certain embodiments, R^{B5} is of the formula:

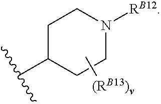

In certain embodiments, R^{B5} is of the formula:

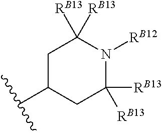

(e.g.,

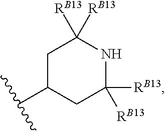

such as

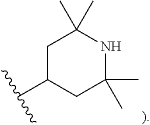 [SMILES: *C1CC(C)(C)NC(C)(C)C1]

In certain embodiments, R^{B12} is H. In certain embodiments, R^{B12} is substituted or unsubstituted C₁₋₆ alkyl. In certain embodiments, R^{B12} is Me. In certain embodiments, R^{B12} is —CF₃, Bn, Et, perfluoroethyl, Pr, perfluoropropyl, Bu, or perfluorobutyl. In certain embodiments, R^{B12} is a nitrogen protecting group (e.g., Bn, Boc, Cbz, Fmoc, trifluoroacetyl, triphenylmethyl, acetyl, or Ts). In certain embodiments, R^{B12} is a warhead. When Formula (II) includes two or more instances of R^{B13}, any two instances of R^{B13} may be the same or different from each other. In certain embodiments, at least one instance of R^{B13} is halogen (e.g., F, Cl, Br, or I). In certain embodiments, at least one instance of R^{B13} is substituted or unsubstituted C₁₋₆ alkyl. In certain embodiments, at least one instance of R^{B13} is Me. In certain embodiments, at least one instance of R^{B13} is substituted methyl (e.g., —CF₃ or Bn). In certain embodiments, at least one instance of R^{B13} is Et. In certain embodiments, at least one instance of R^{B13} is substituted ethyl (e.g., perfluoroethyl). In certain embodiments, at least one instance of R^{B13} is n-Pr. In certain embodiments, at least one instance of R^{B13} is i-Pr. In certain embodiments, at least one instance of R^{B13} is substituted propyl (e.g., perfluoropropyl). In certain embodiments, at least one instance of R^{B13} is Me, Et, or n-Pr. In certain embodiments, at least one instance of R^{B13} is Bu or unsubstituted pentyl. In certain embodiments, at least one instance of R^{B13} is substituted butyl (e.g., perfluorobutyl) or substituted pentyl (e.g., perfluoropentyl). In certain embodiments, at least one instance of R^{B13} is substituted or unsubstituted, 3- to 7-membed, monocyclic carbocyclyl comprising 0, 1, or 2 double bonds in the carbocyclic ring system. In certain embodiments, at least one instance of R^{B13} is substituted or unsubstituted cyclopropyl, substituted or unsubstituted cyclobutyl, or substituted or unsubstituted cyclopentyl. In certain embodiments, at least one instance of R^{B13} is —OR^{b} (e.g., —OH or —O(substituted or unsubstituted C₁₋₆ alkyl) (e.g., —OMe)). In certain embodiments, at least one instance of R^{B13} is —N(R^{b})₂, optionally wherein each instance of R^{b} is independently hydrogen, substituted or unsubstituted C₁₋₆ alkyl, or a nitrogen protecting group (e.g., Bn, Boc, Cbz, Fmoc, trifluoroacetyl, triphenylmethyl, acetyl, or Ts). In certain embodiments, at least one instance of R^{B13} is —NH₂, —NHMe, —NHEt, —N(Me)₂, or —N(Et)₂. In certain embodiments, at least one instance of R^{B13} is halogen, substituted or unsubstituted cyclopropyl, —OR^{b}, or —N(R^{b})₂, wherein each instance of R^{b} is independently hydrogen, substituted or unsubstituted C₁₋₆ alkyl, an oxygen protecting group when attached to an oxygen atom, or a nitrogen protecting group when attached to a nitrogen atom. In certain embodiments, v is 0. In certain embodiments, v is 1. In certain embodiments, v is 2. In certain embodiments, v is 3, 4, 5, 6, 7, or 8. In certain embodiments, v is 9.

Formula (II) includes substituent R^{B5}. In certain embodiments, R^{B5} is of the formula:

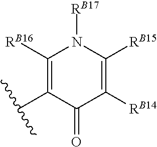

(e.g.,

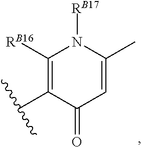

optionally wherein R^{B16} is Et, Pr, or Bu). In certain embodiments, R^{B5} is of the formula:

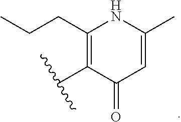 [SMILES: *c1c(CCC)[nH]c(C)cc1=O]

In certain embodiments, R^{B5} is of the formula:

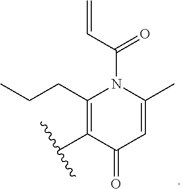 [SMILES: *c1c(CCC)n(C(=O)C=C)c(C)cc1=O]

In certain embodiments, R^{B14} is H. In certain embodiments, R^{B14} is halogen (e.g., F, Cl, Br, or I). In certain embodiments, R^{B14} is substituted or unsubstituted C₁₋₆ alkyl. In certain embodiments, R^{B14} is Me. In certain embodiments, R^{B14} is substituted methyl (e.g., —CF₃ or Bn). In certain embodiments, R^{B14} is Et, substituted ethyl (e.g., perfluoroethyl), Pr, substituted propyl (e.g., perfluoropropyl), Bu, or substituted butyl (e.g., perfluorobutyl). In certain embodiments, R^{B14} is —OR^{b} (e.g., —OH or —O(substituted or unsubstituted C₁₋₆ alkyl) (e.g., —OMe)). In certain embodiments, R^{B14} is —N(R^{b})₂, optionally wherein each instance of R^{b} is independently hydrogen, substituted or unsubstituted C₁₋₆ alkyl, or a nitrogen protecting group (e.g., Bn, Boc, Cbz, Fmoc, trifluoroacetyl, triphenylmethyl, acetyl, or Ts). In certain embodiments, R^{B14} is —NH₂, —NHMe, or —N(Me)₂. In certain embodiments, R^{B15} is H. In certain embodiments, R^{B15} is halogen (e.g., F, Cl, Br, or I). In certain embodiments, R^{B15} is substituted or unsubstituted C₁₋₆ alkyl. In certain embodiments, R^{B15} is Me. In certain embodiments, R^{B15} is substituted methyl (e.g., —CF₃ or Bn). In certain embodiments, R^{B15} is Et, substituted ethyl (e.g., perfluoroethyl), Pr, substituted propyl (e.g., perfluoropropyl), Bu, or substituted butyl (e.g., perfluorobutyl). In certain embodiments, R^{B15} is —OR^{b} (e.g., —OH or —O(substituted or unsubstituted C₁₋₆ alkyl) (e.g., —OMe)). In certain embodiments, R^{B15} is —N(R^{b})₂, optionally wherein each instance of R^{b} is independently hydrogen, substituted or unsubstituted C₁₋₆ alkyl, or a nitrogen protecting group (e.g., Bn, Boc, Cbz, Fmoc, trifluoroacetyl, triphenylmethyl, acetyl, or Ts). In certain embodiments, R^{B15} is —NH₂, —NHMe, or —N(Me)₂. In certain embodiments, R^{B16} is H. In certain embodiments, R^{B16} is halogen (e.g., F, Cl, Br, or I). In certain embodiments, R^{B16} is substituted or unsubstituted C₂₋₆ alkyl. In certain embodiments, R^{B16} is Et. In certain embodiments, R^{B16} is substituted ethyl (e.g., perfluoroethyl). In certain embodiments, R^{B16} is n-Pr. In certain embodiments, R^{B16} is i-Pr. In certain embodiments, R^{B16} is substituted propyl (e.g., perfluoropropyl). In certain embodiments, R^{B16} is Bu or unsubstituted pentyl. In certain embodiments, R^{B16} is substituted butyl (e.g., perfluorobutyl) or substituted pentyl (e.g., perfluoropentyl). In certain embodiments, R^{B16} is substituted or unsubstituted, 3- to 7-membed, monocyclic carbocyclyl comprising 0, 1, or 2 double bonds in the carbocyclic ring system. In certain embodiments, R^{B16} is substituted or unsubstituted cyclopropyl, substituted or unsubstituted cyclobutyl, or substituted or unsubstituted cyclopentyl. In certain embodiments, R^{B16} is —OR^{b} (e.g., —OH or —O(substituted or unsubstituted C₁₋₆ alkyl) (e.g., —OMe)). In certain embodiments, R^{B16} is —N(R^{b})₂, optionally wherein each instance of R^{b} is independently hydrogen, substituted or unsubstituted C₁₋₆ alkyl, or a nitrogen protecting group (e.g., Bn, Boc, Cbz, Fmoc, trifluoroacetyl, triphenylmethyl, acetyl, or Ts). In certain embodiments, R^{B16} is —NH₂, —NHMe, —NHEt, —N(Me)₂, or N(Et)₂. In certain embodiments, R^{B16} is substituted or unsubstituted cyclopropyl or —N(R^{b})₂, wherein each instance of R^{b} is independently hydrogen, substituted or unsubstituted C₁₋₆ alkyl, or a nitrogen protecting group (e.g., Bn, Boc, Cbz, Fmoc, trifluoroacetyl, triphenylmethyl, acetyl, or Ts). In certain embodiments, R^{B17} is H. In certain embodiments, R^{B17} is substituted or unsubstituted acyl. In certain embodiments, R^{B17} is —C(═O)R^{b}, optionally wherein R^{b} is substituted or unsubstituted C₁₋₆ alkyl (e.g., Me) or substituted or unsubstituted C₂₋₆ alkenyl. In certain embodiments, R^{B17} is a warhead. In certain embodiments, R^{B17} is —C(═O)R^{b}, wherein R^{b} is substituted or unsubstituted vinyl. In certain embodiments, R^{B17} is —C(═O)CH═CH₂. In certain embodiments, R^{B17} is —C(═O)OR^{b}, optionally wherein R^{b} is H, substituted or unsubstituted C₁₋₆ alkyl (e.g., Me), or an oxygen protecting group (e.g., silyl, TBDPS, TBDMS, TIPS, TES, TMS, MOM, THP, t-Bu, Bn, allyl, acetyl, pivaloyl, or benzoyl). In certain embodiments, R^{B17} is —C(═O)N(R^{b})₂, optionally wherein each instance of R^{b} is independently H, substituted or unsubstituted C₁₋₆ alkyl (e.g., Me), or a nitrogen protecting group (e.g., Bn, Boc, Cbz, Fmoc, trifluoroacetyl, triphenylmethyl, acetyl, or Ts). In certain embodiments, R^{B17} is substituted or unsubstituted C₁₋₆ alkyl. In certain embodiments, R^{B17} is Me. In certain embodiments, R^{B17} is —CF₃, Bn, Et, perfluoroethyl, Pr, perfluoropropyl, Bu, or perfluorobutyl. In certain embodiments, R^{B17} is a nitrogen protecting group (e.g., Bn, Boc, Cbz, Fmoc, trifluoroacetyl, triphenylmethyl, acetyl, or Ts).

In certain embodiments, the compound of Formula (II) is of the formula:

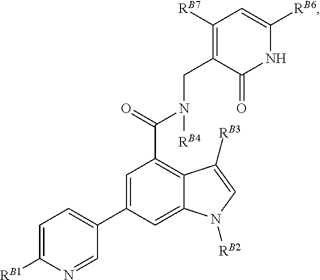

or a pharmaceutically acceptable salt, solvate, hydrate, polymorph, co-crystal, tautomer, stereoisomer, isotopically labeled derivative, or prodrug thereof.

In certain embodiments, the compound of Formula (II) is of the formula:

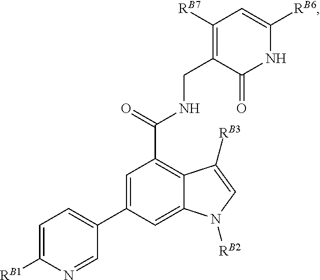

or a pharmaceutically acceptable salt, solvate, hydrate, polymorph, co-crystal, tautomer, stereoisomer, isotopically labeled derivative, or prodrug thereof.

In certain embodiments, the compound of Formula (II) is of the formula:

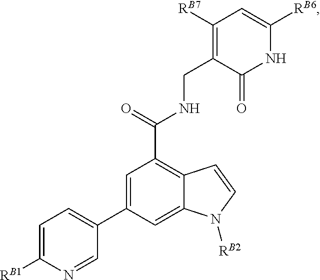

or a pharmaceutically acceptable salt, solvate, hydrate, polymorph, co-crystal, tautomer, stereoisomer, isotopically labeled derivative, or prodrug thereof.

In certain embodiments, the compound of Formula (II) is of the formula:

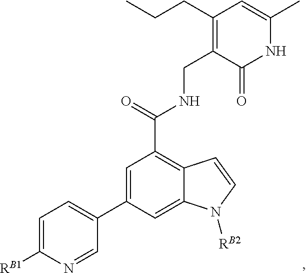

or a pharmaceutically acceptable salt, solvate, hydrate, polymorph, co-crystal, tautomer, stereoisomer, isotopically labeled derivative, or prodrug thereof.

In certain embodiments, the compound of Formula (II) is of the formula:

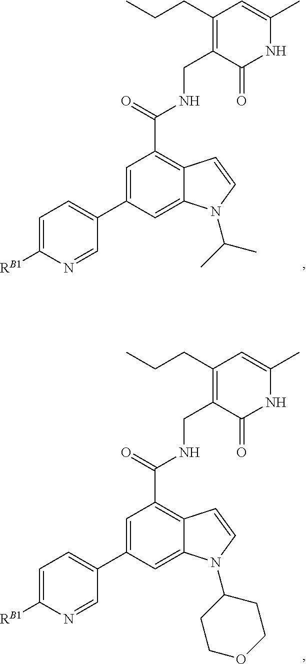

or a pharmaceutically acceptable salt, solvate, hydrate, polymorph, co-crystal, tautomer, stereoisomer, isotopically labeled derivative, or prodrug thereof.

In certain embodiments, the compound of Formula (II) is of the formula:

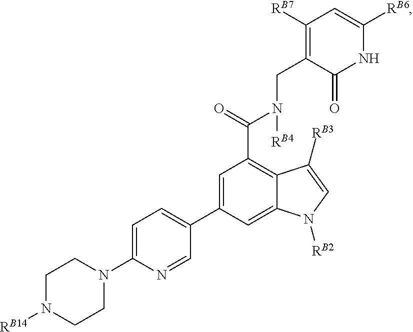

or a pharmaceutically acceptable salt, solvate, hydrate, polymorph, co-crystal, tautomer, stereoisomer, isotopically labeled derivative, or prodrug thereof, wherein R^{B14} is hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, or a nitrogen protecting group.

In certain embodiments, the compound of Formula (II) is of the formula:

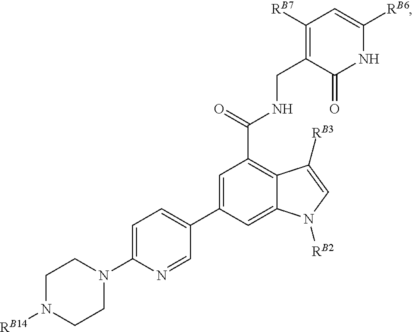

or a pharmaceutically acceptable salt, solvate, hydrate, polymorph, co-crystal, tautomer, stereoisomer, isotopically labeled derivative, or prodrug thereof.

In certain embodiments, the compound of Formula (II) is of the formula:

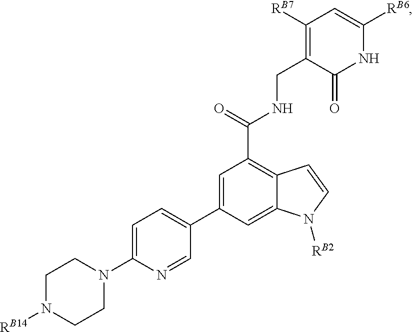

or a pharmaceutically acceptable salt, solvate, hydrate, polymorph, co-crystal, tautomer, stereoisomer, isotopically labeled derivative, or prodrug thereof.

In certain embodiments, the compound of Formula (II) is of the formula:

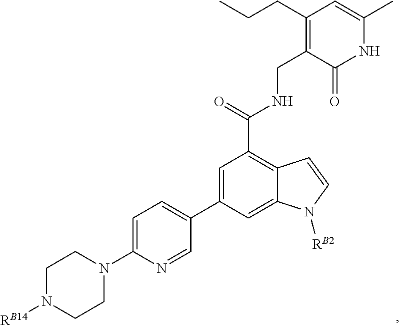

or a pharmaceutically acceptable salt, solvate, hydrate, polymorph, co-crystal, tautomer, stereoisomer, isotopically labeled derivative, or prodrug thereof.

In certain embodiments, the compound of Formula (II) is of the formula:

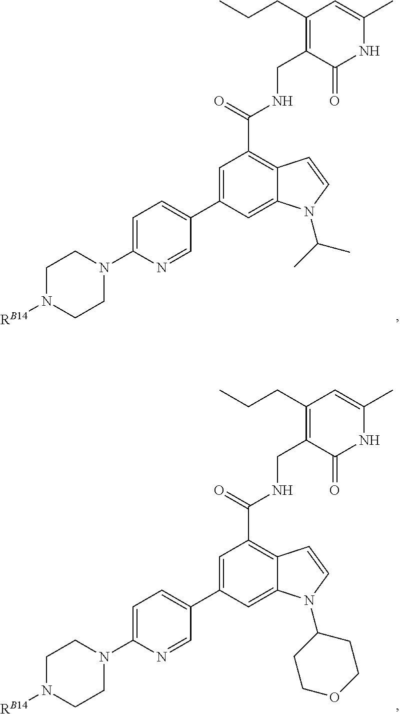

or a pharmaceutically acceptable salt, solvate, hydrate, polymorph, co-crystal, tautomer, stereoisomer, isotopically labeled derivative, or prodrug thereof.

In certain embodiments, the compound of Formula (II) is of the formula:

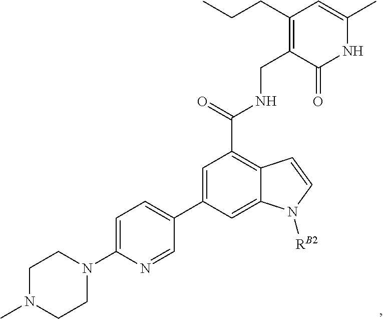

or a pharmaceutically acceptable salt, solvate, hydrate, polymorph, co-crystal, tautomer, stereoisomer, isotopically labeled derivative, or prodrug thereof, wherein R^{B2} is a nitrogen protecting group (e.g., Boc).

In certain embodiments, the compound of Formula (II) is of the formula:

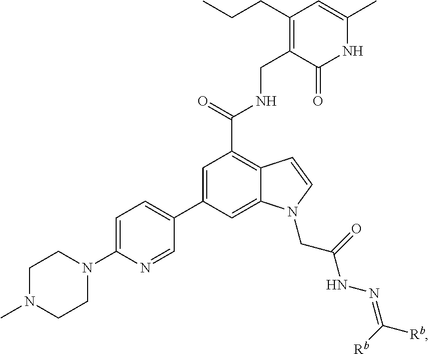

or a pharmaceutically acceptable salt, solvate, hydrate, polymorph, co-crystal, tautomer, stereoisomer, isotopically labeled derivative, or prodrug thereof.

In certain embodiments, the compound of Formula (II) is of the formula:

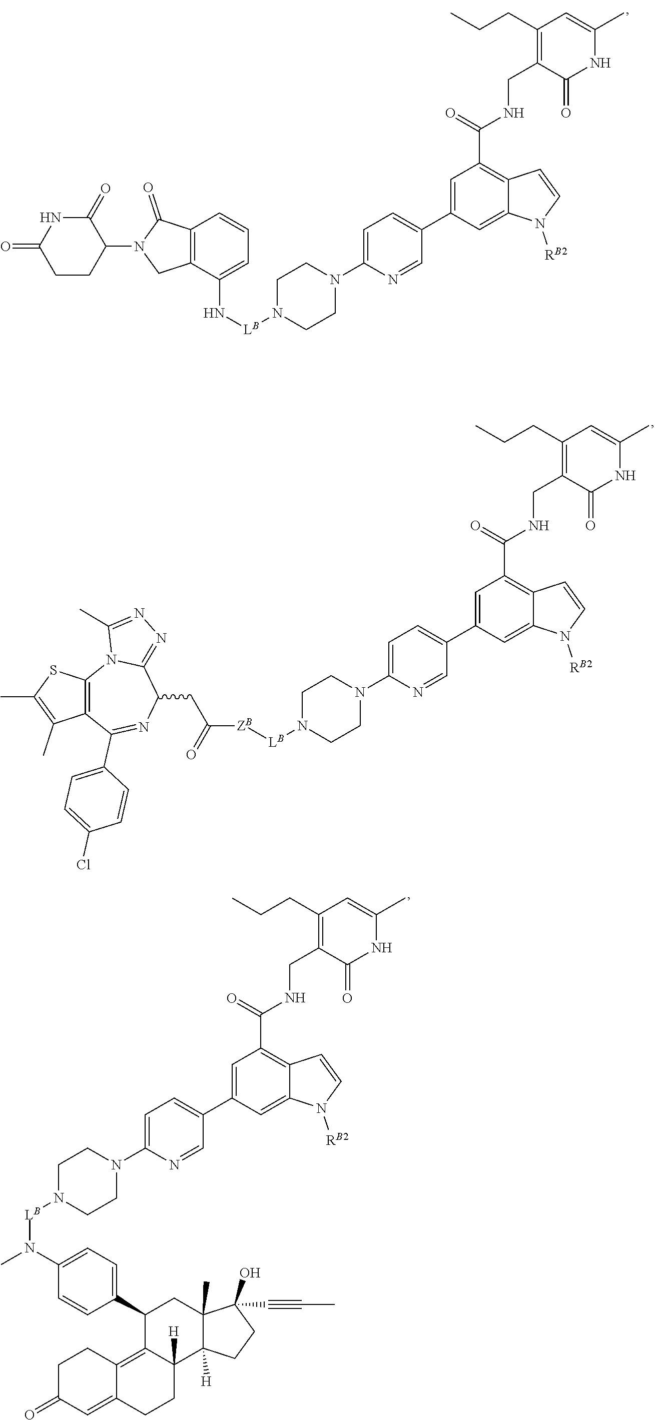

or a pharmaceutically acceptable salt, solvate, hydrate, polymorph, co-crystal, tautomer, stereoisomer, isotopically labeled derivative, or prodrug thereof, optionally wherein each instance of R^{B2} is i-Pr.

In certain embodiments, the compound of Formula (II) is of the formula:

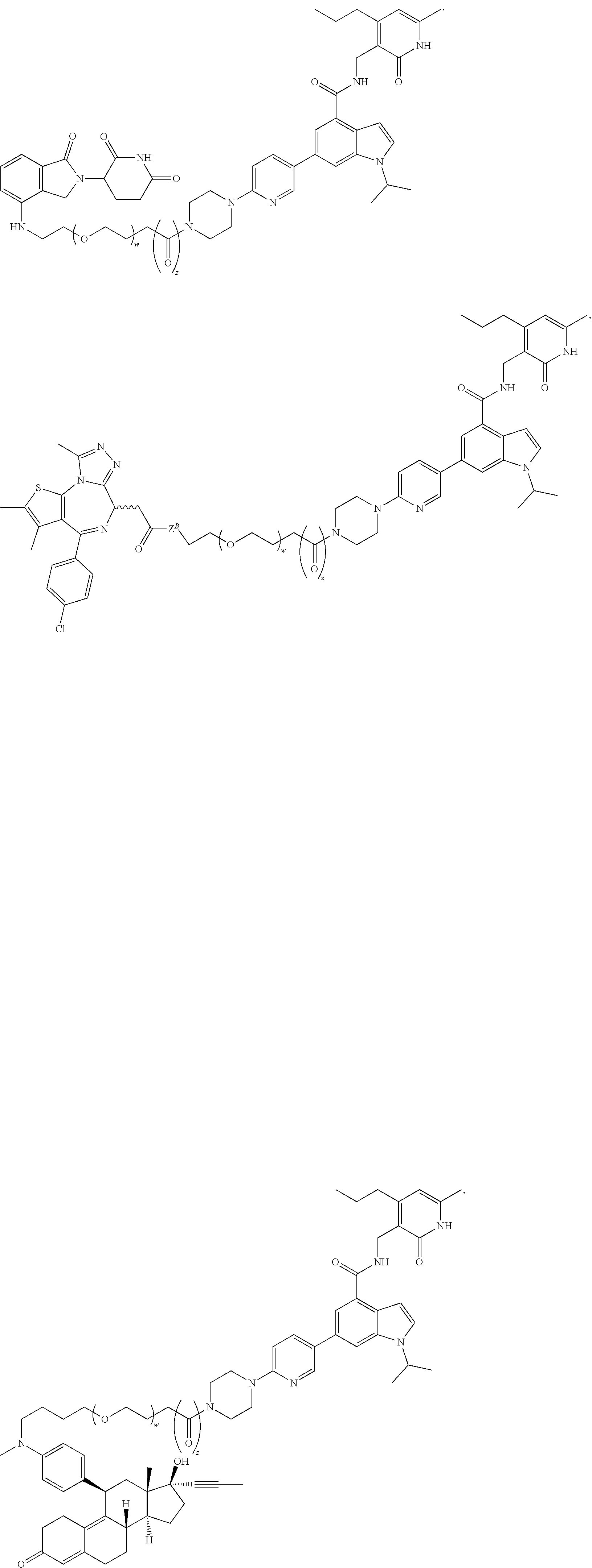

or a pharmaceutically acceptable salt, solvate, hydrate, polymorph, co-crystal, tautomer, stereoisomer, isotopically labeled derivative, or prodrug thereof, wherein each instance of w is independently an integer between 3 and 11, inclusive.

In certain embodiments, the compound of Formula (II) is of the formula:

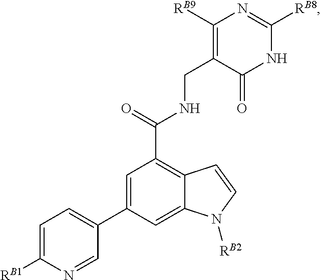

or a pharmaceutically acceptable salt, solvate, hydrate, polymorph, co-crystal, tautomer, stereoisomer, isotopically labeled derivative, or prodrug thereof.

In certain embodiments, the compound of Formula (II) is of the formula:

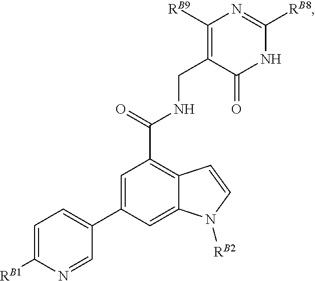

or a pharmaceutically acceptable salt, solvate, hydrate, polymorph, co-crystal, tautomer, stereoisomer, isotopically labeled derivative, or prodrug thereof.

In certain embodiments, the compound of Formula (II) is of the formula:

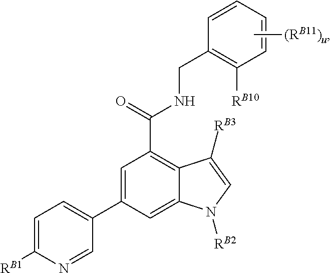

or a pharmaceutically acceptable salt, solvate, hydrate, polymorph, co-crystal, tautomer, stereoisomer, isotopically labeled derivative, or prodrug thereof.

In certain embodiments, the compound of Formula (II) is of the formula:

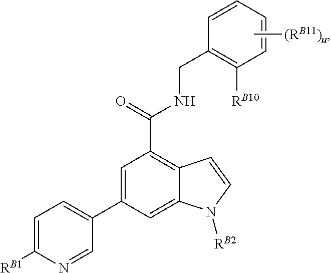

or a pharmaceutically acceptable salt, solvate, hydrate, polymorph, co-crystal, tautomer, stereoisomer, isotopically labeled derivative, or prodrug thereof.

In certain embodiments, the compound of Formula (II) is of the formula:

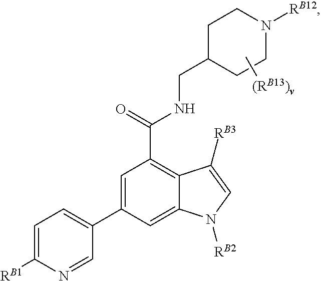

or a pharmaceutically acceptable salt, solvate, hydrate, polymorph, co-crystal, tautomer, stereoisomer, isotopically labeled derivative, or prodrug thereof.

In certain embodiments, the compound of Formula (II) is of the formula:

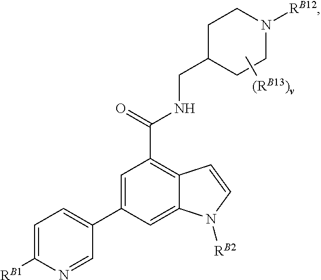

or a pharmaceutically acceptable salt, solvate, hydrate, polymorph, co-crystal, tautomer, stereoisomer, isotopically labeled derivative, or prodrug thereof.

In certain embodiments, when R^{B2} is i-Pr, R^{B3} is hydrogen, and R^{B5} is of Formula (ii-1), then R^{B1} is not Me,

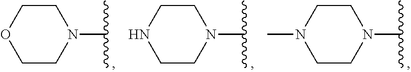

—OMe, or —NH(═O)Me; and when R^{B2} is unsubstituted C₃₋₅ alkyl, R^{B3} is Me or halogen, and R^{B5} is of Formula (ii-1), then R^{B1} is not Me,

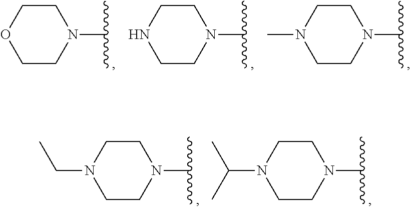

—OMe, —NH₂, —N(Me)₂, —C(═O)OH, —C(═O)OMe, or —NH(═O)Me. In certain embodiments, when R^{B2} is i-Pr, R^{B3} is hydrogen, and R^{B5} is of Formula (ii-1), then R^{B1} is not unsubstituted C₁₋₆ alkyl, —OR^{b}, —NH(═O)R^{b}, or unsubstituted or substituted with one unsubstituted C₁₋₆ alkyl, saturated, 6-membered, monocyclic heterocyclyl, wherein 2 atoms in the heterocyclic ring system are independently oxygen or nitrogen; and when R^{B2} is unsubstituted C₃₋₅ alkyl, R^{B3} is Me or halogen, and R^{B5} is of Formula (ii-1), then R^{B1} is not unsubstituted C₁₋₆ alkyl, —OR^{b}, —N(R^{b})₂, —C(═O)OR^{b}, —NH(═O)R^{b}, or unsubstituted or substituted with one unsubstituted C₁₋₆ alkyl, saturated, 6-membered, monocyclic heterocyclyl, wherein 2 atoms in the heterocyclic ring system are independently oxygen or nitrogen; wherein each instance of R^{b} is independently H or unsubstituted C₁₋₆ alkyl. In certain embodiments, when R^{B5} is of Formula (ii-1), then R^{B2} is not unsubstituted C₃₋₅ alkyl. In certain embodiments, when R^{B3} is hydrogen, and R^{B5} is of Formula (ii-1), then R^{B2} is not i-Pr. In certain embodiments, a compound of Formula (II) is not of the formula:

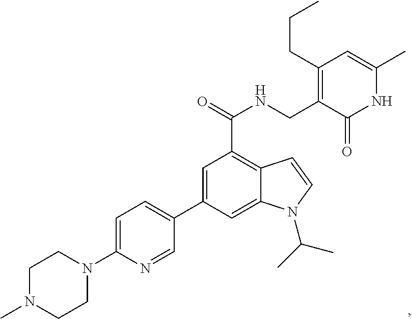

or a pharmaceutically acceptable salt, solvate, hydrate, polymorph, co-crystal, tautomer, stereoisomer, isotopically labeled derivative, or prodrug thereof.

Exemplary compounds of Formula (II) include, but are not limited to:

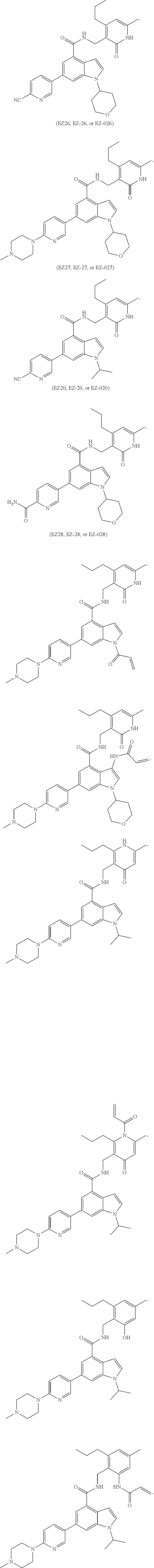

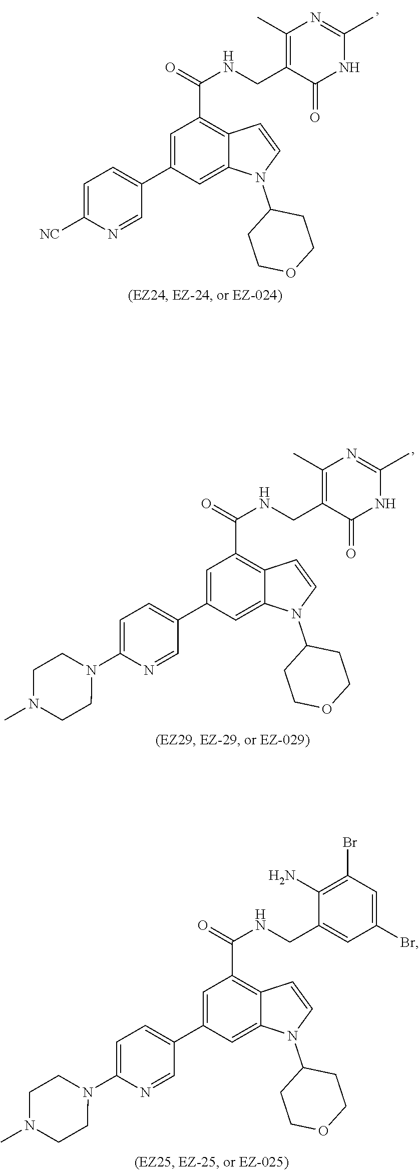

and pharmaceutically acceptable salts, solvates, hydrates, polymorphs, co-crystals, tautomers, stereoisomers, isotopically labeled derivatives, and prodrugs thereof.

Exemplary compounds of Formula (II) further include, but are not limited to:

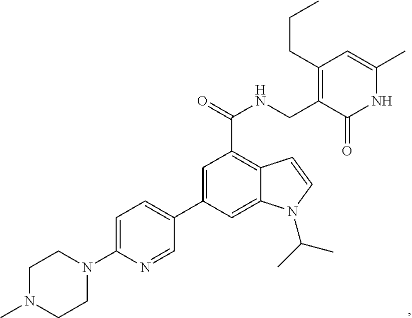

and pharmaceutically acceptable salts, solvates, hydrates, polymorphs, co-crystals, tautomers, stereoisomers, isotopically labeled derivatives, and prodrugs thereof.

In certain embodiments, the EZH2 inhibitor is a compound of the formula:

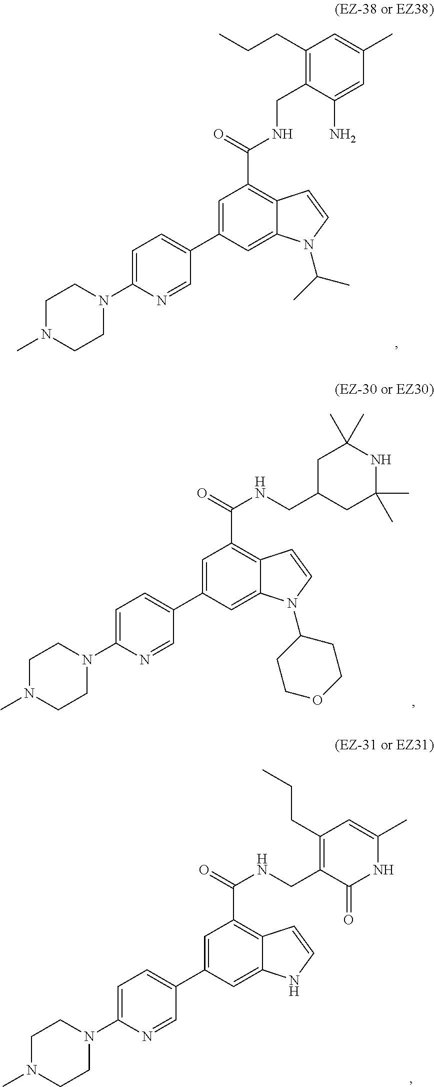

or a pharmaceutically acceptable salt, solvate, hydrate, polymorph, co-crystal, tautomer, stereoisomer, isotopically labeled derivative, or prodrug thereof.

In certain embodiments, the EZH2 inhibitor is a compound of the formula:

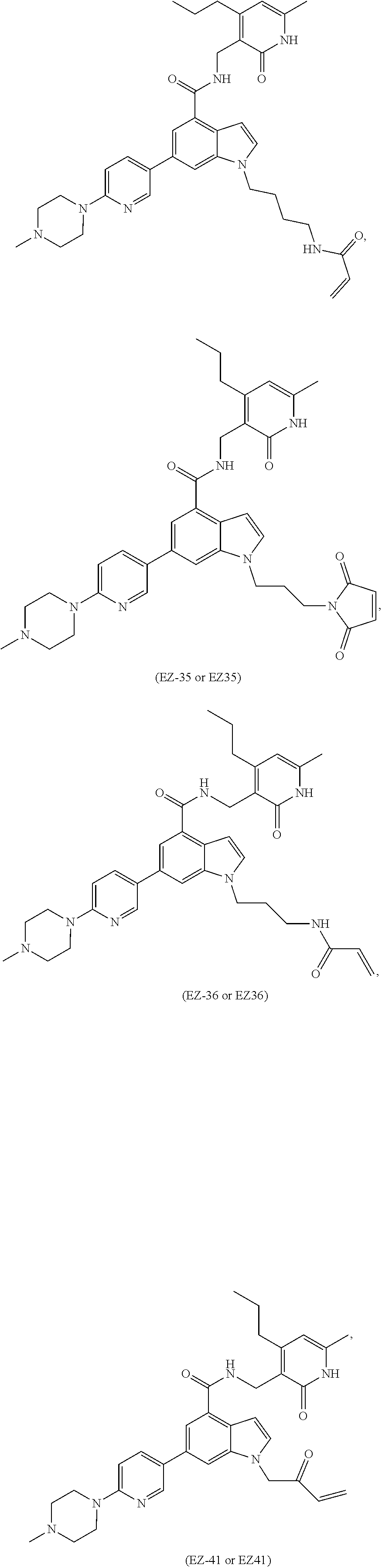

or a pharmaceutically acceptable salt, solvate, hydrate, polymorph, co-crystal, tautomer, stereoisomer, isotopically labeled derivative, or prodrug thereof.

In certain embodiments, a compound of Formula (I) or (II) is a reversible EZH2 inhibitor. In certain embodiments, a compound of Formula (I) or (II) that does not include a warhead is a reversible EZH2 inhibitor. In certain embodiments, a compound of Formula (I) or (II) that does not include a warhead does not form a covalent bond with EZH2.

In certain embodiments, a compound of Formula (I) or (II) is an irreversible EZH2 inhibitor. In certain embodiments, a compound of Formula (I) or (II) that includes one or more warheads is an irreversible EZH2 inhibitor. In certain embodiments, a compound of Formula (I) or (II) that includes one or more warheads forms one or more covalent bonds with EZH2 (e.g., a cysteine residue of EZH2). In certain embodiments, a compound of Formula (I) or (II) that includes one or more warheads forms one or more covalent bonds with EZH2 (e.g., a cysteine residue of EZH2) through a reaction (e.g., a Michael addition) between EZH2 and at least one of the warheads. In certain embodiments, the irreversible EZH2 inhibitor is of the formula:

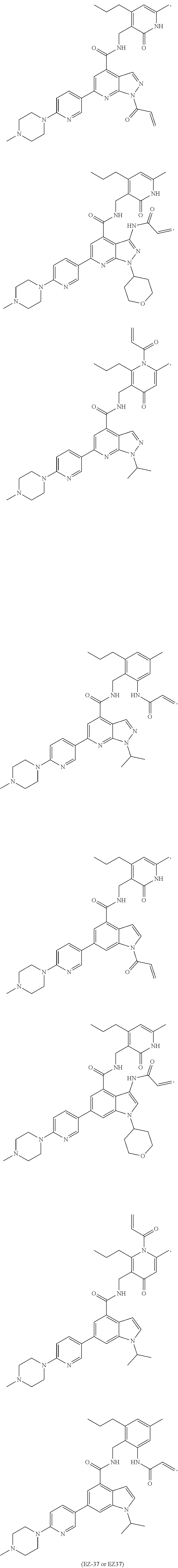

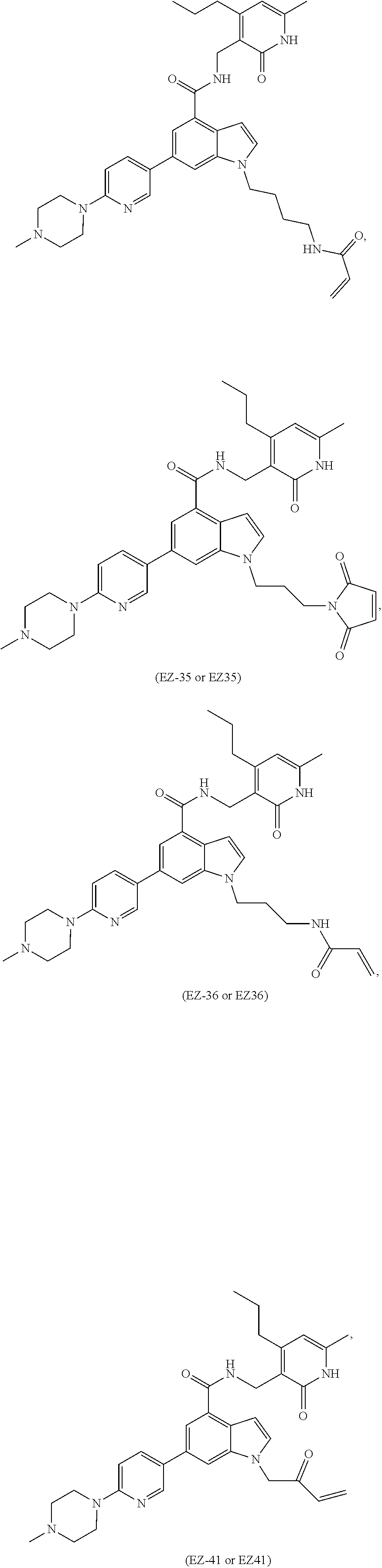

or a pharmaceutically acceptable salt, solvate, hydrate, polymorph, co-crystal, tautomer, stereoisomer, isotopically labeled derivative, or prodrug thereof.

Examples of Bcl6 inhibitors are known in the art. For example, Bcl6 inhibitor disclosed in U.S. Pat. No. 8,338,464, U.S. patent application Ser. No. 13/182,163, PCT Publication number WO2010008436 A1 or PCT Publication number WO2008066887 A2, the contents of which are herein incorporated by reference in their entirety.

In some embodiments, the Bcl6 inhibitor is of the formula:

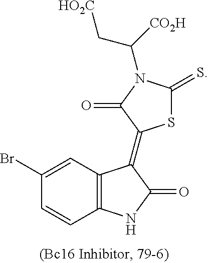 [SMILES: O=C(O)CC(C(=O)O)N1C(=O)/C(=C2/C(=O)Nc3ccc(Br)cc32)SC1=S]

In some embodiments, the Bcl6 inhibitor is a compound having the structure:

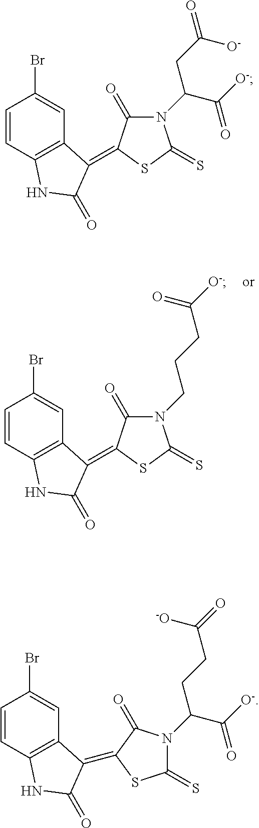

In some embodiments, the BRD4 inhibitor is:

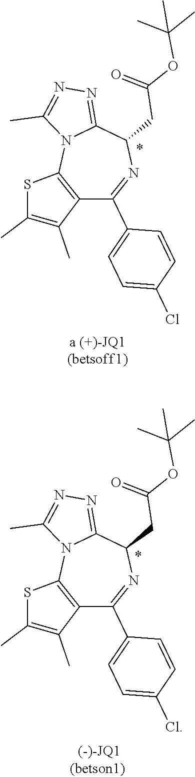

The present invention is further illustrated by the following Examples, which in no way should be construed as further limiting. The entire contents of all of the references (including literature references, issued patents, published patent applications, and co pending patent applications) cited throughout this application are hereby expressly incorporated by reference.

### EXAMPLES

### Example 1

### Methods

B10.BR recipients were conditioned with cytoxan on days −3 and −2 (120 mg/kg/day i.p.). On day −1, recipients received TBI by x-ray (8.3 Gy). B6 donor BM was T-cell depleted with anti-Thy1.2 mAb followed by rabbit complement. T-cells were purified from spleens by incubation with biotin-labeled anti-CD19, anti-CD11b, anti-CD11c, anti-NK1.1 and anti-γδTCR (eBioscience), followed by strepavidin beads and depletion with magnetic column (StemCell Technologies). On day 0, recipients received 10×106 T cell depleted BM cells with or without allogeneic spleen purified T cells (0.075-0.1×106), Weights of individual mice were recorded weekly. Where indicated, recipients in BM and T cell groups were given EZH2 inhibitor (either JQ-EZH2-5 (75 μg/animal/3 times a week) or JQ1 (50 μg/animal/Daily) in 10% Hydroxypropyl-beta-cyclodextrin and DMSO in a 9:1 ratio) or Bcl 79-6 (50 μg/animal/three times a week) in PBS.

### Results

Bcl6 in T-Cells is Necessary for cGVHD Progression

An animal model of cGVHD was prepared according to the methods above. Table 1 shows a graphical representation of the animal model protocol.

TABLE 1

| | | | Conditioning | | | |
|---|---|---|---|---|---|---|
| | | | Treatment | | | |
| | | TBI | Day −2 | | BM | Splenocytes |
| Group | N | Day −1 | and −3 | Recipient | Day 0 | Day 0 |
| 1 | 8 | 830X | Cytoxan | B10.BR | 10⁷ B6 WT | — |
| 2 | 8 | 830X | Cytoxan | B10.BR | 10⁷ B6 WT | 10⁶ B6 WT |
| 3 | 8 | 830X | Cytoxan | B10.BR | 10⁷ B6 WT | 10⁶ B6 |
| | | | | | | Blc6 KO |

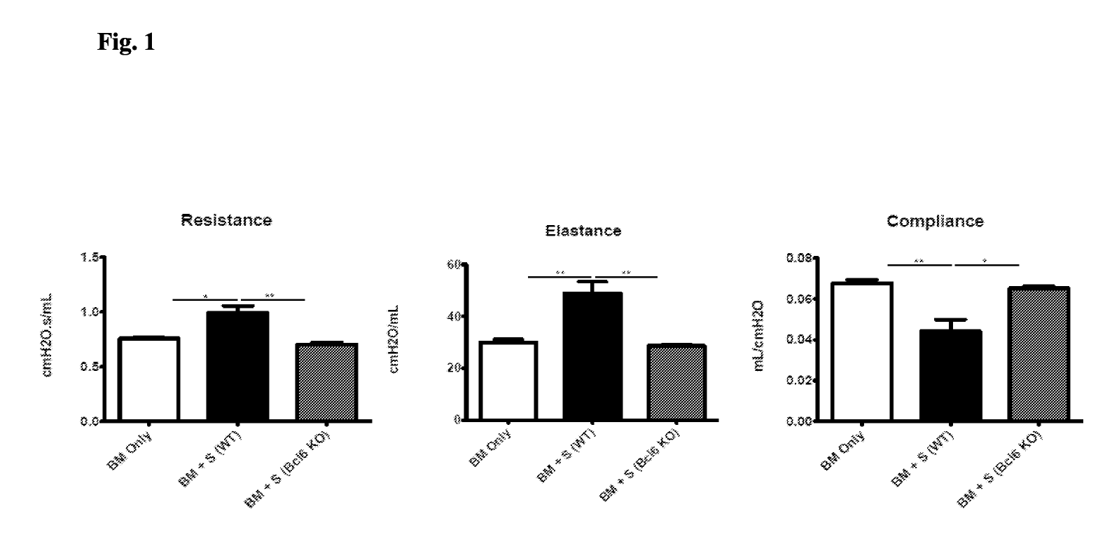

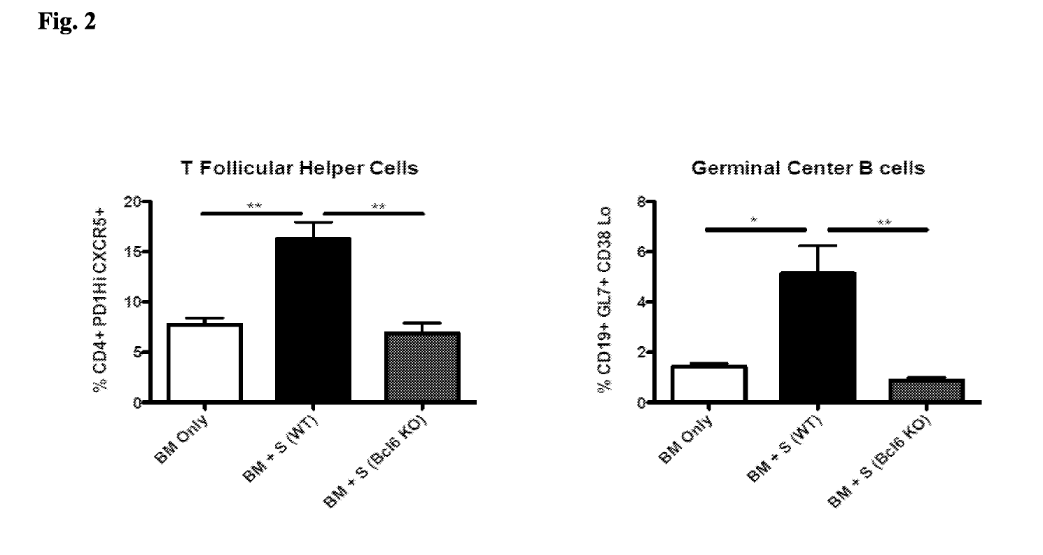

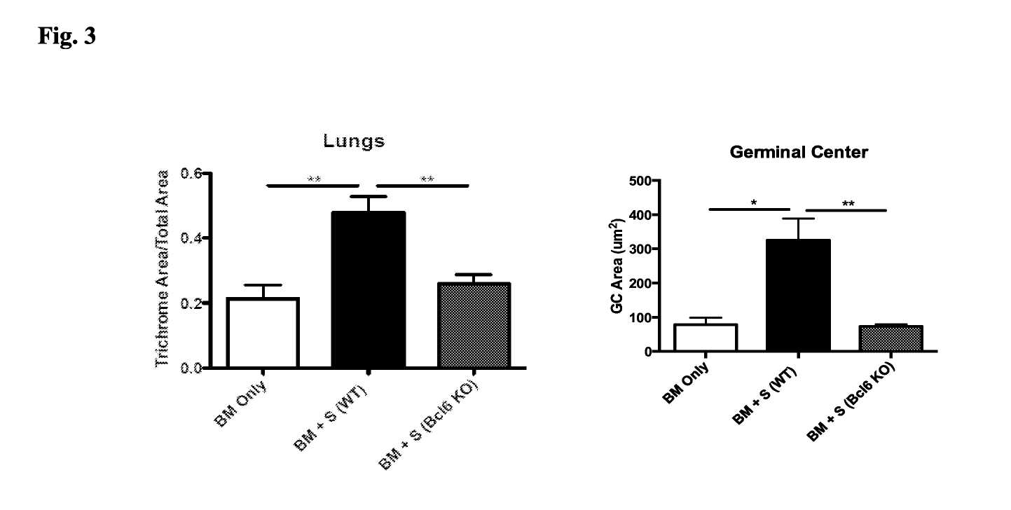

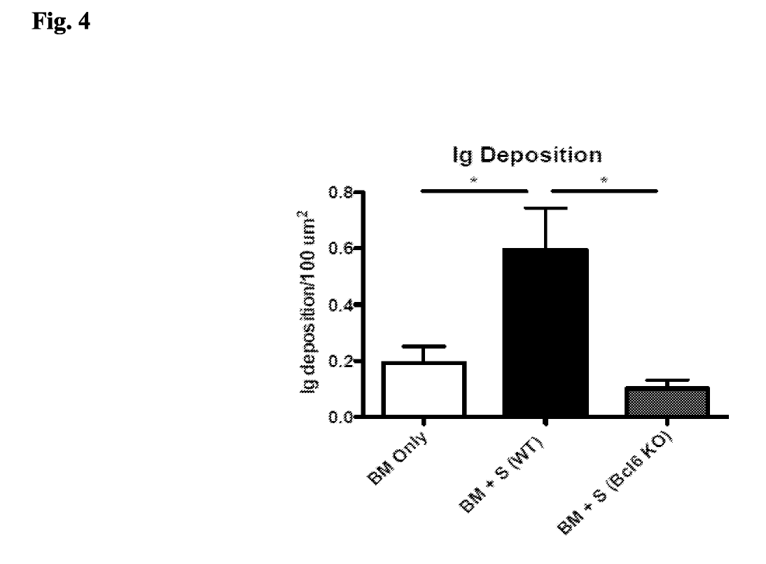

Transplant of wild-type bone marrow (BM) with Bcl6 knock-out T-cells (Bcl6 KO) derived from mouse spleens resulted in improved pulmonary function in the mouse model of cGVHD (FIG. 1). Mice receiving Bcl6 KO cells show decreased lung resistance and elastance and increased lung compliance than mice receiving wild-type T-cells. Mice receiving Bcl6 KO cells also show decreased T follicular helper cells and germinal center B cells compared to mice receiving wild-type T-cells (FIG. 2). The lungs of mice treated with Bcl6 KO cells have decreased collagen and Ig deposition (FIGS. 3-4). Mice receiving Bcl6 KO cells also show decreased size of germinal centers compared to mice receiving wild-type T-cells (FIG. 3). The was decreased in mice receiving Bcl6 KO cells

EZH2 is Necessary in B Cells and T-Cells for Development of cGVHD

Table 2 provides a graphical representation of the animal model protocol used in this experiment.

TABLE 2

| | | | Conditioning | | | Splenocytes |
|---|---|---|---|---|---|---|
| | | | Treatment | | | Day 0 |
| | | TBI | Day −2 | | BM | Purified |
| Group | N | Day −1 | and −3 | Recipient | Day 0 | T-cells |
| 1 | 10 | 830X | 120 mg/kg | B10.BR | 10⁷ B6 WT | — |
| | | | Cytoxan | | FoxP3-GFP | |
| | | | | | (TCD) | |
| 2 | 10 | 830X | 120 mg/kg | B10.BR | 10⁷ B6 WT | 0.1 × 10⁶ B6 |
| | | | Cytoxan | | FoxP3-GFP | WT |
| | | | | | (TCD) | |
| 3 | 10 | 830X | 120 mg/kg | B10.BR | 10⁷ B6 WT | 0.1 × 10⁶ B6 |
| | | | Cytoxan | | (TCD) | EZH2 CD4- |
| | | | | | | Cre |
| 4 | 10 | 830X | 120 mg/kg | B10.BR | 10⁷ B6 EZH2 | — |
| | | | Cytoxan | | CG1-Cre | |
| 5 | 10 | 830X | 120 mg/kg | B10.BR | 10⁷ B6 EZH2 | 0.1 × 10⁶ B6 |
| | | | Cytoxan | | CG1-Cre | WT |

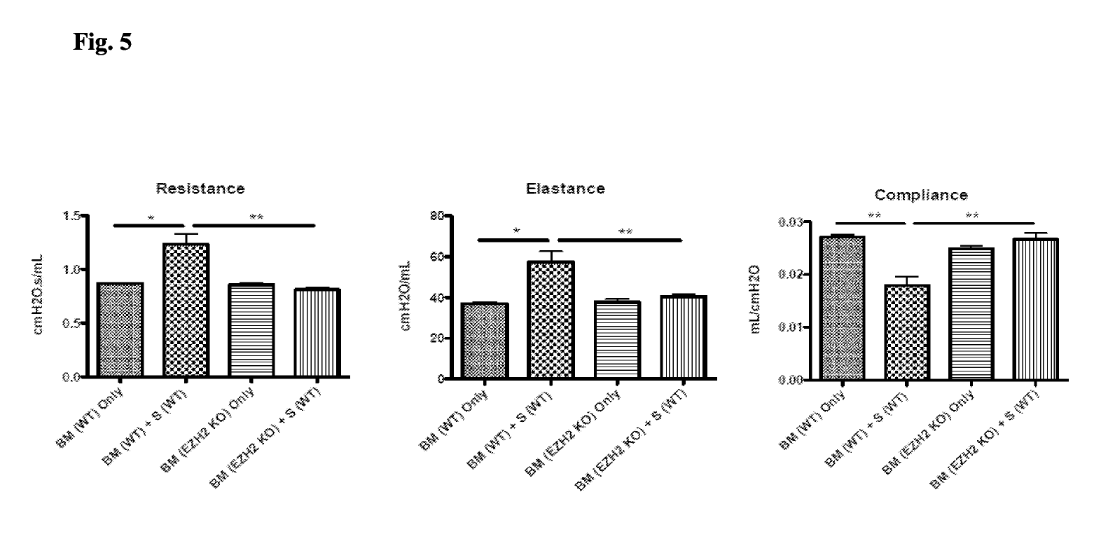

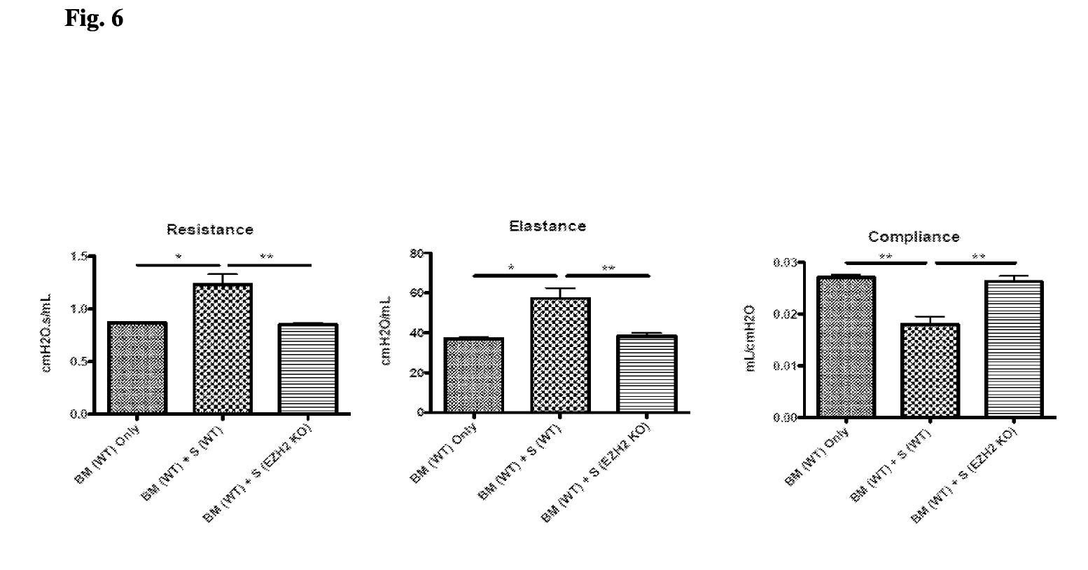

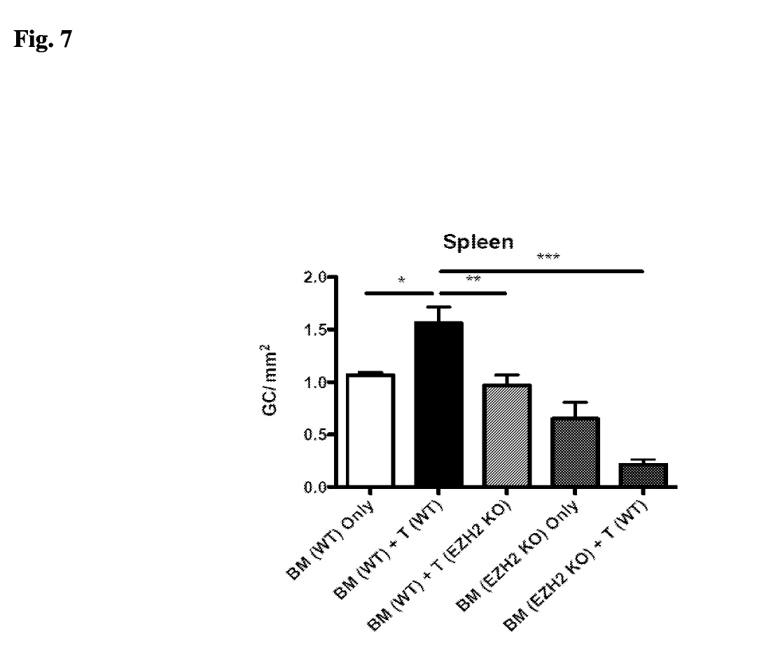

FIG. 5 shows improved pulmonary function in mice transplanted with wild-type spleen-derived T-cells and EZH2 knock out bone marrow (EZH2 KO BM). Mice receiving EZH2 KO BM along with wild-type T-cells have decreased lung resistance and elastance and increased lung compliance compared to mice receiving wild-type bone marrow. A similar improvement in pulmonary function (resistance, elastance and compliance) occurs when mice receive EZH2 KO spleen-derived T-cells and wild-type bone marrow (FIG. 6). Furthermore, mice transplanted with either EZH2 KO BM or EZH2 KO T-cells show a decrease in germinal center formation (FIG. 7). These data indicate that inhibition of EZH2 activity prevents the formation of germinal centers and allo-responses.

EZH2 Inhibitors or Direct Bcl6 Inhibitors Will Prevent the Development of cGVHD

Table 3 provides a graphical representation of the animal model protocol used in this experiment.

TABLE 3

| | | | | | | Splenocytes | |
|---|---|---|---|---|---|---|---|
| | | | Conditioning | | | Day 0 | |
| | | TBI | Treatment | | BM | Purified T- | Treatment |
| Group | N | Day −1 | Day −2 and −3 | Recipient | Day 0 | cells | Day 28-56 |
| 1 | 10 | 830X | 120 mg/kg | B10.BR | 10⁷ B6 | — | — |
| | | | Cytoxan | | WT | | |
| 2 | 15 | 830X | 120 mg/kg | B10.BR | 10⁷ B6 | 0.1 × 10⁶ | Vehicle |
| | | | Cytoxan | | WT | B6 WT | (DMSO + HPCbD) |
| | | | | | | | (PBS) |
| 3 | 10 | 830X | 120 mg/kg | B10.BR | 10⁷ B6 | 0.1 × 10⁶ | 75 mg/Kg JQ5 |
| | | | Cytoxan | | WT | B6 WT | 3 days a week |
| 4 | 10 | 830X | 120 mg/kg | B10.BR | 10⁷ B6 | 0.1 × 10⁶ | 75 mg/KG |
| | | | Cytoxan | | WT | B6 WT | UNC1999 |
| | | | | | | | 3 days a week |
| 5 | 10 | 830X | 120 mg/kg | B10.BR | 10⁷ B6 | 0.1 × 10⁶ | Bcl6 |
| | | | Cytoxan | | WT | B6 WT | Peptidomimetic |
| | | | | | | | every day |

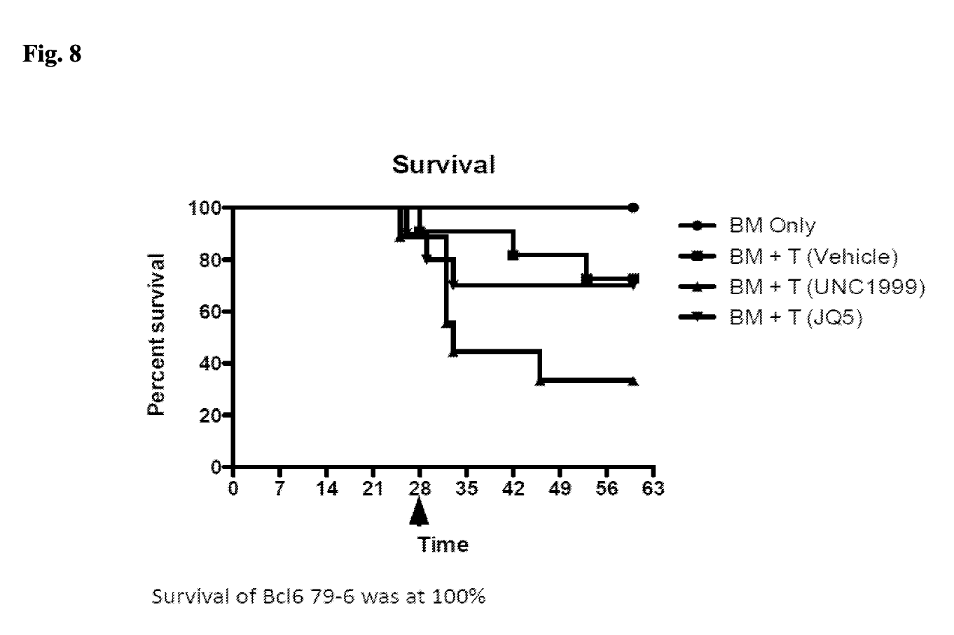

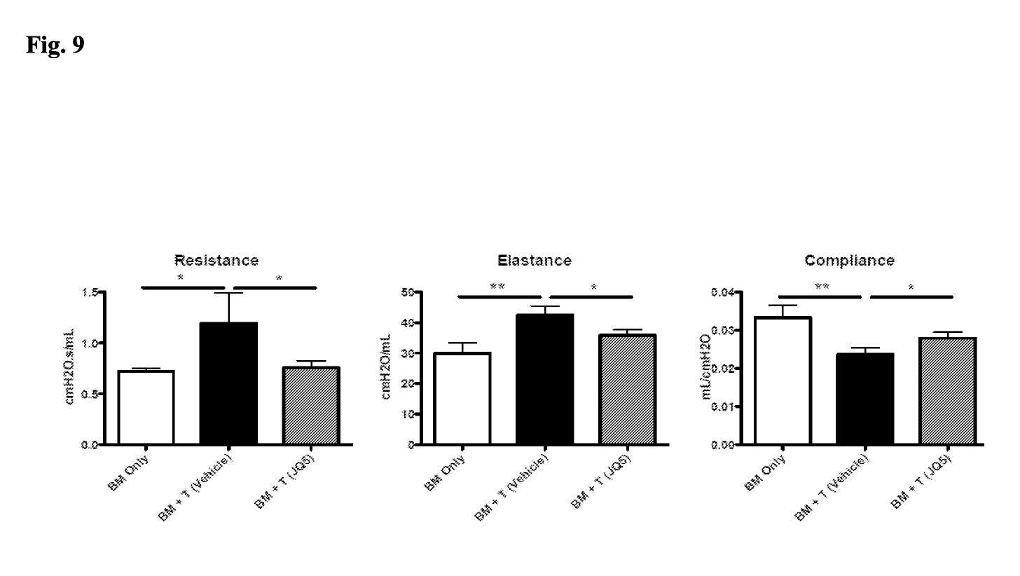

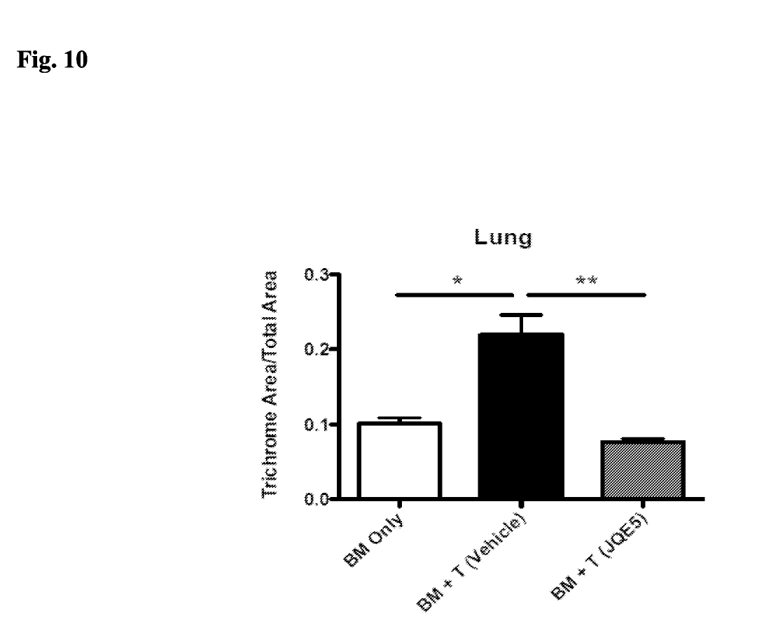

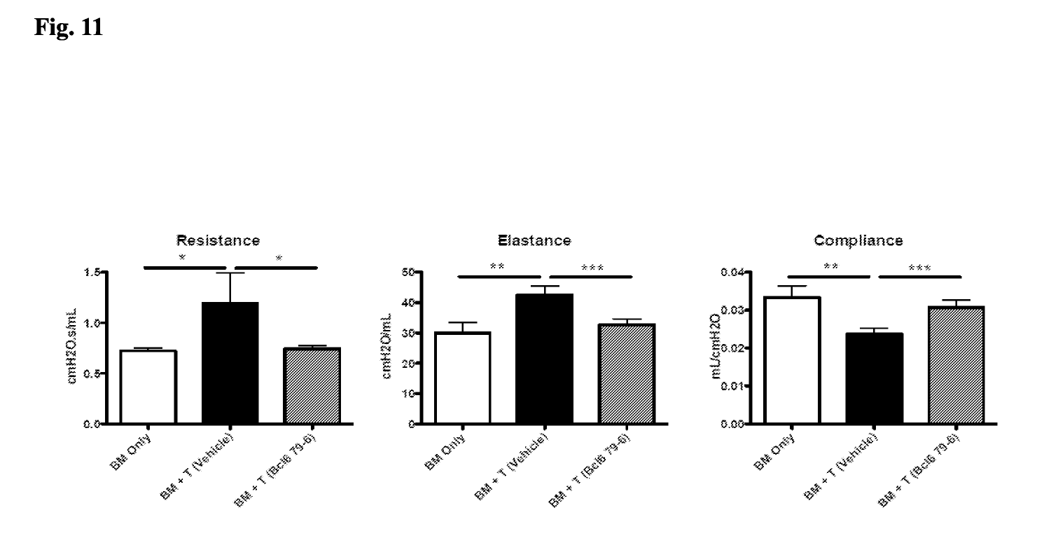

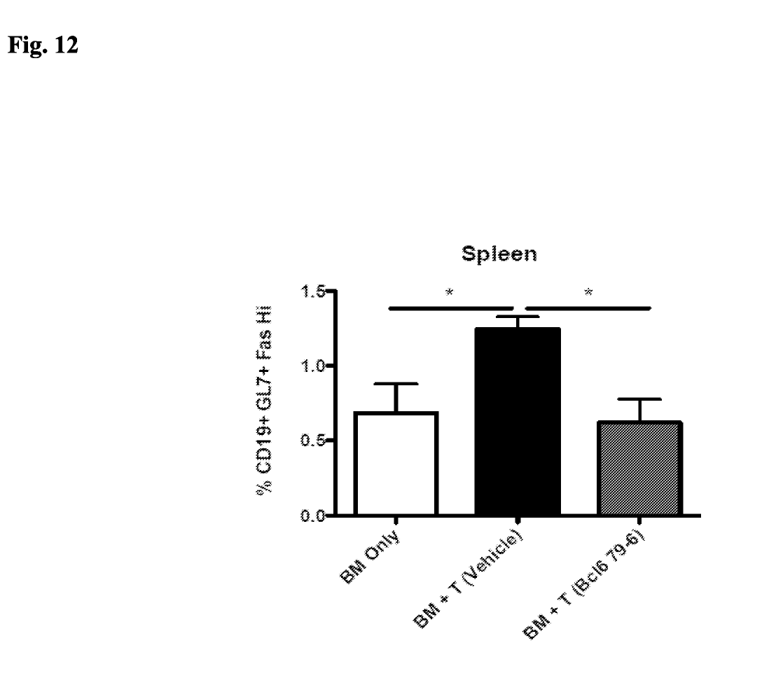

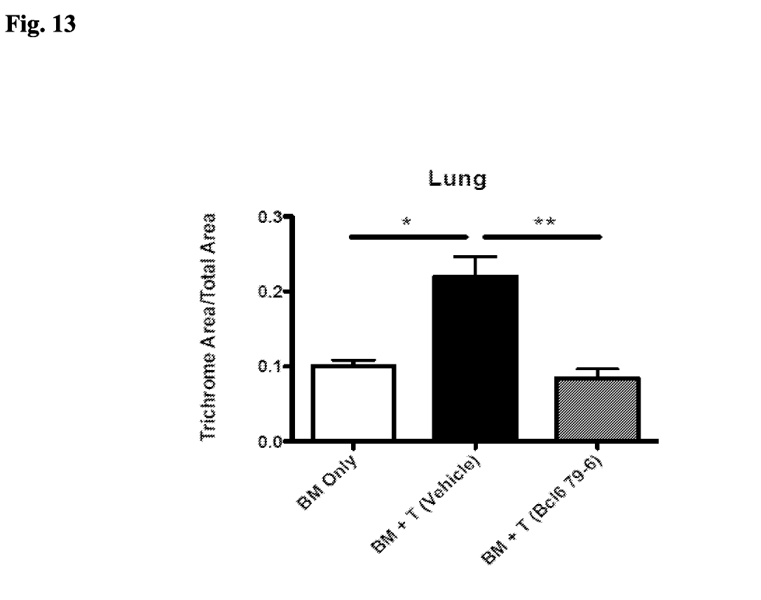

Animals in this experiment were transplanted with wild-type BM and T-cells and then treated with either Vehicle, EZH2 inhibitor (JQ5 or UNC1999) or Bcl6 inhibitor. Administration of UNC1999 was toxic, resulting in less than 40% survival 63 days post-transplant. (FIG. 8). Treatment with EZH2 inhibitor JQ5 resulted in improved pulmonary function, as measured by lung resistance, elastance and compliance when compared to mice treated with Vehicle only (FIG. 9). Mice treated with JQ5 also showed decreased collagen deposition in the lungs (FIG. 10). Treatment of mice with Bcl6 inhibitor (79-6 peptide) also improved pulmonary function compared to Vehicle-treated control mice (FIG. 12). A reduction in germinal center B cells was observed in the spleens of mice treated with Bcl6 inhibitor (FIG. 13). Collagen deposition in the lungs of mice treated with Bcl6 inhibitor was also decreased compared to Vehicle-treated control mice (FIG. 13).

BRD4 Inhibitor JQ1 Will Prevent cGVHD by Inhibiting Germinal Centers.

Table 4 provides a graphical representation of the animal model protocol used in this experiment.

| | | | Conditioning | | | | |
|---|---|---|---|---|---|---|---|
| | | TBI | Treatment | | BM | Splenocytes | Treatment |
| Group | n | Day −1 | Day −2, −3 | Recipient | Day 0 | Day 0 | Day 28-56 |
| 1 | 8 | 830X | 120 mg/kg | B10.BR | 10⁷ B6 WT | — | — |
| | | | Cytoxan | | | | |
| 2 | 8 | 830X | 120 mg/kg | B10.BR | 10⁷ B6 WT | 10⁶ B6 WT | Vehicle |
| | | | Cytoxan | | | | DMSO + HPCbD |
| 3 | 8 | 830X | 120 mg/kg | B10.BR | 10⁷ B6 WT | 10⁶ B6 WT | JQ1 |
| | | | Cytoxan | | | | 50 mg/Kg Daily |

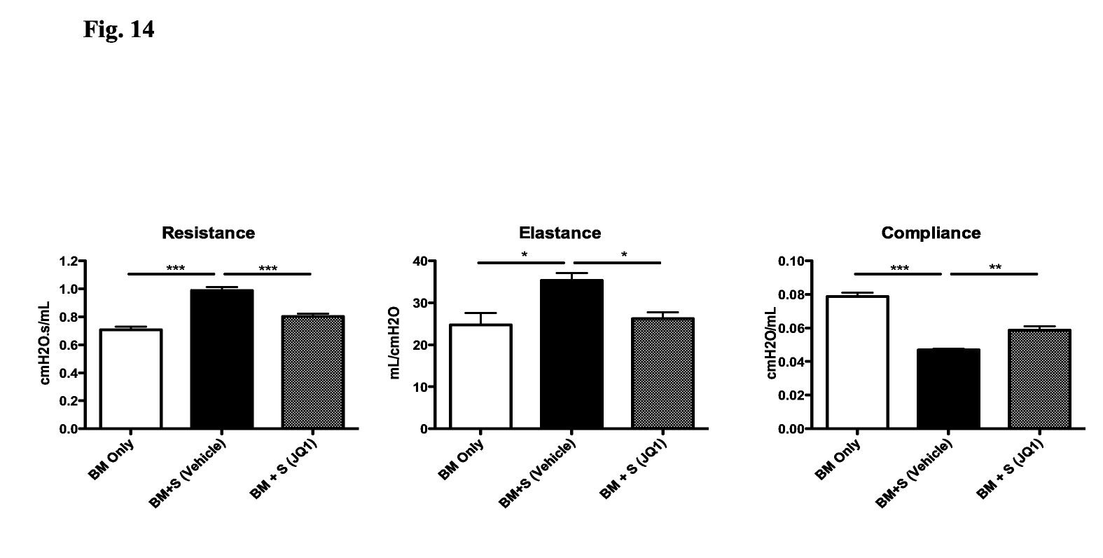

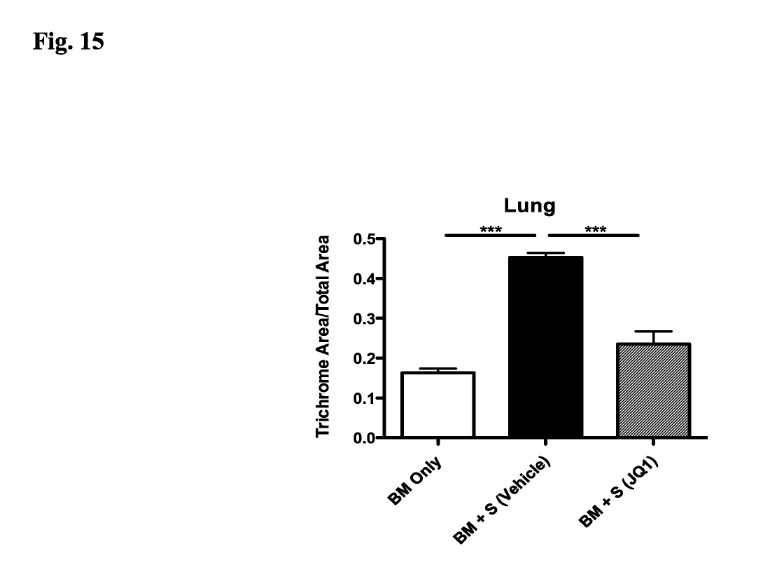

Animals in this experiment were transplanted with wild-type BM and T-cells and then treated with either Vehicle or BRD4 inhibitor (JQ1). Treatment of mice with BRD4 inhibitor (JQ1) improved pulmonary function compared to Vehicle-treated control mice (FIG. 14). Collagen deposition in the lungs of mice treated with BRD4 inhibitor (JQ1) was also decreased compared to Vehicle-treated control mice (FIG. 15).

Similar results were also observed in additional experiments (not shown), where JQ1 treatment decreased resistance and elestance in pulmonary function tests significantly as compared to vehicle-treated control animals. Compliance was significantly increased in these JQ1-treated animals as compared to vehicle-treated control animals.

Taken together, these data suggest that lung disease is improved when mice were treated with JQ1.

### Preparation of the EZH2 Compounds Described Herein

The compounds provided herein can be prepared from readily available starting materials using the following general methods and procedures. For example, compounds of Formula (I) can be prepared according to any one of Schemes 1 to 3, and compounds of Formula (II) can be prepared according to methods similar to the methods shown in any one of Schemes 1 to 3. Where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvents used, but such conditions can be determined by those skilled in the art by routine optimization procedures.

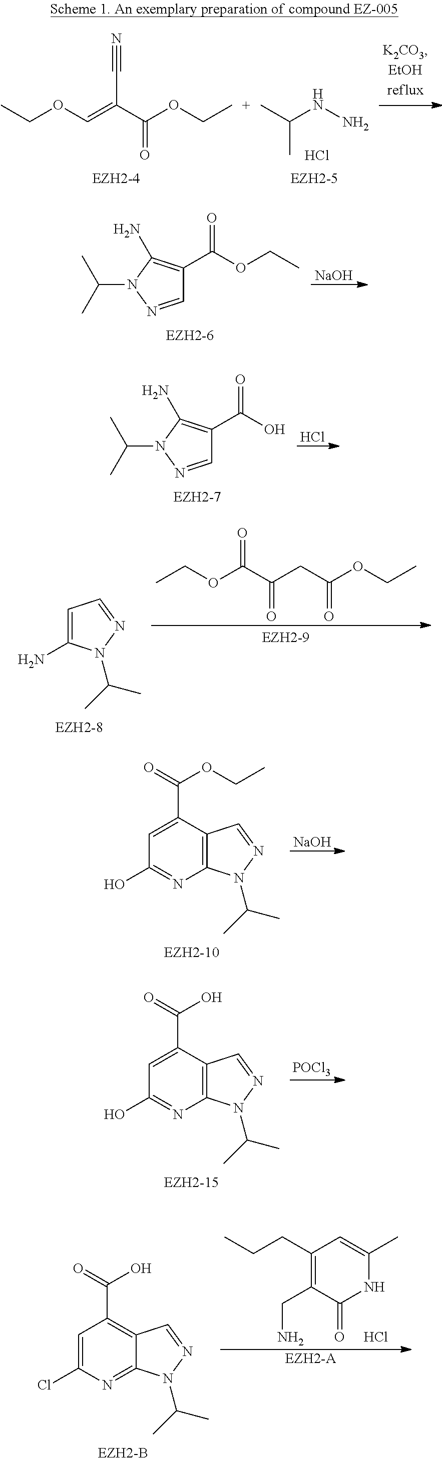

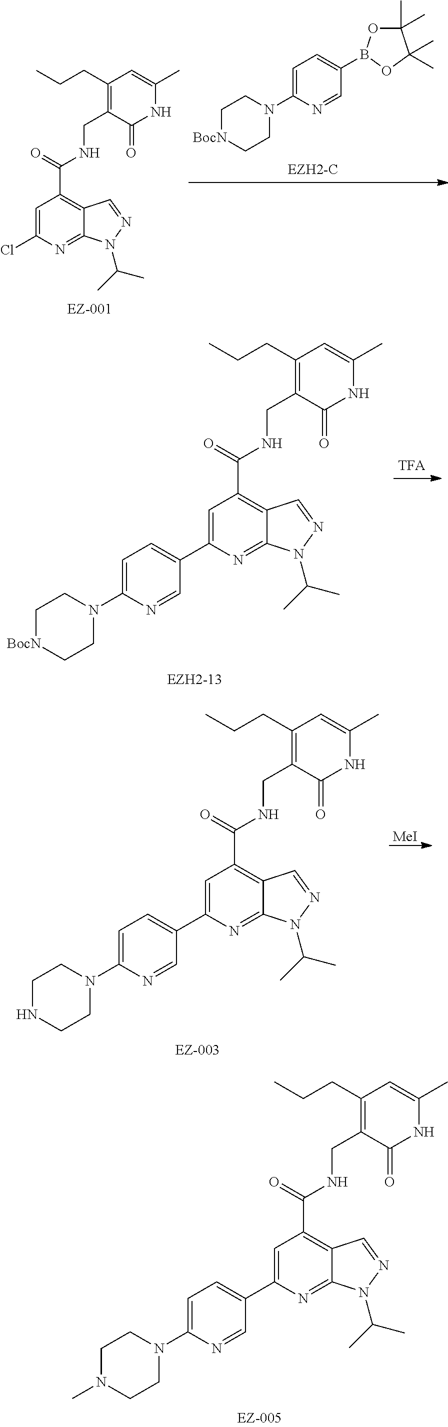

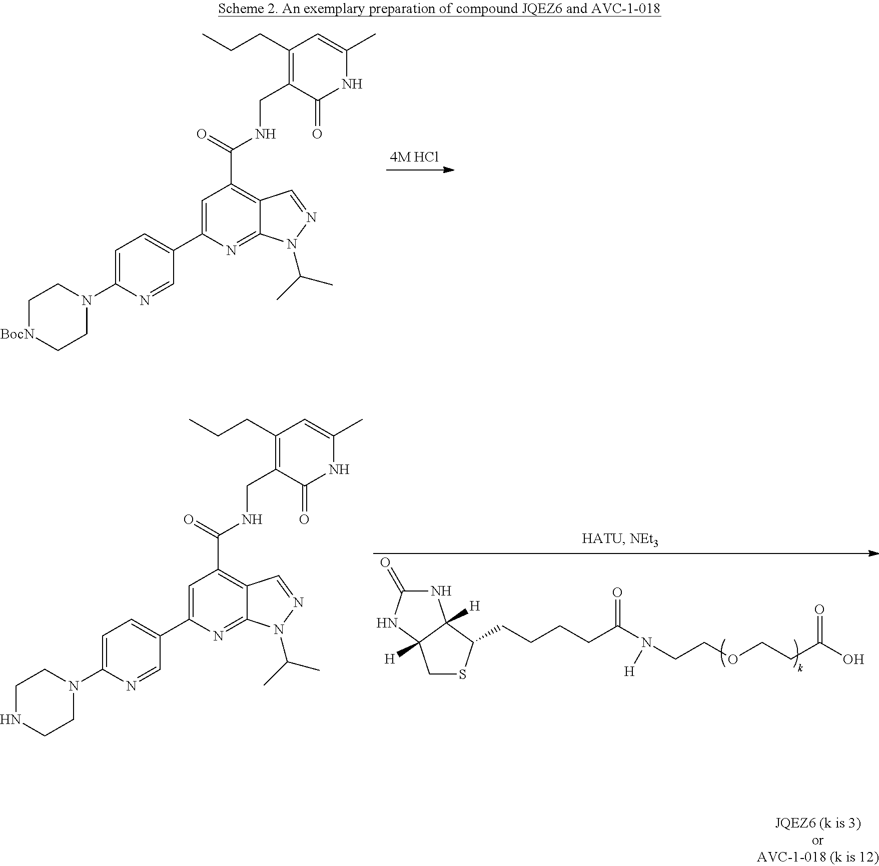

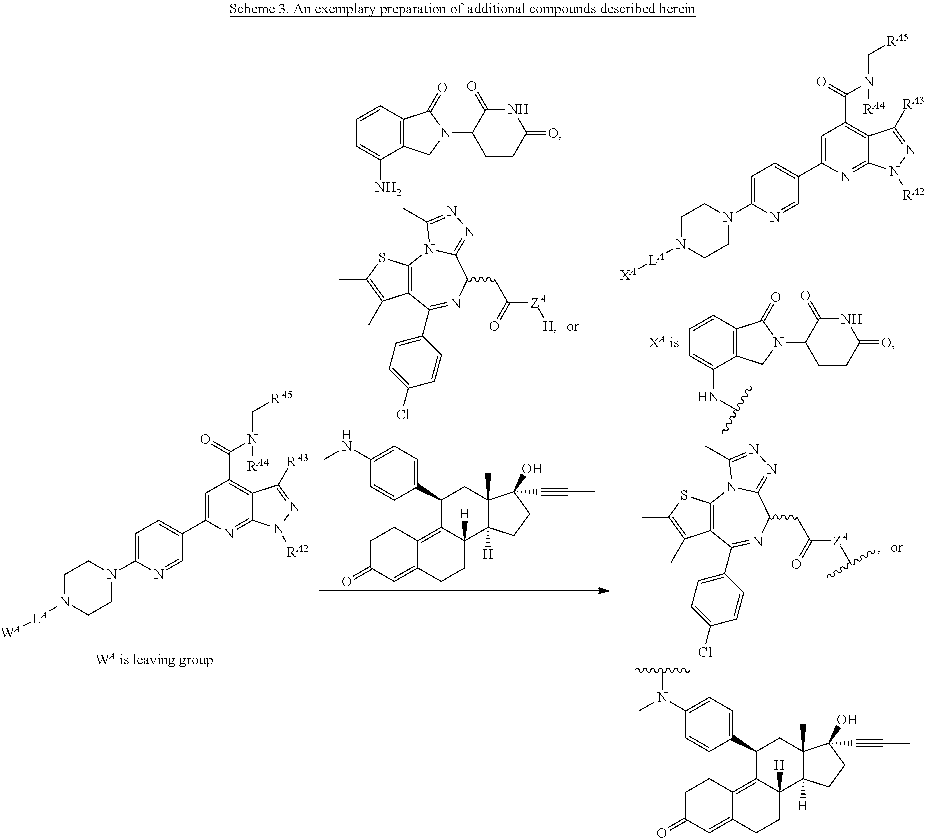

In certain embodiments, the hydrazides described herein are prepared pursuant to the methods described in international PCT application, PCT/US2015/044303, incorporated herein by reference. In certain embodiments, the hydrazides described herein are prepared pursuant to the methods shown in Scheme 4 or 5.

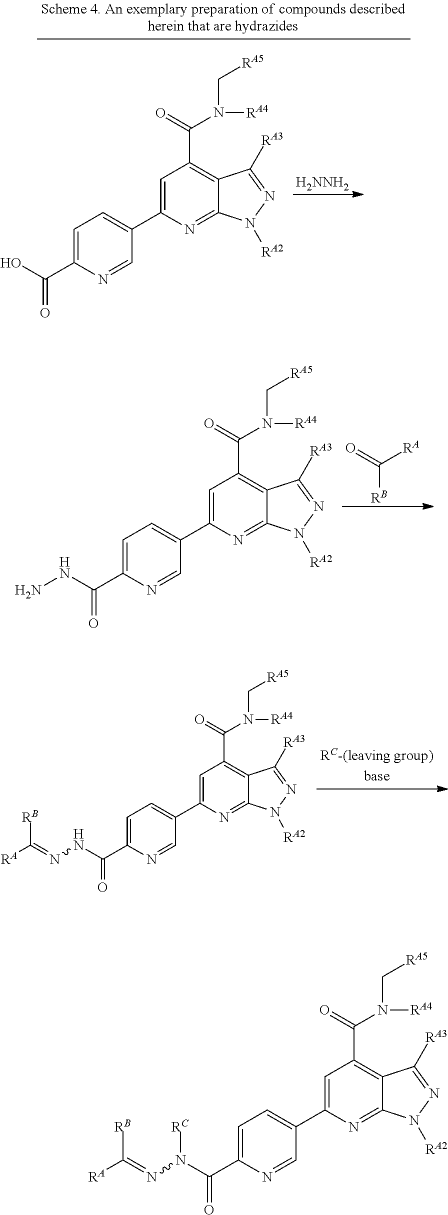

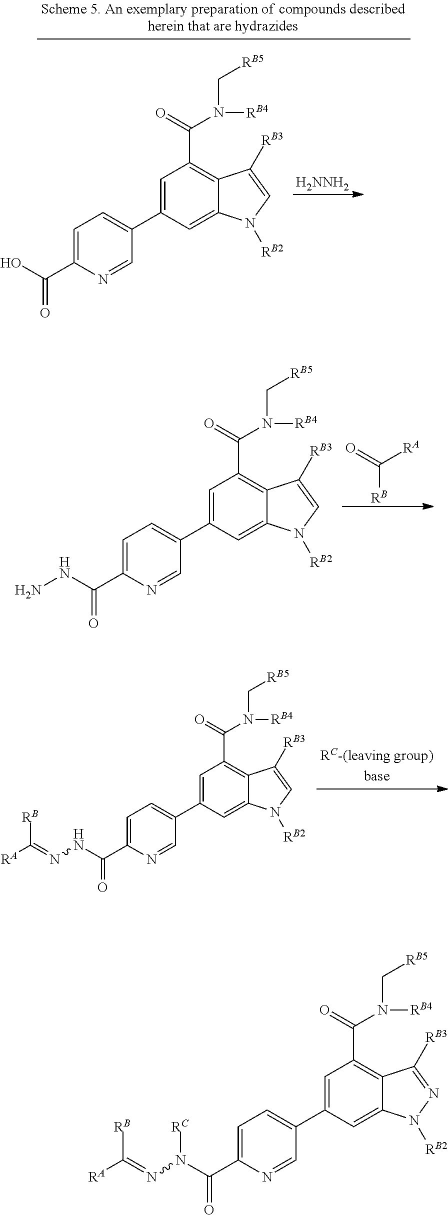

In certain embodiments,

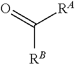

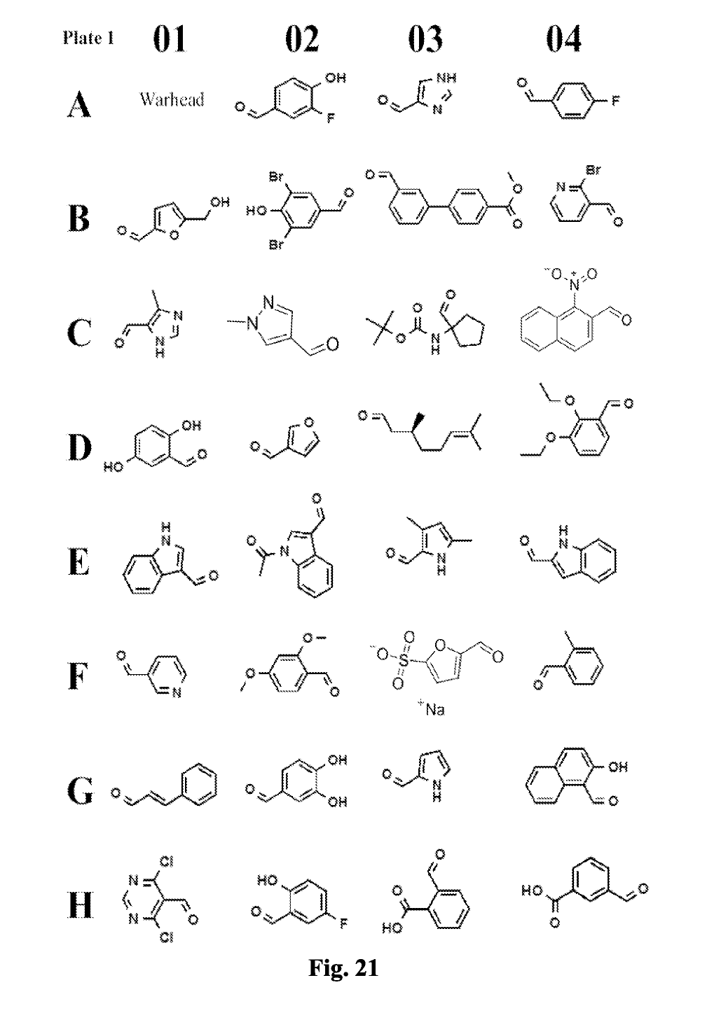

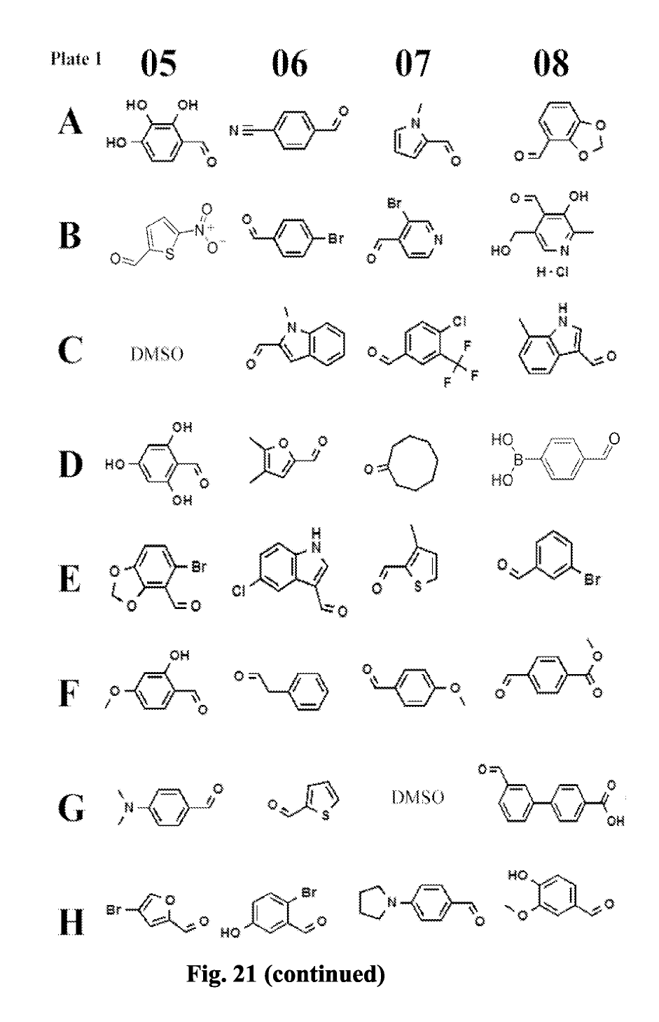

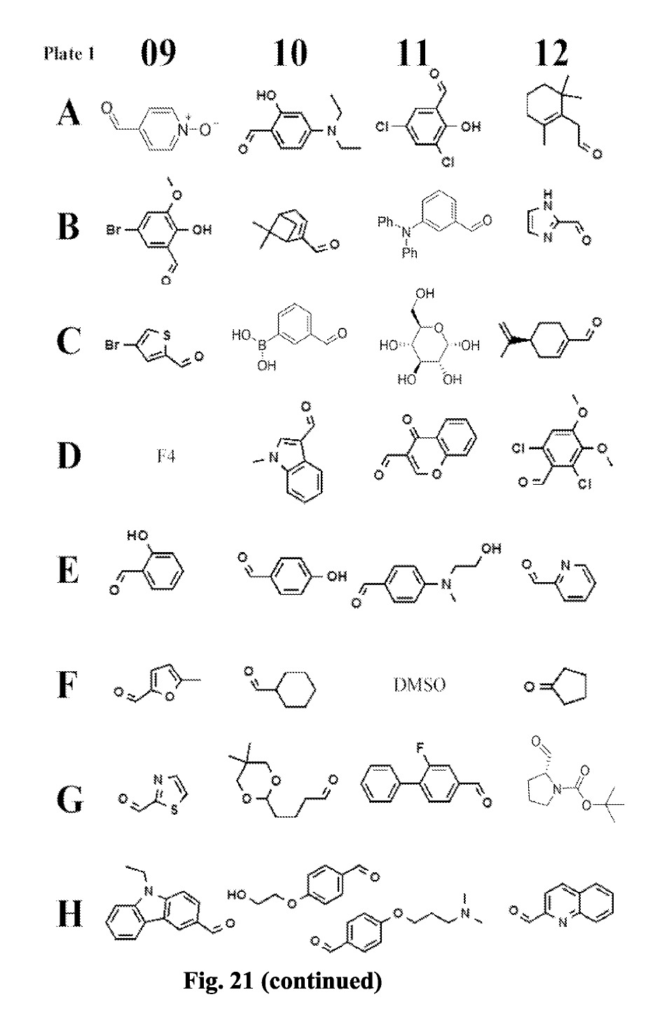

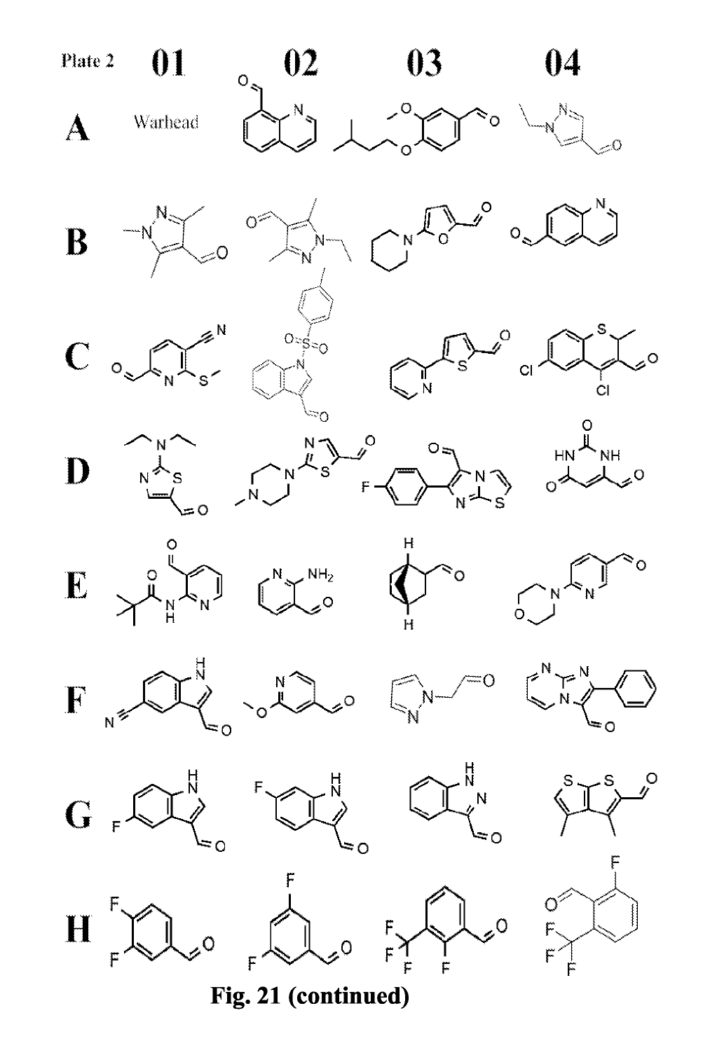

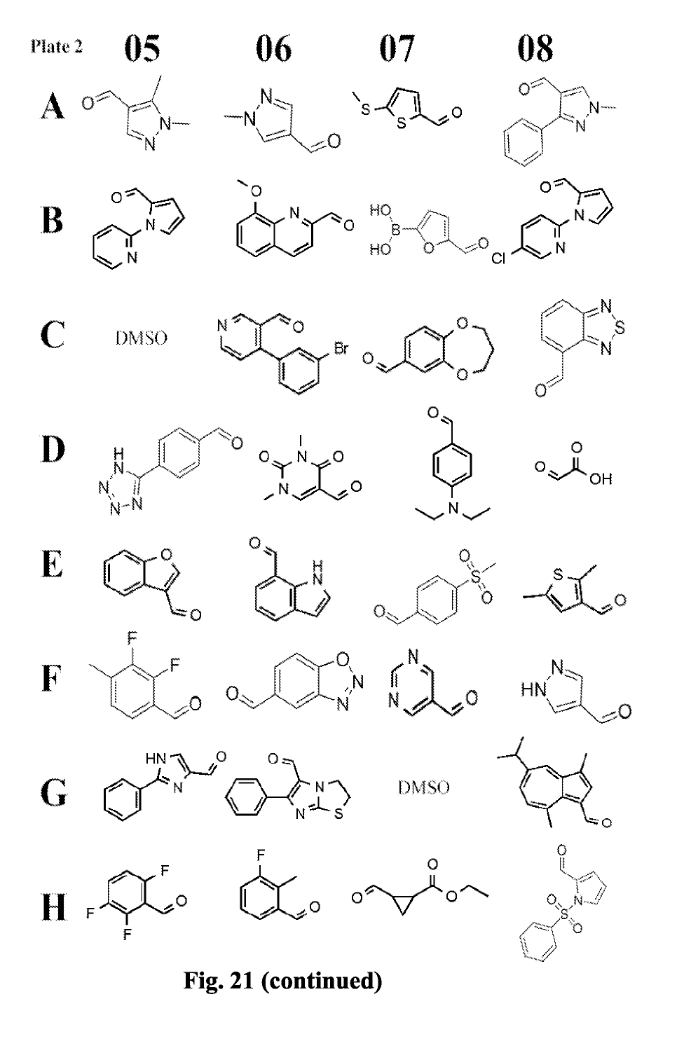

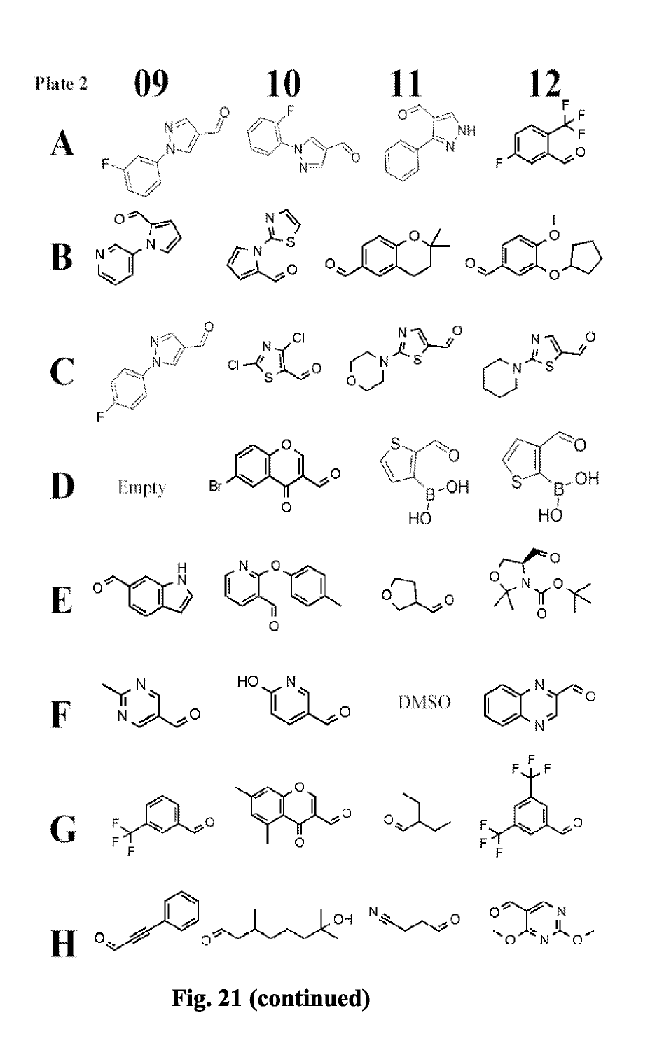

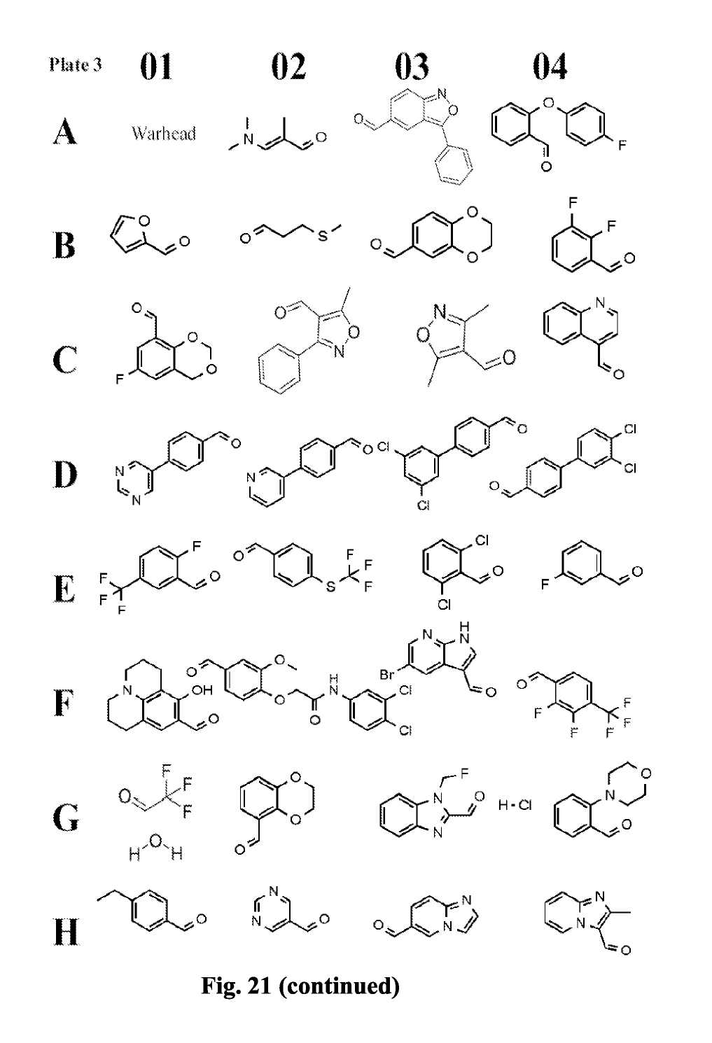

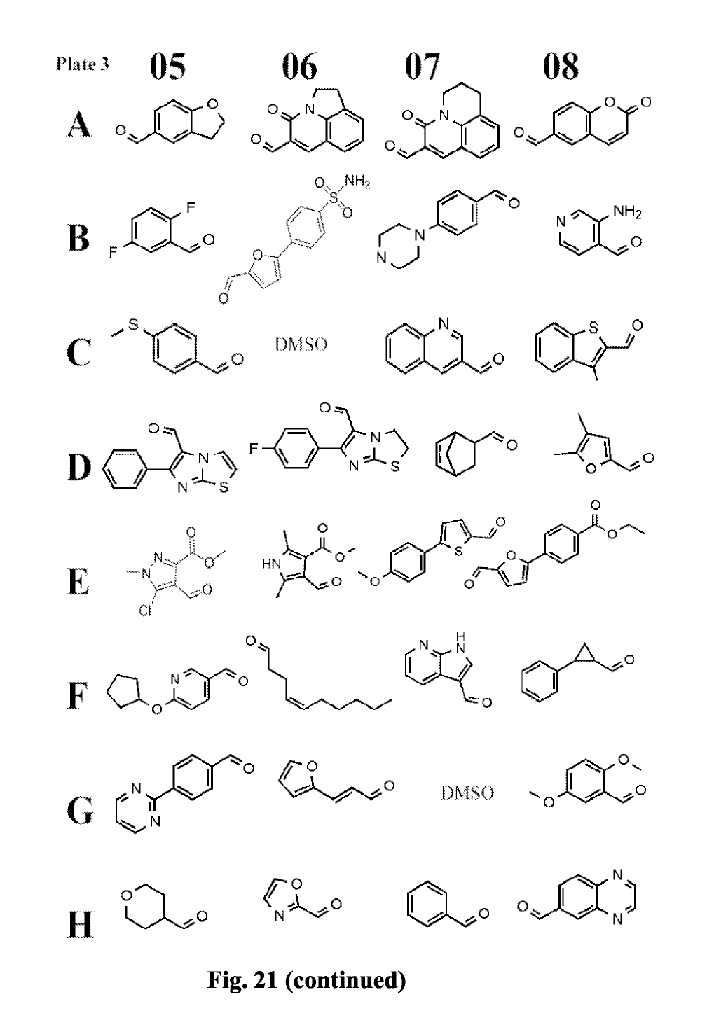

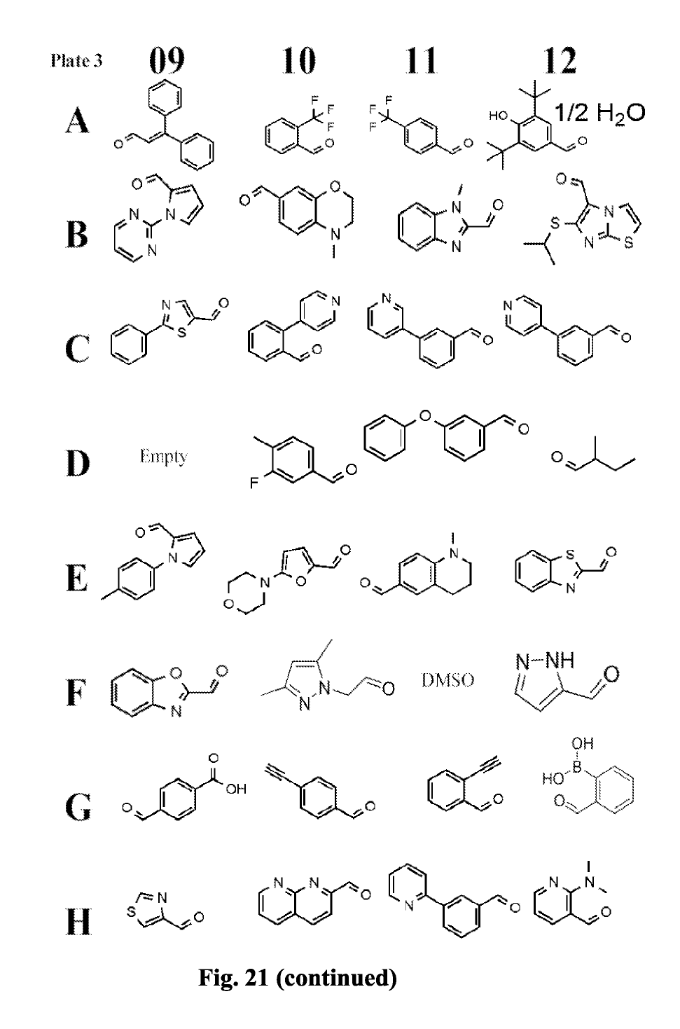

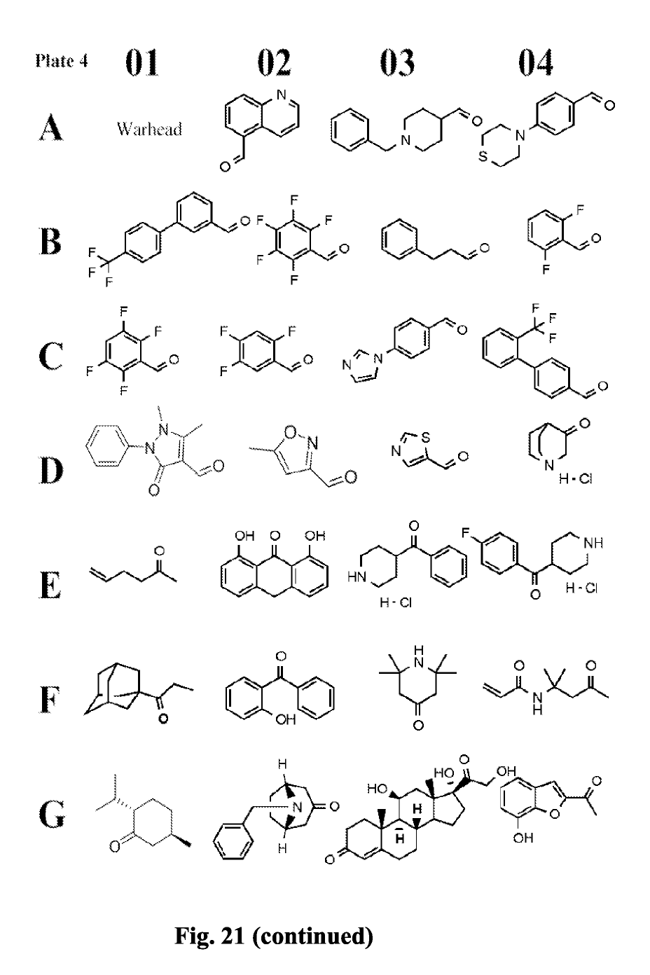

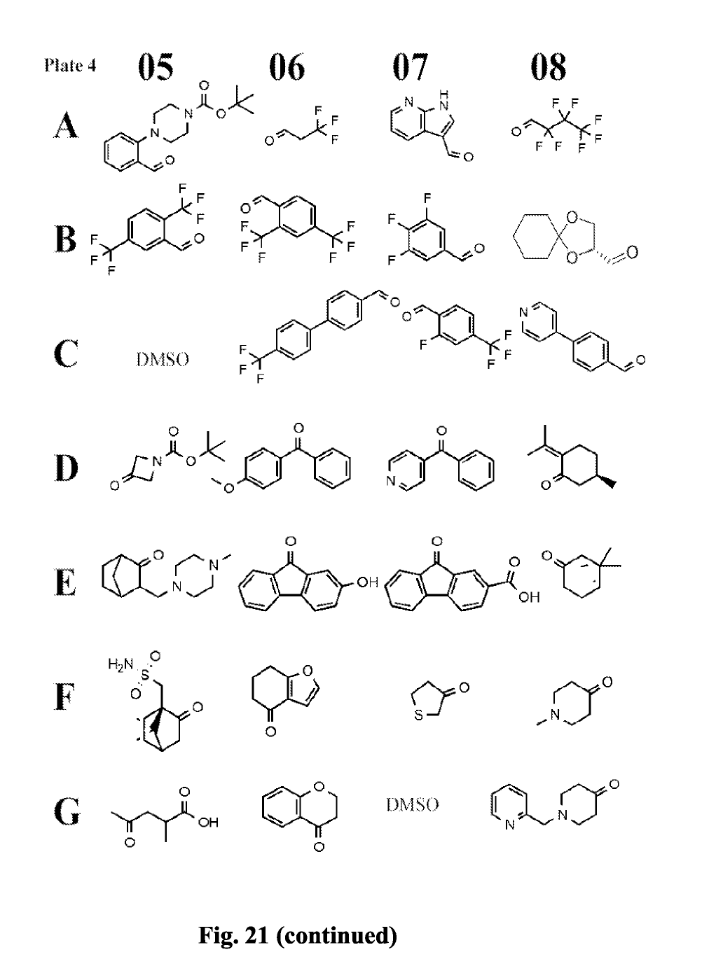

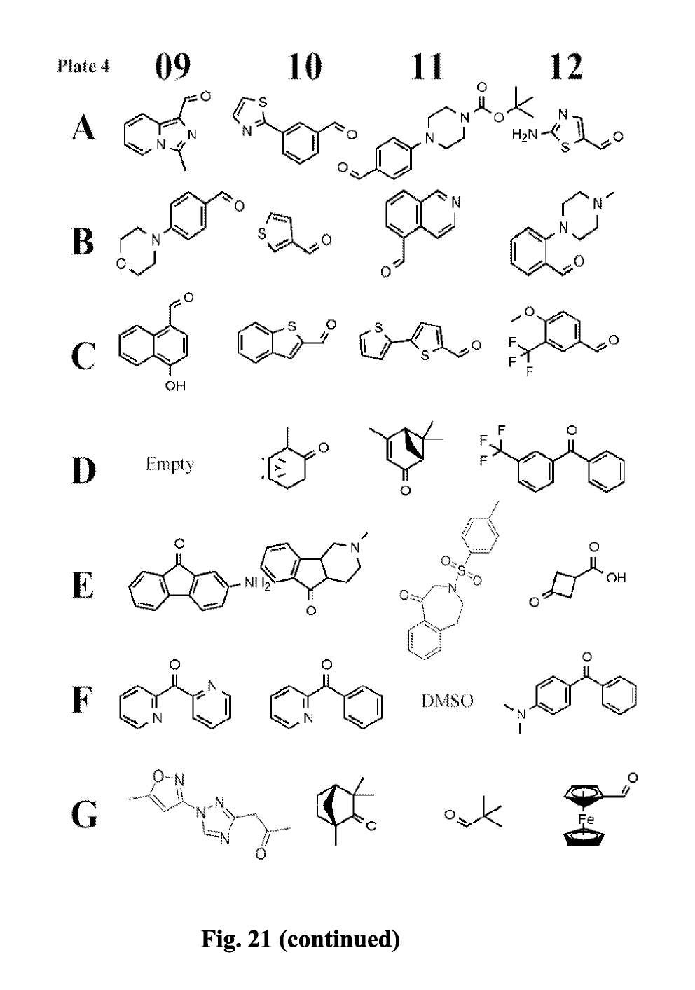

is an aldehyde or ketone shown in FIG. 21.

### Example 2

EZH2 Knockout Bone Marrow and Tregs with and without JQ5 Inhibition

Table 5 provides a graphical representation of the animal model protocol used in this experiment.

TABLE 5

| | | | Conditioning | | | Splenocytes | |
|---|---|---|---|---|---|---|---|
| | | TBI | Treatment | | BM | Day 0 Purified | |
| Group | n | Day −1 | Day −2 and −3 | Recipient | Day 0 | T cells | Treatments |
| 1 | 5 | 830X | 120 mg/kg | B10.BR | 10⁷ B6 | — | — |
| | | | Cytoxan | | WT | | |
| | | | | | (TCD) | | |
| 2 | 10 | 830X | 120 mg/kg | B10.BR | 10⁷ B6 | 0.1 × 10⁶ B6 | Vehicle |
| | | | Cytoxan | | WT | WT | |
| | | | | | (TCD) | | |
| 3 | 10 | 830X | 120 mg/kg | B10.BR | 10⁷ B6 | 0.1 × 10⁶ B6 | JQ5 |
| | | | Cytoxan | | WT | WT | 75 mg/Kg |
| | | | | | (TCD) | | |
| 4 | 5 | 830X | 120 mg/kg | B10.BR | 10⁷ B6 | — | — |
| | | | Cytoxan | | EZH2 | | |
| | | | | | CG1-Cre | | |
| 5 | 10 | 830X | 120 mg/kg | B10.BR | 10⁷ B6 | 0.1 × 10⁶ B6 | — |
| | | | Cytoxan | | EZH2 | WT | |
| | | | | | CG1-Cre | | |
| 6 | 10 | 830X | 120 mg/kg | B10.BR | 10⁷ B6 | 0.8 × 10⁵ B6 | Vehicle |
| | | | Cytoxan | | WT | EZH2 fl/fl X | |
| | | | | | | FoxP3-cre | |
| 7 | 10 | 830X | 120 mg/kg | B10.BR | 10⁷ B6 | 0.8 × 10⁵ B6 | JQ5 |
| | | | Cytoxan | | WT | EZH2 fl/fl X | 75 mg/Kg |
| | | | | | | FoxP3-cre | |

Animals in this experiment were transplanted with wild-type or EZH2 knockout BM and wild-type or EZH2 knockout BM T-cells and then treated with either Vehicle or EZH2 inhibitor (JQ5).

In particular, B10.BR mice were conditioned with 120 mg/Kg of cyclophosphamide on day −3 and −2 and subjected to total body irradiation (830X) on day −1. On Day 0 mice were transplanted with wild type B6 bone marrow with or without splenocytes or purified splenic T cells. Day 28 following transplantation, mice were either given vehicle control or JQ5 (75 mg/Kg 3× a week) until day 56. On Day 60 mice were subjected to forced oscillation technique on the Scireq Flexivent system. Tissues were harvested and analyzed for collagen deposition by trichrome staining, Ig deposition, presence of germinal centers, germinal center B cells, and T follicular helper cells.

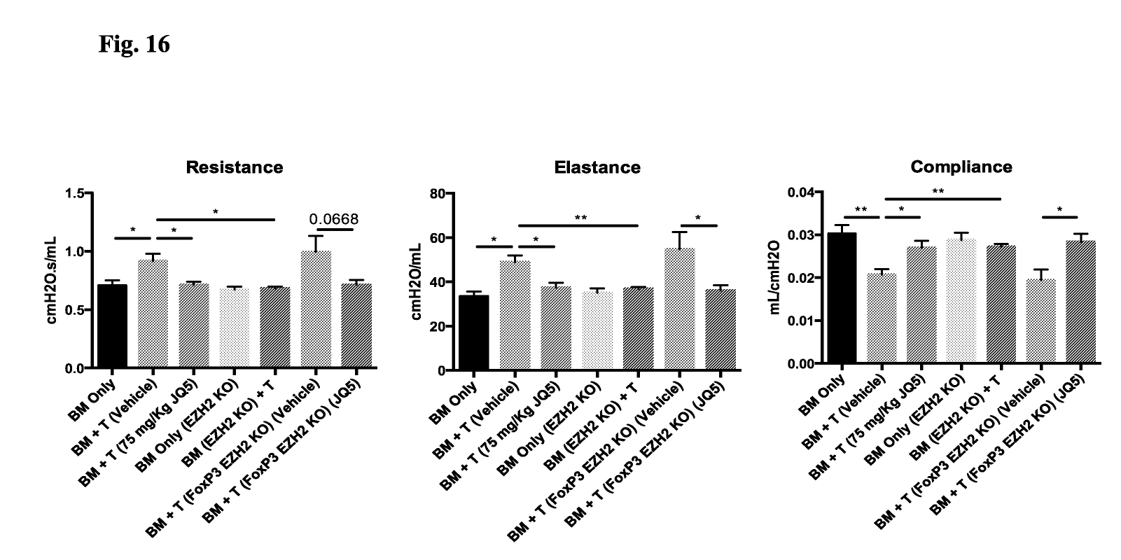

Mice treated with JQ5 had a decrease in resistance and elastance with an increase in compliance, restoring pulmonary function (FIG. 16). EZH2 KO BM showed similar results demonstrating that EZH2 is important in bone marrow derived cells (B cells) to cause disease. When Tregs specifically had EZH2 KO there was increased disease similar to the cGVHD control mice (column 2), but this was overcome with Ezh2 inhibition with JQ5.

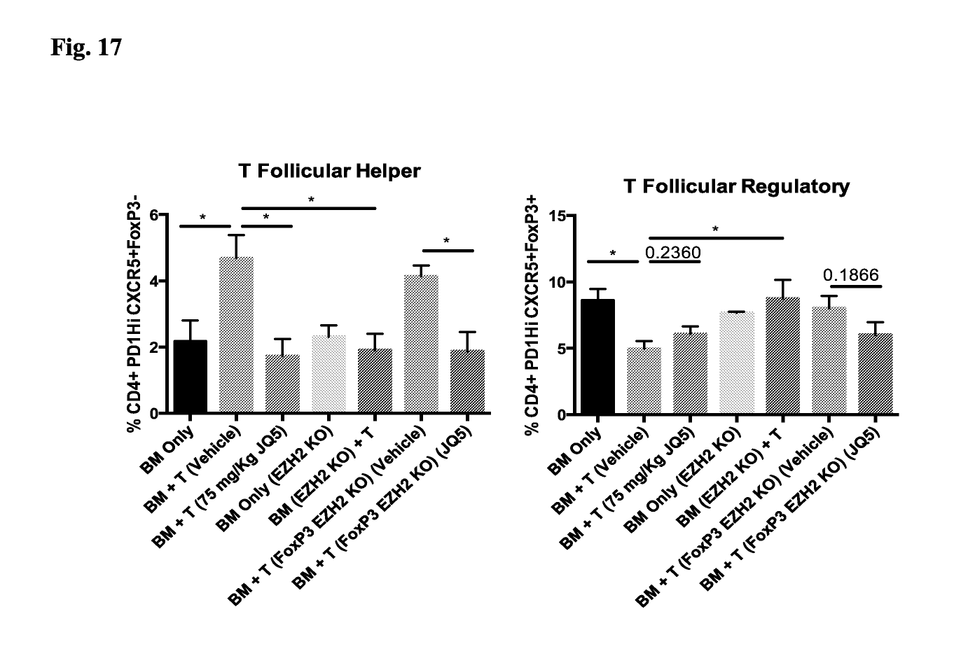

T follicular helper cells were decreased in mice therapeutically treated with JQ5 or in animals that had EZH2 KO BM. T follicular helpers were not decreased in Treg specific KO of EZH2 (FIG. 17). T follicular regulatory cells were decreased in cGVHD, but not increased with JQ5 therapy.

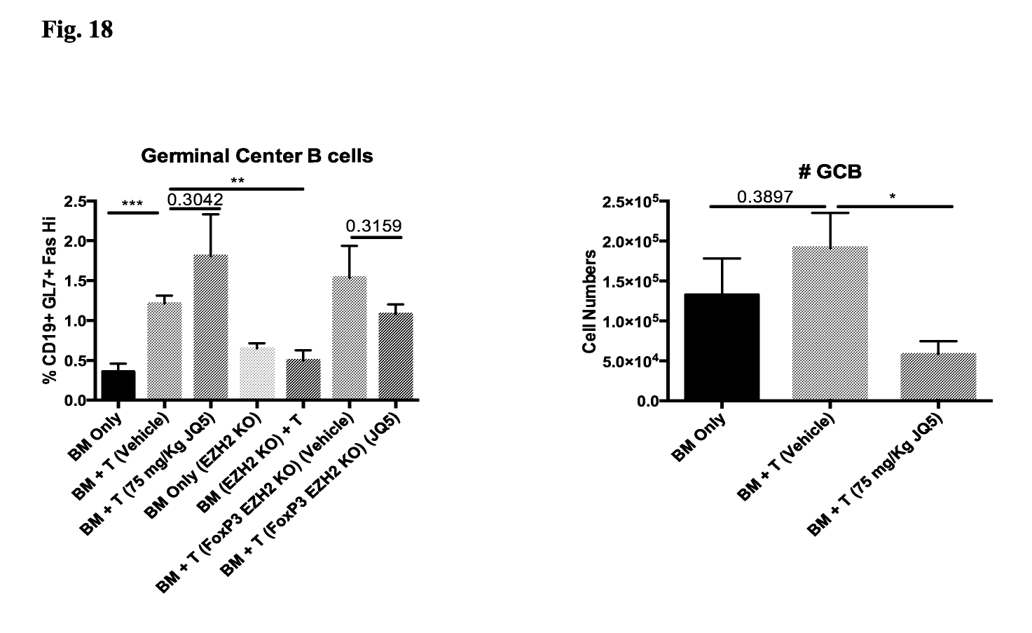

The frequency of Germinal Center B cells was similar to the cGVHD control animals, but the number of Germinal Center B cells in JQ5 treated mice was significantly decreased (FIG. 18).

### Bcl679-6 Therapeutic Intervention

Table 6 provides a graphical representation of the animal model protocol used in this experiment.

TABLE 6

| | | | Conditioning | | | Splenocytes | |
|---|---|---|---|---|---|---|---|
| | | TBI | Treatment | | BM | Purified T Cells | Treatment |
| Group | n | Day −1 | Day −2 and −3 | Recipient | Day 0 | Day 0 | Day 28-56 |
| 1 | 10 | 830X | 120 mg/kg | B10.BR | 10⁷ B6 | — | — |
| | | | Cytoxan | | WT | | |
| 2 | 10 | 830X | 120 mg/kg | B10.BR | 10⁷ B6 | 0.1 × 10⁶ B6 WT | Vehicle |
| | | | Cytoxan | | WT | | |
| 3 | 10 | 830X | 120 mg/kg | B10.BR | 10⁷ B6 | 0.1 × 10⁶ B6 WT | 50 mg/Kg Bcl6 |
| | | | Cytoxan | | WT | | 79-6 |
| | | | | | | | Peptidomimetic |
| | | | | | | | Daily |

Pulmonary function tests were performed on animals treated with Bcl 79-6. The presence of Germinal Center B cells and fibrosis in lungs were also assessed. B10.BR mice were conditioned with 120 mg/Kg of cyclophosphamide on day −3 and −2 and subjected to total body irradiation (830X) on day −1. On Day 0 mice were transplanted with Wild Type B6 Bone marrow with or without splenocytes or purified splenic T cells. Day 28 following transplantation, mice were either given vehicle control or Bcl6 79-6 (50 mg/Kg Daily) until day 56. On Day 60 mice were subjected to forced oscillation technique on the Scireq Flexivent system. Tissues were harvested and analyzed for collagen deposition by trichrome staining, Ig deposition, presence of germinal centers, germinal center B cells and T follicular helper cells.

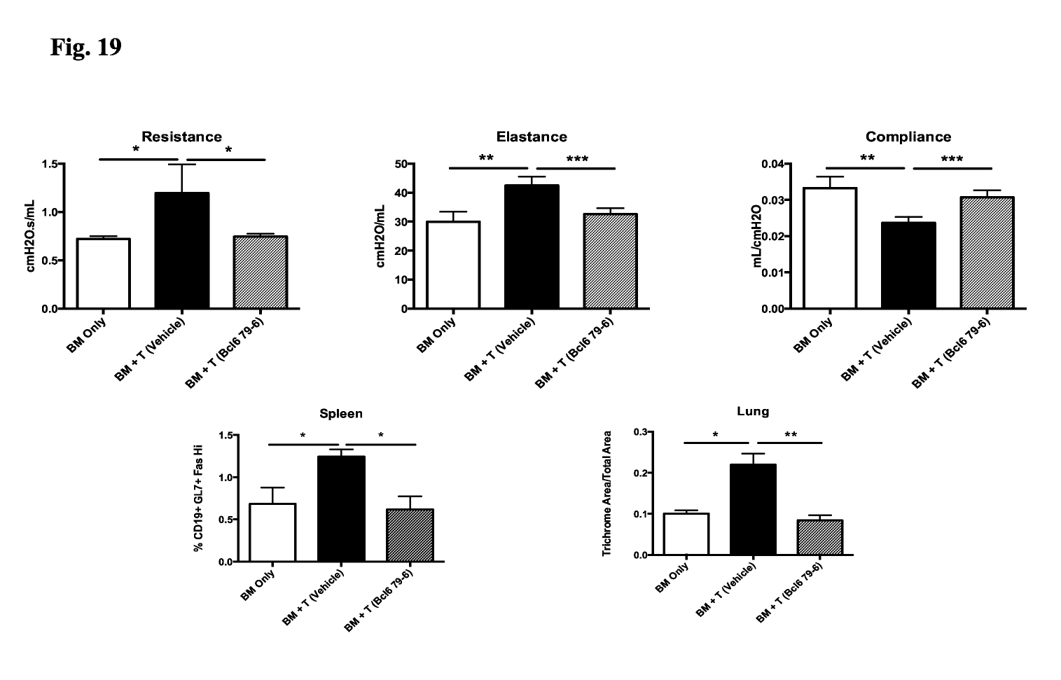

Restored pulmonary function was observed in animals treated with Bcl6 79-6 (FIG. 19). Decreased Germinal Center B cells were observed when mice were treated with Bcl6 79-6 (FIG. 19). Decreased collagen in Trichrome staining was observed when mice were treated with Bcl6 79-6 (FIG. 19).

Bcl6 fl/fl X CD19-Cre Bone Marrow Cells in cGVHD

Table 7 provides a graphical representation of the animal model protocol used in this experiment.

TABLE 7

| | | | Conditioning | | | Splenocytes |
|---|---|---|---|---|---|---|
| | | TBI | Treatment | | BM | (Purified T cells) |
| Group | n | Day −1 | Day −2 and −3 | Recipient | Day 0 | Day 0 |
| 1 | 10 | 830X | 120 mg/kg | B10.BR | 10⁷ B6 Bcl6 floxed | — |
| | | | Cytoxan | | Cre-(ve) | |
| 2 | 10 | 830X | 120 mg/kg | B10.BR | 10⁷ B6 Bcl6 floxed | 0.7 × 10⁵ B6 WT |
| | | | Cytoxan | | Cre-(ve) | |
| 4 | 10 | 830X | 120 mg/kg | B10.BR | 10⁷ B6 CD19- | — |
| | | | Cytoxan | | creXPDL1 floxed | |
| 5 | 10 | 830X | 120 mg/kg | B10.BR | 10⁷ B6 CD19- | 0.7 × 10⁵ B6 WT |
| | | | Cytoxan | | creXBcl6 floxed | |

Animals were treated with Bcl6 KO Bone Marrow derived B cells and subjected to pulmonary function tests. B10.BR mice were conditioned with 120 mg/Kg of cyclophosphamide on day −3 and −2 and subjected to total body irradiation (830X) on day −1. On Day 0 mice were transplanted with Wild Type B6 Bone marrow with or without splenocytes or purified splenic T cells or Bcl6 KO Bone Marrow with or without WT T cells. On Day 60 mice were subjected to forced oscillation technique on the Scireq Flexivent system. Tissues were harvested and analyzed for collagen deposition by trichrome staining, Ig deposition, presence of germinal centers, germinal center B cells and T follicular helper cells.

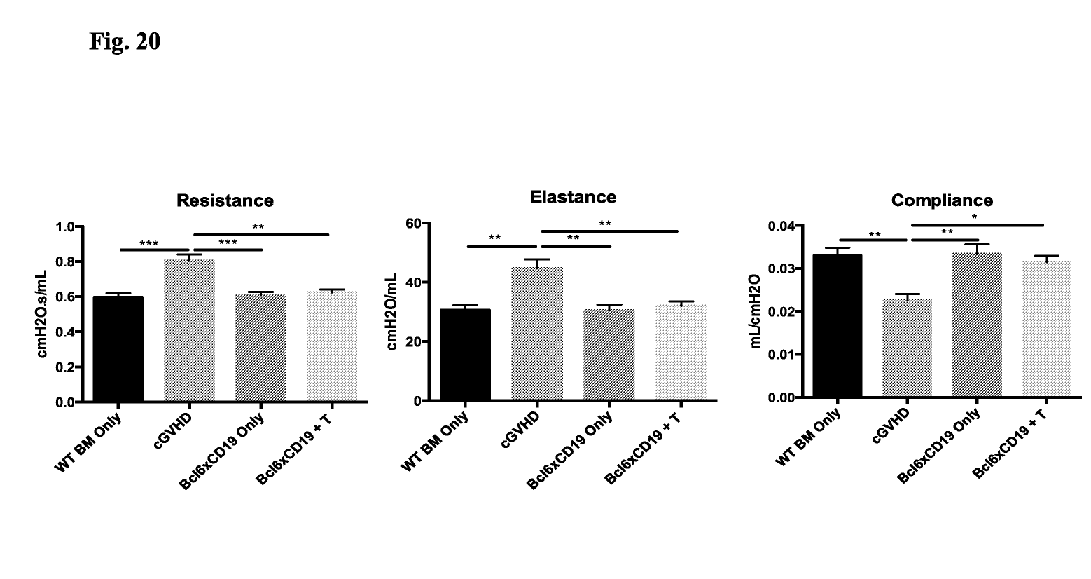

When mice were transplanted with Bone marrow that does not express Bcl6 in B cells they did not develop pathogenic pulmonary function, as demonstrated by a decrease in resistance and elastance compared to the chronic GVHD controls along with an increase in compliance (FIG. 20).

1. A method for treating chronic graft-versus-host disease (cGVHD), the method comprising:
administering to a subject in need thereof an enhancer of zeste homolog 2 (EZH2) inhibitor, a B-cell lymphoma 6 protein (Bcl6) inhibitor and/or a bromodomain-containing protein 4 (BRD4) inhibitor in an amount effective to treat cGVHD.

2. A method for improving pulmonary function in a subject receiving an allogeneic transplant, the method comprising:
administering to a subject in need thereof an enhancer of zeste homolog 2 (EZH2) inhibitor, a B-cell lymphoma 6 protein (Bcl6) inhibitor and/or a bromodomain-containing protein 4 (BRD4) inhibitor in an amount effective to improve pulmonary function.

3. The method of claim 1, wherein the EZH2 inhibitor is a small molecule, peptide, peptide mimetic, protein or a portion thereof, antibody, or nucleic acid.

4. The method of claim 1, wherein the Bcl6 inhibitor is a small molecule, peptide, peptide mimetic, protein or a portion thereof, antibody, or nucleic acid.

5. The method of claim 1, wherein the BRD4 inhibitor is a small molecule, peptide, peptide mimetic, protein or a portion thereof, antibody, or nucleic acid.

6. The method of claim 1, wherein the EZH2 inhibitor is a compound of Formula (I): 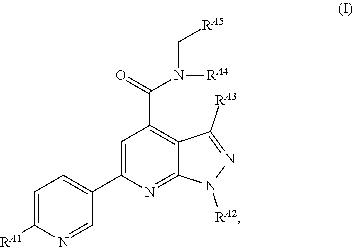
or a pharmaceutically acceptable salt, solvate, hydrate, polymorph, co-crystal, tautomer, stereoisomer, isotopically labeled derivative, or prodrug thereof, wherein:
R^{A1} is halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{a}, —N(R^{a})₂, —SR^{a}, —CN, —SCN, —C(═NR^{a})R^{a}, —C(═NR^{a})OR^{a}, —C(═NR^{a})N(R^{a})₂, —C(═O)R^{a}, —C(═O)OR^{a}, —C(═O)N(R^{a})₂, —NO₂, —NR^{a}C(═O)R^{a}, —NR^{a}C(═O)OR^{a}, —NR^{a}C(═O)N(R^{a})₂, —OC(═O)R^{a}, —OC(═O)OR^{a}, —OC(═O)N(R^{a})₂, or 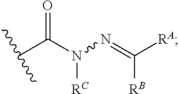
each instance of R^{a} is independently hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, an oxygen protecting group when attached to an oxygen atom, or a sulfur protecting group when attached to a sulfur atom, or two instances of R^{a} are joined to form a substituted or unsubstituted, heterocyclic ring, or substituted or unsubstituted, heteroaryl ring;
R^{A} is hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
R^{B} is hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
or R^{A} and R^{B} are joined to form a substituted or unsubstituted, carbocyclic ring, or a substituted or unsubstituted, heterocyclic ring;
R^{C} is hydrogen, substituted or unsubstituted C₁₋₆ alkyl, or a nitrogen protecting group;
R^{A2} is hydrogen, substituted or unsubstituted acyl, substituted or unsubstituted alkyl, substituted or unsubstituted carbocyclyl, substituted or unsubstituted heterocyclyl, a nitrogen protecting group, or a warhead;
R^{A3} is hydrogen, halogen, substituted or unsubstituted C₁₋₆ alkyl, —OR^{a}, —N(R^{a})₂, or a warhead;
R^{A4} is hydrogen, substituted or unsubstituted C₁₋₆ alkyl, or a nitrogen protecting group; and
R^{A5} is of the formula: 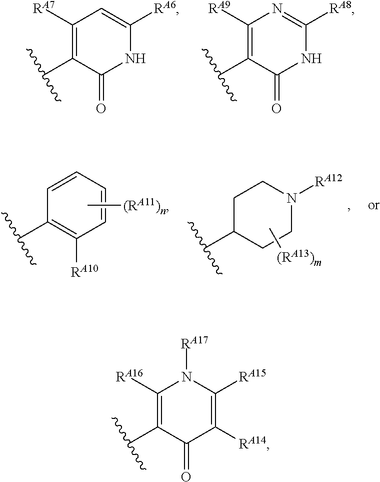
wherein:
R^{A6} is hydrogen, halogen, substituted or unsubstituted C₁₋₆ alkyl, —OR^{a}, or —N(R^{a})₂;
R^{A7} is hydrogen, halogen, substituted or unsubstituted C₂₋₆ alkyl, substituted or unsubstituted, 3- to 7-membed, monocyclic carbocyclyl comprising 0, 1, or 2 double bonds in the carbocyclic ring system, —OR^{a}, or —N(R^{a})₂;
R^{A8} is hydrogen, halogen, substituted or unsubstituted C₁₋₆ alkyl, —OR^{a}, or —N(R^{a})₂;
R^{A9} is hydrogen, halogen, substituted or unsubstituted C₁₋₆ alkyl, substituted or unsubstituted, 3- to 7-membed, monocyclic carbocyclyl comprising 0, 1, or 2 double bonds in the carbocyclic ring system, —OR^{a}, or —N(R^{a})₂;
R^{A10} is —OR^{a}, —N(R^{a})₂, or a warhead;
each instance of R^{A11} is independently halogen, substituted or unsubstituted C₁₋₆ alkyl, substituted or unsubstituted, 3- to 7-membed, monocyclic carbocyclyl comprising 0, 1, or 2 double bonds in the carbocyclic ring system, —OR^{a}, or —N(R^{a})₂;
n is 0, 1, 2, 3, or 4;
R^{A12} is hydrogen, substituted or unsubstituted C₁₋₆ alkyl, a nitrogen protecting group, or a warhead;
each instance of R^{A13} is independently halogen, substituted or unsubstituted C₁₋₆ alkyl, substituted or unsubstituted, 3- to 7-membed, monocyclic carbocyclyl comprising 0, 1, or 2 double bonds in the carbocyclic ring system, —OR^{a}, or —N(R^{a})₂;
m is 0, 1, 2, 3, 4, 5, 6, 7, 8, or 9;
R^{A14} is hydrogen, halogen, substituted or unsubstituted C₁₋₆ alkyl, —OR^{a}, or —N(R^{a})₂;
R^{A15} is hydrogen, halogen, substituted or unsubstituted C₁₋₆ alkyl, —OR^{a}, or —N(R^{a})₂;
R^{A16} is hydrogen, halogen, substituted or unsubstituted C₂₋₆ alkyl, substituted or unsubstituted, 3- to 7-membed, monocyclic carbocyclyl comprising 0, 1, or 2 double bonds in the carbocyclic ring system, —OR^{a}, or —N(R^{a})₂; and
R^{A17} is hydrogen, substituted or unsubstituted acyl, substituted or unsubstituted C₁₋₆ alkyl, a nitrogen protecting group, or a warhead.

7. The method of claim 6, wherein the compound is of the formula: 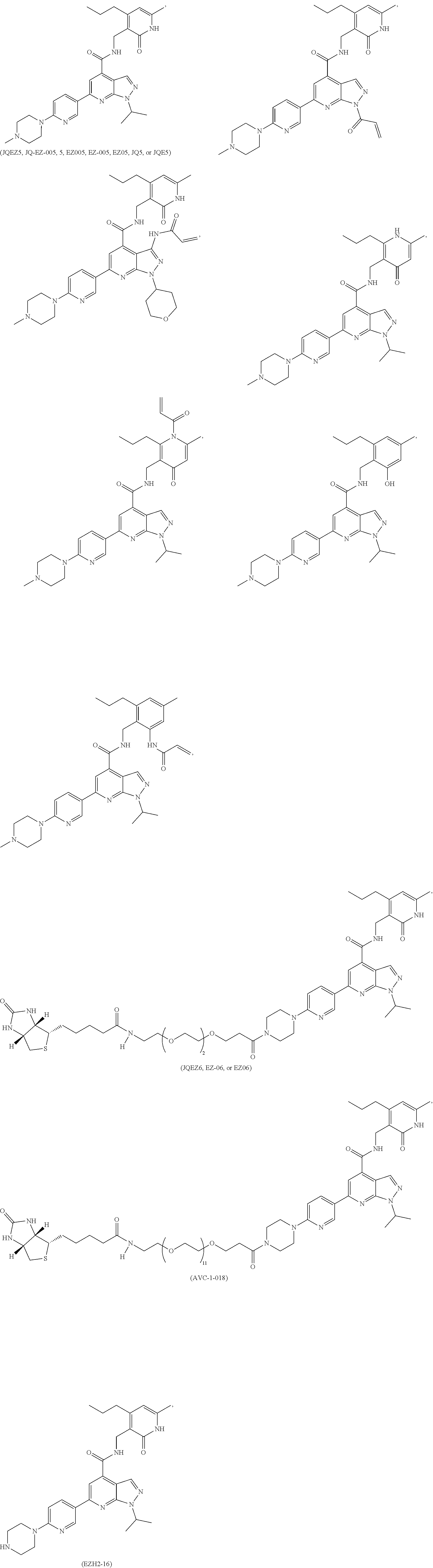
or pharmaceutically acceptable salt, solvate, hydrate, polymorph, co-crystal, tautomer, stereoisomer, isotopically labeled derivative, or prodrug thereof.

8. The method of claim 1, wherein the EZH2 inhibitor is a compound of Formula (II): 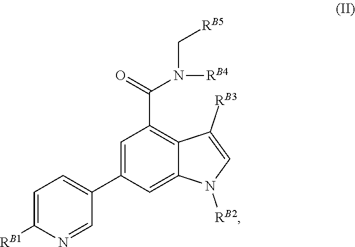
or a pharmaceutically acceptable salt, solvate, hydrate, polymorph, co-crystal, tautomer, stereoisomer, isotopically labeled derivative, and prodrug thereof, wherein:
R^{B1} is halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{b}, —N(R^{b})₂, —SR^{b}, —CN, —SCN, —C(═NR^{b})R^{b}, —C(═NR^{b})OR^{b}, —C(═NR^{b})N(R^{b})₂, —C(═O)R^{b}, —C(═O)OR^{b}, —C(═O)N(R^{b})₂, —NO₂, —NR^{b}C(═O)R^{b}, —NR^{b}C(═O)OR^{b}, —NR^{b}C(═O)N(R^{b})₂, —OC(═O)R^{b}, —OC(═O)OR^{b}, or —OC(═O)N(R^{b})₂, or 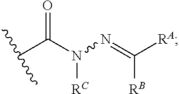
each instance of R^{b} is independently hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, an oxygen protecting group when attached to an oxygen atom, or a sulfur protecting group when attached to a sulfur atom, or two instances of R^{b} are joined to form a substituted or unsubstituted, heterocyclic ring, or substituted or unsubstituted, heteroaryl ring;
R^{A} is hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
R^{B} is hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
or R^{A} and R^{B} are joined to form a substituted or unsubstituted, carbocyclic ring, or a substituted or unsubstituted, heterocyclic ring;
R^{C} is hydrogen, substituted or unsubstituted C₁₋₆ alkyl, or a nitrogen protecting group;
R^{B2} is hydrogen, substituted or unsubstituted acyl, substituted or unsubstituted alkyl, substituted or unsubstituted carbocyclyl, substituted or unsubstituted heterocyclyl, a nitrogen protecting group, or a warhead; and
R^{B3} is hydrogen, halogen, substituted or unsubstituted C₁₋₆ alkyl, —OR^{b}, —N(R^{b})₂, or a warhead;
R^{B4} is hydrogen, substituted or unsubstituted C₁₋₆ alkyl, or a nitrogen protecting group; and
R^{B5} is of the formula: 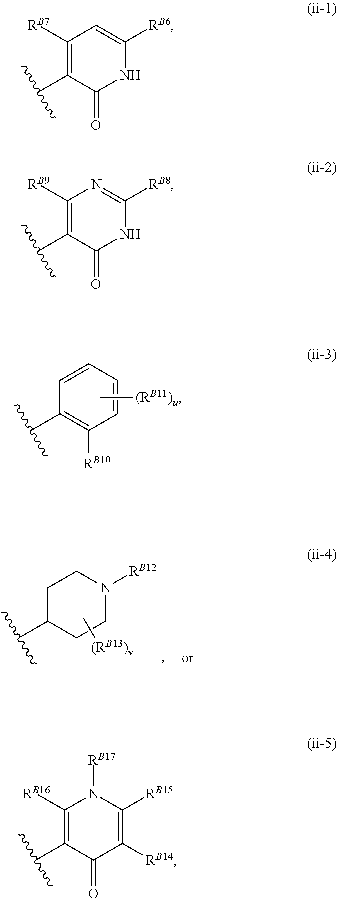
wherein:
R^{B6} is hydrogen, halogen, substituted or unsubstituted C₁₋₆ alkyl, or —N(R^{b})₂;
R^{B7} is hydrogen, halogen, substituted or unsubstituted C₂₋₆ alkyl, or substituted or unsubstituted, 3- to 7-membed, monocyclic carbocyclyl comprising 0, 1, or 2 double bonds in the carbocyclic ring system;
R^{B8} is hydrogen, halogen, substituted or unsubstituted C₁₋₆ alkyl, or —N(R^{b})₂;
R^{B9} is hydrogen, halogen, substituted or unsubstituted C₁₋₆ alkyl, or substituted or unsubstituted, 3- to 7-membed, monocyclic carbocyclyl comprising 0, 1, or 2 double bonds in the carbocyclic ring system;
R^{B10} is —OR^{b}, —N(R^{b})₂, or a warhead;
each instance of R^{B11} is independently halogen, substituted or unsubstituted C₁₋₆ alkyl, substituted or unsubstituted, 3- to 7-membed, monocyclic carbocyclyl comprising 0, 1, or 2 double bonds in the carbocyclic ring system, or —N(R^{b})₂;
u is 0, 1, 2, 3, or 4;
R^{B12} is hydrogen, substituted or unsubstituted C₁₋₆ alkyl, a nitrogen protecting group, or a warhead;
each instance of R^{B13} is independently halogen, substituted or unsubstituted C₁₋₆ alkyl, substituted or unsubstituted, 3- to 7-membed, monocyclic carbocyclyl comprising 0, 1, or 2 double bonds in the carbocyclic ring system, or —N(R^{b})₂;
v is 0, 1, 2, 3, 4, 5, 6, 7, 8, or 9;
R^{B14} is hydrogen, halogen, substituted or unsubstituted C₁₋₆ alkyl, —OR^{b}, or —N(R^{b})₂;
R^{B15} is hydrogen, halogen, substituted or unsubstituted C₁₋₆ alkyl, —OR^{b}, or —N(R^{b})₂;
R^{B16} is hydrogen, halogen, substituted or unsubstituted C₂₋₆ alkyl, substituted or unsubstituted, 3- to 7-membed, monocyclic carbocyclyl comprising 0, 1, or 2 double bonds in the carbocyclic ring system, —OR^{b}, or —N(R^{b})₂; and
R^{B17} is hydrogen, substituted or unsubstituted acyl, substituted or unsubstituted C₁₋₆ alkyl, a nitrogen protecting group, or a warhead.

9. The method of claim 8, wherein the compound is of formula: [chemical structure] 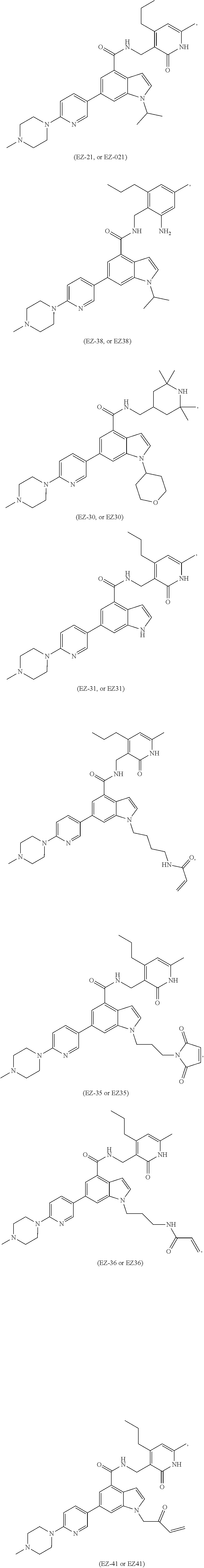
or a pharmaceutically acceptable salt, solvate, hydrate, polymorph, co-crystal, tautomer, stereoisomer, isotopically labeled derivative, or prodrug thereof.

10. The method of claim 1, wherein the Bcl6 inhibitor is a compound of the formula:

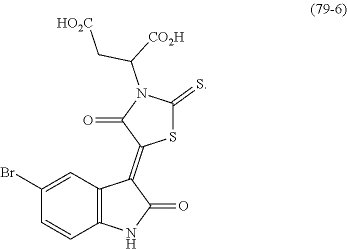 [SMILES: O=C(O)CC(C(=O)O)N1C(=O)/C(=C2/C(=O)Nc3ccc(Br)cc32)SC1=S]

11. The method of claim 1, wherein the BRD4 inhibitor is JQ1 and/or its analog.

12. The method of claim 1, wherein the allogeneic transplant is selected from the group consisting of cells, tissue, blood and organ.

13. The method of claim 12, wherein the cells are stem cells, optionally human stem cells.

14. The method of claim 1, wherein the allogeneic transplant comprises non-T-cell-depleted tissue.

15. The method of claim 1, wherein the enhancer of zeste homolog 2 (EZH2) inhibitor, the B-cell lymphoma 6 protein (Bcl6) inhibitor and/or the bromodomain-containing protein 4 (BRD4) inhibitor are administered to the subject prior the allogeneic transplant.

16. The method of claim 1, wherein the enhancer of zeste homolog 2 (EZH2) inhibitor, the B-cell lymphoma 6 protein (Bcl6) inhibitor and/or the bromodomain-containing protein 4 (BRD4) inhibitor are administered to the subject after the allogenic transplant.

17. The method of claim 16, wherein the EZH2 inhibitor, the Bcl6 inhibitor and/or the BRD4 inhibitor are administered to the subject at least one week, one month, two months, three months, four months, five months, six months, seven months, either months, nine months, ten months, eleven months, 1 year, 2 years or 3 years after the allogenic transplant.

18. The method of claim 17, wherein the EZH2 inhibitor, the Bcl6 inhibitor and/or the BRD4 inhibitor are administered to the subject at least 100 days after the allogenic transplant.

19. The method of claim 2, wherein the EZH2 inhibitor is a compound of Formula (I): 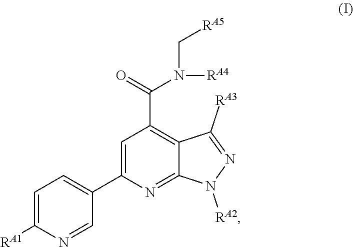
or a pharmaceutically acceptable salt, solvate, hydrate, polymorph, co-crystal, tautomer, stereoisomer, isotopically labeled derivative, or prodrug thereof, wherein:
R^{A1} is halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{a}, —N(R^{a})₂, —SR^{a}, —CN, —SCN, —C(═NR^{a})R^{a}, —C(═NR^{a})OR^{a}, —C(═NR^{a})N(R^{a})₂, —C(═O)R^{a}, —C(═O)OR^{a}, —C(═O)N(R^{a})₂, —NO₂, —NR^{a}C(═O)R^{a}, —NR^{a}C(═O)OR^{a}, —NR^{a}C(═O)N(R^{a})₂, —OC(═O)R^{a}, —OC(═O)OR^{a}, —OC(═O)N(R^{a})₂, or 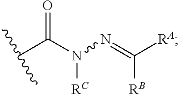
each instance of R^{a} is independently hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, an oxygen protecting group when attached to an oxygen atom, or a sulfur protecting group when attached to a sulfur atom, or two instances of R^{a} are joined to form a substituted or unsubstituted, heterocyclic ring, or substituted or unsubstituted, heteroaryl ring;
R^{A} is hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
R^{B} is hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
or R^{A} and R^{B} are joined to form a substituted or unsubstituted, carbocyclic ring, or a substituted or unsubstituted, heterocyclic ring;
R^{C} is hydrogen, substituted or unsubstituted C₁₋₆ alkyl, or a nitrogen protecting group;
R^{A2} is hydrogen, substituted or unsubstituted acyl, substituted or unsubstituted alkyl, substituted or unsubstituted carbocyclyl, substituted or unsubstituted heterocyclyl, a nitrogen protecting group, or a warhead;
R^{A3} is hydrogen, halogen, substituted or unsubstituted C₁₋₆ alkyl, —OR^{a}, —N(R^{a})₂, or a warhead;
R^{A4} is hydrogen, substituted or unsubstituted C₁₋₆ alkyl, or a nitrogen protecting group; and
R^{A5} is of the formula: 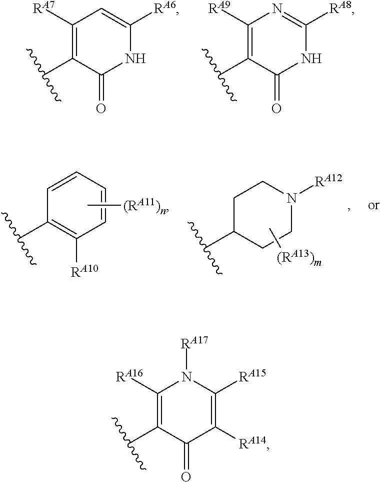
wherein:
R^{A6} is hydrogen, halogen, substituted or unsubstituted C₁₋₆ alkyl, —OR^{a}, or —N(R^{a})₂;
R^{A7} is hydrogen, halogen, substituted or unsubstituted C₂₋₆ alkyl, substituted or unsubstituted, 3- to 7-membed, monocyclic carbocyclyl comprising 0, 1, or 2 double bonds in the carbocyclic ring system, —OR^{a}, or —N(R^{a})₂;
R^{A8} is hydrogen, halogen, substituted or unsubstituted C₁₋₆ alkyl, —OR^{a}, or —N(R^{a})₂;
R^{A9} is hydrogen, halogen, substituted or unsubstituted C₁₋₆ alkyl, substituted or unsubstituted, 3- to 7-membed, monocyclic carbocyclyl comprising 0, 1, or 2 double bonds in the carbocyclic ring system, —OR^{a}, or —N(R^{a})₂;
R^{A10} is —OR^{a}, —N(R^{a})₂, or a warhead;
each instance of R^{A11} is independently halogen, substituted or unsubstituted C₁₋₆ alkyl, substituted or unsubstituted, 3- to 7-membed, monocyclic carbocyclyl comprising 0, 1, or 2 double bonds in the carbocyclic ring system, —OR^{a}, or —N(R^{a})₂;
n is 0, 1, 2, 3, or 4;
R^{A12} is hydrogen, substituted or unsubstituted C₁₋₆ alkyl, a nitrogen protecting group, or a warhead;
each instance of R^{A13} is independently halogen, substituted or unsubstituted C₁₋₆ alkyl, substituted or unsubstituted, 3- to 7-membed, monocyclic carbocyclyl comprising 0, 1, or 2 double bonds in the carbocyclic ring system, —OR^{a}, or —N(R^{a})₂;
m is 0, 1, 2, 3, 4, 5, 6, 7, 8, or 9;
R^{A14} is hydrogen, halogen, substituted or unsubstituted C₁₋₆ alkyl, —OR^{a}, or —N(R^{a})₂;
R^{A15} is hydrogen, halogen, substituted or unsubstituted C₁₋₆ alkyl, —OR^{a}, or —N(R^{a})₂;
R^{A16} is hydrogen, halogen, substituted or unsubstituted C₂₋₆ alkyl, substituted or unsubstituted, 3- to 7-membed, monocyclic carbocyclyl comprising 0, 1, or 2 double bonds in the carbocyclic ring system, —OR^{a}, or —N(R^{a})₂; and
R^{A17} is hydrogen, substituted or unsubstituted acyl, substituted or unsubstituted C₁₋₆ alkyl, a nitrogen protecting group, or a warhead.

20. The method of claim 2, wherein the EZH2 inhibitor is a compound of Formula (II): 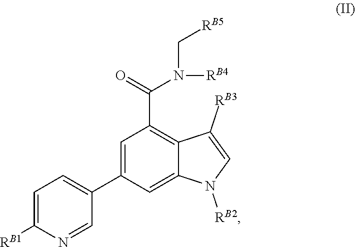
or a pharmaceutically acceptable salt, solvate, hydrate, polymorph, co-crystal, tautomer, stereoisomer, isotopically labeled derivative, and prodrug thereof, wherein:
R^{B1} is halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{b}, —N(R^{b})₂, —SR^{b}, —CN, —SCN, —C(═NR^{b})R^{b}, —C(═NR^{b})OR^{b}, —C(═NR^{b})N(R^{b})₂, —C(═O)R^{b}, —C(═O)OR^{b}, —C(═O)N(R^{b})₂, —NO₂, —NR^{b}C(═O)R^{b}, —NR^{b}C(═O)OR^{b}, —NR^{b}C(═O)N(R^{b})₂, —OC(═O)R^{b}, —OC(═O)OR^{b}, or —OC(═O)N(R^{b})₂, or 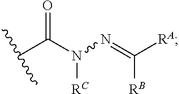
each instance of R^{b} is independently hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, an oxygen protecting group when attached to an oxygen atom, or a sulfur protecting group when attached to a sulfur atom, or two instances of R^{b} are joined to form a substituted or unsubstituted, heterocyclic ring, or substituted or unsubstituted, heteroaryl ring;
R^{A} is hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
R^{B} is hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
or R^{A} and R^{B} are joined to form a substituted or unsubstituted, carbocyclic ring, or a substituted or unsubstituted, heterocyclic ring;
R^{C} is hydrogen, substituted or unsubstituted C₁₋₆ alkyl, or a nitrogen protecting group;
R^{B2} is hydrogen, substituted or unsubstituted acyl, substituted or unsubstituted alkyl, substituted or unsubstituted carbocyclyl, substituted or unsubstituted heterocyclyl, a nitrogen protecting group, or a warhead; and
R^{B3} is hydrogen, halogen, substituted or unsubstituted C₁₋₆ alkyl, —OR^{b}, —N(R^{b})₂, or a warhead;
R^{B4} is hydrogen, substituted or unsubstituted C₁₋₆ alkyl, or a nitrogen protecting group; and
R^{B5} is of the formula: 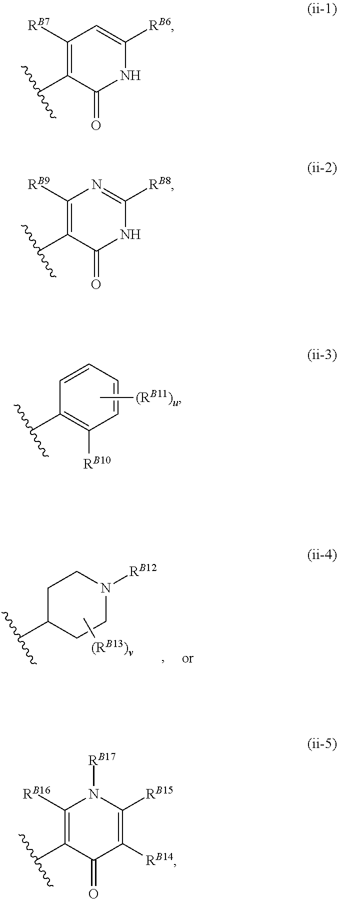
wherein:
R^{B6} is hydrogen, halogen, substituted or unsubstituted C₁₋₆ alkyl, or —N(R^{b})₂;
R^{B7} is hydrogen, halogen, substituted or unsubstituted C₂₋₆ alkyl, or substituted or unsubstituted, 3- to 7-membed, monocyclic carbocyclyl comprising 0, 1, or 2 double bonds in the carbocyclic ring system;
R^{B8} is hydrogen, halogen, substituted or unsubstituted C₁₋₆ alkyl, or —N(R^{b})₂;
R^{B9} is hydrogen, halogen, substituted or unsubstituted C₁₋₆ alkyl, or substituted or unsubstituted, 3- to 7-membed, monocyclic carbocyclyl comprising 0, 1, or 2 double bonds in the carbocyclic ring system;
R^{B10} is —OR^{b}, —N(R^{b})₂, or a warhead;
each instance of R^{B11} is independently halogen, substituted or unsubstituted C₁₋₆ alkyl, substituted or unsubstituted, 3- to 7-membed, monocyclic carbocyclyl comprising 0, 1, or 2 double bonds in the carbocyclic ring system, or —N(R^{b})₂;
u is 0, 1, 2, 3, or 4;
R^{B12} is hydrogen, substituted or unsubstituted C₁₋₆ alkyl, a nitrogen protecting group, or a warhead;
each instance of R^{B13} is independently halogen, substituted or unsubstituted C₁₋₆ alkyl, substituted or unsubstituted, 3- to 7-membed, monocyclic carbocyclyl comprising 0, 1, or 2 double bonds in the carbocyclic ring system, or —N(R^{b})₂;
v is 0, 1, 2, 3, 4, 5, 6, 7, 8, or 9;
R^{B14} is hydrogen, halogen, substituted or unsubstituted C₁₋₆ alkyl, —OR^{b}, or —N(R^{b})₂;
R^{B15} is hydrogen, halogen, substituted or unsubstituted C₁₋₆ alkyl, —OR^{b}, or —N(R^{b})₂;
R^{B16} is hydrogen, halogen, substituted or unsubstituted C₂₋₆ alkyl, substituted or unsubstituted, 3- to 7-membed, monocyclic carbocyclyl comprising 0, 1, or 2 double bonds in the carbocyclic ring system, —OR^{b}, or —N(R^{b})₂; and
R^{B17} is hydrogen, substituted or unsubstituted acyl, substituted or unsubstituted C₁₋₆ alkyl, a nitrogen protecting group, or a warhead.
